(19) United States
(12) Patent Application Publication
(10) Pub. No.: US 2010/0160311 A1
(43) Pub. Date: Jun. 24, 2010

(54) Fungicidal Azolopyrimidines, Process for Their Preparation and Their Use For Controlling Harmful Fungi, and Also Compositions Comprising Them

(75) Inventors: Jochen Dietz, Mannheim (DE); Wassilios Grammenos, Ludwigshafen (GR); Bernd Müller, Frankenthan (DE); Jan Klaas Lohmann, Ludwigshafen (DE); Jens Renner, Bad Durkheim (DE); Sarah Ulmschneider, Bad Durkheim (DE); Marianna Vrettou, Mannheim (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/373,241

(22) PCT Filed: Jul. 5, 2007
(86) PCT No.: PCT/EP2007/056785

§ 371 Date: Jan. 9, 2009

(30) Foreign Application Priority Data
Jul. 13, 2006 (EP) 06117101.3

(51) Int. Cl. A01N 43/90; C07D 487/04; C07D 413/14; C07D 403/14; A01P 3/00

(52) U.S. Cl. 514/233.2; 544/263; 544/281; 544/117; 544/118; 544/238; 514/259.1; 514/259.31; 514/252.02

(57) ABSTRACT

The present invention relates to azolopyrimidines of the formula I

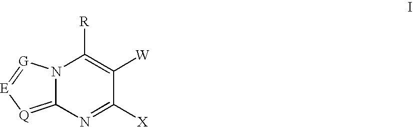

in which the substituents are defined according to the description,
to processes and intermediates for preparing these compounds, to compositions comprising them and to their use for controlling phytopathogenic harmful fungi.

The present invention relates to azolopyrimidines of the formula I

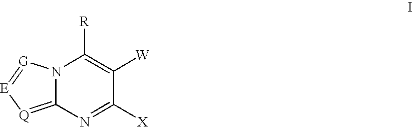

in which the substituents are as defined below:
G, E, Q a) G is N; E is C—W² and Q is N or C—W³;
   b) G is C—W¹; E is C—W² and Q is N; or
   c) G is C—W¹; E is N and Q is C—W³;
W¹, W², W³ are each independently of one another hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl or C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di-(C₁-C₄-alkyl)aminocarbonyl, C₁-C₄-alkoximinocarbonyl, hydroximinoalkyl, CR¹⁰R¹¹OR¹², C(R¹³)═NR¹⁴;
   R¹⁰, R¹¹, R¹² independently of one another are hydrogen, C₁-C₆-alkyl, C₃-C₆-cycloalkyl, C₁-C₈-alkoxy-C₁-C₈-alkyl, C₂-C₈-alkenyl, C₂-C₈-alkynyl, benzyl;
   R¹¹ and R¹² together may be oxy-C₁-C₅-alkylenoxy, where the carbon chain may be substituted by one to three groups selected from the group consisting of methyl, ethyl, hydroxyl, methoxy, ethoxy, hydroxymethyl, methoxymethyl, ethoxymethyl;
   R¹³ is hydrogen or C₁-C₈-alkyl;
   R¹⁴ is C₁-C₈-alkyl, C₃-C₆-cycloalkyl, phenyl, phenylamino, where the phenyl groups may be substituted by one to five groups R^{b};
R is NR¹R² or C₁-C₁₀-alkyl, C₁-C₁₀-haloalkynyl, C₂-C₁₀-alkenyl, C₂-C₁₀-halo-alkenyl, C₂-C₁₀-alkynyl, C₂-C₁₀-haloalkynyl, C₃-C₁₂-cycloalkenyl, C₃-C₁₂-halocycloalkenyl, phenyl, halophenyl, naphthyl, halonaphthyl or a five-, six-, seven-, eight-, nine- or ten-membered saturated, partially unsaturated or aromatic heterocycle which is attached via carbon, may be partially or fully halogenated and contains one, two, three or four heteroatoms from the group consisting of oxygen, nitrogen and sulfur; where R may contain one, two, three or four identical or different groups R^{a} independently of one another selected from the group consisting of:
   R^{a} cyano, nitro, hydroxyl, carboxyl, C₁-C₆-alkyl, C₂-C₆-alkynyl, C₃-C₆-cycloalkyl, C₃-C₆-cycloalkenyl, C₁-C₆-alkoxy, C₂-C₆-alkenyloxyl, C₃-C₆-alkynyloxy, C₃-C₆-cycloalkoxy, C₃-C₆-cycloalkenyloxy, C(O)R^{Π}, C(O)OR^{Π}, C(S)OR^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, C₁-C₆-alkylene, oxy-C₁-C₄-alkylene, oxy-C₁-C₃-alkyleneoxy, where divalent groups may be attached to the same atom or to adjacent atoms, phenyl, naphthyl, a five-, six-, seven-, eight-, nine- or ten-membered saturated, partially unsaturated or aromatic heterocycle which contains one, two, three or four heteroatoms from the group consisting of O, N and S;
      R^{Π} is C₁-C₈-alkyl, C₃-C₈-alkenyl, C₃-C₈-alkynyl, phenyl, naphthyl, a five-, six-, seven-, eight-, nine- or ten-membered saturated, partially unsaturated or aromatic heterocycle which contains one, two, three or four heteroatoms from the group consisting of O, N and S, C₃-C₆-cycloalkyl or C₃-C₆-cycloalkenyl, which groups R^{Π} may be partially or fully halogenated;
      where the aliphatic, alicyclic or aromatic groups in the abovementioned groups R^{a} and R^{Π} for their part may carry one, two or three groups R^{b}:
      R^{b} is cyano, nitro, hydroxyl, mercapto, amino, carboxyl, alkyl, alkenyl, alkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, where the alkyl groups in these radicals contain 1 to 6 carbon atoms and said alkenyl or alkynyl groups in these radicals contain 2 to 8 carbon atoms; cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members, and the hetaryl radicals contain 5 or 6 ring members, where the cyclic systems may be partially or fully halogenated and/or may be substituted by alkyl or haloalkyl groups;
R¹, R² independently of one another are hydrogen, C₁-C₁₂-alkyl, C₂-C₁₂-alkenyl, C₂-C₁₂-alkynyl, C₃-C₈-cycloalkyl, C₃-C₆-cycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle which contains one, two, three or four heteroatoms from the group consisting of O, N and S or Z—Y—(CR⁷R⁸)ₚ—(CR⁵R⁶)_{q}—CR³R⁴-#, in which # is the point of attachment to the nitrogen atom and:
   R³, R⁴, R⁵, R⁶, R⁷, R⁸ independently of one another are hydrogen, halogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle which contains one, two, three or four heteroatoms from the group consisting of O, N and S, which cyclic groups may be partially or fully halogenated and/or may be substituted by one or more groups R^{Π},

R⁵ and R³ or R⁷ together with the atoms to which these radicals are attached may also _{form} a five-, six-, seven-, eight-, nine- or ten-membered saturated or partially unsaturated ring which, in addition to carbon atoms, may contain one, two or three heteroatoms from the group consisting of O, N and S as ring member and/or may carry one or more substituents R^{a};

R³ may combine with R⁴, R⁵ may combine with R⁶, R⁷ may combine with R⁸, in each case to represent oxygen, thus forming carbonyl groups, and form a C₂-C₅-alkylene or alkenylene or alkynylene chain which may be interrupted by one, two or three heteroatoms from the group consisting of O, N and S, thus forming spiro groups;

R¹ and R³ together with the nitrogen atom to which they are attached may form a five-, six-, seven-, eight-, nine- or ten-membered saturated or partially unsaturated heterocycle which, in addition to carbon atoms, may contain one, two or three further heteroatoms from the group consisting of O, N and S as ring member;

R³, R⁴, R⁵, R⁶, R⁷, R⁸ may independently of one another be partially or fully halogenated;

R¹ to R⁸ may each independently carry one, two, three or four identical or different groups R^{a};
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{Π})NR^{A}R^{B}, C(S)—C₁-C₄-alkylene-NR^{A}C(NR^{Π})NR^{A}R^{B}, C(NR^{Π})—C₁-C₄-alkylene-NR^{A}C(NR^{Π})NR^{A}R^{B}, phenyl, naphthyl, a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may be substituted by one or more groups R^{b};
   R^{A}, R^{B} independently of one another are hydrogen, C₂-alkenyl, C₂-alkynyl or one of the groups mentioned under R^{Π}, where R^{A} and R^{B} together with the nitrogen atom to which they are attached or R^{A} and R^{Π} together with the carbon and heteroatoms via which they are attached may also form a three- to ten-membered saturated, partially unsaturated or aromatic mono- or bicyclic ring which, in addition to carbon atoms, may contain one, two or three further heteroatoms from the group consisting of O, N and S as ring member, or may carry one or more oxo groups and/or one or more substituents R^{b}; or
      Z and R⁶ or R⁸ may also form a five- or six-membered saturated or partially unsaturated ring which, in addition to carbon atoms and Y, may contain one or two further heteroatoms from the group consisting of N and S as ring member and/or may carry one or more substituents R^{a}, as defined below;
         the group Z may be partially or fully halogenated and/or carry one, two or three groups R^{b};
   R¹ and R² together with the nitrogen atom to which they are attached may also form a five-, six-, seven-, eight-, nine- or ten-membered saturated, partially unsaturated or aromatic mono- or bicyclic heterocycle which may be partially or fully halogenated and, in addition to carbon atoms, may contain one, two or three further heteroatoms from the group consisting of O, N and S as ring member and which may carry one, two or three substituents selected from the group consisting of R^{a}, Z—Y-# and Z—Y—(CR⁵R⁶)_{q}—CR³R⁴-#, where # is the point of attachment to the heterocycle;
   p is zero, 1, 2, 3, 4 or 5;
   q is zero or 1;
W is phenyl or five- or six-membered heteroaryl which, in addition to carbon atoms, contains one, two or three further heteroatoms from the group consisting of O, N and S as ring member, where the ring systems, in addition to groups Lₘ, carry at least one substituent P¹,
   P¹ is Y¹—Y²-T;
      Y¹ is CR^{A}R^{B}, C(=T²)O, C(=T²)NR^{A}, O, OC(=T²), NR^{A} or S(O)ᵣ;
      Y² is C₁-C₈-alkylene, C₂-C₈-alkenylene, C₂-C₈-alkynylene, where Y² may be interrupted by one, two or three heteroatoms from the group consisting of NR^{A}, O, S(O)ᵣ;
      r is 0, 1 or 2;
      T is YR, YR^{A}, NR^{A}R^{B}, YNR^{A}R^{B}, C(NOR^{A})R^{B}, S(O)ᵣR^{A}, N(R^{A})-T¹-C(=T²)-T³, T¹-C(=T²)-[(Y²)_{q}—C(=T²)]ₚ-T³, T¹-C(=T²)-[Y²-T¹-C(=T²)]ₚ-T³, T¹-C(=T²)-[T¹-Y²—C(=T²)]ₚ-T³ or T¹-C(=T²)-[NR^{A}—(NR^{B})_{q}—C(=T²)]ₚ-T³;
      T¹ is a direct bond, O, S, NR^{A};
      T² is Y, NR^{A};
      T³ is R, R^{B}, R^{Π}, YR^{B}, NR^{A}R^{B};
   where the carbon atoms in the group P¹ may be partially or fully halogenated and/or may be substituted by one or more groups R^{b};
   L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{φ}, C(S)—R^{φ}, S(O)ᵣ—R^{φ}; C₁-C₈-alkenyloxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynyl-carbonyl, C₃-C₆-cycloalkylcarbonyl, or a five-, six-, seven-, eight-, nine- or ten-membered saturated, partially unsaturated or aromatic heterocycle which contains one, two, three or four heteroatoms from the group consisting of O, N and S;
      R¹⁰⁰ is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, di-C₁-C₄-alkylamino; where the groups R^{φ} may be substituted by one, two or three identical or different groups R^{b}, as defined above;
   m is zero, 1, 2, 3, 4 or 5;
X is halogen, cyano, C₁-C₄-alkyl, C₁-C₄-haloalkyl, C₁-C₄-alkoxy or C₁-C₄-haloalkoxy, amino, C₁-C₄-alkylamino or di-C₁-C₄-alkylamino;
   and agriculturally acceptable salts thereof.

Moreover, the invention relates to processes and intermediates for preparing these compounds, to compositions comprising them and to their use for controlling phytopathogenic harmful fungi.

6-Phenyl-7-aminotriazolopyrimidines are known in a general manner from EP-A 550 113 and WO 99/48893. Triazolopyrimidines which are substituted in positions 5 and 7 by groups attached via carbon are known from WO 03/004465. WO 02/002563 describes certain 6-phenyltriazolopyrimidines as fungicidally and pharmaceutically active. WO 2005/030775 describes pharmaceutically active 6-phenyl-7-haloalkylaminotriazolopyrimidines. 5-Halo-7-aminopyrazolopyrimidines which are substituted in the 6-position by a heterocycle are known in a general manner from WO 05/000851.

In many cases, the activity of the known compounds is unsatisfactory. Based on this, it is an object of the present invention to provide compounds having improved activity and/or a broader activity spectrum.

Accordingly, we have found the compounds defined at the outset. Furthermore, we have found processes and intermediates for their preparation, compositions comprising them and methods for controlling harmful fungi using the compounds I.

The compounds according to the invention differ from those described in the cited publication by the substituent P¹ on the group W.

The compounds according to the invention can be obtained by various routes. If R in formula I is NR¹R², the compounds are prepared by reacting an aminoazole of the formula II with appropriately substituted phenylmalonates of the formula III in which R″ is alkyl, preferably C₁-C₆-alkyl, in particular methyl or ethyl.

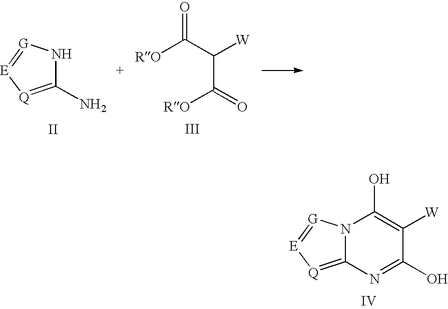

This reaction is usually carried out at temperatures of from 80° C. to 250° C., preferably from 120° C. to 180° C., without solvent or in an inert organic solvent in the presence of a base [cf. EP-A 770 615] or in the presence of acetic acid under the conditions known from Adv. Het. Chem. Vol. 57, p. 81ff. (1993).

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethylacetamide. Particularly preferably, the reaction is carried out without solvent or in chlorobenzene, xylene, dimethyl sulfoxide, N-methylpyrrolidone. It is also possible to use mixtures of the solvents mentioned.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Preference is given to using tertiary amines, such as diisopropylethylamine, tributylamine, N-methylmorpholine or N-methylpiperidine.

The bases are generally employed in catalytic amounts; however, they can also be used in equimolar amounts, in excess or, if appropriate, as solvent.

The starting materials are generally reacted with one another in equimolar amounts. In terms of yield it may be advantageous to use an excess of base and the malonate III, based on the triazole.

Advantageously, the malonates of the formula III are obtained by reacting appropriately substituted bromoaromatic compounds with dialkyl malonates under Cu(I) catalysis [cf. Chemistry Letters, pp. 367-370, 1981; EP-A 10 02 788].

Alternatively, the malonates of the formula III can be constructed according to the scheme below under generally known conditions [cf.: March, Advanced Organic Chemistry, 3rd ed., p. 792ff, J. Wiley & Sons, New York (1985)]:

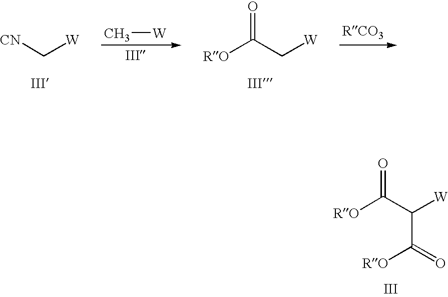

These reactions are usually carried out at temperatures of from −100° C. to +200° C., preferably from +20° C. to +100° C., in an inert organic solvent in the presence of a base [cf. U.S. Pat. No. 4,454,158; Bioorgan. & Med. Chem. Lett. Vol. 15, p. 2970 (2005); Organ. Proc. Res. & Develop., Vol. 8, p. 411 (2004); J. Am. Chem. Soc, Vol. 125, p. 13948 (2003); Ann. Pharm. Fr., Vol. 60, p. 314 (2004); Pharmazie, Vol. 44, p. 115 (1989)].

Under the conditions known from WO-A 94/20501, the dihydroxyazolopyrimidines of the formula IV are converted into the dihaloazolopyrimidines of the formula V in which Y is a halogen atom, preferably a bromine or a chlorine atom, in particular a chlorine atom. The halogenating agent [HAL] used is advantageously a chlorinating agent or a brominating agent, such as phosphorus oxybromide or phosphorus oxychloride, if appropriate in the presence of a solvent.

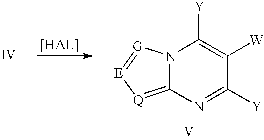

This reaction is usually carried out at from 0° C. to 150° C., preferably at from 80° C. to 125° C. [cf. EP-A 770 615].

Dihaloazolopyrimidines of the formula V are prepared using amines of the formula VI in which the variables are as defined for formula I.

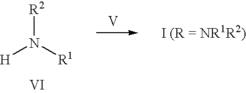

This reaction is advantageously carried out at from 0° C. to 70° C., preferably from 10° C. to 35° C., preferably in the presence of an inert solvent, such as an ether, for example dioxane, diethyl ether or, in particular, tetrahydrofuran, a halogenated hydrocarbon, such as dichloromethane, or an aromatic hydrocarbon, such as, for example, toluene [cf. WO 05/000851].

The use of a base, such as a tertiary amine, for example triethylamine, or an inorganic amine, such as potassium carbonate, is preferred; it is also possible for excess amine of the formula VI to serve as base.

Thus, using the 5,7-dichloroazolopyrimidines known from the publication mentioned at the outset, it is possible to obtain the 5-chloroazolopyrimidines of the formula I. They are a preferred subject matter of the invention. Other 5,7-dihaloazolopyrimidines can be obtained analogously to the literature cited.

Amines of the formula VI are known from the literature, can be prepared by known methods or are commercially available.

Compounds of the formula I in which R is NR¹R² and X is C₁-C₄-alkyl or C₁-C₄-haloalkyl can be obtained in an advantageous manner by the synthesis route below:

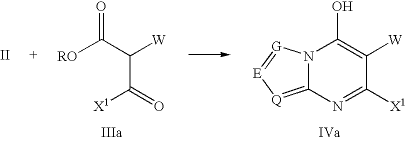

Starting with the ketoesters IIla, the 5-alkyl-7-hydroxyazolopyrimidines IVa are obtained. In formulae IIIa and IVa, X¹ is C₁-C₄-alkyl or C₁-C₄-haloalkyl. The starting materials IIIa are advantageously prepared using the conditions described in EP-A 10 02 788 [cf. Chem. Pharm. Bull., 9, 801, (1961)].

The 5-alkyl-7-hydroxyazolopyrimidines obtained in this manner are reacted with halogenating agents [HAL] under the conditions described further above to give the 7-haloazolopyrimidines of the formula Va in which Hal is a halogen atom. Preference is given to using chlorinating or brominating agents, such as phosphorus oxybromide, phosphorus oxychloride, thionyl chloride, thionyl bromide or sulfuryl chloride. The reaction can be carried out neat or in the presence of a solvent. Customary reaction temperatures are from 0 to 150° C. or. preferably. from 80 to 125° C.

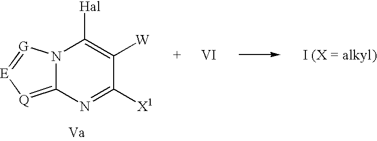

The reaction of Va with amines VI is carried out under the conditions described further above.

Alternatively, compounds of the formula I in which X is C₁-C₄-alkyl can also be prepared from compounds I in which X is halogen, in particular chlorine, and malonates of the formula IIIb. In formula IIIb, X″ is hydrogen or C₁-C₃-alkyl and R# is C_{1-C}₄-alkyl. They are converted into compounds of the formula VII and decarboxylated to give compounds I [cf. U.S. Pat. No. 5,994,360]. The compounds of the formula VII are novel.

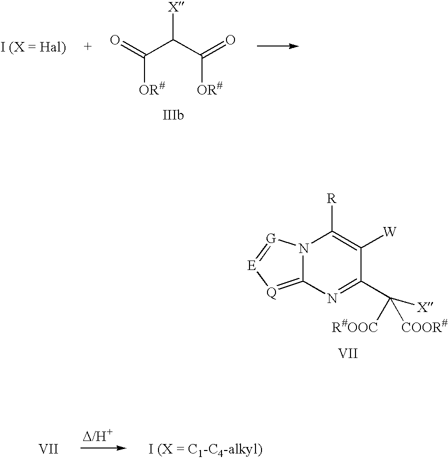

The malonates IIIb are known from the literature [J. Am. Chem. Soc., Vol. 64, 2714 (1942); J. Org. Chem., Vol. 39, 2172 (1974); Helv. Chim. Acta, Vol. 61, 1565 (1978)] or can be prepared in accordance with the literature cited.

The subsequent hydrolysis of the ester VII is carried out under generally customary conditions; depending on the various structural elements, alkaline or acidic hydrolysis of the compounds VII may be advantageous. Under the conditions of ester hydrolysis, there may already be complete or partial decarboxylation to I.

The decarboxylation is usually carried out at temperatures of from 20° C. to 180° C., preferably from 50° C. to 120° C., in an inert solvent, if appropriate in the presence of an acid.

Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; particularly preferably, the reaction is carried out in hydrochloric acid or acetic acid. It is also possible to use mixtures of the solvents mentioned.

If R in formula I is a group attached via carbon (R′ in formula Ia) and X is alkyl or haloalkyl, the compounds are prepared by reacting an aminoazole of the formula II with appropriately substituted 1,3-diketones of the formula IIIc in which R is a group attached via carbon according to formula I and X″ is alkyl or haloalkyl, preferably C₁-C₆-alkyl, in particular methyl or ethyl.

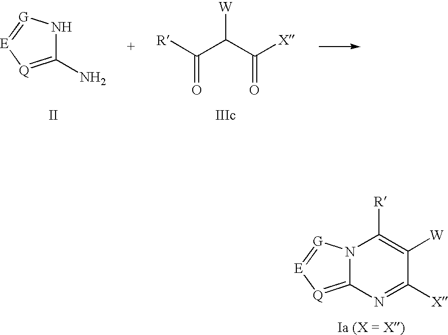

This reaction is advantageously carried out under the conditions described further above for the reaction of the compounds II with III.

Alternatively, compounds of the formula I in which R in formula I is a group attached via carbon and X is halogen, in particular chlorine, can also be prepared from dihalo compounds of the formula Va

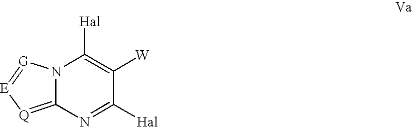

in which Hal is halogen, in particular chlorine, under the conditions known from WO 03/004465.

Compounds of the formula I in which X is cyano, alkoxy or haloalkoxy can be obtained in an advantageous manner by reacting compounds I in which X is halogen, preferably chlorine, with compounds M-X′ (formula VIII). Depending on the meaning of the group X′ to be introduced, the compound IV is an inorganic cyanide, an alkoxide or a haloalkoxide. The reaction is advantageously carried out in the presence of an inert solvent. The cation M in formula VIII is of little importance; for practical reasons, ammonium, tetraalkylammonium or alkali metal or alkaline earth metal salts are usually preferred.

I(X=halogen)+M-X′→I(X=X′) VIII

The reaction temperature is usually from 0 to 120° C., preferably from 10 to 40° C. [cf. J. Heterocycl. Chem., Vol. 12, pp. 861-863 (1975)].

Suitable solvents include ethers, such as dioxane, diethyl ether and, preferably, tetrahydrofuran, halogenated hydrocarbons, such as dichloromethane, and aromatic hydrocarbons, such as toluene.

Compounds of the formula I in which X is C₁-C₄-alkyl can also be obtained by coupling 5-haloazolopyrimidines of the formula I with organometallic reagents of the formula VIIIa. In one embodiment of this process, the reaction is carried out under transition metal catalysis, such as Ni or Pd catalysis.

I(X=Hal)+M^{y}(—X″)_{y}→I(X=C₁-C₄-alkyl) VIIIa

In formula VIIIa, M is a metal ion of valency Y, such as, for example, B, Zn or Sn, and X″ is C₁-C₃-alkyl. This reaction can be carried out, for example, analogously to the following methods: J. Chem. Soc. Perkin Trans. 1, 1187 (1994), ibid. 1, 2345 (1996); WO 99/41255; Aust. J. Chem., Vol. 43, 733 (1990); J. Org. Chem., Vol. 43, 358 (1978); J. Chem. Soc. Chem. Commun. 866 (1979); Tetrahedron Lett., Vol. 34, 8267 (1993); ibid., Vol. 33, 413 (1992).

Compounds of the formula I in which R is NR¹R², where R¹ is Z—Y—(CR⁷R⁸)ₚ—(CR⁵R⁶)_{q}—CR³R⁴-# can alternatively also be prepared from hydroxyl- or mercaptoazolopyrimidines of the formula I′.

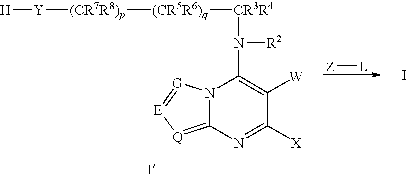

To this end, the 7-hydroxy- or mercaptoaminoazolopyrimidine of the formula I′ is reacted with an alkylating or acylating agent Z-L, L being a nucleophilically removable group. Halides, in particular chlorides and bromides, carboxylic anhydrides, such as, for example, acetic anhydride, or carbonyl chlorides, carboxylic acids in combination with coupling agents, such as, for example, dicyclohexylcarbodiimide, or acids, such as, for example, HCl, are usually employed. The reaction conditions suitable for the etherification or esterification are generally known to the person skilled in the art [cf.: Organikum, VEB Deutscher Verlag der Wissenschaften, Berlin (1981)]. Some of the compounds of the formula Ia are known from the documents cited at the outset.

Moreover, compounds of the formula I can be obtained from the corresponding precursors which, instead of group P¹, carry a nucleophilically exchangeable group on group W. The group P¹ is then introduced by nucleophilic substitution [cf. WO 05/30775].

Alternatively, compounds of the formula I in which P¹ is a group attached via oxygen can be prepared from analogous hydroxyl compounds (formula IX) which for their part can be obtained by ether cleavage from known compounds [cf. WO 99/48893]. In this case, group P¹ is introduced by nucleophilic substitution of the hydroxyl group under basic conditions.

These hydroxyl compounds correspond to the formula I in which W, in addition to group Lₘ, is substituted by a hydroxyl group (formula IX). They are novel.

Compounds of the formula I in which P¹ is a group attached via nitrogen can be prepared in an advantageous manner from precursors in which the group W carries an amino group, which may be obtainable, if appropriate, from the corresponding nitro-substituted compounds by reduction.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds I can not be obtained by the routes described above, they can be prepared by derivatization of other compounds I.

If the synthesis yields mixtures of isomers, a separation is generally not necessarily required since in some cases the individual isomers can be interconverted during preparation for application or during application (for example under the action of light, acid or bases). Such conversions may also take place after application, for example in the case of the treatment of plants in the treated plants or in the harmful fungus to be controlled.

In the definition of the symbols given in the above formulae, collective terms were used which are generally representative for the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoro-prop-2-yl;

alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;

a five- or six-membered saturated, partially unsaturated or aromatic heterocycle which contains one, two, three or four heteroatoms from the group consisting of O, N and S:
non-aromatic saturated or partially unsaturated 5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydro-pyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: divalent branched or unbranched chains of 2 to 8 CH₂ groups, for example CH₂CH₂, CH₂CH₂CH₂, CH(CH₃)CH₂, CH₂CH₂CH₂CH₂, CH₂CH₂CH(CH₃), CH₂CH(CH₃)CH₂, CH₂CH₂CH₂CH₂CH₂, CH₂CH₂CH₂CH₂CH₂CH₂, CH₂CH₂CH₂CH₂CH₂CH₂CH₂ and CH₂CH₂CH₂CH₂CH₂CH₂CH₂CH₂,

oxyalkylene: divalent branched or unbranched chains of 2 to 4 CH₂ groups where one valency is attached via an oxygen atom to the skeleton, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached via an oxygen atom to the skeleton, for example OCH₂O, OCH₂CH₂O and OCH₂CH₂CH₂O.

According to the present invention, agriculturally acceptable salts include in particular the salts of those cations or the acid addition salts of those acids whose cations and anions, respectively, have no adverse effect on the pesticidal action of the pyrimidines according to the invention.

Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄)-alkylsulfonium, and sulfoxonium ions, preferably tri(C₁-C₄)-alkylsulfoxonium.

Anions of useful acid addition salts are, for example, chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids or C₁-C₄-haloalkanoic acids, preferably formate, acetate, propionate, butyrate or trilfuoroacetate. They can be formed by reacting the compounds according to the invention with an acid of the corresponding anion, preferably hydrochloric acid, hydrobromic acid, sulfuric acid, phosphoric acid or nitric acid.

The scope of the present invention includes the (R)- and (S)-isomers and the racemates of compounds of the formula I having chiral centers.

As a result of hindered rotation of asymmetrically substituted groups, atrope isomers of compounds of the formula I may be present. They also form part of the subject matter of the invention.

The embodiments of the intermediates with respect to the variables correspond to those of the formula I.

With a view to the intended use of the azolopyrimidines of the formula I, particular preference is given to the following meanings of the substituents, in each case on their own or in combination:

one embodiment relates to compounds I in which R is NR¹R². These compounds correspond to the formula I.a.

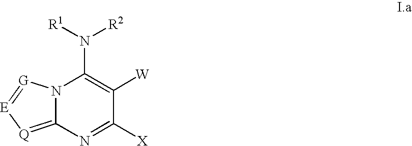

A preferred embodiment of the group NR¹R² is di-C₁-C₄-alkylamino.

A further embodiment relates to compounds I.a in which R¹ is C₁-C₆-alkyl, C₂-C₆-alkenyl or, C₂-C₆-alkynyl.

A further embodiment relates to compounds I.a in which R¹ is C₃-C₆-cycloalkyl which may be substituted by C₁-C₄-alkyl.

In the case of halogen-free alkyl or alkenyl groups having a center of chirality in R¹ or R², preference is given to the (R)-configured isomers.

A further embodiment relates to compounds I.a in which R¹ and R² together with the nitrogen atom to which they are attached form a pyrrolidinyl, piperidinyl, morpholinyl or thiomorpholinyl ring, in particular a piperidinyl ring which is optionally substituted by one to three halogen, C₁-C₄-alkyl or C₁-C₄-haloalkyl, in particular C₁-C₄-alkyl, groups. Particular preference is given to the compounds in which R¹ and R² together with the nitrogen atom to which they are attached form a pyrrolidine or 4-methylpiperidine ring.

A further embodiment relates to compounds I.a in which R¹ and R² together with the nitrogen atom to which they are attached form a pyrazole ring which is optionally substituted by one or two halogen, C₁-C₄-alkyl or C₁-C₄-haloalkyl groups, in particular by 3,5-dimethyl or 3,5-di(trifluoromethyl), preferably by 3,5-dimethyl.

A further embodiment relates to compounds I in which R¹ is CH(CH₃)—CH₂CH₃, CH(CH₃)—CH(CH₃)₂, CH(CH₃)—C(CH₃)₃, CH₂C(CH₃)═CH₂, CH₂CH═CH₂, cyclopentyl or cyclohexyl; R² is hydrogen, methyl or ethyl; or R¹ and R² together are —(CH₂)₂CH(CH₃)(CH₂)₂— or —(CH₂)₂O(CH₂)₂—.

A further embodiment relates to compounds I.a in which the groups R¹ and R² including their substituents are halogen-free.

One embodiment relates to compounds I.a in which R² is hydrogen.

A further embodiment relates to compounds I.a in which R² is methyl or ethyl.

A further embodiment relates to compounds I.a in which R² is CH₃, CH₂CH₃, propyl, butyl, CH₂CN, CH₂CH═CH₂, CH₂C≡CH, CH₂CH₂OH, CH₂CH₂OCH₃ or CH₂CH₂OCH₂CH₃.

One embodiment of the compounds of the formula I.a relates to those in which the group R¹ is Z—Y—(CR⁷R⁸)ₚ—(CR⁵R⁶)_{q}—CR³R⁴-#. They correspond to the formulae I.a¹ and I.a²:

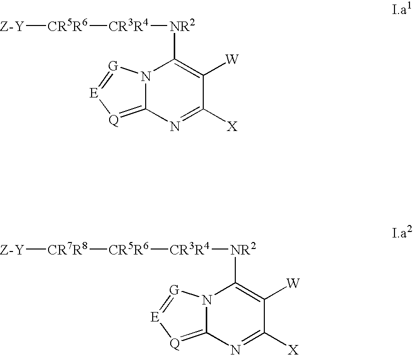

in which the variables are as defined above.

A further embodiment relates to compounds I.a in which the group NR¹R² is ethylglycinole, leucinole, tert-leucinole, valinole, norvalinole, methioninole, phenylalaninole, lysinole, argininole, histidinole, asparaginole, glutaminole, serinole, isoleucinole, cysteinole, hydroxymethylpiperidine, cis-2-hydroxymethyl-4-methylpiperidine, trans-2-hydroxymethyl-4-methylpiperidine, cyclohexylglycinole, cyclopentylglycinole, butylglycinole, pentylglycinole, cis-2-aminocyclohexanole, trans-2-aminocyclohexanole, cis-2-aminocyclopentanole, trans-2-aminocyclopentanole, cis-1-amino-2-hydroxyindane or trans-1-amino-2-hydroxyindane.

A further embodiment relates to compounds I.a in which R³ is hydrogen or straight-chain or branched C₁-C₈-alkyl, C₃-C₈-alkenyl or C₃-C₆-cycloalkyl, in particular C₁-C₆-alkyl or C₃-C₆-cycloalkyl, such as hydrogen, CH₃, CH₂CH₃, propyl, butyl, preferably isopropyl, isobutyl, tert-butyl, sec-pentyl, cyclopropyl or cyclopentyl, in particular hydrogen or tert-butyl.

In one embodiment, the group R³ is branched at the a-carbon atom.

In one embodiment, the group R³ is substituted by groups attached via heteroatoms, such as halogen, alkoxy, alkylthio, amino, alkylamino, dialkylamino or formyl, carboxyl, alkoxycarbonyl, alkoxythiocarbonyl or alkenyl, alkynyl groups or C₂-C₅-alkylene, where both valencies are attached to the same carbon atom.

In a further embodiment, the group R³ is substituted by C₃-C₆-cycloalkyl or C₃-C₈-cycloalkenyl.

In a further embodiment, the group R³ is substituted by C(O)R^{A}, C(O)OR^{A}, C(S)OR^{A}, C(O)NR^{A}R^{B}, C(S)NR^{A}R^{B}, C(NR^{A})R^{B}, C(O)SR^{Π} or C(S)SR^{Π}.

R^{Π} is preferably C₁-C₈-alkyl or C₃-C₆-cycloalkyl, which groups may be partially or fully halogenated.

In a further embodiment, the group R³ is substituted by a five-, six-, seven-, eight-, nine- or ten-membered saturated, partially unsaturated or aromatic heterocycle which contains one, two, three or four heteroatoms from the group consisting of O, N and S.

A further embodiment relates to compounds I in which R⁴ is hydrogen, straight-chain or branched C₁-C₈-alkyl or C₃-C₆-cycloalkyl, in particular hydrogen, C₁-C₆-alkyl or C₃-C₆-cycloalkyl, preferably hydrogen, isopropyl, tert-butyl. If R⁴ is an alkyl group, R⁴ preferably has the same meaning as R³.

In a further embodiment of the compounds of the formula I, R³ and R⁴ together form a C₃-C₆-alkylene group, in particular a C₃-C₄-alkylene group, where the carbon chains may be substituted by groups attached via heteroatoms, such as halogen, alkoxy, alkylthio, amino, alkylamino, dialkylamino or alkoxycarbonyl.

In a further embodiment of the compounds of the formula I, R³ and R⁴ together form a C₃-C₆-alkylene group, in particular a C₃-C₄-alkylene group, where the carbon chains are interrupted by one or two heteroatoms from the group consisting of O, N and S and may be substituted by groups attached via heteroatoms, such as halogen, alkoxy, alkylthio, amino, alkylamino, dialkylamino or alkoxycarbonyl.

In a further embodiment of the compounds of the formula I, R⁴, R⁵, R⁶, R⁷ and R⁸ are each hydrogen or C₁-C₄-alkyl, preferably hydrogen, methyl or ethyl, in particular R⁵, R⁶, R⁷ and R⁸ may be substituted corresponding to group R³.

In a further embodiment of the compounds of the formula I, R³ and R⁵ together form a C₃-C₆-alkylene, C₃-C₆-oxyalkylene or C₂-C₅-oxyalkylenoxy group, in particular a C₃-C₄-alkylene group.

In a further embodiment of the compounds of the formula I, R⁵ and R⁶ and/or R⁷ and R⁸ in each case together form a C₃-C₆-alkylene, C₃-C₆-oxyalkylene or C₂-C₅-oxyalkylenoxy group, in particular a C₃-C₄-alkylene group.

In a further embodiment of the compounds of the formula I, the index q has the value zero.

A further embodiment relates to compounds I in which the index q is 1.

A further embodiment relates to compounds I in which the index p is zero or 1, in particular zero.

In a further embodiment of the compounds of the formula I, R⁵ and R⁶ are preferably hydrogen if the index p has the value zero.

In a further embodiment of the compounds of the formula I, R⁷ is not hydrogen and R⁸ is hydrogen if the index p has the value zero.

In a further embodiment of the compounds of the formula I the index p has the value zero or 1 and the index q has the value 1.

In a further prepared embodiment of the compounds of the formula I, Y is oxygen.

In a further embodiment of the compounds of the formula I, Z is a monovalent group.

A further embodiment relates to compounds I in which Z is C₁-C₄-alkylcarbonyl, in particular acetyl, n-propan-1-one, 2-methylpropan-1-one or butan-1-one.

A further embodiment relates to compounds I in which Z is carboxyl or formyl.

A further embodiment relates to compounds I in which Z is hydrogen.

A further embodiment relates to compounds I in which Z is carboxyl.

A further embodiment relates to compounds I in which Z is formyl.

A further embodiment relates to compounds I in which Z is C₁-C₈-alkyl.

A further embodiment relates to compounds I in which Z is C₁-C₈-haloalkyl.

A further embodiment relates to compounds I in which Z is C₂-C₈-alkenyl.

A further embodiment relates to compounds I in which Z is C₂-C₈-haloalkenyl.

A further embodiment relates to compounds I in which Z is C₂-C₈-alkynyl.

A further embodiment relates to compounds I in which Z is C₂-C₈-haloalkynyl.

A further embodiment relates to compounds I in which Z is C₃-C₆-cycloalkyl.

A further embodiment relates to compounds I in which Z is C₃-C₈-cycloalkenyl.

A further embodiment relates to compounds I in which Z is C(O)R^{Π}.

A further embodiment relates to compounds I in which Z is C(O)OR^{Π}.

A further embodiment relates to compounds I in which Z is C(S)OR^{Π}.

A further embodiment relates to compounds I in which Z is C(O)SR^{Π}.

A further embodiment relates to compounds I in which Z is C(S)SR^{Π}.

A further embodiment relates to compounds I in which Z is C(NR^{A})SR^{Π}.

A further embodiment relates to compounds I in which Z is C(S)R^{Π}.

A further embodiment relates to compounds I in which Z is C(NR^{Π})NR^{A}R^{B}.

A further embodiment relates to compounds I in which Z is C(NR^{Π})R^{A}.

A further embodiment relates to compounds I in which Z is C(NR^{Π})OR^{A}.

A further embodiment relates to compounds I in which Z is C(O)NR^{A}R^{B}.

A further embodiment relates to compounds I in which Z is C(S)NR^{A}R^{B}.

A further embodiment relates to compounds I in which Z is C₁-C₈-alkylsulfinyl.

A further embodiment relates to compounds I in which Z is C₁-C₈-alkylthio.

A further embodiment relates to compounds I in which Z is C₁-C₈-alkylsulfonyl.

A further embodiment relates to compounds I in which Z is C(O)—C₁-C₄-alkylene-NR^{A}C(NR^{Π})NR^{A}R^{B}.

A further embodiment relates to compounds I in which Z is C(S)—C₁-C₄-alkylene-NR^{A}C(NR^{Π})NR^{A}R^{B}.

A further embodiment relates to compounds I in which Z is C(NR^{Π})—C₁-C₄-alkylene-NR^{A}C(NR^{Π})NR^{A}R^{B}.

A further embodiment relates to compounds I in which Z is phenyl.

A further embodiment relates to compounds I in which Z is naphthyl.

A further embodiment relates to compounds I in which Z is a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group.

The groups Z mentioned above may be substituted by one or more groups R^{b}.

In a further embodiment, the group Z is substituted by one, two, three or four groups R^{b}, such as halogen, or basic or acidic groups, such as NR^{A}R^{B}, guaninyl, amidyl, hydroxyl, carboxyl or sulfatyl.

A further embodiment relates to compounds I in which R is a group attached via carbon. These compounds correspond to the formula I.b in which R′ is a group R attached via carbon.

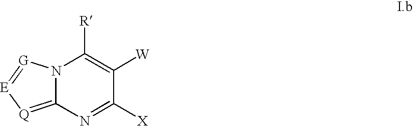

One embodiment relates to compounds I.b in which R′ is C₁-C₁₀-alkyl, C₁-C₁₀-halo-alkyl, C₂-C₁₀-alkenyl, C₂-C₁₀-haloalkenyl, C₂-C₁₀-alkynyl, C₂-C₁₀-haloalkynyl, C₃-C₁₂-cycloalkenyl, C₃-C₁₂-halocycloalkenyl, naphthyl or halonaphthyl or a five-, six-, seven-, eight-, nine- or ten-membered saturated, partially unsaturated or aromatic heterocycle which is attached via carbon and contains one, two, three or four heteroatoms from the group consisting of oxygen, nitrogen and sulfur.

A further embodiment relates to compounds I.b in which R′ is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₂-cycloalkenyl or C₃-C₁₂-halocycloalkenyl or a five-, six-, seven-, eight-, nine- or ten-membered saturated, partially unsaturated or aromatic heterocycle which is attached via carbon and contains one, two, three or four heteroatoms from the group consisting of oxygen, nitrogen and sulfur; where R′ may contain one, two, three or four identical or different groups R^{a}, as defined herein.

According to a further embodiment of the invention, R′ is C₁-C₁₀-alkyl, in particular C₃-C₈-alkyl, which may be partially or fully halogenated and/or substituted by one, two or three R^{a}. Here, R^{a} is preferably selected from the group consisting of cyano, C₁-C₆-alkyl, C₂-C₆-alkenyl, C₂-C₆-alkynyl, C₁-C₆-alkoxycarbonyl, C₁-C₆-alkoximino, C₂-C₆-alkenyloximino, C₂-C₆alkynyloximino, C₃-C₆-cycloalkyl or C₅-C₆-cycloalkenyl, where the aliphatic and/or alicyclic groups for their part may be substituted by one, two or three groups R^{b}. Here, the radicals R^{b} are preferably each independently cyano, C₁-C₆-alkyl, C₂-C₆-alkenyl, C₂-C₆alkynyl, C₁-C₆alkoxy or C₁-C₆alkylcarbonyl.

According to one aspect of this embodiment, R′ is C₁-C₁₀-haloalkyl, in particular C₃-C₈-haloalkyl.

According to a further embodiment of the invention, R′ is C₂-C₁₀-alkenyl, in particular C₃-C₈-alkenyl, which is optionally substituted by one, two or three R^{a}, as defined herein.

According to a further embodiment of the invention, R′ is C₂-C₁₀-alkynyl, in particular C₃-C₈-alkynyl, which is optionally substituted by one, two or three R^{a}, as defined herein.

According to a further embodiment of the invention, R′ is C₃-C₁₂-cycloalkenyl, in particular C₅-C₁₀-cycloalkenyl, especially C₅- or C₆-cycloalkenyl, which is optionally substituted by one, two or three R^{a}, as defined herein. According to one aspect of this embodiment according to the invention, the cycloalkenyl group is mono-, di- or trisubstituted by C₁-C₄-alkyl, such as, for example, methyl and/or ethyl.

According to a further embodiment of the invention, R′ is a five-, six-, seven-, eight-, nine- or ten-membered saturated, partially unsaturated or aromatic heterocycle which is attached via carbon to the azolopyrimidine skeleton and which contains one, two, three or four heteroatoms from the group consisting of oxygen, nitrogen and sulfur, where the heterocycle is unsubstituted or substituted by one, two, three or four identical or different substituents R^{a} as defined herein. According to a preferred aspect of this embodiment, R′ is an optionally substituted five- or six-membered saturated or aromatic heterocycle which is attached via carbon to the azolopyrimidine skeleton.

If R′ carries one, two, three or four, preferably one, two or three, identical or different groups R^{a}, R^{a} is preferably selected from the group consisting of cyano, C₁-C₆-alkyl, C₂-C₆-alkenyl, C₂-C₆-alkynyl, C₁-C₆-alkoxy, C₁-C₆-alkoxycarbonyl, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or may carry one, two or three groups R^{b}.

If R^{a} carries at least one group R^{b}, R^{b} is preferably selected from the group consisting of cyano, C₁-C₆-alkyl, C₂-C₆-alkenyl, C₂-C₆-alkynyl, C₁-C₆-alkylcarbonyl and C₁-C₆-alkoxy.

A further embodiment relates to compounds I.b in which R′ is C₁-C₈-alkyl, in particular branched C₃-C₈-alkyl, C₃-C₈-alkenyl, in particular branched C₃-C₈-alkenyl or C₅-C₆-cycloalkenyl which may have a C₁-C₄-alkyl group.

One embodiment relates to compounds I in which W is phenyl substituted by P¹ and Lₘ.

Suitable groups for Lₘ are in particular the following groups: halogen, such as fluorine or chlorine; cyano; nitro; alkoxycarbonyl; aminocarbonyl; C₁-C₄-alkyl, such as methyl; C₁-C₄-haloalkyl, such as trifluoromethyl; C₁-C₄-alkoxy, such as methoxy.

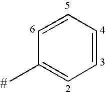

Embodiments of group W relate in particular to phenyl groups which, in addition to group P¹, may have the following substitution:

position 2: fluorine, chlorine, methyl; position 3: hydrogen, fluorine, methoxy; position 4: hydrogen, fluorine, chlorine, methyl, methoxy, cyano, nitro, alkoxycarbonyl, aminocarbonyl, haloalkyl, particularly preferably fluorine, chlorine, methyl, methoxy, cyano; position 5: hydrogen, fluorine, chlorine, methyl; particularly preferably hydrogen, fluorine; position 6: hydrogen, fluorine, chlorine, methyl; particularly preferably hydrogen, fluorine.

Group P¹ is preferably located in position 3, 4 or 5.

In two embodiments of the compounds I, the phenyl group substituted by groups P¹ and Lₘ is group A or B.

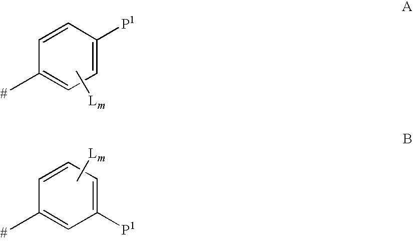

In a further embodiment of the compounds I, in particular in groups A and B, Lₘ is one of the following combinations of substituents: 2-Cl; 2-F; 2,6-Cl₂; 2,6-F₂; 2-F, 6-CH₃; 2-F, 6-Cl; 2,4,6-F₃; 2,6-F₂-4-OCH₃; 2-Cl-4-OCH₃; 2-Cl, 6-CH₃; 2-CH₃-4-F; 2-CF₃; 2-OCH₃, 6-F; 2,4-F₂; 2-F-4-Cl; 2-Cl, 4-F; 2-Cl, 5-F; 2,3-F₂; 2,5-F₂; 2,3,4-F₃; 2CH₃; 2,4-(CH₃)₂; 2-CH₃-4-Cl; 2-CH₃, 5-F; 2-F, 4-CH₃; 2,6-(CH₃)₂; 2,4,6-(CH₃)₃; 2,6-F₂, 4-CH₃.

In a preferred embodiment of the compounds I, in particular in group A, Lₘ is one of the following combinations of substituents: 2-F; 2-Cl; 2-CH₃; 2,6-F₂; 2-F, 6-Cl; 2-F, 6-CH₃.

The compounds of the formula I which carry group A or B correspond to formulae I.A and I.B, respectively.

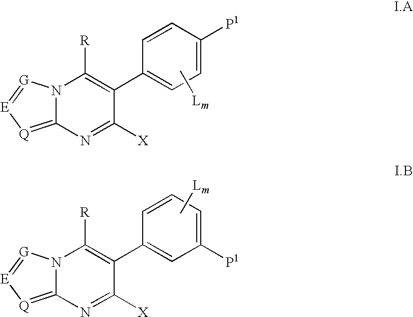

One embodiment relates to compounds I in which W is heteroaryl which is substituted by P¹ and Lₘ and contains one to four nitrogen atoms or one to three nitrogen atoms and one sulfur or oxygen atom.

In one embodiment, the group W is heteroaryl which is substituted by P¹ and Lₘ and attached via a nitrogen atom.

In a further embodiment, the group W is heteroaryl which is substituted by P¹ and Lₘ and attached via a carbon atom.

One embodiment relates to compounds I in which W is 5-membered heteroaryl which is substituted by P¹ and Lₘ and contains one to four nitrogen atoms or one to three nitrogen atoms and one sulfur or oxygen atom.

A further embodiment relates to compounds I in which W is pyrrole, furan, thiophene, pyrazole, isoxazole, isothiazole, imidazole, oxazole, thiazole, 1,2,3-triazole or 1,2,4-triazole.

A further embodiment relates to compounds I in which W is thiophene, pyrazole or thiazole.

One embodiment relates to compounds I in which W is 6-membered heteroaryl which is substituted by P¹ and Lₘ and contains one to three or one to four nitrogen atoms.

A further embodiment relates to compounds I in which W is pyridine, pyrimidine, pyridazine or pyrazine.

One embodiment relates to compounds I in which W is pyridyl which is attached in the 2-, 3- or 4-position and which may be mono- to tetrasubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl. One embodiment of the compounds of the formula I relates to those of the formulae I.C and I.D.

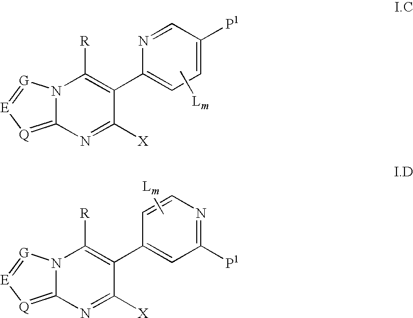

A further embodiment relates to compounds I in which W is pyrimidyl which is attached in the 2- or 4-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl. One embodiment of the compounds of the formula I relates to those of the formulae I.E and I.F.

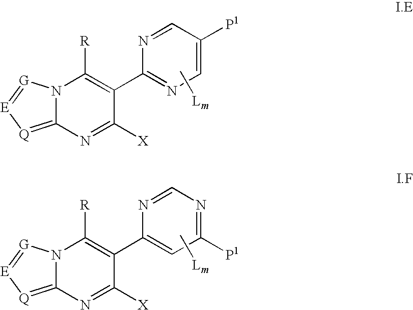

A further embodiment relates to compounds I in which W is thienyl which is attached in the 2- or 3-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl. One embodiment of the compounds of the formula I relates to those of the formulae I.G and I.H.

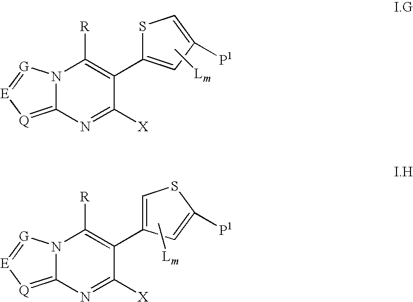

A further embodiment relates to compounds I in which W is thiazolyl which is attached in the 2-, 4- or 5-position and may be substituted by Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl or trifluoromethyl. One embodiment of the compounds of the formula I relates to those of the formulae I.I and I.J.

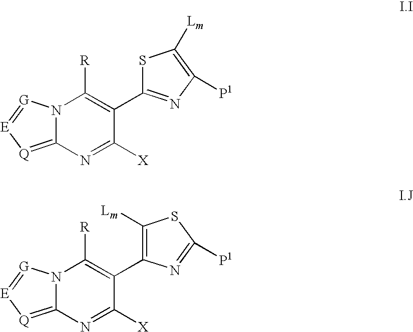

A further embodiment relates to compounds I in which W is imidazolyl which is attached in the 4- or 5-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl. One embodiment of the compounds of the formula I relates to those of the formulae I.K and I.L.

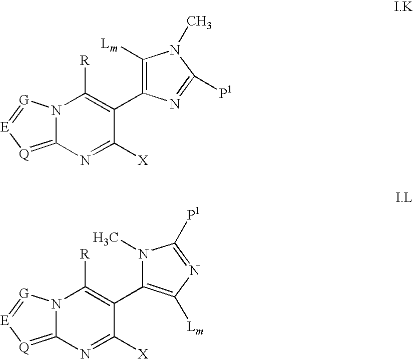

A further embodiment relates to compounds I in which W is pyrazolyl which is attached in the 1-, 3-, 4- or 5-position and may be mono- to trisubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl. One embodiment of the compounds of the formula I relates to those of the formulae I.M, I.N and I.O.

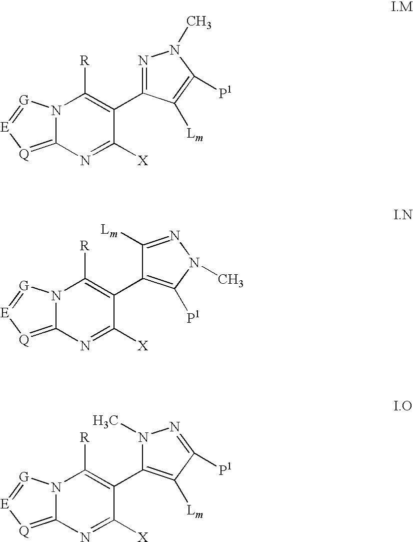

A further embodiment relates to compounds I in which W is oxazolyl which is attached in the 2-, 3- or 4-position and may be mono- or disubstituted by identical or different Lₘ, which here is preferably fluorine, chlorine, bromine, cyano, nitro, methyl, ethyl, methoxy, methylthio, hydroximinomethyl, hydroximinoethyl, methoximinomethyl, methoximinoethyl and/or trifluoromethyl.

One embodiment of the compounds of the formula I relates to those of the formulae I.P and I.Q

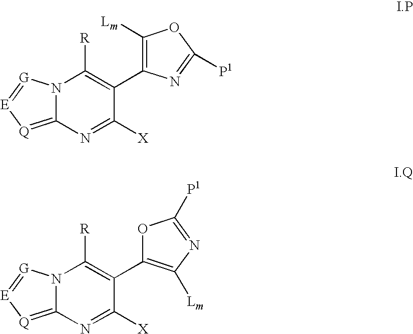

In a preferred embodiment of the compounds I, in particular the formulae I.A to I.Q, at least one group L is located ortho to the point of attachment of group W to the azolopyrimidine skeleton, in particular chlorine, fluorine or methyl.

In a further embodiment, a heteroatom of the heteroaromatic radical W is located ortho to the point of attachment.

The index m is, if structurally possible, preferably 1 to 4, in particular 1 or 2, where the groups L may be identical or different. If the heteroaromatic groups W carry, in addition to a group P¹, further substituents, these are preferably selected from the group consisting of: fluorine, chlorine, methyl, methoxy, cyano, nitro, alkoxycarbonyl, aminocarbonyl and haloalkyl. In a further embodiment, the optional substituents Lₘ are selected from the group consisting of fluorine, chlorine, methyl and methoxy. In a further embodiment, the optional substituents Lₘ are selected from the group consisting of chlorine, methyl and methoxy. A further embodiment relates to heteroaromatic groups W which, in addition to a group P¹, are substituted by chlorine.

In one embodiment of group P¹, Y¹ is CR^{A}R^{B}.

In a further embodiment of group P¹, Y¹ is C(O)NR^{A}.

In a further embodiment of group P¹, Y¹ is oxygen.

In a further embodiment of group P¹, Y¹ is NR^{A}.

In a further embodiment of group P¹, Y¹ is sulfur.

In one embodiment of group P¹, Y² is C₁-C₈-alkylene.

In a further embodiment of group P¹, Y² is C₂-C₈-alkenylene.

In a further embodiment of group P¹, Y² is C₂-C₈-alkynylene.

In a further embodiment of group P¹, Y² is, in particular, C₁-C₈-alkylene which may be interrupted by heteroatoms. Suitable heteroatoms are in particular oxygen and NR^{A}, where in this respect R^{A} is preferably hydrogen or methyl.

A preferred embodiment of Y² relates to straight-chain or singly branched C₁-C₄-alkylene, in particular ethylene and n-propylene.

A further embodiment of group Y² relates to C₃-C₆-alkenylene.

One embodiment relates to compounds I in which T is OH or C₁-C₄-alkoxy.

A further embodiment relates to compounds I in which T is OC(O)R^{Π}, where R^{Π} is preferably C₁-C₄-alkyl, such as methyl.

A further embodiment relates to compounds I in which T is NR^{A}R^{B}, where R^{A} and R^{B} independently of one another are preferably hydrogen or C₁-C₄-alkyl, such as methyl or ethyl. In one embodiment, the group is dimethylamino.

Further embodiments of group T relate to NR^{A}R^{B} and ONR^{A}R^{B} in which R^{A} and R^{B} together with the nitrogen atom to which they are attached form a saturated, partially unsaturated or aromatic five- or six-membered heterocycle which may be mono- or polysubstituted by halogen, C₁-C₄-alkyl or C₁-C₄-haloalkyl and/or which may have one to four, in particular one to three, particularly preferably one or two, carbonyl groups. From among these, particular preference is given to 1-piperazine, 1-piperidine, 1-morpholine, 1-pyrrolidine, 1-pyrazole, 1-pyrrolidin-2-one, 1-pyrrolidinedione, 1,2,4-triazole, 1-pyrrole and 1-pyrrole-2,5-dione.

A further embodiment of group T relates to OR in which R is C₁-C₄-alkyl or a five- or six-membered preferably aromatic heterocycle which is attached via carbon, as defined at the outset, and which may be substituted by one, two or three groups selected from the group consisting of halogen, C₁-C₄-alkyl and C₁-C₄-haloalkyl. Here, R is preferably: methyl, ethyl, n-propyl, pyridine, pyridazine, pyrimidine, in particular 2-pyridine, 4-pyrimidine, 3-pyridazine, which may be halogenated.

Further embodiments of group T relate to OR, OR^{A}, OC(O)R^{A}, C(O)OR^{A}, OC(O)OR^{A}, C(NOR^{A})R^{B}, N(R^{A})C(O)R^{B}, N(R^{A})C(O)OR^{B}, N(R^{A})C(O)—Y²—C (O)R^{A}, OC(O)—Y²—C(O)R^{A}, N(R^{A})C(O)—Y²—C(O)OR^{A}, OC(O)—Y²—C(O)OR^{A}, N(R^{A})C(O)-T¹-C(O)R^{A}, OC(O)-T¹-C(O)R^{A}, N(R^{A})C(O)-T¹-C(O)OR^{A}, OC(O)-T¹-C(O)OR^{A},

R^{A} and R^{B} in group T are preferably hydrogen, unsubstituted or substituted phenyl, C₁-C₄-alkyl or C₁-C₄-haloalkyl, which aliphatic groups may be substituted by halogen or hydroxyl, in particular hydrogen, methyl, ethyl, propyl or halomethyl, particularly preferably hydrogen, methyl or ethyl.

A further embodiment relates to compounds I in which T is C(═NR^{Π})R^{A}, where R^{Π} is preferably C₁-C₄-alkoxy, such as methoxy, and R^{A} is preferably hydrogen or C₁-C₄-alkyl, such as methyl.

A further embodiment relates to compounds I in which T is T¹-C(=T²)T³, preferably —O—C(═O)OR^{A}.

A further embodiment relates to compounds I in which T is S(O)ᵣR^{A}.

One embodiment relates to compounds I in which X is chlorine or bromine, in particular chlorine.

A further embodiment relates to compounds I in which X is cyano, alkyl or alkoxy, in particular cyano, methyl or methoxy.

One embodiment relates to compounds I in which G is N; E is C—W² and Q is N. These compounds correspond to formula I.1.

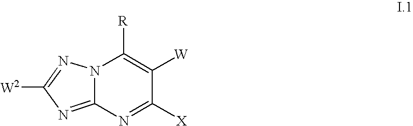

A further embodiment relates to compounds I in which G is N; E is C—W² and Q is C—W³. These compounds correspond to formula I.2.

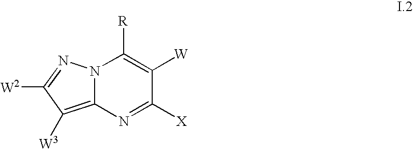

A further embodiment relates to compounds I in which G is C—W¹; E is C—W² and Q is N. These compounds correspond to formula I.3.

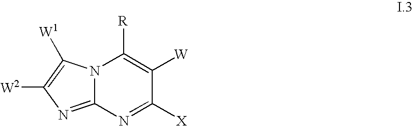

A further embodiment relates to compounds I in which G is C—W¹; E is N and Q is C—W³. These compounds correspond to formula I.4.

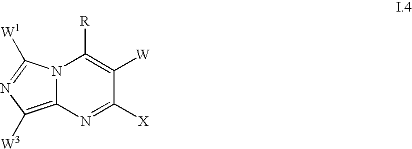

In one embodiment of the compounds I, W¹ is hydrogen, fluorine, chlorine or bromine, in particular hydrogen.

In one embodiment of the compounds I, W² is hydrogen, cyano, fluorine, chlorine, bromine, iodine, nitro, formyl, haloalkyl having 1 to 4 carbon atoms and 1 to 9 fluorine, chlorine and/or bromine atoms, alkyl having 1 to 4 carbon atoms, cycloalkyl having 3 to 6 carbon atoms, thiocarbamoyl, alkoxycarbonyl having 1 to 4 carbon atoms in the alkoxy moiety, alkylcarbonyl having 1 to 4 carbon atoms in the alkyl moiety, hydroximinoalkyl having 1 to 4 carbon atoms in the alkyl moiety or is alkoxyiminoalkyl having 1 to 4 carbon atoms in the alkoxy moiety and 1 to 4 carbon atoms in the alkyl moiety, in particular hydrogen, amino or C₁-C₄-alkyl, preferably hydrogen.

In one embodiment of the compounds I, W³ is hydrogen, cyano, fluorine, chlorine, bromine, iodine, nitro, formyl, haloalkyl having 1 to 4 carbon atoms and 1 to 9 fluorine, chlorine and/or bromine atoms, alkyl having 1 to 4 carbon atoms, cycloalkyl having 3 to 6 carbon atoms, thiocarbamoyl, alkoxycarbonyl having 1 to 4 carbon atoms in the alkoxy moiety, alkylcarbonyl having 1 to 4 carbon atoms in the alkyl moiety, hydroximinoalkyl having 1 to 4 carbon atoms in the alkyl moiety or is alkoxyiminoalkyl having 1 to 4 carbon atoms in the alkoxy moiety and 1 to 4 carbon atoms in the alkyl moiety.

In a further embodiment of the compounds I, W³ is CR¹⁰R¹¹OR¹².

In a further embodiment of the compounds I, W³ is C(R¹³)═NR¹⁴.

Further embodiments of the compounds I correspond to the formulae:

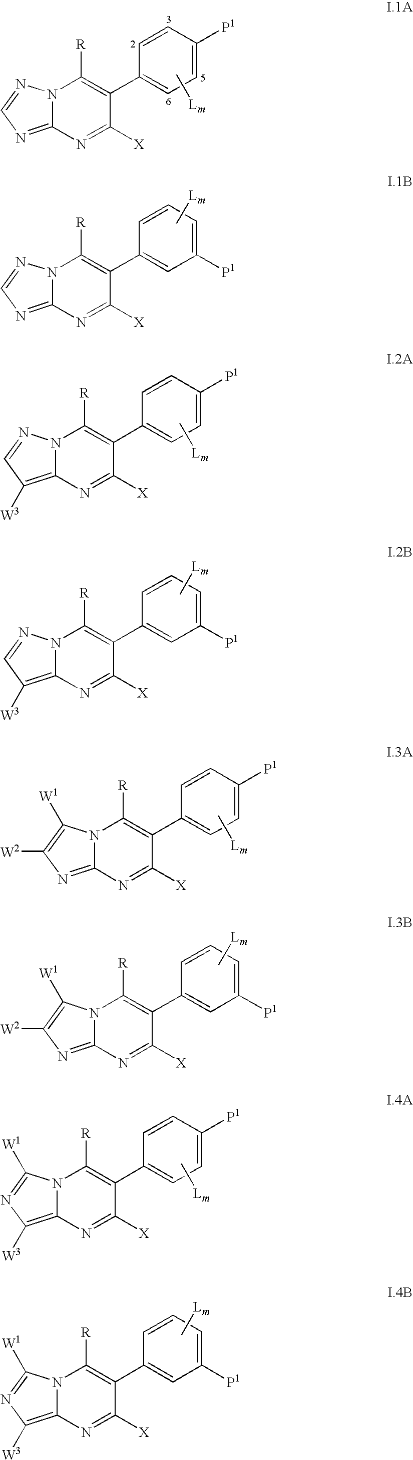

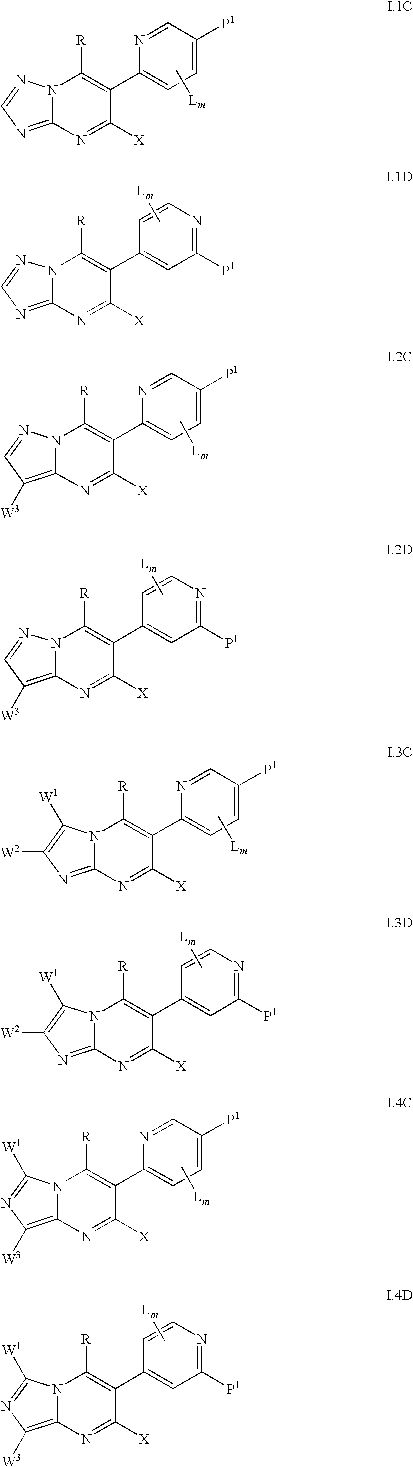

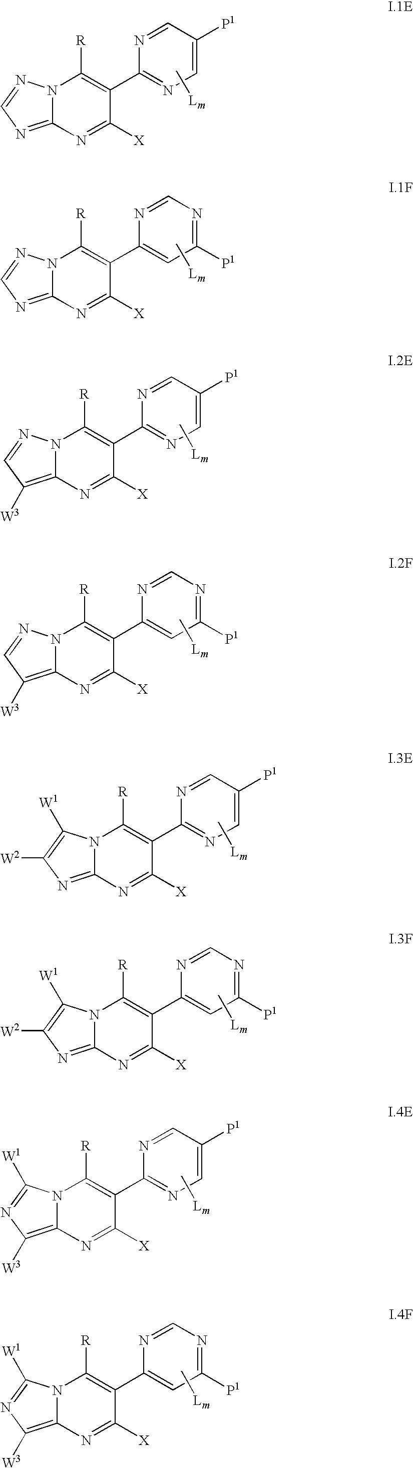

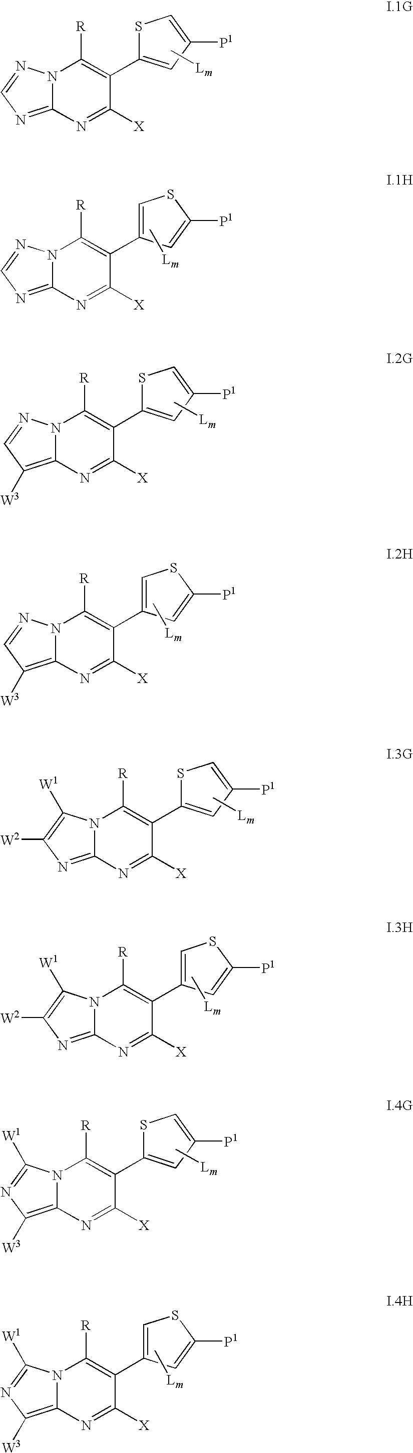

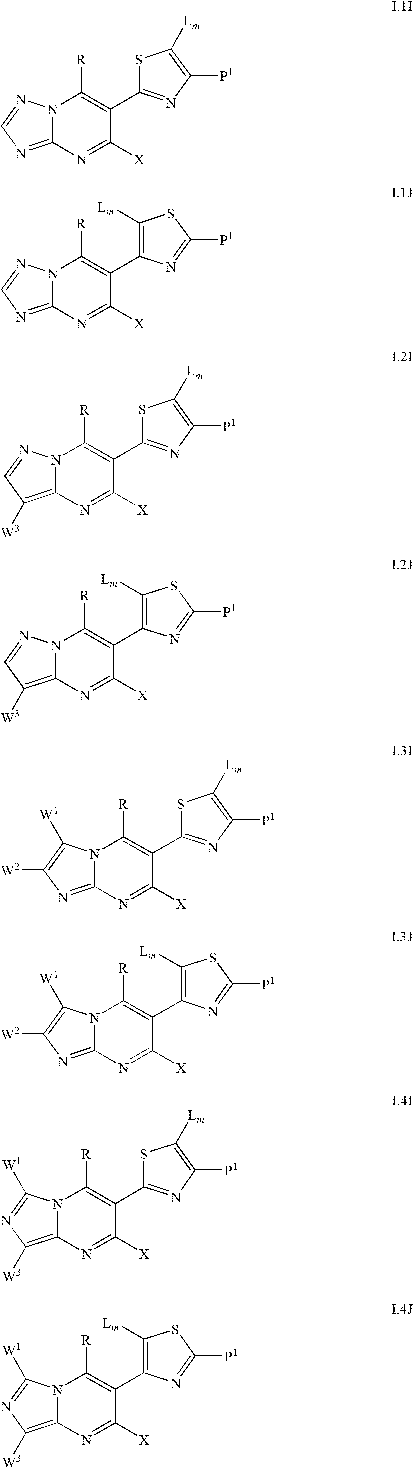

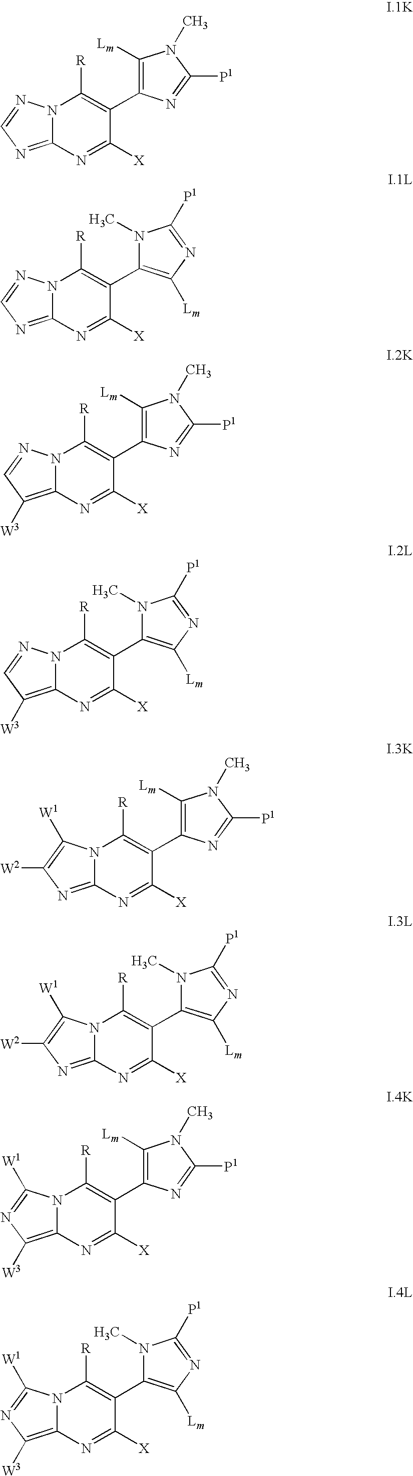

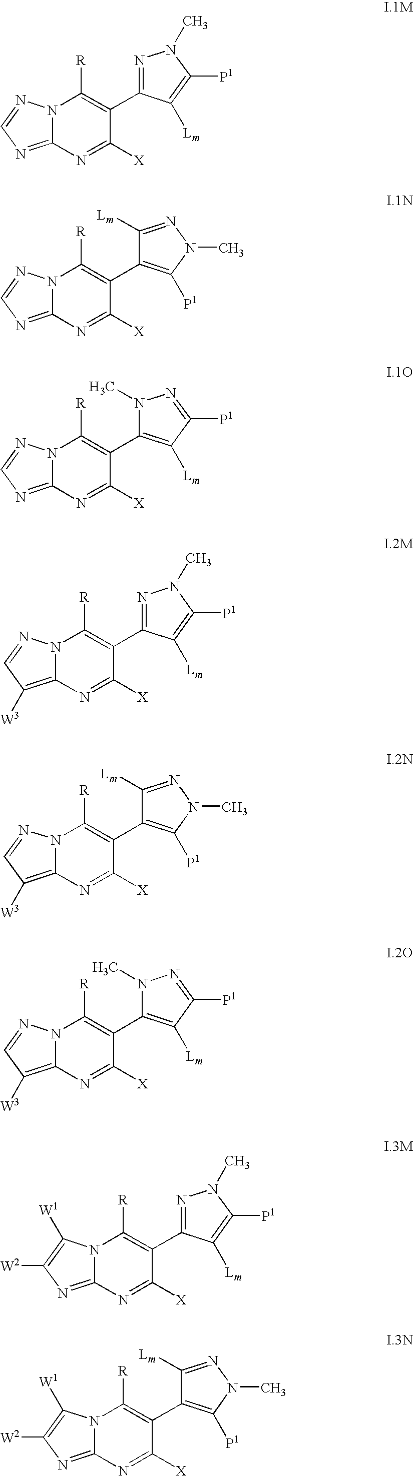

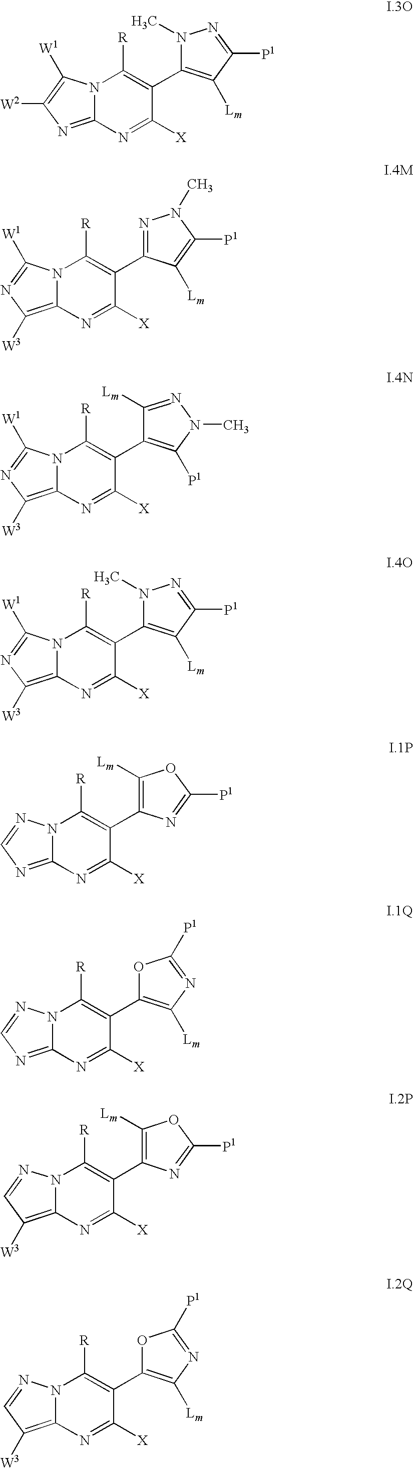

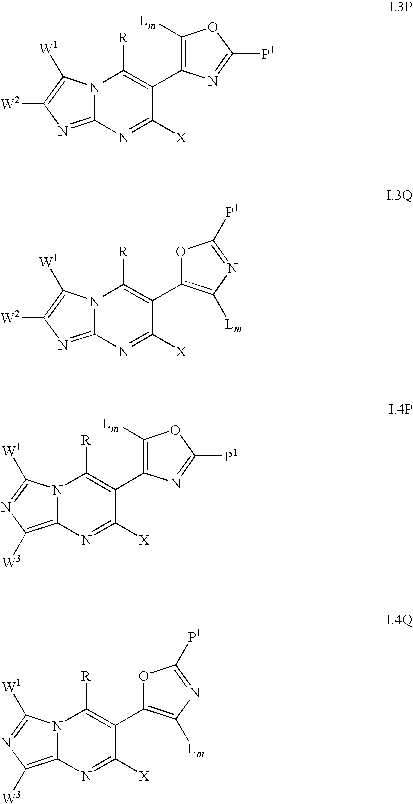

in which the embodiments of the variables correspond to formula I.

In particular with a view to their use, preference is given to the compounds I compiled in the tables below. The groups mentioned for a substituent in the tables are furthermore per se, independently of the combination in which they are mentioned, a particularly preferred embodiment of the substituent in question.

Tables 1 to 1254—compounds of the formula I.1A in which X is Cl, Lₘ and P¹ have the respective given meaning and R for a compound corresponds in each case to one row of Table A

TABLE

| | Lₘ | P¹ |
|---|---|---|
| 1 | 2-F | C(O)NH—(CH₂)₂—OH |
| 2 | 2-F | C(O)NH—(CH₂)₂—NH₂ |
| 3 | 2-F | C(O)NH—(CH₂)₂—NHCH₃ |
| 4 | 2-F | C(O)NH—(CH₂)₂—N(CH₃)₂ |
| 5 | 2-F | C(O)NH—(CH₂)₂—O—C(O)H |
| 6 | 2-F | C(O)NH—(CH₂)₃—OH |
| 7 | 2-F | C(O)NH—(CH₂)₃—NH₂ |
| 8 | 2-F | C(O)NH—(CH₂)₃—NHCH₃ |
| 9 | 2-F | C(O)NH—(CH₂)₃—N(CH₃)₂ |
| 10 | 2-F | C(O)NH—(CH₂)₃—O—C(O)H |
| 11 | 2-F | C(O)NH—CH(CH₃)—CH₂—OH |
| 12 | 2-F | C(O)NH—CH(CH₃)—CH₂—NH₂ |
| 13 | 2-F | C(O)NH—CH(CH₃)—CH₂—NHCH₃ |
| 14 | 2-F | C(O)NH—CH(CH₃)—CH₂—N(CH₃)₂ |
| 15 | 2-F | C(O)NH—CH(CH₃)—CH₂—O—C(O)H |
| 16 | 2-F | C(O)NH—CH₂—CH(CH₃)—OH |
| 17 | 2-F | C(O)NH—CH₂—CH(CH₃)—NH₂ |
| 18 | 2-F | C(O)NH—CH₂—CH(CH₃)—NHCH₃ |
| 19 | 2-F | C(O)NH—CH₂—CH(CH₃)—N(CH₃)₂ |
| 20 | 2-F | C(O)NH—CH₂—CH(CH₃)—O—C(O)H |
| 21 | 2-F | C(O)NH—(CH₂)₂—O—(CH₂)₂—OH |
| 22 | 2-F | C(O)NH—(CH₂)₂—O—(CH₂)₂—NH₂ |
| 23 | 2-F | C(O)NH—(CH₂)₂—O—(CH₂)₂—NHCH₃ |
| 24 | 2-F | C(O)NH—(CH₂)₂—O—(CH₂)₂—N(CH₃)₂ |
| 25 | 2-F | C(O)NH—(CH₂)₂—O—(CH₂)₂—O—C(O)H |
| 26 | 2-F | C(O)NH—(CH₂)₂—NH—(CH₂)₂—OH |
| 27 | 2-F | C(O)NH—(CH₂)₂—NH—(CH₂)₂—NH₂ |
| 28 | 2-F | C(O)NH—(CH₂)₂—NH—(CH₂)₂—NHCH₃ |
| 29 | 2-F | C(O)NH—(CH₂)₂—NH—(CH₂)₂—N(CH₃)₂ |
| 30 | 2-F | C(O)NH—(CH₂)₂—NH—(CH₂)₂—O—C(O)H |
| 31 | 2-F | C(O)NH—(CH₂)₂—N(CH₃)—(CH₂)₂—OH |
| 32 | 2-F | C(O)NH—(CH₂)₂—N(CH₃)—(CH₂)₂—NH₂ |
| 33 | 2-F | C(O)NH—(CH₂)₂—N(CH₃)—(CH₂)₂—NHCH₃ |
| 34 | 2-F | C(O)NH—(CH₂)₂—N(CH₃)—(CH₂)₂—N(CH₃)₂ |
| 35 | 2-F | C(O)NH—(CH₂)₂—N(CH₃)—(CH₂)₂—O—C(O)H |
| 36 | 2-F | C(O)N(CH₃)—(CH₂)₂—OH |
| 37 | 2-F | C(O)N(CH₃)—(CH₂)₂—NH₂ |
| 38 | 2-F | C(O)N(CH₃)—(CH₂)₂—NHCH₃ |
| 39 | 2-F | C(O)N(CH₃)—(CH₂)₂—N(CH₃)₂ |
| 40 | 2-F | C(O)N(CH₃)—(CH₂)₂—O—C(O)H |
| 41 | 2-F | C(O)N(CH₃)—(CH₂)₃—OH |
| 42 | 2-F | C(O)N(CH₃)—(CH₂)₃—NH₂ |
| 43 | 2-F | C(O)N(CH₃)—(CH₂)₃—NHCH₃ |
| 44 | 2-F | C(O)N(CH₃)—(CH₂)₃—N(CH₃)₂ |
| 45 | 2-F | C(O)N(CH₃)—(CH₂)₃—O—C(O)H |
| 46 | 2-F | C(O)N(CH₃)—CH(CH₃)—CH₂—OH |
| 47 | 2-F | C(O)N(CH₃)—CH(CH₃)—CH₂—NH₂ |
| 48 | 2-F | C(O)N(CH₃)—CH(CH₃)—CH₂—NHCH₃ |
| 49 | 2-F | C(O)N(CH₃)—CH(CH₃)—CH₂—N(CH₃)₂ |
| 50 | 2-F | C(O)N(CH₃)—CH(CH₃)—CH₂—O—C(O)H |
| 51 | 2-F | C(O)N(CH₃)—CH₂—CH(CH₃)—OH |
| 52 | 2-F | C(O)N(CH₃)—CH₂—CH(CH₃)—NH₂ |
| 53 | 2-F | C(O)N(CH₃)—CH₂—CH(CH₃)—NHCH₃ |
| 54 | 2-F | C(O)N(CH₃)—CH₂—CH(CH₃)—N(CH₃)₂ |
| 55 | 2-F | C(O)N(CH₃)—(CH₂)₂—O—(CH₂)₂—OH |
| 56 | 2-F | C(O)N(CH₃)—(CH₂)₂—O—(CH₂)₂—NH₂ |
| 57 | 2-F | C(O)N(CH₃)—(CH₂)₂—O—(CH₂)₂—NHCH₃ |
| 58 | 2-F | C(O)N(CH₃)—(CH₂)₂—O—(CH₂)₂—N(CH₃)₂ |
| 59 | 2-F | C(O)N(CH₃)—(CH₂)₂—O—(CH₂)₂—O—C(O)H |
| 60 | 2-F | C(O)N(CH₃)—(CH₂)₂—NH—(CH₂)₂—OH |
| 61 | 2-F | C(O)N(CH₃)—(CH₂)₂—NH—(CH₂)₂—NH₂ |
| 62 | 2-F | C(O)N(CH₃)—(CH₂)₂—NH—(CH₂)₂—NHCH₃ |
| 63 | 2-F | C(O)N(CH₃)—(CH₂)₂—NH—(CH₂)₂—N(CH₃)₂ |
| 64 | 2-F | C(O)N(CH₃)—(CH₂)₂—NH—(CH₂)₂—O—C(O)H |
| 65 | 2-F | C(O)N(CH₃)—(CH₂)₂—N(CH₃)—(CH₂)₂—OH |
| 66 | 2-F | C(O)N(CH₃)—(CH₂)₂—N(CH₃)—(CH₂)₂—NH₂ |
| 67 | 2-F | C(O)N(CH₃)—(CH₂)₂—N(CH₃)—(CH₂)₂—NHCH₃ |
| 68 | 2-F | C(O)N(CH₃)—(CH₂)₂—N(CH₃)—(CH₂)₂—N(CH₃)₂ |
| 69 | 2-F | C(O)N(CH₃)—(CH₂)₂—N(CH₃)—(CH₂)₂—O— |
| | | C(O)H |
| 70 | 2-F | C(O)O—(CH₂)₂—OH |
| 71 | 2-F | C(O)O—(CH₂)₂—NH₂ |
| 72 | 2-F | C(O)O—(CH₂)₂—NHCH₃ |
| 73 | 2-F | C(O)O—(CH₂)₂—N(CH₃)₂ |
| 74 | 2-F | C(O)O—(CH₂)₂—O—C(O)H |
| 75 | 2-F | C(O)O—(CH₂)₃—OH |
| 76 | 2-F | C(O)O—(CH₂)₃—NH₂ |
| 77 | 2-F | C(O)O—(CH₂)₃—NHCH₃ |
| 78 | 2-F | C(O)O—(CH₂)₃—N(CH₃)₂ |
| 79 | 2-F | C(O)O—(CH₂)₃—O—C(O)H |
| 80 | 2-F | C(O)O—CH(CH₃)—CH₂—OH |
| 81 | 2-F | C(O)O—CH(CH₃)—CH₂—NH₂ |
| 82 | 2-F | C(O)O—CH(CH₃)—CH₂—NHCH₃ |
| 83 | 2-F | C(O)O—CH(CH₃)—CH₂—N(CH₃)₂ |
| 84 | 2-F | C(O)O—CH(CH₃)—CH₂—O—C(O)H |
| 85 | 2-F | C(O)O—CH₂—CH(CH₃)—OH |
| 86 | 2-F | C(O)O—CH₂—CH(CH₃)—NH₂ |
| 87 | 2-F | C(O)O—CH₂—CH(CH₃)—NHCH₃ |
| 88 | 2-F | C(O)O—CH₂—CH(CH₃)—N(CH₃)₂ |
| 89 | 2-F | C(O)O—CH₂—CH(CH₃)—O—C(O)H |
| 90 | 2-F | C(O)O—(CH₂)₂—O—(CH₂)₂—OH |
| 91 | 2-F | C(O)O—(CH₂)₂—O—(CH₂)₂—NH₂ |
| 92 | 2-F | C(O)O—(CH₂)₂—O—(CH₂)₂—NHCH₃ |
| 93 | 2-F | C(O)O—(CH₂)₂—O—(CH₂)₂—N(CH₃)₂ |
| 94 | 2-F | C(O)O—(CH₂)₂—O—(CH₂)₂—O—C(O)H |
| 95 | 2-F | C(O)O—(CH₂)₂—NH—(CH₂)₂—OH |
| 96 | 2-F | C(O)O—(CH₂)₂—NH—(CH₂)₂—NH₂ |
| 97 | 2-F | C(O)O—(CH₂)₂—NH—(CH₂)₂—NHCH₃ |
| 98 | 2-F | C(O)O—(CH₂)₂—NH—(CH₂)₂—N(CH₃)₂ |
| 99 | 2-F | C(O)O—(CH₂)₂—NH—(CH₂)₂—O—C(O)H |
| 100 | 2-F | C(O)O—(CH₂)₂—N(CH₃)—(CH₂)₂—OH |
| 101 | 2-F | C(O)O—(CH₂)₂—N(CH₃)—(CH₂)₂—NH₂ |
| 102 | 2-F | C(O)O—(CH₂)₂—N(CH₃)—(CH₂)₂—NHCH₃ |
| 103 | 2-F | C(O)O—(CH₂)₂—N(CH₃)—(CH₂)₂—N(CH₃)₂ |
| 104 | 2-F | C(O)O—(CH₂)₂—N(CH₃)—(CH₂)₂—O—C(O)H |
| 105 | 2-F | O—(CH₂)₂—OH |
| 106 | 2-F | O—(CH₂)₂—NH₂ |
| 107 | 2-F | O—(CH₂)₂—NHCH₃ |
| 108 | 2-F | O—(CH₂)₂—N(CH₃)₂ |
| 109 | 2-F | O—(CH₂)₂—O—C(O)H |
| 110 | 2-F | O—(CH₂)₃—OH |
| 111 | 2-F | O—(CH₂)₃—NH₂ |
| 112 | 2-F | O—(CH₂)₃—NHCH₃ |
| 113 | 2-F | O—(CH₂)₃—N(CH₃)₂ |
| 114 | 2-F | O—(CH₂)₃—O—C(O)H |
| 115 | 2-F | O—CH(CH₃)—CH₂—OH |
| 116 | 2-F | O—CH(CH₃)—CH₂—NH₂ |
| 117 | 2-F | O—CH(CH₃)—CH₂—NHCH₃ |
| 118 | 2-F | O—CH(CH₃)—CH₂—N(CH₃)₂ |
| 119 | 2-F | O—CH(CH₃)—CH₂—O—C(O)H |
| 120 | 2-F | O—CH₂—CH(CH₃)—OH |
| 121 | 2-F | O—CH₂—CH(CH₃)—NH₂ |
| 122 | 2-F | O—CH₂—CH(CH₃)—NHCH₃ |
| 123 | 2-F | O—CH₂—CH(CH₃)—N(CH₃)₂ |
| 124 | 2-F | O—CH₂—CH(CH₃)—O—C(O)H |
| 125 | 2-F | O—(CH₂)₂—O—(CH₂)₂—OH |
| 126 | 2-F | O—(CH₂)₂—O—(CH₂)₂—NH₂ |
| 127 | 2-F | O—(CH₂)₂—O—(CH₂)₂—NHCH₃ |
| 128 | 2-F | O—(CH₂)₂—O—(CH₂)₂—N(CH₃)₂ |
| 129 | 2-F | O—(CH₂)₂—O—(CH₂)₂—O—C(O)H |
| 130 | 2-F | O—(CH₂)₂—NH—(CH₂)₂—OH |
| 131 | 2-F | O—(CH₂)₂—NH—(CH₂)₂—NH₂ |
| 132 | 2-F | O—(CH₂)₂—NH—(CH₂)₂—NHCH₃ |
| 133 | 2-F | O—(CH₂)₂—NH—(CH₂)₂—N(CH₃)₂ |
| 134 | 2-F | O—(CH₂)₂—NH—(CH₂)₂—O—C(O)H |
| 135 | 2-F | O—(CH₂)₂—N(CH₃)—(CH₂)₂—OH |
| 136 | 2-F | O—(CH₂)₂—N(CH₃)—(CH₂)₂—NH₂ |
| 137 | 2-F | O—(CH₂)₂—N(CH₃)—(CH₂)₂—NHCH₃ |
| 138 | 2-F | O—(CH₂)₂—N(CH₃)—(CH₂)₂—N(CH₃)₂ |
| 139 | 2-F | O—(CH₂)₂—N(CH₃)—(CH₂)₂—O—C(O)H |
| 140 | 2-F | NH—(CH₂)₂—OH |
| 141 | 2-F | NH—(CH₂)₂—NH₂ |
| 142 | 2-F | NH—(CH₂)₂—NHCH₃ |
| 143 | 2-F | NH—(CH₂)₂—N(CH₃)₂ |
| 144 | 2-F | NH—(CH₂)₂—O—C(O)H |
| 145 | 2-F | NH—(CH₂)₃—OH |
| 146 | 2-F | NH—(CH₂)₃—NH₂ |
| 147 | 2-F | NH—(CH₂)₃—NHCH₃ |
| 148 | 2-F | NH—(CH₂)₃—N(CH₃)₂ |
| 149 | 2-F | NH—(CH₂)₃—O—C(O)H |
| 150 | 2-F | NH—CH(CH₃)—CH₂—OH |
| 151 | 2-F | NH—CH(CH₃)—CH₂—NH₂ |
| 152 | 2-F | NH—CH(CH₃)—CH₂—NHCH₃ |
| 153 | 2-F | NH—CH(CH₃)—CH₂—N(CH₃)₂ |
| 154 | 2-F | NH—CH(CH₃)—CH₂—O—C(O)H |
| 155 | 2-F | NH—CH₂—CH(CH₃)—OH |
| 156 | 2-F | NH—CH₂—CH(CH₃)—NH₂ |
| 157 | 2-F | NH—CH₂—CH(CH₃)—NHCH₃ |
| 158 | 2-F | NH—CH₂—CH(CH₃)—N(CH₃)₂ |
| 159 | 2-F | NH—CH₂—CH(CH₃)—O—C(O)H |
| 160 | 2-F | NH—(CH₂)₂—O—(CH₂)₂—OH |
| 161 | 2-F | NH—(CH₂)₂—O—(CH₂)₂—NH₂ |
| 162 | 2-F | NH—(CH₂)₂—O—(CH₂)₂—NHCH₃ |
| 163 | 2-F | NH—(CH₂)₂—O—(CH₂)₂—N(CH₃)₂ |
| 164 | 2-F | NH—(CH₂)₂—O—(CH₂)₂—O—C(O)H |
| 165 | 2-F | NH—(CH₂)₂—NH—(CH₂)₂—OH |
| 166 | 2-F | NH—(CH₂)₂—NH—(CH₂)₂—NH₂ |
| 167 | 2-F | NH—(CH₂)₂—NH—(CH₂)₂—NHCH₃ |
| 168 | 2-F | NH—(CH₂)₂—NH—(CH₂)₂—N(CH₃)₂ |
| 169 | 2-F | NH—(CH₂)₂—NH—(CH₂)₂—O—C(O)H |
| 170 | 2-F | NH—(CH₂)₂—N(CH₃)—(CH₂)₂—OH |
| 171 | 2-F | NH—(CH₂)₂—N(CH₃)—(CH₂)₂—NH₂ |
| 172 | 2-F | NH—(CH₂)₂—N(CH₃)—(CH₂)₂—NHCH₃ |
| 173 | 2-F | NH—(CH₂)₂—N(CH₃)—(CH₂)₂—N(CH₃)₂ |
| 174 | 2-F | NH—(CH₂)₂—N(CH₃)—(CH₂)₂—O—C(O)H |
| 175 | 2-F | N(CH₃)—(CH₂)₂—OH |
| 176 | 2-F | N(CH₃)—(CH₂)₂—NH₂ |
| 177 | 2-F | N(CH₃)—(CH₂)₂—NHCH₃ |
| 178 | 2-F | N(CH₃)—(CH₂)₂—N(CH₃)₂ |
| 179 | 2-F | N(CH₃)—(CH₂)₂—O—C(O)H |
| 180 | 2-F | N(CH₃)—(CH₂)₃—OH |
| 181 | 2-F | N(CH₃)—(CH₂)₃—NH₂ |
| 182 | 2-F | N(CH₃)—(CH₂)₃—NHCH₃ |
| 183 | 2-F | N(CH₃)—(CH₂)₃—N(CH₃)₂ |
| 184 | 2-F | N(CH₃)—(CH₂)₃—O—C(O)H |
| 185 | 2-F | N(CH₃)—CH(CH₃)—CH₂—OH |
| 186 | 2-F | N(CH₃)—CH(CH₃)—CH₂—NH₂ |
| 187 | 2-F | N(CH₃)—CH(CH₃)—CH₂—NHCH₃ |
| 188 | 2-F | N(CH₃)—CH(CH₃)—CH₂—N(CH₃)₂ |
| 189 | 2-F | N(CH₃)—CH(CH₃)—CH₂—O—C(O)H |
| 190 | 2-F | N(CH₃)—CH₂—CH(CH₃)—OH |
| 191 | 2-F | N(CH₃)—CH₂—CH(CH₃)—NH₂ |
| 192 | 2-F | N(CH₃)—CH₂—CH(CH₃)—NHCH₃ |
| 193 | 2-F | N(CH₃)—CH₂—CH(CH₃)—N(CH₃)₂ |
| 194 | 2-F | N(CH₃)—CH₂—CH(CH₃)—O—C(O)H |
| 195 | 2-F | N(CH₃)—(CH₂)₂—O—(CH₂)₂—OH |
| 196 | 2-F | N(CH₃)—(CH₂)₂—O—(CH₂)₂—NH₂ |
| 197 | 2-F | N(CH₃)—(CH₂)₂—O—(CH₂)₂—NHCH₃ |
| 198 | 2-F | N(CH₃)—(CH₂)₂—O—(CH₂)₂—N(CH₃)₂ |
| 199 | 2-F | N(CH₃)—(CH₂)₂—O—(CH₂)₂—O—C(O)H |
| 200 | 2-F | N(CH₃)—(CH₂)₂—NH—(CH₂)₂—OH |
| 201 | 2-F | N(CH₃)—(CH₂)₂—NH—(CH₂)₂—NH₂ |
| 202 | 2-F | N(CH₃)—(CH₂)₂—NH—(CH₂)₂—NHCH₃ |
| 203 | 2-F | N(CH₃)—(CH₂)₂—NH—(CH₂)₂—N(CH₃)₂ |
| 204 | 2-F | N(CH₃)—(CH₂)₂—NH—(CH₂)₂—O—C(O)H |
| 205 | 2-F | N(CH₃)—(CH₂)₂—N(CH₃)—(CH₂)₂—OH |
| 206 | 2-F | N(CH₃)—(CH₂)₂—N(CH₃)—(CH₂)₂—NH₂ |
| 207 | 2-F | N(CH₃)—(CH₂)₂—N(CH₃)—(CH₂)₂—NHCH₃ |
| 208 | 2-F | N(CH₃)—(CH₂)₂—N(CH₃)—(CH₂)₂—N(CH₃)₂ |
| 209 | 2-F | N(CH₃)—(CH₂)₂—N(CH₃)—(CH₂)₂—O—C(O)H |
| 210 | 2-Cl | C(O)NH—(CH₂)₂—OH |
| 211 | 2-Cl | C(O)NH—(CH₂)₂—NH₂ |
| 212 | 2-Cl | C(O)NH—(CH₂)₂—NHCH₃ |
| 213 | 2-Cl | C(O)NH—(CH₂)₂—N(CH₃)₂ |
| 214 | 2-Cl | C(O)NH—(CH₂)₂—O—C(O)H |
| 215 | 2-Cl | C(O)NH—(CH₂)₃—OH |
| 216 | 2-Cl | C(O)NH—(CH₂)₃—NH₂ |
| 217 | 2-Cl | C(O)NH—(CH₂)₃—NHCH₃ |
| 218 | 2-Cl | C(O)NH—(CH₂)₃—N(CH₃)₂ |
| 219 | 2-Cl | C(O)NH—(CH₂)₃—O—C(O)H |
| 220 | 2-Cl | C(O)NH—CH(CH₃)—CH₂—OH |
| 221 | 2-Cl | C(O)NH—CH(CH₃)—CH₂—NH₂ |
| 222 | 2-Cl | C(O)NH—CH(CH₃)—CH₂—NHCH₃ |
| 223 | 2-Cl | C(O)NH—CH(CH₃)—CH₂—N(CH₃)₂ |
| 224 | 2-Cl | C(O)NH—CH(CH₃)—CH₂—O—C(O)H |
| 225 | 2-Cl | C(O)NH—CH₂—CH(CH₃)—OH |
| 226 | 2-Cl | C(O)NH—CH₂—CH(CH₃)—NH₂ |
| 227 | 2-Cl | C(O)NH—CH₂—CH(CH₃)—NHCH₃ |
| 228 | 2-Cl | C(O)NH—CH₂—CH(CH₃)—N(CH₃)₂ |
| 229 | 2-Cl | C(O)NH—CH₂—CH(CH₃)—O—C(O)H |
| 230 | 2-Cl | C(O)NH—(CH₂)₂—O—(CH₂)₂—OH |
| 231 | 2-Cl | C(O)NH—(CH₂)₂—O—(CH₂)₂—NH₂ |
| 232 | 2-Cl | C(O)NH—(CH₂)₂—O—(CH₂)₂—NHCH₃ |
| 233 | 2-Cl | C(O)NH—(CH₂)₂—O—(CH₂)₂—N(CH₃)₂ |
| 234 | 2-Cl | C(O)NH—(CH₂)₂—O—(CH₂)₂—O—C(O)H |
| 235 | 2-Cl | C(O)NH—(CH₂)₂—NH—(CH₂)₂—OH |
| 236 | 2-Cl | C(O)NH—(CH₂)₂—NH—(CH₂)₂—NH₂ |
| 237 | 2-Cl | C(O)NH—(CH₂)₂—NH—(CH₂)₂—NHCH₃ |
| 238 | 2-Cl | C(O)NH—(CH₂)₂—NH—(CH₂)₂—N(CH₃)₂ |
| 239 | 2-Cl | C(O)NH—(CH₂)₂—NH—(CH₂)₂—O—C(O)H |
| 240 | 2-Cl | C(O)NH—(CH₂)₂—N(CH₃)—(CH₂)₂—OH |
| 241 | 2-Cl | C(O)NH—(CH₂)₂—N(CH₃)—(CH₂)₂—NH₂ |
| 242 | 2-Cl | C(O)NH—(CH₂)₂—N(CH₃)—(CH₂)₂—NHCH₃ |
| 243 | 2-Cl | C(O)NH—(CH₂)₂—N(CH₃)—(CH₂)₂—N(CH₃)₂ |
| 244 | 2-Cl | C(O)NH—(CH₂)₂—N(CH₃)—(CH₂)₂—O—C(O)H |
| 245 | 2-Cl | C(O)N(CH₃)—(CH₂)₂—OH |
| 246 | 2-Cl | C(O)N(CH₃)—(CH₂)₂—NH₂ |
| 247 | 2-Cl | C(O)N(CH₃)—(CH₂)₂—NHCH₃ |
| 248 | 2-Cl | C(O)N(CH₃)—(CH₂)₂—N(CH₃)₂ |
| 249 | 2-Cl | C(O)N(CH₃)—(CH₂)₂—O—C(O)H |
| 250 | 2-Cl | C(O)N(CH₃)—(CH₂)₃—OH |
| 251 | 2-Cl | C(O)N(CH₃)—(CH₂)₃—NH₂ |
| 252 | 2-Cl | C(O)N(CH₃)—(CH₂)₃—NHCH₃ |
| 253 | 2-Cl | C(O)N(CH₃)—(CH₂)₃—N(CH₃)₂ |
| 254 | 2-Cl | C(O)N(CH₃)—(CH₂)₃—O—C(O)H |
| 255 | 2-Cl | C(O)N(CH₃)—CH(CH₃)—CH₂—OH |
| 256 | 2-Cl | C(O)N(CH₃)—CH(CH₃)—CH₂—NH₂ |
| 257 | 2-Cl | C(O)N(CH₃)—CH(CH₃)—CH₂—NHCH₃ |
| 258 | 2-Cl | C(O)N(CH₃)—CH(CH₃)—CH₂—N(CH₃)₂ |
| 259 | 2-Cl | C(O)N(CH₃)—CH(CH₃)—CH₂—O—C(O)H |
| 260 | 2-Cl | C(O)N(CH₃)—CH₂—CH(CH₃)—OH |
| 261 | 2-Cl | C(O)N(CH₃)—CH₂—CH(CH₃)—NH₂ |
| 262 | 2-Cl | C(O)N(CH₃)—CH₂—CH(CH₃)—NHCH₃ |
| 263 | 2-Cl | C(O)N(CH₃)—CH₂—CH(CH₃)—N(CH₃)₂ |
| 264 | 2-Cl | C(O)N(CH₃)—(CH₂)₂—O—(CH₂)₂—OH |
| 265 | 2-Cl | C(O)N(CH₃)—(CH₂)₂—O—(CH₂)₂—NH₂ |
| 266 | 2-Cl | C(O)N(CH₃)—(CH₂)₂—O—(CH₂)₂—NHCH₃ |
| 267 | 2-Cl | C(O)N(CH₃)—(CH₂)₂—O—(CH₂)₂—N(CH₃)₂ |
| 268 | 2-Cl | C(O)N(CH₃)—(CH₂)₂—O—(CH₂)₂—O—C(O)H |
| 269 | 2-Cl | C(O)N(CH₃)—(CH₂)₂—NH—(CH₂)₂—OH |
| 270 | 2-Cl | C(O)N(CH₃)—(CH₂)₂—NH—(CH₂)₂—NH₂ |
| 271 | 2-Cl | C(O)N(CH₃)—(CH₂)₂—NH—(CH₂)₂—NHCH₃ |
| 272 | 2-Cl | C(O)N(CH₃)—(CH₂)₂—NH—(CH₂)₂—N(CH₃)₂ |
| 273 | 2-Cl | C(O)N(CH₃)—(CH₂)₂—NH—(CH₂)₂—O—C(O)H |
| 274 | 2-Cl | C(O)N(CH₃)—(CH₂)₂—N(CH₃)—(CH₂)₂—OH |
| 275 | 2-Cl | C(O)N(CH₃)—(CH₂)₂—N(CH₃)—(CH₂)₂—NH₂ |
| 276 | 2-Cl | C(O)N(CH₃)—(CH₂)₂—N(CH₃)—(CH₂)₂—NHCH₃ |
| 277 | 2-Cl | C(O)N(CH₃)—(CH₂)₂—N(CH₃)—(CH₂)₂—N(CH₃)₂ |
| 278 | 2-Cl | C(O)N(CH₃)—(CH₂)₂—N(CH₃)—(CH₂)₂—O— |
| | | C(O)H |
| 279 | 2-Cl | C(O)O—(CH₂)₂—OH |
| 280 | 2-Cl | C(O)O—(CH₂)₂—NH₂ |
| 281 | 2-Cl | C(O)O—(CH₂)₂—NHCH₃ |
| 282 | 2-Cl | C(O)O—(CH₂)₂—N(CH₃)₂ |
| 283 | 2-Cl | C(O)O—(CH₂)₂—O—C(O)H |
| 284 | 2-Cl | C(O)O—(CH₂)₃—OH |
| 285 | 2-Cl | C(O)O—(CH₂)₃—NH₂ |
| 286 | 2-Cl | C(O)O—(CH₂)₃—NHCH₃ |
| 287 | 2-Cl | C(O)O—(CH₂)₃—N(CH₃)₂ |
| 288 | 2-Cl | C(O)O—(CH₂)₃—O—C(O)H |
| 289 | 2-Cl | C(O)O—CH(CH₃)—CH₂—OH |
| 290 | 2-Cl | C(O)O—CH(CH₃)—CH₂—NH₂ |
| 291 | 2-Cl | C(O)O—CH(CH₃)—CH₂—NHCH₃ |
| 292 | 2-Cl | C(O)O—CH(CH₃)—CH₂—N(CH₃)₂ |
| 293 | 2-Cl | C(O)O—CH(CH₃)—CH₂—O—C(O)H |
| 294 | 2-Cl | C(O)O—CH₂—CH(CH₃)—OH |
| 295 | 2-Cl | C(O)O—CH₂—CH(CH₃)—NH₂ |
| 296 | 2-Cl | C(O)O—CH₂—CH(CH₃)—NHCH₃ |
| 297 | 2-Cl | C(O)O—CH₂—CH(CH₃)—N(CH₃)₂ |
| 298 | 2-Cl | C(O)O—CH₂—CH(CH₃)—O—C(O)H |
| 299 | 2-Cl | C(O)O—(CH₂)₂—O—(CH₂)₂—OH |
| 300 | 2-Cl | C(O)O—(CH₂)₂—O—(CH₂)₂—NH₂ |
| 301 | 2-Cl | C(O)O—(CH₂)₂—O—(CH₂)₂—NHCH₃ |
| 302 | 2-Cl | C(O)O—(CH₂)₂—O—(CH₂)₂—N(CH₃)₂ |
| 303 | 2-Cl | C(O)O—(CH₂)₂—O—(CH₂)₂—O—C(O)H |
| 304 | 2-Cl | C(O)O—(CH₂)₂—NH—(CH₂)₂—OH |
| 305 | 2-Cl | C(O)O—(CH₂)₂—NH—(CH₂)₂—NH₂ |
| 306 | 2-Cl | C(O)O—(CH₂)₂—NH—(CH₂)₂—NHCH₃ |
| 307 | 2-Cl | C(O)O—(CH₂)₂—NH—(CH₂)₂—N(CH₃)₂ |
| 308 | 2-Cl | C(O)O—(CH₂)₂—NH—(CH₂)₂—O—C(O)H |
| 309 | 2-Cl | C(O)O—(CH₂)₂—N(CH₃)—(CH₂)₂—OH |
| 310 | 2-Cl | C(O)O—(CH₂)₂—N(CH₃)—(CH₂)₂—NH₂ |
| 311 | 2-Cl | C(O)O—(CH₂)₂—N(CH₃)—(CH₂)₂—NHCH₃ |
| 312 | 2-Cl | C(O)O—(CH₂)₂—N(CH₃)—(CH₂)₂—N(CH₃)₂ |
| 313 | 2-Cl | C(O)O—(CH₂)₂—N(CH₃)—(CH₂)₂—O—C(O)H |
| 314 | 2-Cl | O—(CH₂)₂—OH |
| 315 | 2-Cl | O—(CH₂)₂—NH₂ |
| 316 | 2-Cl | O—(CH₂)₂—NHCH₃ |
| 317 | 2-Cl | O—(CH₂)₂—N(CH₃)₂ |
| 318 | 2-Cl | O—(CH₂)₂—O—C(O)H |
| 319 | 2-Cl | O—(CH₂)₃—OH |
| 320 | 2-Cl | O—(CH₂)₃—NH₂ |
| 321 | 2-Cl | O—(CH₂)₃—NHCH₃ |
| 322 | 2-Cl | O—(CH₂)₃—N(CH₃)₂ |
| 323 | 2-Cl | O—(CH₂)₃—O—C(O)H |
| 324 | 2-Cl | O—CH(CH₃)—CH₂—OH |
| 325 | 2-Cl | O—CH(CH₃)—CH₂—NH₂ |
| 326 | 2-Cl | O—CH(CH₃)—CH₂—NHCH₃ |
| 327 | 2-Cl | O—CH(CH₃)—CH₂—N(CH₃)₂ |
| 328 | 2-Cl | O—CH(CH₃)—CH₂—O—C(O)H |
| 329 | 2-Cl | O—CH₂—CH(CH₃)—OH |
| 330 | 2-Cl | O—CH₂—CH(CH₃)—NH₂ |
| 331 | 2-Cl | O—CH₂—CH(CH₃)—NHCH₃ |
| 332 | 2-Cl | O—CH₂—CH(CH₃)—N(CH₃)₂ |
| 333 | 2-Cl | O—CH₂—CH(CH₃)—O—C(O)H |
| 334 | 2-Cl | O—(CH₂)₂—O—(CH₂)₂—OH |
| 335 | 2-Cl | O—(CH₂)₂—O—(CH₂)₂—NH₂ |
| 336 | 2-Cl | O—(CH₂)₂—O—(CH₂)₂—NHCH₃ |
| 337 | 2-Cl | O—(CH₂)₂—O—(CH₂)₂—N(CH₃)₂ |
| 338 | 2-Cl | O—(CH₂)₂—O—(CH₂)₂—O—C(O)H |
| 339 | 2-Cl | O—(CH₂)₂—NH—(CH₂)₂—OH |
| 340 | 2-Cl | O—(CH₂)₂—NH—(CH₂)₂—NH₂ |
| 341 | 2-Cl | O—(CH₂)₂—NH—(CH₂)₂—NHCH₃ |
| 342 | 2-Cl | O—(CH₂)₂—NH—(CH₂)₂—N(CH₃)₂ |
| 343 | 2-Cl | O—(CH₂)₂—NH—(CH₂)₂—O—C(O)H |
| 344 | 2-Cl | O—(CH₂)₂—N(CH₃)—(CH₂)₂—OH |
| 345 | 2-Cl | O—(CH₂)₂—N(CH₃)—(CH₂)₂—NH₂ |
| 346 | 2-Cl | O—(CH₂)₂—N(CH₃)—(CH₂)₂—NHCH₃ |
| 347 | 2-Cl | O—(CH₂)₂—N(CH₃)—(CH₂)₂—N(CH₃)₂ |
| 348 | 2-Cl | O—(CH₂)₂—N(CH₃)—(CH₂)₂—O—C(O)H |
| 349 | 2-Cl | NH—(CH₂)₂—OH |
| 350 | 2-Cl | NH—(CH₂)₂—NH₂ |
| 351 | 2-Cl | NH—(CH₂)₂—NHCH₃ |
| 352 | 2-Cl | NH—(CH₂)₂—N(CH₃)₂ |
| 353 | 2-Cl | NH—(CH₂)₂—O—C(O)H |
| 354 | 2-Cl | NH—(CH₂)₃—OH |
| 355 | 2-Cl | NH—(CH₂)₃—NH₂ |
| 356 | 2-Cl | NH—(CH₂)₃—NHCH₃ |
| 357 | 2-Cl | NH—(CH₂)₃—N(CH₃)₂ |
| 358 | 2-Cl | NH—(CH₂)₃—O—C(O)H |
| 359 | 2-Cl | NH—CH(CH₃)—CH₂—OH |
| 360 | 2-Cl | NH—CH(CH₃)—CH₂—NH₂ |
| 361 | 2-Cl | NH—CH(CH₃)—CH₂—NHCH₃ |
| 362 | 2-Cl | NH—CH(CH₃)—CH₂—N(CH₃)₂ |
| 363 | 2-Cl | NH—CH(CH₃)—CH₂—O—C(O)H |
| 364 | 2-Cl | NH—CH₂—CH(CH₃)—OH |
| 365 | 2-Cl | NH—CH₂—CH(CH₃)—NH₂ |
| 366 | 2-Cl | NH—CH₂—CH(CH₃)—NHCH₃ |
| 367 | 2-Cl | NH—CH₂—CH(CH₃)—N(CH₃)₂ |
| 368 | 2-Cl | NH—CH₂—CH(CH₃)—O—C(O)H |
| 369 | 2-Cl | NH—(CH₂)₂—O—(CH₂)₂—OH |
| 370 | 2-Cl | NH—(CH₂)₂—O—(CH₂)₂—NH₂ |
| 371 | 2-Cl | NH—(CH₂)₂—O—(CH₂)₂—NHCH₃ |
| 372 | 2-Cl | NH—(CH₂)₂—O—(CH₂)₂—N(CH₃)₂ |
| 373 | 2-Cl | NH—(CH₂)₂—O—(CH₂)₂—O—C(O)H |
| 374 | 2-Cl | NH—(CH₂)₂—NH—(CH₂)₂—OH |
| 375 | 2-Cl | NH—(CH₂)₂—NH—(CH₂)₂—NH₂ |
| 376 | 2-Cl | NH—(CH₂)₂—NH—(CH₂)₂—NHCH₃ |
| 377 | 2-Cl | NH—(CH₂)₂—NH—(CH₂)₂—N(CH₃)₂ |
| 378 | 2-Cl | NH—(CH₂)₂—NH—(CH₂)₂—O—C(O)H |
| 379 | 2-Cl | NH—(CH₂)₂—N(CH₃)—(CH₂)₂—OH |
| 380 | 2-Cl | NH—(CH₂)₂—N(CH₃)—(CH₂)₂—NH₂ |
| 381 | 2-Cl | NH—(CH₂)₂—N(CH₃)—(CH₂)₂—NHCH₃ |
| 382 | 2-Cl | NH—(CH₂)₂—N(CH₃)—(CH₂)₂—N(CH₃)₂ |
| 383 | 2-Cl | NH—(CH₂)₂—N(CH₃)—(CH₂)₂—O—C(O)H |
| 384 | 2-Cl | N(CH₃)—(CH₂)₂—OH |
| 385 | 2-Cl | N(CH₃)—(CH₂)₂—NH₂ |
| 386 | 2-Cl | N(CH₃)—(CH₂)₂—NHCH₃ |
| 387 | 2-Cl | N(CH₃)—(CH₂)₂—N(CH₃)₂ |
| 388 | 2-Cl | N(CH₃)—(CH₂)₂—O—C(O)H |
| 389 | 2-Cl | N(CH₃)—(CH₂)₃—OH |
| 390 | 2-Cl | N(CH₃)—(CH₂)₃—NH₂ |
| 391 | 2-Cl | N(CH₃)—(CH₂)₃—NHCH₃ |
| 392 | 2-Cl | N(CH₃)—(CH₂)₃—N(CH₃)₂ |
| 393 | 2-Cl | N(CH₃)—(CH₂)₃—O—C(O)H |
| 394 | 2-Cl | N(CH₃)—CH(CH₃)—CH₂—OH |
| 395 | 2-Cl | N(CH₃)—CH(CH₃)—CH₂—NH₂ |
| 396 | 2-Cl | N(CH₃)—CH(CH₃)—CH₂—NHCH₃ |
| 397 | 2-Cl | N(CH₃)—CH(CH₃)—CH₂—N(CH₃)₂ |
| 398 | 2-Cl | N(CH₃)—CH(CH₃)—CH₂—O—C(O)H |
| 399 | 2-Cl | N(CH₃)—CH₂—CH(CH₃)—OH |
| 400 | 2-Cl | N(CH₃)—CH₂—CH(CH₃)—NH₂ |
| 401 | 2-Cl | N(CH₃)—CH₂—CH(CH₃)—NHCH₃ |
| 402 | 2-Cl | N(CH₃)—CH₂—CH(CH₃)—N(CH₃)₂ |
| 403 | 2-Cl | N(CH₃)—CH₂—CH(CH₃)—O—C(O)H |
| 404 | 2-Cl | N(CH₃)—(CH₂)₂—O—(CH₂)₂—OH |
| 405 | 2-Cl | N(CH₃)—(CH₂)₂—O—(CH₂)₂—NH₂ |
| 406 | 2-Cl | N(CH₃)—(CH₂)₂—O—(CH₂)₂—NHCH₃ |
| 407 | 2-Cl | N(CH₃)—(CH₂)₂—O—(CH₂)₂—N(CH₃)₂ |
| 408 | 2-Cl | N(CH₃)—(CH₂)₂—O—(CH₂)₂—O—C(O)H |
| 409 | 2-Cl | N(CH₃)—(CH₂)₂—NH—(CH₂)₂—OH |
| 410 | 2-Cl | N(CH₃)—(CH₂)₂—NH—(CH₂)₂—NH₂ |
| 411 | 2-Cl | N(CH₃)—(CH₂)₂—NH—(CH₂)₂—NHCH₃ |
| 412 | 2-Cl | N(CH₃)—(CH₂)₂—NH—(CH₂)₂—N(CH₃)₂ |
| 413 | 2-Cl | N(CH₃)—(CH₂)₂—NH—(CH₂)₂—O—C(O)H |
| 414 | 2-Cl | N(CH₃)—(CH₂)₂—N(CH₃)—(CH₂)₂—OH |
| 415 | 2-Cl | N(CH₃)—(CH₂)₂—N(CH₃)—(CH₂)₂—NH₂ |
| 416 | 2-Cl | N(CH₃)—(CH₂)₂—N(CH₃)—(CH₂)₂—NHCH₃ |
| 417 | 2-Cl | N(CH₃)—(CH₂)₂—N(CH₃)—(CH₂)₂—N(CH₃)₂ |
| 418 | 2-Cl | N(CH₃)—(CH₂)₂—N(CH₃)—(CH₂)₂—O—C(O)H |
| 419 | 2-CH3 | C(O)NH—(CH₂)₂—OH |
| 420 | 2-CH3 | C(O)NH—(CH₂)₂—NH₂ |
| 421 | 2-CH3 | C(O)NH—(CH₂)₂—NHCH₃ |
| 422 | 2-CH3 | C(O)NH—(CH₂)₂—N(CH₃)₂ |
| 423 | 2-CH3 | C(O)NH—(CH₂)₂—O—C(O)H |
| 424 | 2-CH3 | C(O)NH—(CH₂)₃—OH |
| 425 | 2-CH3 | C(O)NH—(CH₂)₃—NH₂ |
| 426 | 2-CH3 | C(O)NH—(CH₂)₃—NHCH₃ |
| 427 | 2-CH3 | C(O)NH—(CH₂)₃—N(CH₃)₂ |
| 428 | 2-CH3 | C(O)NH—(CH₂)₃—O—C(O)H |
| 429 | 2-CH3 | C(O)NH—CH(CH₃)—CH₂—OH |
| 430 | 2-CH3 | C(O)NH—CH(CH₃)—CH₂—NH₂ |
| 431 | 2-CH3 | C(O)NH—CH(CH₃)—CH₂—NHCH₃ |
| 432 | 2-CH3 | C(O)NH—CH(CH₃)—CH₂—N(CH₃)₂ |
| 433 | 2-CH3 | C(O)NH—CH(CH₃)—CH₂—O—C(O)H |
| 434 | 2-CH3 | C(O)NH—CH₂—CH(CH₃)—OH |
| 435 | 2-CH3 | C(O)NH—CH₂—CH(CH₃)—NH₂ |
| 436 | 2-CH3 | C(O)NH—CH₂—CH(CH₃)—NHCH₃ |
| 437 | 2-CH3 | C(O)NH—CH₂—CH(CH₃)—N(CH₃)₂ |
| 438 | 2-CH3 | C(O)NH—CH₂—CH(CH₃)—O—C(O)H |
| 439 | 2-CH3 | C(O)NH—(CH₂)₂—O—(CH₂)₂—OH |
| 440 | 2-CH3 | C(O)NH—(CH₂)₂—O—(CH₂)₂—NH₂ |
| 441 | 2-CH3 | C(O)NH—(CH₂)₂—O—(CH₂)₂—NHCH₃ |
| 442 | 2-CH3 | C(O)NH—(CH₂)₂—O—(CH₂)₂—N(CH₃)₂ |
| 443 | 2-CH3 | C(O)NH—(CH₂)₂—O—(CH₂)₂—O—C(O)H |
| 444 | 2-CH3 | C(O)NH—(CH₂)₂—NH—(CH₂)₂—OH |
| 445 | 2-CH3 | C(O)NH—(CH₂)₂—NH—(CH₂)₂—NH₂ |
| 446 | 2-CH3 | C(O)NH—(CH₂)₂—NH—(CH₂)₂—NHCH₃ |
| 447 | 2-CH3 | C(O)NH—(CH₂)₂—NH—(CH₂)₂—N(CH₃)₂ |
| 448 | 2-CH3 | C(O)NH—(CH₂)₂—NH—(CH₂)₂—O—C(O)H |
| 449 | 2-CH3 | C(O)NH—(CH₂)₂—N(CH₃)—(CH₂)₂—OH |
| 450 | 2-CH3 | C(O)NH—(CH₂)₂—N(CH₃)—(CH₂)₂—NH₂ |
| 451 | 2-CH3 | C(O)NH—(CH₂)₂—N(CH₃)—(CH₂)₂—NHCH₃ |
| 452 | 2-CH3 | C(O)NH—(CH₂)₂—N(CH₃)—(CH₂)₂—N(CH₃)₂ |
| 453 | 2-CH3 | C(O)NH—(CH₂)₂—N(CH₃)—(CH₂)₂—O—C(O)H |
| 454 | 2-CH3 | C(O)N(CH₃)—(CH₂)₂—OH |
| 455 | 2-CH3 | C(O)N(CH₃)—(CH₂)₂—NH₂ |
| 456 | 2-CH3 | C(O)N(CH₃)—(CH₂)₂—NHCH₃ |
| 457 | 2-CH3 | C(O)N(CH₃)—(CH₂)₂—N(CH₃)₂ |
| 458 | 2-CH3 | C(O)N(CH₃)—(CH₂)₂—O—C(O)H |
| 459 | 2-CH3 | C(O)N(CH₃)—(CH₂)₃—OH |
| 460 | 2-CH3 | C(O)N(CH₃)—(CH₂)₃—NH₂ |
| 461 | 2-CH3 | C(O)N(CH₃)—(CH₂)₃—NHCH₃ |
| 462 | 2-CH3 | C(O)N(CH₃)—(CH₂)₃—N(CH₃)₂ |
| 463 | 2-CH3 | C(O)N(CH₃)—(CH₂)₃—O—C(O)H |
| 464 | 2-CH3 | C(O)N(CH₃)—CH(CH₃)—CH₂—OH |
| 465 | 2-CH3 | C(O)N(CH₃)—CH(CH₃)—CH₂—NH₂ |
| 466 | 2-CH3 | C(O)N(CH₃)—CH(CH₃)—CH₂—NHCH₃ |
| 467 | 2-CH3 | C(O)N(CH₃)—CH(CH₃)—CH₂—N(CH₃)₂ |
| 468 | 2-CH3 | C(O)N(CH₃)—CH(CH₃)—CH₂—O—C(O)H |
| 469 | 2-CH3 | C(O)N(CH₃)—CH₂—CH(CH₃)—OH |
| 470 | 2-CH3 | C(O)N(CH₃)—CH₂—CH(CH₃)—NH₂ |
| 471 | 2-CH3 | C(O)N(CH₃)—CH₂—CH(CH₃)—NHCH₃ |
| 472 | 2-CH3 | C(O)N(CH₃)—CH₂—CH(CH₃)—N(CH₃)₂ |
| 473 | 2-CH3 | C(O)N(CH₃)—(CH₂)₂—O—(CH₂)₂—OH |
| 474 | 2-CH3 | C(O)N(CH₃)—(CH₂)₂—O—(CH₂)₂—NH₂ |
| 475 | 2-CH3 | C(O)N(CH₃)—(CH₂)₂—O—(CH₂)₂—NHCH₃ |
| 476 | 2-CH3 | C(O)N(CH₃)—(CH₂)₂—O—(CH₂)₂—N(CH₃)₂ |
| 477 | 2-CH3 | C(O)N(CH₃)—(CH₂)₂—O—(CH₂)₂—O—C(O)H |
| 478 | 2-CH3 | C(O)N(CH₃)—(CH₂)₂—NH—(CH₂)₂—OH |
| 479 | 2-CH3 | C(O)N(CH₃)—(CH₂)₂—NH—(CH₂)₂—NH₂ |
| 480 | 2-CH3 | C(O)N(CH₃)—(CH₂)₂—NH—(CH₂)₂—NHCH₃ |
| 481 | 2-CH3 | C(O)N(CH₃)—(CH₂)₂—NH—(CH₂)₂—N(CH₃)₂ |
| 482 | 2-CH3 | C(O)N(CH₃)—(CH₂)₂—NH—(CH₂)₂—O—C(O)H |
| 483 | 2-CH3 | C(O)N(CH₃)—(CH₂)₂—N(CH₃)—(CH₂)₂—OH |
| 484 | 2-CH3 | C(O)N(CH₃)—(CH₂)₂—N(CH₃)—(CH₂)₂—NH₂ |
| 485 | 2-CH3 | C(O)N(CH₃)—(CH₂)₂—N(CH₃)—(CH₂)₂—NHCH₃ |
| 486 | 2-CH3 | C(O)N(CH₃)—(CH₂)₂—N(CH₃)—(CH₂)₂—N(CH₃)₂ |
| 487 | 2-CH3 | C(O)N(CH₃)—(CH₂)₂—N(CH₃)—(CH₂)₂—O— |
| | | C(O)H |
| 488 | 2-CH3 | C(O)O—(CH₂)₂—OH |
| 489 | 2-CH3 | C(O)O—(CH₂)₂—NH₂ |
| 490 | 2-CH3 | C(O)O—(CH₂)₂—NHCH₃ |
| 491 | 2-CH3 | C(O)O—(CH₂)₂—N(CH₃)₂ |
| 492 | 2-CH3 | C(O)O—(CH₂)₂—O—C(O)H |
| 493 | 2-CH3 | C(O)O—(CH₂)₃—OH |
| 494 | 2-CH3 | C(O)O—(CH₂)₃—NH₂ |
| 495 | 2-CH3 | C(O)O—(CH₂)₃—NHCH₃ |
| 496 | 2-CH3 | C(O)O—(CH₂)₃—N(CH₃)₂ |
| 497 | 2-CH3 | C(O)O—(CH₂)₃—O—C(O)H |
| 498 | 2-CH3 | C(O)O—CH(CH₃)—CH₂—OH |
| 499 | 2-CH3 | C(O)O—CH(CH₃)—CH₂—NH₂ |
| 500 | 2-CH3 | C(O)O—CH(CH₃)—CH₂—NHCH₃ |
| 501 | 2-CH3 | C(O)O—CH(CH₃)—CH₂—N(CH₃)₂ |
| 502 | 2-CH3 | C(O)O—CH(CH₃)—CH₂—O—C(O)H |
| 503 | 2-CH3 | C(O)O—CH₂—CH(CH₃)—OH |
| 504 | 2-CH3 | C(O)O—CH₂—CH(CH₃)—NH₂ |
| 505 | 2-CH3 | C(O)O—CH₂—CH(CH₃)—NHCH₃ |
| 506 | 2-CH3 | C(O)O—CH₂—CH(CH₃)—N(CH₃)₂ |
| 507 | 2-CH3 | C(O)O—CH₂—CH(CH₃)—O—C(O)H |
| 508 | 2-CH3 | C(O)O—(CH₂)₂—O—(CH₂)₂—OH |
| 509 | 2-CH3 | C(O)O—(CH₂)₂—O—(CH₂)₂—NH₂ |
| 510 | 2-CH3 | C(O)O—(CH₂)₂—O—(CH₂)₂—NHCH₃ |
| 511 | 2-CH3 | C(O)O—(CH₂)₂—O—(CH₂)₂—N(CH₃)₂ |
| 512 | 2-CH3 | C(O)O—(CH₂)₂—O—(CH₂)₂—O—C(O)H |
| 513 | 2-CH3 | C(O)O—(CH₂)₂—NH—(CH₂)₂—OH |
| 514 | 2-CH3 | C(O)O—(CH₂)₂—NH—(CH₂)₂—NH₂ |
| 515 | 2-CH3 | C(O)O—(CH₂)₂—NH—(CH₂)₂—NHCH₃ |
| 516 | 2-CH3 | C(O)O—(CH₂)₂—NH—(CH₂)₂—N(CH₃)₂ |
| 517 | 2-CH3 | C(O)O—(CH₂)₂—NH—(CH₂)₂—O—C(O)H |
| 518 | 2-CH3 | C(O)O—(CH₂)₂—N(CH₃)—(CH₂)₂—OH |
| 519 | 2-CH3 | C(O)O—(CH₂)₂—N(CH₃)—(CH₂)₂—NH₂ |
| 520 | 2-CH3 | C(O)O—(CH₂)₂—N(CH₃)—(CH₂)₂—NHCH₃ |
| 521 | 2-CH3 | C(O)O—(CH₂)₂—N(CH₃)—(CH₂)₂—N(CH₃)₂ |
| 522 | 2-CH3 | C(O)O—(CH₂)₂—N(CH₃)—(CH₂)₂—O—C(O)H |
| 523 | 2-CH3 | O—(CH₂)₂—OH |
| 524 | 2-CH3 | O—(CH₂)₂—NH₂ |
| 525 | 2-CH3 | O—(CH₂)₂—NHCH₃ |
| 526 | 2-CH3 | O—(CH₂)₂—N(CH₃)₂ |
| 527 | 2-CH3 | O—(CH₂)₂—O—C(O)H |
| 528 | 2-CH3 | O—(CH₂)₃—OH |
| 529 | 2-CH3 | O—(CH₂)₃—NH₂ |
| 530 | 2-CH3 | O—(CH₂)₃—NHCH₃ |
| 531 | 2-CH3 | O—(CH₂)₃—N(CH₃)₂ |
| 532 | 2-CH3 | O—(CH₂)₃—O—C(O)H |
| 533 | 2-CH3 | O—CH(CH₃)—CH₂—OH |
| 534 | 2-CH3 | O—CH(CH₃)—CH₂—NH₂ |
| 535 | 2-CH3 | O—CH(CH₃)—CH₂—NHCH₃ |
| 536 | 2-CH3 | O—CH(CH₃)—CH₂—N(CH₃)₂ |
| 537 | 2-CH3 | O—CH(CH₃)—CH₂—O—C(O)H |
| 538 | 2-CH3 | O—CH₂—CH(CH₃)—OH |
| 539 | 2-CH3 | O—CH₂—CH(CH₃)—NH₂ |
| 540 | 2-CH3 | O—CH₂—CH(CH₃)—NHCH₃ |
| 541 | 2-CH3 | O—CH₂—CH(CH₃)—N(CH₃)₂ |
| 542 | 2-CH3 | O—CH₂—CH(CH₃)—O—C(O)H |
| 543 | 2-CH3 | O—(CH₂)₂—O—(CH₂)₂—OH |
| 544 | 2-CH3 | O—(CH₂)₂—O—(CH₂)₂—NH₂ |
| 545 | 2-CH3 | O—(CH₂)₂—O—(CH₂)₂—NHCH₃ |
| 546 | 2-CH3 | O—(CH₂)₂—O—(CH₂)₂—N(CH₃)₂ |
| 547 | 2-CH3 | O—(CH₂)₂—O—(CH₂)₂—O—C(O)H |
| 548 | 2-CH3 | O—(CH₂)₂—NH—(CH₂)₂—OH |
| 549 | 2-CH3 | O—(CH₂)₂—NH—(CH₂)₂—NH₂ |
| 550 | 2-CH3 | O—(CH₂)₂—NH—(CH₂)₂—NHCH₃ |
| 551 | 2-CH3 | O—(CH₂)₂—NH—(CH₂)₂—N(CH₃)₂ |
| 552 | 2-CH3 | O—(CH₂)₂—NH—(CH₂)₂—O—C(O)H |
| 553 | 2-CH3 | O—(CH₂)₂—N(CH₃)—(CH₂)₂—OH |
| 554 | 2-CH3 | O—(CH₂)₂—N(CH₃)—(CH₂)₂—NH₂ |
| 555 | 2-CH3 | O—(CH₂)₂—N(CH₃)—(CH₂)₂—NHCH₃ |
| 556 | 2-CH3 | O—(CH₂)₂—N(CH₃)—(CH₂)₂—N(CH₃)₂ |
| 557 | 2-CH3 | O—(CH₂)₂—N(CH₃)—(CH₂)₂—O—C(O)H |
| 558 | 2-CH3 | NH—(CH₂)₂—OH |
| 559 | 2-CH3 | NH—(CH₂)₂—NH₂ |
| 560 | 2-CH3 | NH—(CH₂)₂—NHCH₃ |
| 561 | 2-CH3 | NH—(CH₂)₂—N(CH₃)₂ |
| 562 | 2-CH3 | NH—(CH₂)₂—O—C(O)H |
| 563 | 2-CH3 | NH—(CH₂)₃—OH |
| 564 | 2-CH3 | NH—(CH₂)₃—NH₂ |
| 565 | 2-CH3 | NH—(CH₂)₃—NHCH₃ |
| 566 | 2-CH3 | NH—(CH₂)₃—N(CH₃)₂ |
| 567 | 2-CH3 | NH—(CH₂)₃—O—C(O)H |
| 568 | 2-CH3 | NH—CH(CH₃)—CH₂—OH |
| 569 | 2-CH3 | NH—CH(CH₃)—CH₂—NH₂ |
| 570 | 2-CH3 | NH—CH(CH₃)—CH₂—NHCH₃ |
| 571 | 2-CH3 | NH—CH(CH₃)—CH₂—N(CH₃)₂ |
| 572 | 2-CH3 | NH—CH(CH₃)—CH₂—O—C(O)H |
| 573 | 2-CH3 | NH—CH₂—CH(CH₃)—OH |
| 574 | 2-CH3 | NH—CH₂—CH(CH₃)—NH₂ |
| 575 | 2-CH3 | NH—CH₂—CH(CH₃)—NHCH₃ |
| 576 | 2-CH3 | NH—CH₂—CH(CH₃)—N(CH₃)₂ |
| 577 | 2-CH3 | NH—CH₂—CH(CH₃)—O—C(O)H |
| 578 | 2-CH3 | NH—(CH₂)₂—O—(CH₂)₂—OH |
| 579 | 2-CH3 | NH—(CH₂)₂—O—(CH₂)₂—NH₂ |
| 580 | 2-CH3 | NH—(CH₂)₂—O—(CH₂)₂—NHCH₃ |
| 581 | 2-CH3 | NH—(CH₂)₂—O—(CH₂)₂—N(CH₃)₂ |
| 582 | 2-CH3 | NH—(CH₂)₂—O—(CH₂)₂—O—C(O)H |
| 583 | 2-CH3 | NH—(CH₂)₂—NH—(CH₂)₂—OH |
| 584 | 2-CH3 | NH—(CH₂)₂—NH—(CH₂)₂—NH₂ |
| 585 | 2-CH3 | NH—(CH₂)₂—NH—(CH₂)₂—NHCH₃ |
| 586 | 2-CH3 | NH—(CH₂)₂—NH—(CH₂)₂—N(CH₃)₂ |
| 587 | 2-CH3 | NH—(CH₂)₂—NH—(CH₂)₂—O—C(O)H |
| 588 | 2-CH3 | NH—(CH₂)₂—N(CH₃)—(CH₂)₂—OH |
| 589 | 2-CH3 | NH—(CH₂)₂—N(CH₃)—(CH₂)₂—NH₂ |
| 590 | 2-CH3 | NH—(CH₂)₂—N(CH₃)—(CH₂)₂—NHCH₃ |
| 591 | 2-CH3 | NH—(CH₂)₂—N(CH₃)—(CH₂)₂—N(CH₃)₂ |
| 592 | 2-CH3 | NH—(CH₂)₂—N(CH₃)—(CH₂)₂—O—C(O)H |
| 593 | 2-CH3 | N(CH₃)—(CH₂)₂—OH |
| 594 | 2-CH3 | N(CH₃)—(CH₂)₂—NH₂ |
| 595 | 2-CH3 | N(CH₃)—(CH₂)₂—NHCH₃ |
| 596 | 2-CH3 | N(CH₃)—(CH₂)₂—N(CH₃)₂ |
| 597 | 2-CH3 | N(CH₃)—(CH₂)₂—O—C(O)H |
| 598 | 2-CH3 | N(CH₃)—(CH₂)₃—OH |
| 599 | 2-CH3 | N(CH₃)—(CH₂)₃—NH₂ |
| 600 | 2-CH3 | N(CH₃)—(CH₂)₃—NHCH₃ |
| 601 | 2-CH3 | N(CH₃)—(CH₂)₃—N(CH₃)₂ |
| 602 | 2-CH3 | N(CH₃)—(CH₂)₃—O—C(O)H |
| 603 | 2-CH3 | N(CH₃)—CH(CH₃)—CH₂—OH |
| 604 | 2-CH3 | N(CH₃)—CH(CH₃)—CH₂—NH₂ |
| 605 | 2-CH3 | N(CH₃)—CH(CH₃)—CH₂—NHCH₃ |
| 606 | 2-CH3 | N(CH₃)—CH(CH₃)—CH₂—N(CH₃)₂ |
| 607 | 2-CH3 | N(CH₃)—CH(CH₃)—CH₂—O—C(O)H |
| 608 | 2-CH3 | N(CH₃)—CH₂—CH(CH₃)—OH |
| 609 | 2-CH3 | N(CH₃)—CH₂—CH(CH₃)—NH₂ |
| 610 | 2-CH3 | N(CH₃)—CH₂—CH(CH₃)—NHCH₃ |
| 611 | 2-CH3 | N(CH₃)—CH₂—CH(CH₃)—N(CH₃)₂ |
| 612 | 2-CH3 | N(CH₃)—CH₂—CH(CH₃)—O—C(O)H |
| 613 | 2-CH3 | N(CH₃)—(CH₂)₂—O—(CH₂)₂—OH |
| 614 | 2-CH3 | N(CH₃)—(CH₂)₂—O—(CH₂)₂—NH₂ |
| 615 | 2-CH3 | N(CH₃)—(CH₂)₂—O—(CH₂)₂—NHCH₃ |
| 616 | 2-CH3 | N(CH₃)—(CH₂)₂—O—(CH₂)₂—N(CH₃)₂ |
| 617 | 2-CH3 | N(CH₃)—(CH₂)₂—O—(CH₂)₂—O—C(O)H |
| 618 | 2-CH3 | N(CH₃)—(CH₂)₂—NH—(CH₂)₂—OH |
| 619 | 2-CH3 | N(CH₃)—(CH₂)₂—NH—(CH₂)₂—NH₂ |
| 620 | 2-CH3 | N(CH₃)—(CH₂)₂—NH—(CH₂)₂—NHCH₃ |
| 621 | 2-CH3 | N(CH₃)—(CH₂)₂—NH—(CH₂)₂—N(CH₃)₂ |
| 622 | 2-CH3 | N(CH₃)—(CH₂)₂—NH—(CH₂)₂—O—C(O)H |
| 623 | 2-CH3 | N(CH₃)—(CH₂)₂—N(CH₃)—(CH₂)₂—OH |
| 624 | 2-CH3 | N(CH₃)—(CH₂)₂—N(CH₃)—(CH₂)₂—NH₂ |
| 625 | 2-CH3 | N(CH₃)—(CH₂)₂—N(CH₃)—(CH₂)₂—NHCH₃ |
| 626 | 2-CH3 | N(CH₃)—(CH₂)₂—N(CH₃)—(CH₂)₂—N(CH₃)₂ |
| 627 | 2-CH3 | N(CH₃)—(CH₂)₂—N(CH₃)—(CH₂)₂—O—C(O)H |
| 628 | 2,6-F2 | C(O)NH—(CH₂)₂—OH |
| 629 | 2,6-F2 | C(O)NH—(CH₂)₂—NH₂ |
| 630 | 2,6-F2 | C(O)NH—(CH₂)₂—NHCH₃ |
| 631 | 2,6-F2 | C(O)NH—(CH₂)₂—N(CH₃)₂ |
| 632 | 2,6-F2 | C(O)NH—(CH₂)₂—O—C(O)H |
| 633 | 2,6-F2 | C(O)NH—(CH₂)₃—OH |
| 634 | 2,6-F2 | C(O)NH—(CH₂)₃—NH₂ |
| 635 | 2,6-F2 | C(O)NH—(CH₂)₃—NHCH₃ |
| 636 | 2,6-F2 | C(O)NH—(CH₂)₃—N(CH₃)₂ |
| 637 | 2,6-F2 | C(O)NH—(CH₂)₃—O—C(O)H |
| 638 | 2,6-F2 | C(O)NH—CH(CH₃)—CH₂—OH |
| 639 | 2,6-F2 | C(O)NH—CH(CH₃)—CH₂—NH₂ |
| 640 | 2,6-F2 | C(O)NH—CH(CH₃)—CH₂—NHCH₃ |
| 641 | 2,6-F2 | C(O)NH—CH(CH₃)—CH₂—N(CH₃)₂ |
| 642 | 2,6-F2 | C(O)NH—CH(CH₃)—CH₂—O—C(O)H |
| 643 | 2,6-F2 | C(O)NH—CH₂—CH(CH₃)—OH |
| 644 | 2,6-F2 | C(O)NH—CH₂—CH(CH₃)—NH₂ |
| 645 | 2,6-F2 | C(O)NH—CH₂—CH(CH₃)—NHCH₃ |
| 646 | 2,6-F2 | C(O)NH—CH₂—CH(CH₃)—N(CH₃)₂ |
| 647 | 2,6-F2 | C(O)NH—CH₂—CH(CH₃)—O—C(O)H |
| 648 | 2,6-F2 | C(O)NH—(CH₂)₂—O—(CH₂)₂—OH |
| 649 | 2,6-F2 | C(O)NH—(CH₂)₂—O—(CH₂)₂—NH₂ |
| 650 | 2,6-F2 | C(O)NH—(CH₂)₂—O—(CH₂)₂—NHCH₃ |
| 651 | 2,6-F2 | C(O)NH—(CH₂)₂—O—(CH₂)₂—N(CH₃)₂ |
| 652 | 2,6-F2 | C(O)NH—(CH₂)₂—O—(CH₂)₂—O—C(O)H |
| 653 | 2,6-F2 | C(O)NH—(CH₂)₂—NH—(CH₂)₂—OH |
| 654 | 2,6-F2 | C(O)NH—(CH₂)₂—NH—(CH₂)₂—NH₂ |
| 655 | 2,6-F2 | C(O)NH—(CH₂)₂—NH—(CH₂)₂—NHCH₃ |
| 656 | 2,6-F2 | C(O)NH—(CH₂)₂—NH—(CH₂)₂—N(CH₃)₂ |
| 657 | 2,6-F2 | C(O)NH—(CH₂)₂—NH—(CH₂)₂—O—C(O)H |
| 658 | 2,6-F2 | C(O)NH—(CH₂)₂—N(CH₃)—(CH₂)₂—OH |
| 659 | 2,6-F2 | C(O)NH—(CH₂)₂—N(CH₃)—(CH₂)₂—NH₂ |
| 660 | 2,6-F2 | C(O)NH—(CH₂)₂—N(CH₃)—(CH₂)₂—NHCH₃ |
| 661 | 2,6-F2 | C(O)NH—(CH₂)₂—N(CH₃)—(CH₂)₂—N(CH₃)₂ |
| 662 | 2,6-F2 | C(O)NH—(CH₂)₂—N(CH₃)—(CH₂)₂—O—C(O)H |
| 663 | 2,6-F2 | C(O)N(CH₃)—(CH₂)₂—OH |
| 664 | 2,6-F2 | C(O)N(CH₃)—(CH₂)₂—NH₂ |
| 665 | 2,6-F2 | C(O)N(CH₃)—(CH₂)₂—NHCH₃ |
| 666 | 2,6-F2 | C(O)N(CH₃)—(CH₂)₂—N(CH₃)₂ |
| 667 | 2,6-F2 | C(O)N(CH₃)—(CH₂)₂—O—C(O)H |
| 668 | 2,6-F2 | C(O)N(CH₃)—(CH₂)₃—OH |
| 669 | 2,6-F2 | C(O)N(CH₃)—(CH₂)₃—NH₂ |
| 670 | 2,6-F2 | C(O)N(CH₃)—(CH₂)₃—NHCH₃ |
| 671 | 2,6-F2 | C(O)N(CH₃)—(CH₂)₃—N(CH₃)₂ |
| 672 | 2,6-F2 | C(O)N(CH₃)—(CH₂)₃—O—C(O)H |
| 673 | 2,6-F2 | C(O)N(CH₃)—CH(CH₃)—CH₂—OH |
| 674 | 2,6-F2 | C(O)N(CH₃)—CH(CH₃)—CH₂—NH₂ |
| 675 | 2,6-F2 | C(O)N(CH₃)—CH(CH₃)—CH₂—NHCH₃ |
| 676 | 2,6-F2 | C(O)N(CH₃)—CH(CH₃)—CH₂—N(CH₃)₂ |
| 677 | 2,6-F2 | C(O)N(CH₃)—CH(CH₃)—CH₂—O—C(O)H |
| 678 | 2,6-F2 | C(O)N(CH₃)—CH₂—CH(CH₃)—OH |
| 679 | 2,6-F2 | C(O)N(CH₃)—CH₂—CH(CH₃)—NH₂ |
| 680 | 2,6-F2 | C(O)N(CH₃)—CH₂—CH(CH₃)—NHCH₃ |
| 681 | 2,6-F2 | C(O)N(CH₃)—CH₂—CH(CH₃)—N(CH₃)₂ |
| 682 | 2,6-F2 | C(O)N(CH₃)—(CH₂)₂—O—(CH₂)₂—OH |
| 683 | 2,6-F2 | C(O)N(CH₃)—(CH₂)₂—O—(CH₂)₂—NH₂ |
| 684 | 2,6-F2 | C(O)N(CH₃)—(CH₂)₂—O—(CH₂)₂—NHCH₃ |
| 685 | 2,6-F2 | C(O)N(CH₃)—(CH₂)₂—O—(CH₂)₂—N(CH₃)₂ |
| 686 | 2,6-F2 | C(O)N(CH₃)—(CH₂)₂—O—(CH₂)₂—O—C(O)H |
| 687 | 2,6-F2 | C(O)N(CH₃)—(CH₂)₂—NH—(CH₂)₂—OH |
| 688 | 2,6-F2 | C(O)N(CH₃)—(CH₂)₂—NH—(CH₂)₂—NH₂ |
| 689 | 2,6-F2 | C(O)N(CH₃)—(CH₂)₂—NH—(CH₂)₂—NHCH₃ |
| 690 | 2,6-F2 | C(O)N(CH₃)—(CH₂)₂—NH—(CH₂)₂—N(CH₃)₂ |
| 691 | 2,6-F2 | C(O)N(CH₃)—(CH₂)₂—NH—(CH₂)₂—O—C(O)H |
| 692 | 2,6-F2 | C(O)N(CH₃)—(CH₂)₂—N(CH₃)—(CH₂)₂—OH |
| 693 | 2,6-F2 | C(O)N(CH₃)—(CH₂)₂—N(CH₃)—(CH₂)₂—NH₂ |
| 694 | 2,6-F2 | C(O)N(CH₃)—(CH₂)₂—N(CH₃)—(CH₂)₂—NHCH₃ |
| 695 | 2,6-F2 | C(O)N(CH₃)—(CH₂)₂—N(CH₃)—(CH₂)₂—N(CH₃)₂ |
| 696 | 2,6-F2 | C(O)N(CH₃)—(CH₂)₂—N(CH₃)—(CH₂)₂—O— |
| | | C(O)H |
| 697 | 2,6-F2 | C(O)O—(CH₂)₂—OH |
| 698 | 2,6-F2 | C(O)O—(CH₂)₂—NH₂ |
| 699 | 2,6-F2 | C(O)O—(CH₂)₂—NHCH₃ |
| 700 | 2,6-F2 | C(O)O—(CH₂)₂—N(CH₃)₂ |
| 701 | 2,6-F2 | C(O)O—(CH₂)₂—O—C(O)H |
| 702 | 2,6-F2 | C(O)O—(CH₂)₃—OH |
| 703 | 2,6-F2 | C(O)O—(CH₂)₃—NH₂ |
| 704 | 2,6-F2 | C(O)O—(CH₂)₃—NHCH₃ |
| 705 | 2,6-F2 | C(O)O—(CH₂)₃—N(CH₃)₂ |
| 706 | 2,6-F2 | C(O)O—(CH₂)₃—O—C(O)H |
| 707 | 2,6-F2 | C(O)O—CH(CH₃)—CH₂—OH |
| 708 | 2,6-F2 | C(O)O—CH(CH₃)—CH₂—NH₂ |
| 709 | 2,6-F2 | C(O)O—CH(CH₃)—CH₂—NHCH₃ |
| 710 | 2,6-F2 | C(O)O—CH(CH₃)—CH₂—N(CH₃)₂ |
| 711 | 2,6-F2 | C(O)O—CH(CH₃)—CH₂—O—C(O)H |
| 712 | 2,6-F2 | C(O)O—CH₂—CH(CH₃)—OH |
| 713 | 2,6-F2 | C(O)O—CH₂—CH(CH₃)—NH₂ |
| 714 | 2,6-F2 | C(O)O—CH₂—CH(CH₃)—NHCH₃ |
| 715 | 2,6-F2 | C(O)O—CH₂—CH(CH₃)—N(CH₃)₂ |
| 716 | 2,6-F2 | C(O)O—CH₂—CH(CH₃)—O—C(O)H |
| 717 | 2,6-F2 | C(O)O—(CH₂)₂—O—(CH₂)₂—OH |
| 718 | 2,6-F2 | C(O)O—(CH₂)₂—O—(CH₂)₂—NH₂ |
| 719 | 2,6-F2 | C(O)O—(CH₂)₂—O—(CH₂)₂—NHCH₃ |
| 720 | 2,6-F2 | C(O)O—(CH₂)₂—O—(CH₂)₂—N(CH₃)₂ |
| 721 | 2,6-F2 | C(O)O—(CH₂)₂—O—(CH₂)₂—O—C(O)H |
| 722 | 2,6-F2 | C(O)O—(CH₂)₂—NH—(CH₂)₂—OH |
| 723 | 2,6-F2 | C(O)O—(CH₂)₂—NH—(CH₂)₂—NH₂ |
| 724 | 2,6-F2 | C(O)O—(CH₂)₂—NH—(CH₂)₂—NHCH₃ |
| 725 | 2,6-F2 | C(O)O—(CH₂)₂—NH—(CH₂)₂—N(CH₃)₂ |
| 726 | 2,6-F2 | C(O)O—(CH₂)₂—NH—(CH₂)₂—O—C(O)H |
| 727 | 2,6-F2 | C(O)O—(CH₂)₂—N(CH₃)—(CH₂)₂—OH |
| 728 | 2,6-F2 | C(O)O—(CH₂)₂—N(CH₃)—(CH₂)₂—NH₂ |
| 729 | 2,6-F2 | C(O)O—(CH₂)₂—N(CH₃)—(CH₂)₂—NHCH₃ |
| 730 | 2,6-F2 | C(O)O—(CH₂)₂—N(CH₃)—(CH₂)₂—N(CH₃)₂ |
| 731 | 2,6-F2 | C(O)O—(CH₂)₂—N(CH₃)—(CH₂)₂—O—C(O)H |
| 732 | 2,6-F2 | O—(CH₂)₂—OH |
| 733 | 2,6-F2 | O—(CH₂)₂—NH₂ |
| 734 | 2,6-F2 | O—(CH₂)₂—NHCH₃ |
| 735 | 2,6-F2 | O—(CH₂)₂—N(CH₃)₂ |
| 736 | 2,6-F2 | O—(CH₂)₂—O—C(O)H |
| 737 | 2,6-F2 | O—(CH₂)₃—OH |
| 738 | 2,6-F2 | O—(CH₂)₃—NH₂ |
| 739 | 2,6-F2 | O—(CH₂)₃—NHCH₃ |
| 740 | 2,6-F2 | O—(CH₂)₃—N(CH₃)₂ |
| 741 | 2,6-F2 | O—(CH₂)₃—O—C(O)H |
| 742 | 2,6-F2 | O—CH(CH₃)—CH₂—OH |
| 743 | 2,6-F2 | O—CH(CH₃)—CH₂—NH₂ |
| 744 | 2,6-F2 | O—CH(CH₃)—CH₂—NHCH₃ |
| 745 | 2,6-F2 | O—CH(CH₃)—CH₂—N(CH₃)₂ |
| 746 | 2,6-F2 | O—CH(CH₃)—CH₂—O—C(O)H |
| 747 | 2,6-F2 | O—CH₂—CH(CH₃)—OH |
| 748 | 2,6-F2 | O—CH₂—CH(CH₃)—NH₂ |
| 749 | 2,6-F2 | O—CH₂—CH(CH₃)—NHCH₃ |
| 750 | 2,6-F2 | O—CH₂—CH(CH₃)—N(CH₃)₂ |
| 751 | 2,6-F2 | O—CH₂—CH(CH₃)—O—C(O)H |
| 752 | 2,6-F2 | O—(CH₂)₂—O—(CH₂)₂—OH |
| 753 | 2,6-F2 | O—(CH₂)₂—O—(CH₂)₂—NH₂ |
| 754 | 2,6-F2 | O—(CH₂)₂—O—(CH₂)₂—NHCH₃ |
| 755 | 2,6-F2 | O—(CH₂)₂—O—(CH₂)₂—N(CH₃)₂ |
| 756 | 2,6-F2 | O—(CH₂)₂—O—(CH₂)₂—O—C(O)H |
| 757 | 2,6-F2 | O—(CH₂)₂—NH—(CH₂)₂—OH |
| 758 | 2,6-F2 | O—(CH₂)₂—NH—(CH₂)₂—NH₂ |
| 759 | 2,6-F2 | O—(CH₂)₂—NH—(CH₂)₂—NHCH₃ |
| 760 | 2,6-F2 | O—(CH₂)₂—NH—(CH₂)₂—N(CH₃)₂ |
| 761 | 2,6-F2 | O—(CH₂)₂—NH—(CH₂)₂—O—C(O)H |
| 762 | 2,6-F2 | O—(CH₂)₂—N(CH₃)—(CH₂)₂—OH |
| 763 | 2,6-F2 | O—(CH₂)₂—N(CH₃)—(CH₂)₂—NH₂ |
| 764 | 2,6-F2 | O—(CH₂)₂—N(CH₃)—(CH₂)₂—NHCH₃ |
| 765 | 2,6-F2 | O—(CH₂)₂—N(CH₃)—(CH₂)₂—N(CH₃)₂ |
| 766 | 2,6-F2 | O—(CH₂)₂—N(CH₃)—(CH₂)₂—O—C(O)H |
| 767 | 2,6-F2 | NH—(CH₂)₂—OH |
| 768 | 2,6-F2 | NH—(CH₂)₂—NH₂ |
| 769 | 2,6-F2 | NH—(CH₂)₂—NHCH₃ |
| 770 | 2,6-F2 | NH—(CH₂)₂—N(CH₃)₂ |
| 771 | 2,6-F2 | NH—(CH₂)₂—O—C(O)H |
| 772 | 2,6-F2 | NH—(CH₂)₃—OH |
| 773 | 2,6-F2 | NH—(CH₂)₃—NH₂ |
| 774 | 2,6-F2 | NH—(CH₂)₃—NHCH₃ |
| 775 | 2,6-F2 | NH—(CH₂)₃—N(CH₃)₂ |
| 776 | 2,6-F2 | NH—(CH₂)₃—O—C(O)H |
| 777 | 2,6-F2 | NH—CH(CH₃)—CH₂—OH |
| 778 | 2,6-F2 | NH—CH(CH₃)—CH₂—NH₂ |
| 779 | 2,6-F2 | NH—CH(CH₃)—CH₂—NHCH₃ |
| 780 | 2,6-F2 | NH—CH(CH₃)—CH₂—N(CH₃)₂ |
| 781 | 2,6-F2 | NH—CH(CH₃)—CH₂—O—C(O)H |
| 782 | 2,6-F2 | NH—CH₂—CH(CH₃)—OH |
| 783 | 2,6-F2 | NH—CH₂—CH(CH₃)—NH₂ |
| 784 | 2,6-F2 | NH—CH₂—CH(CH₃)—NHCH₃ |
| 785 | 2,6-F2 | NH—CH₂—CH(CH₃)—N(CH₃)₂ |
| 786 | 2,6-F2 | NH—CH₂—CH(CH₃)—O—C(O)H |
| 787 | 2,6-F2 | NH—(CH₂)₂—O—(CH₂)₂—OH |
| 788 | 2,6-F2 | NH—(CH₂)₂—O—(CH₂)₂—NH₂ |
| 789 | 2,6-F2 | NH—(CH₂)₂—O—(CH₂)₂—NHCH₃ |
| 790 | 2,6-F2 | NH—(CH₂)₂—O—(CH₂)₂—N(CH₃)₂ |
| 791 | 2,6-F2 | NH—(CH₂)₂—O—(CH₂)₂—O—C(O)H |
| 792 | 2,6-F2 | NH—(CH₂)₂—NH—(CH₂)₂—OH |
| 793 | 2,6-F2 | NH—(CH₂)₂—NH—(CH₂)₂—NH₂ |
| 794 | 2,6-F2 | NH—(CH₂)₂—NH—(CH₂)₂—NHCH₃ |
| 795 | 2,6-F2 | NH—(CH₂)₂—NH—(CH₂)₂—N(CH₃)₂ |
| 796 | 2,6-F2 | NH—(CH₂)₂—NH—(CH₂)₂—O—C(O)H |
| 797 | 2,6-F2 | NH—(CH₂)₂—N(CH₃)—(CH₂)₂—OH |
| 798 | 2,6-F2 | NH—(CH₂)₂—N(CH₃)—(CH₂)₂—NH₂ |
| 799 | 2,6-F2 | NH—(CH₂)₂—N(CH₃)—(CH₂)₂—NHCH₃ |
| 800 | 2,6-F2 | NH—(CH₂)₂—N(CH₃)—(CH₂)₂—N(CH₃)₂ |
| 801 | 2,6-F2 | NH—(CH₂)₂—N(CH₃)—(CH₂)₂—O—C(O)H |
| 802 | 2,6-F2 | N(CH₃)—(CH₂)₂—OH |
| 803 | 2,6-F2 | N(CH₃)—(CH₂)₂—NH₂ |
| 804 | 2,6-F2 | N(CH₃)—(CH₂)₂—NHCH₃ |
| 805 | 2,6-F2 | N(CH₃)—(CH₂)₂—N(CH₃)₂ |
| 806 | 2,6-F2 | N(CH₃)—(CH₂)₂—O—C(O)H |
| 807 | 2,6-F2 | N(CH₃)—(CH₂)₃—OH |
| 808 | 2,6-F2 | N(CH₃)—(CH₂)₃—NH₂ |
| 809 | 2,6-F2 | N(CH₃)—(CH₂)₃—NHCH₃ |
| 810 | 2,6-F2 | N(CH₃)—(CH₂)₃—N(CH₃)₂ |
| 811 | 2,6-F2 | N(CH₃)—(CH₂)₃—O—C(O)H |
| 812 | 2,6-F2 | N(CH₃)—CH(CH₃)—CH₂—OH |
| 813 | 2,6-F2 | N(CH₃)—CH(CH₃)—CH₂—NH₂ |
| 814 | 2,6-F2 | N(CH₃)—CH(CH₃)—CH₂—NHCH₃ |
| 815 | 2,6-F2 | N(CH₃)—CH(CH₃)—CH₂—N(CH₃)₂ |
| 816 | 2,6-F2 | N(CH₃)—CH(CH₃)—CH₂—O—C(O)H |
| 817 | 2,6-F2 | N(CH₃)—CH₂—CH(CH₃)—OH |
| 818 | 2,6-F2 | N(CH₃)—CH₂—CH(CH₃)—NH₂ |
| 819 | 2,6-F2 | N(CH₃)—CH₂—CH(CH₃)—NHCH₃ |
| 820 | 2,6-F2 | N(CH₃)—CH₂—CH(CH₃)—N(CH₃)₂ |
| 821 | 2,6-F2 | N(CH₃)—CH₂—CH(CH₃)—O—C(O)H |
| 822 | 2,6-F2 | N(CH₃)—(CH₂)₂—O—(CH₂)₂—OH |
| 823 | 2,6-F2 | N(CH₃)—(CH₂)₂—O—(CH₂)₂—NH₂ |
| 824 | 2,6-F2 | N(CH₃)—(CH₂)₂—O—(CH₂)₂—NHCH₃ |
| 825 | 2,6-F2 | N(CH₃)—(CH₂)₂—O—(CH₂)₂—N(CH₃)₂ |
| 826 | 2,6-F2 | N(CH₃)—(CH₂)₂—O—(CH₂)₂—O—C(O)H |
| 827 | 2,6-F2 | N(CH₃)—(CH₂)₂—NH—(CH₂)₂—OH |
| 828 | 2,6-F2 | N(CH₃)—(CH₂)₂—NH—(CH₂)₂—NH₂ |
| 829 | 2,6-F2 | N(CH₃)—(CH₂)₂—NH—(CH₂)₂—NHCH₃ |
| 830 | 2,6-F2 | N(CH₃)—(CH₂)₂—NH—(CH₂)₂—N(CH₃)₂ |
| 831 | 2,6-F2 | N(CH₃)—(CH₂)₂—NH—(CH₂)₂—O—C(O)H |
| 832 | 2,6-F2 | N(CH₃)—(CH₂)₂—N(CH₃)—(CH₂)₂—OH |
| 833 | 2,6-F2 | N(CH₃)—(CH₂)₂—N(CH₃)—(CH₂)₂—NH₂ |
| 834 | 2,6-F2 | N(CH₃)—(CH₂)₂—N(CH₃)—(CH₂)₂—NHCH₃ |
| 835 | 2,6-F2 | N(CH₃)—(CH₂)₂—N(CH₃)—(CH₂)₂—N(CH₃)₂ |
| 836 | 2,6-F2 | N(CH₃)—(CH₂)₂—N(CH₃)—(CH₂)₂—O—C(O)H |
| 837 | 2-F,6-Cl | C(O)NH—(CH₂)₂—OH |
| 838 | 2-F,6-Cl | C(O)NH—(CH₂)₂—NH₂ |
| 839 | 2-F,6-Cl | C(O)NH—(CH₂)₂—NHCH₃ |
| 840 | 2-F,6-Cl | C(O)NH—(CH₂)₂—N(CH₃)₂ |
| 841 | 2-F,6-Cl | C(O)NH—(CH₂)₂—O—C(O)H |
| 842 | 2-F,6-Cl | C(O)NH—(CH₂)₃—OH |
| 843 | 2-F,6-Cl | C(O)NH—(CH₂)₃—NH₂ |
| 844 | 2-F,6-Cl | C(O)NH—(CH₂)₃—NHCH₃ |
| 845 | 2-F,6-Cl | C(O)NH—(CH₂)₃—N(CH₃)₂ |
| 846 | 2-F,6-Cl | C(O)NH—(CH₂)₃—O—C(O)H |
| 847 | 2-F,6-Cl | C(O)NH—CH(CH₃)—CH₂—OH |
| 848 | 2-F,6-Cl | C(O)NH—CH(CH₃)—CH₂—NH₂ |
| 849 | 2-F,6-Cl | C(O)NH—CH(CH₃)—CH₂—NHCH₃ |
| 850 | 2-F,6-Cl | C(O)NH—CH(CH₃)—CH₂—N(CH₃)₂ |
| 851 | 2-F,6-Cl | C(O)NH—CH(CH₃)—CH₂—O—C(O)H |
| 852 | 2-F,6-Cl | C(O)NH—CH₂—CH(CH₃)—OH |
| 853 | 2-F,6-Cl | C(O)NH—CH₂—CH(CH₃)—NH₂ |
| 854 | 2-F,6-Cl | C(O)NH—CH₂—CH(CH₃)—NHCH₃ |
| 855 | 2-F,6-Cl | C(O)NH—CH₂—CH(CH₃)—N(CH₃)₂ |
| 856 | 2-F,6-Cl | C(O)NH—CH₂—CH(CH₃)—O—C(O)H |
| 857 | 2-F,6-Cl | C(O)NH—(CH₂)₂—O—(CH₂)₂—OH |
| 858 | 2-F,6-Cl | C(O)NH—(CH₂)₂—O—(CH₂)₂—NH₂ |
| 859 | 2-F,6-Cl | C(O)NH—(CH₂)₂—O—(CH₂)₂—NHCH₃ |
| 860 | 2-F,6-Cl | C(O)NH—(CH₂)₂—O—(CH₂)₂—N(CH₃)₂ |
| 861 | 2-F,6-Cl | C(O)NH—(CH₂)₂—O—(CH₂)₂—O—C(O)H |
| 862 | 2-F,6-Cl | C(O)NH—(CH₂)₂—NH—(CH₂)₂—OH |
| 863 | 2-F,6-Cl | C(O)NH—(CH₂)₂—NH—(CH₂)₂—NH₂ |
| 864 | 2-F,6-Cl | C(O)NH—(CH₂)₂—NH—(CH₂)₂—NHCH₃ |
| 865 | 2-F,6-Cl | C(O)NH—(CH₂)₂—NH—(CH₂)₂—N(CH₃)₂ |
| 866 | 2-F,6-Cl | C(O)NH—(CH₂)₂—NH—(CH₂)₂—O—C(O)H |
| 867 | 2-F,6-Cl | C(O)NH—(CH₂)₂—N(CH₃)—(CH₂)₂—OH |
| 868 | 2-F,6-Cl | C(O)NH—(CH₂)₂—N(CH₃)—(CH₂)₂—NH₂ |
| 869 | 2-F,6-Cl | C(O)NH—(CH₂)₂—N(CH₃)—(CH₂)₂—NHCH₃ |
| 870 | 2-F,6-Cl | C(O)NH—(CH₂)₂—N(CH₃)—(CH₂)₂—N(CH₃)₂ |
| 871 | 2-F,6-Cl | C(O)NH—(CH₂)₂—N(CH₃)—(CH₂)₂—O—C(O)H |
| 872 | 2-F,6-Cl | C(O)N(CH₃)—(CH₂)₂—OH |
| 873 | 2-F,6-Cl | C(O)N(CH₃)—(CH₂)₂—NH₂ |
| 874 | 2-F,6-Cl | C(O)N(CH₃)—(CH₂)₂—NHCH₃ |
| 875 | 2-F,6-Cl | C(O)N(CH₃)—(CH₂)₂—N(CH₃)₂ |
| 876 | 2-F,6-Cl | C(O)N(CH₃)—(CH₂)₂—O—C(O)H |
| 877 | 2-F,6-Cl | C(O)N(CH₃)—(CH₂)₃—OH |
| 878 | 2-F,6-Cl | C(O)N(CH₃)—(CH₂)₃—NH₂ |
| 879 | 2-F,6-Cl | C(O)N(CH₃)—(CH₂)₃—NHCH₃ |
| 880 | 2-F,6-Cl | C(O)N(CH₃)—(CH₂)₃—N(CH₃)₂ |
| 881 | 2-F,6-Cl | C(O)N(CH₃)—(CH₂)₃—O—C(O)H |
| 882 | 2-F,6-Cl | C(O)N(CH₃)—CH(CH₃)—CH₂—OH |
| 883 | 2-F,6-Cl | C(O)N(CH₃)—CH(CH₃)—CH₂—NH₂ |
| 884 | 2-F,6-Cl | C(O)N(CH₃)—CH(CH₃)—CH₂—NHCH₃ |
| 885 | 2-F,6-Cl | C(O)N(CH₃)—CH(CH₃)—CH₂—N(CH₃)₂ |
| 886 | 2-F,6-Cl | C(O)N(CH₃)—CH(CH₃)—CH₂—O—C(O)H |
| 887 | 2-F,6-Cl | C(O)N(CH₃)—CH₂—CH(CH₃)—OH |
| 888 | 2-F,6-Cl | C(O)N(CH₃)—CH₂—CH(CH₃)—NH₂ |
| 889 | 2-F,6-Cl | C(O)N(CH₃)—CH₂—CH(CH₃)—NHCH₃ |
| 890 | 2-F,6-Cl | C(O)N(CH₃)—CH₂—CH(CH₃)—N(CH₃)₂ |
| 891 | 2-F,6-Cl | C(O)N(CH₃)—(CH₂)₂—O—(CH₂)₂—OH |
| 892 | 2-F,6-Cl | C(O)N(CH₃)—(CH₂)₂—O—(CH₂)₂—NH₂ |
| 893 | 2-F,6-Cl | C(O)N(CH₃)—(CH₂)₂—O—(CH₂)₂—NHCH₃ |
| 894 | 2-F,6-Cl | C(O)N(CH₃)—(CH₂)₂—O—(CH₂)₂—N(CH₃)₂ |
| 895 | 2-F,6-Cl | C(O)N(CH₃)—(CH₂)₂—O—(CH₂)₂—O—C(O)H |
| 896 | 2-F,6-Cl | C(O)N(CH₃)—(CH₂)₂—NH—(CH₂)₂—OH |
| 897 | 2-F,6-Cl | C(O)N(CH₃)—(CH₂)₂—NH—(CH₂)₂—NH₂ |
| 898 | 2-F,6-Cl | C(O)N(CH₃)—(CH₂)₂—NH—(CH₂)₂—NHCH₃ |
| 899 | 2-F,6-Cl | C(O)N(CH₃)—(CH₂)₂—NH—(CH₂)₂—N(CH₃)₂ |
| 900 | 2-F,6-Cl | C(O)N(CH₃)—(CH₂)₂—NH—(CH₂)₂—O—C(O)H |
| 901 | 2-F,6-Cl | C(O)N(CH₃)—(CH₂)₂—N(CH₃)—(CH₂)₂—OH |
| 902 | 2-F,6-Cl | C(O)N(CH₃)—(CH₂)₂—N(CH₃)—(CH₂)₂—NH₂ |
| 903 | 2-F,6-Cl | C(O)N(CH₃)—(CH₂)₂—N(CH₃)—(CH₂)₂—NHCH₃ |
| 904 | 2-F,6-Cl | C(O)N(CH₃)—(CH₂)₂—N(CH₃)—(CH₂)₂—N(CH₃)₂ |
| 905 | 2-F,6-Cl | C(O)N(CH₃)—(CH₂)₂—N(CH₃)—(CH₂)₂—O— |
| | | C(O)H |
| 906 | 2-F,6-Cl | C(O)O—(CH₂)₂—OH |
| 907 | 2-F,6-Cl | C(O)O—(CH₂)₂—NH₂ |
| 908 | 2-F,6-Cl | C(O)O—(CH₂)₂—NHCH₃ |
| 909 | 2-F,6-Cl | C(O)O—(CH₂)₂—N(CH₃)₂ |
| 910 | 2-F,6-Cl | C(O)O—(CH₂)₂—O—C(O)H |
| 911 | 2-F,6-Cl | C(O)O—(CH₂)₃—OH |
| 912 | 2-F,6-Cl | C(O)O—(CH₂)₃—NH₂ |
| 913 | 2-F,6-Cl | C(O)O—(CH₂)₃—NHCH₃ |
| 914 | 2-F,6-Cl | C(O)O—(CH₂)₃—N(CH₃)₂ |
| 915 | 2-F,6-Cl | C(O)O—(CH₂)₃—O—C(O)H |
| 916 | 2-F,6-Cl | C(O)O—CH(CH₃)—CH₂—OH |
| 917 | 2-F,6-Cl | C(O)O—CH(CH₃)—CH₂—NH₂ |
| 918 | 2-F,6-Cl | C(O)O—CH(CH₃)—CH₂—NHCH₃ |
| 919 | 2-F,6-Cl | C(O)O—CH(CH₃)—CH₂—N(CH₃)₂ |
| 920 | 2-F,6-Cl | C(O)O—CH(CH₃)—CH₂—O—C(O)H |
| 921 | 2-F,6-Cl | C(O)O—CH₂—CH(CH₃)—OH |
| 922 | 2-F,6-Cl | C(O)O—CH₂—CH(CH₃)—NH₂ |
| 923 | 2-F,6-Cl | C(O)O—CH₂—CH(CH₃)—NHCH₃ |
| 924 | 2-F,6-Cl | C(O)O—CH₂—CH(CH₃)—N(CH₃)₂ |
| 925 | 2-F,6-Cl | C(O)O—CH₂—CH(CH₃)—O—C(O)H |
| 926 | 2-F,6-Cl | C(O)O—(CH₂)₂—O—(CH₂)₂—OH |
| 927 | 2-F,6-Cl | C(O)O—(CH₂)₂—O—(CH₂)₂—NH₂ |
| 928 | 2-F,6-Cl | C(O)O—(CH₂)₂—O—(CH₂)₂—NHCH₃ |
| 929 | 2-F,6-Cl | C(O)O—(CH₂)₂—O—(CH₂)₂—N(CH₃)₂ |
| 930 | 2-F,6-Cl | C(O)O—(CH₂)₂—O—(CH₂)₂—O—C(O)H |
| 931 | 2-F,6-Cl | C(O)O—(CH₂)₂—NH—(CH₂)₂—OH |
| 932 | 2-F,6-Cl | C(O)O—(CH₂)₂—NH—(CH₂)₂—NH₂ |
| 933 | 2-F,6-Cl | C(O)O—(CH₂)₂—NH—(CH₂)₂—NHCH₃ |
| 934 | 2-F,6-Cl | C(O)O—(CH₂)₂—NH—(CH₂)₂—N(CH₃)₂ |
| 935 | 2-F,6-Cl | C(O)O—(CH₂)₂—NH—(CH₂)₂—O—C(O)H |
| 936 | 2-F,6-Cl | C(O)O—(CH₂)₂—N(CH₃)—(CH₂)₂—OH |
| 937 | 2-F,6-Cl | C(O)O—(CH₂)₂—N(CH₃)—(CH₂)₂—NH₂ |
| 938 | 2-F,6-Cl | C(O)O—(CH₂)₂—N(CH₃)—(CH₂)₂—NHCH₃ |
| 939 | 2-F,6-Cl | C(O)O—(CH₂)₂—N(CH₃)—(CH₂)₂—N(CH₃)₂ |
| 940 | 2-F,6-Cl | C(O)O—(CH₂)₂—N(CH₃)—(CH₂)₂—O—C(O)H |
| 941 | 2-F,6-Cl | O—(CH₂)₂—OH |
| 942 | 2-F,6-Cl | O—(CH₂)₂—NH₂ |
| 943 | 2-F,6-Cl | O—(CH₂)₂—NHCH₃ |
| 944 | 2-F,6-Cl | O—(CH₂)₂—N(CH₃)₂ |
| 945 | 2-F,6-Cl | O—(CH₂)₂—O—C(O)H |
| 946 | 2-F,6-Cl | O—(CH₂)₃—OH |
| 947 | 2-F,6-Cl | O—(CH₂)₃—NH₂ |
| 948 | 2-F,6-Cl | O—(CH₂)₃—NHCH₃ |
| 949 | 2-F,6-Cl | O—(CH₂)₃—N(CH₃)₂ |
| 950 | 2-F,6-Cl | O—(CH₂)₃—O—C(O)H |
| 951 | 2-F,6-Cl | O—CH(CH₃)—CH₂—OH |
| 952 | 2-F,6-Cl | O—CH(CH₃)—CH₂—NH₂ |
| 953 | 2-F,6-Cl | O—CH(CH₃)—CH₂—NHCH₃ |
| 954 | 2-F,6-Cl | O—CH(CH₃)—CH₂—N(CH₃)₂ |
| 955 | 2-F,6-Cl | O—CH(CH₃)—CH₂—O—C(O)H |
| 956 | 2-F,6-Cl | O—CH₂—CH(CH₃)—OH |
| 957 | 2-F,6-Cl | O—CH₂—CH(CH₃)—NH₂ |
| 958 | 2-F,6-Cl | O—CH₂—CH(CH₃)—NHCH₃ |
| 959 | 2-F,6-Cl | O—CH₂—CH(CH₃)—N(CH₃)₂ |
| 960 | 2-F,6-Cl | O—CH₂—CH(CH₃)—O—C(O)H |
| 961 | 2-F,6-Cl | O—(CH₂)₂—O—(CH₂)₂—OH |
| 962 | 2-F,6-Cl | O—(CH₂)₂—O—(CH₂)₂—NH₂ |
| 963 | 2-F,6-Cl | O—(CH₂)₂—O—(CH₂)₂—NHCH₃ |
| 964 | 2-F,6-Cl | O—(CH₂)₂—O—(CH₂)₂—N(CH₃)₂ |
| 965 | 2-F,6-Cl | O—(CH₂)₂—O—(CH₂)₂—O—C(O)H |
| 966 | 2-F,6-Cl | O—(CH₂)₂—NH—(CH₂)₂—OH |
| 967 | 2-F,6-Cl | O—(CH₂)₂—NH—(CH₂)₂—NH₂ |
| 968 | 2-F,6-Cl | O—(CH₂)₂—NH—(CH₂)₂—NHCH₃ |
| 969 | 2-F,6-Cl | O—(CH₂)₂—NH—(CH₂)₂—N(CH₃)₂ |
| 970 | 2-F,6-Cl | O—(CH₂)₂—NH—(CH₂)₂—O—C(O)H |
| 971 | 2-F,6-Cl | O—(CH₂)₂—N(CH₃)—(CH₂)₂—OH |
| 972 | 2-F,6-Cl | O—(CH₂)₂—N(CH₃)—(CH₂)₂—NH₂ |
| 973 | 2-F,6-Cl | O—(CH₂)₂—N(CH₃)—(CH₂)₂—NHCH₃ |
| 974 | 2-F,6-Cl | O—(CH₂)₂—N(CH₃)—(CH₂)₂—N(CH₃)₂ |
| 975 | 2-F,6-Cl | O—(CH₂)₂—N(CH₃)—(CH₂)₂—O—C(O)H |
| 976 | 2-F,6-Cl | NH—(CH₂)₂—OH |
| 977 | 2-F,6-Cl | NH—(CH₂)₂—NH₂ |
| 978 | 2-F,6-Cl | NH—(CH₂)₂—NHCH₃ |
| 979 | 2-F,6-Cl | NH—(CH₂)₂—N(CH₃)₂ |
| 980 | 2-F,6-Cl | NH—(CH₂)₂—O—C(O)H |
| 981 | 2-F,6-Cl | NH—(CH₂)₃—OH |
| 982 | 2-F,6-Cl | NH—(CH₂)₃—NH₂ |
| 983 | 2-F,6-Cl | NH—(CH₂)₃—NHCH₃ |
| 984 | 2-F,6-Cl | NH—(CH₂)₃—N(CH₃)₂ |
| 985 | 2-F,6-Cl | NH—(CH₂)₃—O—C(O)H |
| 986 | 2-F,6-Cl | NH—CH(CH₃)—CH₂—OH |
| 987 | 2-F,6-Cl | NH—CH(CH₃)—CH₂—NH₂ |
| 988 | 2-F,6-Cl | NH—CH(CH₃)—CH₂—NHCH₃ |
| 989 | 2-F,6-Cl | NH—CH(CH₃)—CH₂—N(CH₃)₂ |
| 990 | 2-F,6-Cl | NH—CH(CH₃)—CH₂—O—C(O)H |
| 991 | 2-F,6-Cl | NH—CH₂—CH(CH₃)—OH |
| 992 | 2-F,6-Cl | NH—CH₂—CH(CH₃)—NH₂ |
| 993 | 2-F,6-Cl | NH—CH₂—CH(CH₃)—NHCH₃ |
| 994 | 2-F,6-Cl | NH—CH₂—CH(CH₃)—N(CH₃)₂ |
| 995 | 2-F,6-Cl | NH—CH₂—CH(CH₃)—O—C(O)H |
| 996 | 2-F,6-Cl | NH—(CH₂)₂—O—(CH₂)₂—OH |
| 997 | 2-F,6-Cl | NH—(CH₂)₂—O—(CH₂)₂—NH₂ |
| 998 | 2-F,6-Cl | NH—(CH₂)₂—O—(CH₂)₂—NHCH₃ |
| 999 | 2-F,6-Cl | NH—(CH₂)₂—O—(CH₂)₂—N(CH₃)₂ |
| 1000 | 2-F,6-Cl | NH—(CH₂)₂—O—(CH₂)₂—O—C(O)H |
| 1001 | 2-F,6-Cl | NH—(CH₂)₂—NH—(CH₂)₂—OH |
| 1002 | 2-F,6-Cl | NH—(CH₂)₂—NH—(CH₂)₂—NH₂ |
| 1003 | 2-F,6-Cl | NH—(CH₂)₂—NH—(CH₂)₂—NHCH₃ |
| 1004 | 2-F,6-Cl | NH—(CH₂)₂—NH—(CH₂)₂—N(CH₃)₂ |
| 1005 | 2-F,6-Cl | NH—(CH₂)₂—NH—(CH₂)₂—O—C(O)H |
| 1006 | 2-F,6-Cl | NH—(CH₂)₂—N(CH₃)—(CH₂)₂—OH |
| 1007 | 2-F,6-Cl | NH—(CH₂)₂—N(CH₃)—(CH₂)₂—NH₂ |
| 1008 | 2-F,6-Cl | NH—(CH₂)₂—N(CH₃)—(CH₂)₂—NHCH₃ |
| 1009 | 2-F,6-Cl | NH—(CH₂)₂—N(CH₃)—(CH₂)₂—N(CH₃)₂ |
| 1010 | 2-F,6-Cl | NH—(CH₂)₂—N(CH₃)—(CH₂)₂—O—C(O)H |
| 1011 | 2-F,6-Cl | N(CH₃)—(CH₂)₂—OH |
| 1012 | 2-F,6-Cl | N(CH₃)—(CH₂)₂—NH₂ |
| 1013 | 2-F,6-Cl | N(CH₃)—(CH₂)₂—NHCH₃ |
| 1014 | 2-F,6-Cl | N(CH₃)—(CH₂)₂—N(CH₃)₂ |
| 1015 | 2-F,6-Cl | N(CH₃)—(CH₂)₂—O—C(O)H |
| 1016 | 2-F,6-Cl | N(CH₃)—(CH₂)₃—OH |
| 1017 | 2-F,6-Cl | N(CH₃)—(CH₂)₃—NH₂ |
| 1018 | 2-F,6-Cl | N(CH₃)—(CH₂)₃—NHCH₃ |
| 1019 | 2-F,6-Cl | N(CH₃)—(CH₂)₃—N(CH₃)₂ |
| 1020 | 2-F,6-Cl | N(CH₃)—(CH₂)₃—O—C(O)H |
| 1021 | 2-F,6-Cl | N(CH₃)—CH(CH₃)—CH₂—OH |
| 1022 | 2-F,6-Cl | N(CH₃)—CH(CH₃)—CH₂—NH₂ |
| 1023 | 2-F,6-Cl | N(CH₃)—CH(CH₃)—CH₂—NHCH₃ |
| 1024 | 2-F,6-Cl | N(CH₃)—CH(CH₃)—CH₂—N(CH₃)₂ |
| 1025 | 2-F,6-Cl | N(CH₃)—CH(CH₃)—CH₂—O—C(O)H |
| 1026 | 2-F,6-Cl | N(CH₃)—CH₂—CH(CH₃)—OH |
| 1027 | 2-F,6-Cl | N(CH₃)—CH₂—CH(CH₃)—NH₂ |
| 1028 | 2-F,6-Cl | N(CH₃)—CH₂—CH(CH₃)—NHCH₃ |
| 1029 | 2-F,6-Cl | N(CH₃)—CH₂—CH(CH₃)—N(CH₃)₂ |
| 1030 | 2-F,6-Cl | N(CH₃)—CH₂—CH(CH₃)—O—C(O)H |
| 1031 | 2-F,6-Cl | N(CH₃)—(CH₂)₂—O—(CH₂)₂—OH |
| 1032 | 2-F,6-Cl | N(CH₃)—(CH₂)₂—O—(CH₂)₂—NH₂ |
| 1033 | 2-F,6-Cl | N(CH₃)—(CH₂)₂—O—(CH₂)₂—NHCH₃ |
| 1034 | 2-F,6-Cl | N(CH₃)—(CH₂)₂—O—(CH₂)₂—N(CH₃)₂ |
| 1035 | 2-F,6-Cl | N(CH₃)—(CH₂)₂—O—(CH₂)₂—O—C(O)H |
| 1036 | 2-F,6-Cl | N(CH₃)—(CH₂)₂—NH—(CH₂)₂—OH |
| 1037 | 2-F,6-Cl | N(CH₃)—(CH₂)₂—NH—(CH₂)₂—NH₂ |
| 1038 | 2-F,6-Cl | N(CH₃)—(CH₂)₂—NH—(CH₂)₂—NHCH₃ |
| 1039 | 2-F,6-Cl | N(CH₃)—(CH₂)₂—NH—(CH₂)₂—N(CH₃)₂ |
| 1040 | 2-F,6-Cl | N(CH₃)—(CH₂)₂—NH—(CH₂)₂—O—C(O)H |
| 1041 | 2-F,6-Cl | N(CH₃)—(CH₂)₂—N(CH₃)—(CH₂)₂—OH |
| 1042 | 2-F,6-Cl | N(CH₃)—(CH₂)₂—N(CH₃)—(CH₂)₂—NH₂ |
| 1043 | 2-F,6-Cl | N(CH₃)—(CH₂)₂—N(CH₃)—(CH₂)₂—NHCH₃ |
| 1044 | 2-F,6-Cl | N(CH₃)—(CH₂)₂—N(CH₃)—(CH₂)₂—N(CH₃)₂ |
| 1045 | 2-F,6-Cl | N(CH₃)—(CH₂)₂—N(CH₃)—(CH₂)₂—O—C(O)H |
| 1046 | 2-F,6-CH3 | C(O)NH—(CH₂)₂—OH |
| 1047 | 2-F,6-CH3 | C(O)NH—(CH₂)₂—NH₂ |
| 1048 | 2-F,6-CH3 | C(O)NH—(CH₂)₂—NHCH₃ |
| 1049 | 2-F,6-CH3 | C(O)NH—(CH₂)₂—N(CH₃)₂ |
| 1050 | 2-F,6-CH3 | C(O)NH—(CH₂)₂—O—C(O)H |
| 1051 | 2-F,6-CH3 | C(O)NH—(CH₂)₃—OH |
| 1052 | 2-F,6-CH3 | C(O)NH—(CH₂)₃—NH₂ |
| 1053 | 2-F,6-CH3 | C(O)NH—(CH₂)₃—NHCH₃ |
| 1054 | 2-F,6-CH3 | C(O)NH—(CH₂)₃—N(CH₃)₂ |
| 1055 | 2-F,6-CH3 | C(O)NH—(CH₂)₃—O—C(O)H |
| 1056 | 2-F,6-CH3 | C(O)NH—CH(CH₃)—CH₂—OH |
| 1057 | 2-F,6-CH3 | C(O)NH—CH(CH₃)—CH₂—NH₂ |
| 1058 | 2-F,6-CH3 | C(O)NH—CH(CH₃)—CH₂—NHCH₃ |
| 1059 | 2-F,6-CH3 | C(O)NH—CH(CH₃)—CH₂—N(CH₃)₂ |
| 1060 | 2-F,6-CH3 | C(O)NH—CH(CH₃)—CH₂—O—C(O)H |
| 1061 | 2-F,6-CH3 | C(O)NH—CH₂—CH(CH₃)—OH |
| 1062 | 2-F,6-CH3 | C(O)NH—CH₂—CH(CH₃)—NH₂ |
| 1063 | 2-F,6-CH3 | C(O)NH—CH₂—CH(CH₃)—NHCH₃ |
| 1064 | 2-F,6-CH3 | C(O)NH—CH₂—CH(CH₃)—N(CH₃)₂ |
| 1065 | 2-F,6-CH3 | C(O)NH—CH₂—CH(CH₃)—O—C(O)H |
| 1066 | 2-F,6-CH3 | C(O)NH—(CH₂)₂—O—(CH₂)₂—OH |
| 1067 | 2-F,6-CH3 | C(O)NH—(CH₂)₂—O—(CH₂)₂—NH₂ |
| 1068 | 2-F,6-CH3 | C(O)NH—(CH₂)₂—O—(CH₂)₂—NHCH₃ |
| 1069 | 2-F,6-CH3 | C(O)NH—(CH₂)₂—O—(CH₂)₂—N(CH₃)₂ |
| 1070 | 2-F,6-CH3 | C(O)NH—(CH₂)₂—O—(CH₂)₂—O—C(O)H |
| 1071 | 2-F,6-CH3 | C(O)NH—(CH₂)₂—NH—(CH₂)₂—OH |
| 1072 | 2-F,6-CH3 | C(O)NH—(CH₂)₂—NH—(CH₂)₂—NH₂ |
| 1073 | 2-F,6-CH3 | C(O)NH—(CH₂)₂—NH—(CH₂)₂—NHCH₃ |
| 1074 | 2-F,6-CH3 | C(O)NH—(CH₂)₂—NH—(CH₂)₂—N(CH₃)₂ |
| 1075 | 2-F,6-CH3 | C(O)NH—(CH₂)₂—NH—(CH₂)₂—O—C(O)H |
| 1076 | 2-F,6-CH3 | C(O)NH—(CH₂)₂—N(CH₃)—(CH₂)₂—OH |
| 1077 | 2-F,6-CH3 | C(O)NH—(CH₂)₂—N(CH₃)—(CH₂)₂—NH₂ |
| 1078 | 2-F,6-CH3 | C(O)NH—(CH₂)₂—N(CH₃)—(CH₂)₂—NHCH₃ |
| 1079 | 2-F,6-CH3 | C(O)NH—(CH₂)₂—N(CH₃)—(CH₂)₂—N(CH₃)₂ |
| 1080 | 2-F,6-CH3 | C(O)NH—(CH₂)₂—N(CH₃)—(CH₂)₂—O—C(O)H |
| 1081 | 2-F,6-CH3 | C(O)N(CH₃)—(CH₂)₂—OH |
| 1082 | 2-F,6-CH3 | C(O)N(CH₃)—(CH₂)₂—NH₂ |
| 1083 | 2-F,6-CH3 | C(O)N(CH₃)—(CH₂)₂—NHCH₃ |
| 1084 | 2-F,6-CH3 | C(O)N(CH₃)—(CH₂)₂—N(CH₃)₂ |
| 1085 | 2-F,6-CH3 | C(O)N(CH₃)—(CH₂)₂—O—C(O)H |
| 1086 | 2-F,6-CH3 | C(O)N(CH₃)—(CH₂)₃—OH |
| 1087 | 2-F,6-CH3 | C(O)N(CH₃)—(CH₂)₃—NH₂ |
| 1088 | 2-F,6-CH3 | C(O)N(CH₃)—(CH₂)₃—NHCH₃ |
| 1089 | 2-F,6-CH3 | C(O)N(CH₃)—(CH₂)₃—N(CH₃)₂ |
| 1090 | 2-F,6-CH3 | C(O)N(CH₃)—(CH₂)₃—O—C(O)H |
| 1091 | 2-F,6-CH3 | C(O)N(CH₃)—CH(CH₃)—CH₂—OH |
| 1092 | 2-F,6-CH3 | C(O)N(CH₃)—CH(CH₃)—CH₂—NH₂ |
| 1093 | 2-F,6-CH3 | C(O)N(CH₃)—CH(CH₃)—CH₂—NHCH₃ |
| 1094 | 2-F,6-CH3 | C(O)N(CH₃)—CH(CH₃)—CH₂—N(CH₃)₂ |
| 1095 | 2-F,6-CH3 | C(O)N(CH₃)—CH(CH₃)—CH₂—O—C(O)H |
| 1096 | 2-F,6-CH3 | C(O)N(CH₃)—CH₂—CH(CH₃)—OH |
| 1097 | 2-F,6-CH3 | C(O)N(CH₃)—CH₂—CH(CH₃)—NH₂ |
| 1098 | 2-F,6-CH3 | C(O)N(CH₃)—CH₂—CH(CH₃)—NHCH₃ |
| 1099 | 2-F,6-CH3 | C(O)N(CH₃)—CH₂—CH(CH₃)—N(CH₃)₂ |
| 1100 | 2-F,6-CH3 | C(O)N(CH₃)—(CH₂)₂—O—(CH₂)₂—OH |
| 1101 | 2-F,6-CH3 | C(O)N(CH₃)—(CH₂)₂—O—(CH₂)₂—NH₂ |
| 1102 | 2-F,6-CH3 | C(O)N(CH₃)—(CH₂)₂—O—(CH₂)₂—NHCH₃ |
| 1103 | 2-F,6-CH3 | C(O)N(CH₃)—(CH₂)₂—O—(CH₂)₂—N(CH₃)₂ |
| 1104 | 2-F,6-CH3 | C(O)N(CH₃)—(CH₂)₂—O—(CH₂)₂—O—C(O)H |
| 1105 | 2-F,6-CH3 | C(O)N(CH₃)—(CH₂)₂—NH—(CH₂)₂—OH |
| 1106 | 2-F,6-CH3 | C(O)N(CH₃)—(CH₂)₂—NH—(CH₂)₂—NH₂ |
| 1107 | 2-F,6-CH3 | C(O)N(CH₃)—(CH₂)₂—NH—(CH₂)₂—NHCH₃ |
| 1108 | 2-F,6-CH3 | C(O)N(CH₃)—(CH₂)₂—NH—(CH₂)₂—N(CH₃)₂ |
| 1109 | 2-F,6-CH3 | C(O)N(CH₃)—(CH₂)₂—NH—(CH₂)₂—O—C(O)H |
| 1110 | 2-F,6-CH3 | C(O)N(CH₃)—(CH₂)₂—N(CH₃)—(CH₂)₂—OH |
| 1111 | 2-F,6-CH3 | C(O)N(CH₃)—(CH₂)₂—N(CH₃)—(CH₂)₂—NH₂ |
| 1112 | 2-F,6-CH3 | C(O)N(CH₃)—(CH₂)₂—N(CH₃)—(CH₂)₂—NHCH₃ |
| 1113 | 2-F,6-CH3 | C(O)N(CH₃)—(CH₂)₂—N(CH₃)—(CH₂)₂—N(CH₃)₂ |
| 1114 | 2-F,6-CH3 | C(O)N(CH₃)—(CH₂)₂—N(CH₃)—(CH₂)₂—O— |
| | | C(O)H |
| 1115 | 2-F,6-CH3 | C(O)O—(CH₂)₂—OH |
| 1116 | 2-F,6-CH3 | C(O)O—(CH₂)₂—NH₂ |
| 1117 | 2-F,6-CH3 | C(O)O—(CH₂)₂—NHCH₃ |
| 1118 | 2-F,6-CH3 | C(O)O—(CH₂)₂—N(CH₃)₂ |
| 1119 | 2-F,6-CH3 | C(O)O—(CH₂)₂—O—C(O)H |
| 1120 | 2-F,6-CH3 | C(O)O—(CH₂)₃—OH |
| 1121 | 2-F,6-CH3 | C(O)O—(CH₂)₃—NH₂ |
| 1122 | 2-F,6-CH3 | C(O)O—(CH₂)₃—NHCH₃ |
| 1123 | 2-F,6-CH3 | C(O)O—(CH₂)₃—N(CH₃)₂ |
| 1124 | 2-F,6-CH3 | C(O)O—(CH₂)₃—O—C(O)H |
| 1125 | 2-F,6-CH3 | C(O)O—CH(CH₃)—CH₂—OH |
| 1126 | 2-F,6-CH3 | C(O)O—CH(CH₃)—CH₂—NH₂ |
| 1127 | 2-F,6-CH3 | C(O)O—CH(CH₃)—CH₂—NHCH₃ |
| 1128 | 2-F,6-CH3 | C(O)O—CH(CH₃)—CH₂—N(CH₃)₂ |
| 1129 | 2-F,6-CH3 | C(O)O—CH(CH₃)—CH₂—O—C(O)H |
| 1130 | 2-F,6-CH3 | C(O)O—CH₂—CH(CH₃)—OH |
| 1131 | 2-F,6-CH3 | C(O)O—CH₂—CH(CH₃)—NH₂ |
| 1132 | 2-F,6-CH3 | C(O)O—CH₂—CH(CH₃)—NHCH₃ |
| 1133 | 2-F,6-CH3 | C(O)O—CH₂—CH(CH₃)—N(CH₃)₂ |
| 1134 | 2-F,6-CH3 | C(O)O—CH₂—CH(CH₃)—O—C(O)H |
| 1135 | 2-F,6-CH3 | C(O)O—(CH₂)₂—O—(CH₂)₂—OH |
| 1136 | 2-F,6-CH3 | C(O)O—(CH₂)₂—O—(CH₂)₂—NH₂ |
| 1137 | 2-F,6-CH3 | C(O)O—(CH₂)₂—O—(CH₂)₂—NHCH₃ |
| 1138 | 2-F,6-CH3 | C(O)O—(CH₂)₂—O—(CH₂)₂—N(CH₃)₂ |
| 1139 | 2-F,6-CH3 | C(O)O—(CH₂)₂—O—(CH₂)₂—O—C(O)H |
| 1140 | 2-F,6-CH3 | C(O)O—(CH₂)₂—NH—(CH₂)₂—OH |
| 1141 | 2-F,6-CH3 | C(O)O—(CH₂)₂—NH—(CH₂)₂—NH₂ |
| 1142 | 2-F,6-CH3 | C(O)O—(CH₂)₂—NH—(CH₂)₂—NHCH₃ |
| 1143 | 2-F,6-CH3 | C(O)O—(CH₂)₂—NH—(CH₂)₂—N(CH₃)₂ |
| 1144 | 2-F,6-CH3 | C(O)O—(CH₂)₂—NH—(CH₂)₂—O—C(O)H |
| 1145 | 2-F,6-CH3 | C(O)O—(CH₂)₂—N(CH₃)—(CH₂)₂—OH |
| 1146 | 2-F,6-CH3 | C(O)O—(CH₂)₂—N(CH₃)—(CH₂)₂—NH₂ |
| 1147 | 2-F,6-CH3 | C(O)O—(CH₂)₂—N(CH₃)—(CH₂)₂—NHCH₃ |
| 1148 | 2-F,6-CH3 | C(O)O—(CH₂)₂—N(CH₃)—(CH₂)₂—N(CH₃)₂ |
| 1149 | 2-F,6-CH3 | C(O)O—(CH₂)₂—N(CH₃)—(CH₂)₂—O—C(O)H |
| 1150 | 2-F,6-CH3 | O—(CH₂)₂—OH |
| 1151 | 2-F,6-CH3 | O—(CH₂)₂—NH₂ |
| 1152 | 2-F,6-CH3 | O—(CH₂)₂—NHCH₃ |
| 1153 | 2-F,6-CH3 | O—(CH₂)₂—N(CH₃)₂ |
| 1154 | 2-F,6-CH3 | O—(CH₂)₂—O—C(O)H |
| 1155 | 2-F,6-CH3 | O—(CH₂)₃—OH |
| 1156 | 2-F,6-CH3 | O—(CH₂)₃—NH₂ |
| 1157 | 2-F,6-CH3 | O—(CH₂)₃—NHCH₃ |
| 1158 | 2-F,6-CH3 | O—(CH₂)₃—N(CH₃)₂ |
| 1159 | 2-F,6-CH3 | O—(CH₂)₃—O—C(O)H |
| 1160 | 2-F,6-CH3 | O—CH(CH₃)—CH₂—OH |
| 1161 | 2-F,6-CH3 | O—CH(CH₃)—CH₂—NH₂ |
| 1162 | 2-F,6-CH3 | O—CH(CH₃)—CH₂—NHCH₃ |
| 1163 | 2-F,6-CH3 | O—CH(CH₃)—CH₂—N(CH₃)₂ |
| 1164 | 2-F,6-CH3 | O—CH(CH₃)—CH₂—O—C(O)H |
| 1165 | 2-F,6-CH3 | O—CH₂—CH(CH₃)—OH |
| 1166 | 2-F,6-CH3 | O—CH₂—CH(CH₃)—NH₂ |
| 1167 | 2-F,6-CH3 | O—CH₂—CH(CH₃)—NHCH₃ |
| 1168 | 2-F,6-CH3 | O—CH₂—CH(CH₃)—N(CH₃)₂ |
| 1169 | 2-F,6-CH3 | O—CH₂—CH(CH₃)—O—C(O)H |
| 1170 | 2-F,6-CH3 | O—(CH₂)₂—O—(CH₂)₂—OH |
| 1171 | 2-F,6-CH3 | O—(CH₂)₂—O—(CH₂)₂—NH₂ |
| 1172 | 2-F,6-CH3 | O—(CH₂)₂—O—(CH₂)₂—NHCH₃ |
| 1173 | 2-F,6-CH3 | O—(CH₂)₂—O—(CH₂)₂—N(CH₃)₂ |
| 1174 | 2-F,6-CH3 | O—(CH₂)₂—O—(CH₂)₂—O—C(O)H |
| 1175 | 2-F,6-CH3 | O—(CH₂)₂—NH—(CH₂)₂—OH |
| 1176 | 2-F,6-CH3 | O—(CH₂)₂—NH—(CH₂)₂—NH₂ |
| 1177 | 2-F,6-CH3 | O—(CH₂)₂—NH—(CH₂)₂—NHCH₃ |
| 1178 | 2-F,6-CH3 | O—(CH₂)₂—NH—(CH₂)₂—N(CH₃)₂ |
| 1179 | 2-F,6-CH3 | O—(CH₂)₂—NH—(CH₂)₂—O—C(O)H |
| 1180 | 2-F,6-CH3 | O—(CH₂)₂—N(CH₃)—(CH₂)₂—OH |
| 1181 | 2-F,6-CH3 | O—(CH₂)₂—N(CH₃)—(CH₂)₂—NH₂ |
| 1182 | 2-F,6-CH3 | O—(CH₂)₂—N(CH₃)—(CH₂)₂—NHCH₃ |
| 1183 | 2-F,6-CH3 | O—(CH₂)₂—N(CH₃)—(CH₂)₂—N(CH₃)₂ |
| 1184 | 2-F,6-CH3 | O—(CH₂)₂—N(CH₃)—(CH₂)₂—O—C(O)H |
| 1185 | 2-F,6-CH3 | NH—(CH₂)₂—OH |
| 1186 | 2-F,6-CH3 | NH—(CH₂)₂—NH₂ |
| 1187 | 2-F,6-CH3 | NH—(CH₂)₂—NHCH₃ |
| 1188 | 2-F,6-CH3 | NH—(CH₂)₂—N(CH₃)₂ |
| 1189 | 2-F,6-CH3 | NH—(CH₂)₂—O—C(O)H |
| 1190 | 2-F,6-CH3 | NH—(CH₂)₃—OH |
| 1191 | 2-F,6-CH3 | NH—(CH₂)₃—NH₂ |
| 1192 | 2-F,6-CH3 | NH—(CH₂)₃—NHCH₃ |
| 1193 | 2-F,6-CH3 | NH—(CH₂)₃—N(CH₃)₂ |
| 1194 | 2-F,6-CH3 | NH—(CH₂)₃—O—C(O)H |
| 1195 | 2-F,6-CH3 | NH—CH(CH₃)—CH₂—OH |
| 1196 | 2-F,6-CH3 | NH—CH(CH₃)—CH₂—NH₂ |
| 1197 | 2-F,6-CH3 | NH—CH(CH₃)—CH₂—NHCH₃ |
| 1198 | 2-F,6-CH3 | NH—CH(CH₃)—CH₂—N(CH₃)₂ |
| 1199 | 2-F,6-CH3 | NH—CH(CH₃)—CH₂—O—C(O)H |
| 1200 | 2-F,6-CH3 | NH—CH₂—CH(CH₃)—OH |
| 1201 | 2-F,6-CH3 | NH—CH₂—CH(CH₃)—NH₂ |
| 1202 | 2-F,6-CH3 | NH—CH₂—CH(CH₃)—NHCH₃ |
| 1203 | 2-F,6-CH3 | NH—CH₂—CH(CH₃)—N(CH₃)₂ |
| 1204 | 2-F,6-CH3 | NH—CH₂—CH(CH₃)—O—C(O)H |
| 1205 | 2-F,6-CH3 | NH—(CH₂)₂—O—(CH₂)₂—OH |
| 1206 | 2-F,6-CH3 | NH—(CH₂)₂—O—(CH₂)₂—NH₂ |
| 1207 | 2-F,6-CH3 | NH—(CH₂)₂—O—(CH₂)₂—NHCH₃ |
| 1208 | 2-F,6-CH3 | NH—(CH₂)₂—O—(CH₂)₂—N(CH₃)₂ |
| 1209 | 2-F,6-CH3 | NH—(CH₂)₂—O—(CH₂)₂—O—C(O)H |
| 1210 | 2-F,6-CH3 | NH—(CH₂)₂—NH—(CH₂)₂—OH |
| 1211 | 2-F,6-CH3 | NH—(CH₂)₂—NH—(CH₂)₂—NH₂ |
| 1212 | 2-F,6-CH3 | NH—(CH₂)₂—NH—(CH₂)₂—NHCH₃ |
| 1213 | 2-F,6-CH3 | NH—(CH₂)₂—NH—(CH₂)₂—N(CH₃)₂ |
| 1214 | 2-F,6-CH3 | NH—(CH₂)₂—NH—(CH₂)₂—O—C(O)H |
| 1215 | 2-F,6-CH3 | NH—(CH₂)₂—N(CH₃)—(CH₂)₂—OH |
| 1216 | 2-F,6-CH3 | NH—(CH₂)₂—N(CH₃)—(CH₂)₂—NH₂ |
| 1217 | 2-F,6-CH3 | NH—(CH₂)₂—N(CH₃)—(CH₂)₂—NHCH₃ |
| 1218 | 2-F,6-CH3 | NH—(CH₂)₂—N(CH₃)—(CH₂)₂—N(CH₃)₂ |
| 1219 | 2-F,6-CH3 | NH—(CH₂)₂—N(CH₃)—(CH₂)₂—O—C(O)H |
| 1220 | 2-F,6-CH3 | N(CH₃)—(CH₂)₂—OH |
| 1221 | 2-F,6-CH3 | N(CH₃)—(CH₂)₂—NH₂ |
| 1222 | 2-F,6-CH3 | N(CH₃)—(CH₂)₂—NHCH₃ |
| 1223 | 2-F,6-CH3 | N(CH₃)—(CH₂)₂—N(CH₃)₂ |
| 1224 | 2-F,6-CH3 | N(CH₃)—(CH₂)₂—O—C(O)H |
| 1225 | 2-F,6-CH3 | N(CH₃)—(CH₂)₃—OH |
| 1226 | 2-F,6-CH3 | N(CH₃)—(CH₂)₃—NH₂ |
| 1227 | 2-F,6-CH3 | N(CH₃)—(CH₂)₃—NHCH₃ |
| 1228 | 2-F,6-CH3 | N(CH₃)—(CH₂)₃—N(CH₃)₂ |
| 1229 | 2-F,6-CH3 | N(CH₃)—(CH₂)₃—O—C(O)H |
| 1230 | 2-F,6-CH3 | N(CH₃)—CH(CH₃)—CH₂—OH |
| 1231 | 2-F,6-CH3 | N(CH₃)—CH(CH₃)—CH₂—NH₂ |
| 1232 | 2-F,6-CH3 | N(CH₃)—CH(CH₃)—CH₂—NHCH₃ |
| 1233 | 2-F,6-CH3 | N(CH₃)—CH(CH₃)—CH₂—N(CH₃)₂ |
| 1234 | 2-F,6-CH3 | N(CH₃)—CH(CH₃)—CH₂—O—C(O)H |
| 1235 | 2-F,6-CH3 | N(CH₃)—CH₂—CH(CH₃)—OH |
| 1236 | 2-F,6-CH3 | N(CH₃)—CH₂—CH(CH₃)—NH₂ |
| 1237 | 2-F,6-CH3 | N(CH₃)—CH₂—CH(CH₃)—NHCH₃ |
| 1238 | 2-F,6-CH3 | N(CH₃)—CH₂—CH(CH₃)—N(CH₃)₂ |
| 1239 | 2-F,6-CH3 | N(CH₃)—CH₂—CH(CH₃)—O—C(O)H |
| 1240 | 2-F,6-CH3 | N(CH₃)—(CH₂)₂—O—(CH₂)₂—OH |
| 1241 | 2-F,6-CH3 | N(CH₃)—(CH₂)₂—O—(CH₂)₂—NH₂ |
| 1242 | 2-F,6-CH3 | N(CH₃)—(CH₂)₂—O—(CH₂)₂—NHCH₃ |
| 1243 | 2-F,6-CH3 | N(CH₃)—(CH₂)₂—O—(CH₂)₂—N(CH₃)₂ |
| 1244 | 2-F,6-CH3 | N(CH₃)—(CH₂)₂—O—(CH₂)₂—O—C(O)H |
| 1245 | 2-F,6-CH3 | N(CH₃)—(CH₂)₂—NH—(CH₂)₂—OH |
| 1246 | 2-F,6-CH3 | N(CH₃)—(CH₂)₂—NH—(CH₂)₂—NH₂ |
| 1247 | 2-F,6-CH3 | N(CH₃)—(CH₂)₂—NH—(CH₂)₂—NHCH₃ |
| 1248 | 2-F,6-CH3 | N(CH₃)—(CH₂)₂—NH—(CH₂)₂—N(CH₃)₂ |
| 1249 | 2-F,6-CH3 | N(CH₃)—(CH₂)₂—NH—(CH₂)₂—O—C(O)H |
| 1250 | 2-F,6-CH3 | N(CH₃)—(CH₂)₂—N(CH₃)—(CH₂)₂—OH |
| 1251 | 2-F,6-CH3 | N(CH₃)—(CH₂)₂—N(CH₃)—(CH₂)₂—NH₂ |
| 1252 | 2-F,6-CH3 | N(CH₃)—(CH₂)₂—N(CH₃)—(CH₂)₂—NHCH₃ |
| 1253 | 2-F,6-CH3 | N(CH₃)—(CH₂)₂—N(CH₃)—(CH₂)₂—N(CH₃)₂ |
| 1254 | 2-F,6-CH3 | N(CH₃)—(CH₂)₂—N(CH₃)—(CH₂)₂—O—C(O)H |

TABLE A

| No. | R |
|---|---|
| A-1 | NHCH₃ |
| A-2 | N(CH₃)₂ |
| A-3 | NHCH₂CH₃ |
| A-4 | N(CH₃)CH₂CH₃ |
| A-5 | N(CH₂CH₃)CH₂CH₃ |
| A-6 | NHCH₂CH₂CH₃ |
| A-7 | N(CH₃)CH₂CH₂CH₃ |
| A-8 | N(CH₂CH₃)CH₂CH₂CH₃ |
| A-9 | N(CH₂CH₂CH₃)₂ |
| A-10 | NHCH(CH₃)₂ |
| A-11 | N(CH₃)CH(CH₃)₂ |
| A-12 | N(CH₂CH₃)—CH(CH₃)₂ |
| A-13 | N(CH₂CH₂CH₃)—CH(CH₃)₂ |
| A-14 | NHCH₂CH₂CH₂CH₃ |
| A-15 | N(CH₃)CH₂CH₂CH₂CH₃ |
| A-16 | N(CH₂CH₃)—CH₂CH₂CH₂CH₃ |
| A-17 | N(CH₂CH₂CH₃)—CH₂CH₂CH₂CH₃ |
| A-18 | N(CH₂CH₂CH₂CH₃)₂ |
| A-19 | (±)NHCH(CH₃)CH₂CH₃ |
| A-20 | (±)N(CH₃)—CH(CH₃)CH₂CH₃ |
| A-21 | (±)N(CH₂CH₃)—CH(CH₃)CH₂CH₃ |
| A-22 | (±)N(n-C₃H₇)—CH(CH₃)CH₂CH₃ |
| A-23 | (S)NHCH(CH₃)CH₂CH₃ |
| A-24 | (S)N(CH₃)—CH(CH₃)CH₂CH₃ |
| A-25 | (S)N(CH₂CH₃)—CH(CH₃)CH₂CH₃ |
| A-26 | (S)N(n-C₃H₇)—CH(CH₃)CH₂CH₃ |
| A-27 | (R)NHCH(CH₃)CH₂CH₃ |
| A-28 | (R)N(CH₃)—CH(CH₃)CH₂CH₃ |
| A-29 | (R)N(CH₂CH₃)—CH(CH₃)CH₂CH₃ |
| A-30 | (R)N(n-C₃H₇)—CH(CH₃)CH₂CH₃ |
| A-31 | (±)NHCH(CH₃)CH(CH₃)₂ |
| A-32 | (±)N(CH₃)—CH(CH₃)CH(CH₃)₂ |
| A-33 | (±)N(CH₂CH₃)—CH(CH₃)CH(CH₃)₂ |
| A-34 | (±)N(n-C₃H₇)—CH(CH₃)CH(CH₃)₂ |
| A-35 | (S)NHCH(CH₃)CH(CH₃)₂ |
| A-36 | (S)N(CH₃)—CH(CH₃)CH(CH₃)₂ |
| A-37 | (S)N(CH₂CH₃)—CH(CH₃)CH(CH₃)₂ |
| A-38 | (S)N(n-C₃H₇)—CH(CH₃)CH(CH₃)₂ |
| A-39 | (R)NHCH(CH₃)—CH(CH₃)₂ |
| A-40 | (R)N(CH₃)—CH(CH₃)—CH(CH₃)₂ |
| A-41 | (R)N(CH₂CH₃)—CH(CH₃)CH(CH₃)₂ |
| A-42 | (R)N(n-C₃H₇)CH(CH₃)CH(CH₃)₂ |
| A-43 | (±)NHCH(CH₃)C(CH₃)₃ |
| A-44 | (±)N(CH₃)—CH(CH₃)C(CH₃)₃ |
| A-45 | (±)N(CH₂CH₃)—CH(CH₃)C(CH₃)₃ |
| A-46 | (±)N(n-C₃H₇)—CH(CH₃)C(CH₃)₃ |
| A-47 | (S)NHCH(CH₃)C(CH₃)₃ |
| A-48 | (S)N(CH₃)—CH(CH₃)C(CH₃)₃ |
| A-49 | (S)N(CH₂CH₃)—CH(CH₃)C(CH₃)₃ |
| A-50 | (S)N(n-C₃H₇)CH(CH₃)C(CH₃)₃ |
| A-51 | (R)NHCH(CH₃)C(CH₃)₃ |
| A-52 | (R)N(CH₃)—CH(CH₃)C(CH₃)₃ |
| A-53 | (R)N(CH₂CH₃)—CH(CH₃)C(CH₃)₃ |
| A-54 | (R)N(n-C₃H₇)CH(CH₃)C(CH₃)₃ |
| A-55 | NHCH₂C(CH₃)═CH₂ |
| A-56 | N(CH₃)—CH₂C(CH₃)═CH₂ |
| A-57 | N(CH₂CH₃)—CH₂C(CH₃)═CH₂ |
| A-58 | N(CH₂CH₂CH₃)—CH₂C(CH₃)═CH₂ |
| A-59 | NHCH₂CH═CH₂ |
| A-60 | N(CH₃)—CH₂CH═CH₂ |
| A-61 | N(CH₂CH₃)—CH₂CH═CH₂ |
| A-62 | N(n-C₃H₇)—CH₂CH═CH₂ |
| A-63 | NHCH(CH₃)CH═CH₂ |
| A-64 | N(CH₃)—CH(CH₃)CH═CH₂ |
| A-65 | N(CH₂CH₃)—CH(CH₃)CH═CH₂ |
| A-66 | N(n-C₃H₇)—CH(CH₃)CH═CH₂ |
| A-67 | NHCH(CH₃)C(CH₃)═CH₂ |
| A-68 | N(CH₃)—CH(CH₃)C(CH₃)═CH₂ |
| A-69 | N(CH₂CH₃)—CH(CH₃)C(CH₃)═CH₂ |
| A-70 | N(n-C₃H₇)—CH(CH₃)C(CH₃)═CH₂ |
| A-71 | NHCH₂C≡CH |
| A-72 | N(CH₃)—CH₂C≡CH |
| A-73 | N(CH₂CH₃)—CH₂C≡CH |
| A-74 | N(CH₂CH₂CH₃)—CH₂C≡CH |
| A-75 | NH-c-C₅H₉ |
| A-76 | N(CH₃)-c-C₅H₉ |
| A-77 | N(CH₂CH₃)-c-C₅H₉ |
| A-78 | N(CH₂CH₂CH₃)-c-C₅H₉ |
| A-79 | NH-c-C₆H₁₁ |
| A-80 | N(CH₃)-c-C₆H₁₁ |
| A-81 | N(CH₂CH₃)-c-C₆H₁₁ |
| A-82 | N(CH₂CH₂CH₃)-c-C₆H₁₁ |
| A-83 | NH—CH₂C₆H₅ |
| A-84 | N(CH₃)—CH₂C₆H₅ |
| A-85 | N(CH₂CH₃)—CH₂C₆H₅ |
| A-86 | N(CH₂CH₂CH₃)—CH₂C₆H₅ |
| A-87 | NH—CH₂CH₂OH |
| A-88 | N(CH₃)—CH₂CH₂OH |
| A-89 | N(CH₂CH₃)—CH₂CH₂OH |
| A-90 | N(n-C₃H₇)—CH₂CH₂OH |
| A-91 | NH—CH(CH₃)CH₂OH |
| A-92 | N(CH₃)—CH(CH₃)CH₂OH |
| A-93 | N(CH₂CH₃)—CH(CH₃)CH₂OH |
| A-94 | N(n-C₃H₇)—CH(CH₃)CH₂OH |
| A-95 | NH—CH(C₂H₅)CH₂OH |
| A-96 | N(CH₃)—CH(C₂H₅)CH₂OH |
| A-97 | N(C₂H₅)—CH(C₂H₅)CH₂OH |
| A-98 | N(n-C₃H₇)—CH(C₂H₅)CH₂OH |
| A-99 | NH—CH(n-C₃H₇)CH₂OH |
| A-100 | N(CH₃)—CH(n-C₃H₇)CH₂OH |
| A-101 | N(C₂H₅)—CH(n-C₃H₇)CH₂OH |
| A-102 | N(n-C₃H₇)—CH(n-C₃H₇)CH₂OH |
| A-103 | NH—CH(i-C₃H₇)CH₂OH |
| A-104 | N(CH₃)—CH(i-C₃H₇)CH₂OH |
| A-105 | N(C₂H₅)—CH(i-C₃H₇)CH₂OH |
| A-106 | N(n-C₃H₇)—CH(i-C₃H₇)CH₂OH |
| A-107 | NH—CH(n-C₄H₉)CH₂OH |
| A-108 | N(CH₃)—CH(n-C₄H₉)CH₂OH |
| A-109 | N(C₂H₅)—CH(n-C₄H₉)CH₂OH |
| A-110 | N(n-C₃H₇)—CH(n-C₄H₉)CH₂OH |
| A-111 | NH—CH(i-C₄H₉)CH₂OH |
| A-112 | N(CH₃)—CH(i-C₄H₉)CH₂OH |
| A-113 | N(C₂H₅)—CH(i-C₄H₉)CH₂OH |
| A-114 | N(n-C₃H₇)—CH(i-C₄H₉)CH₂OH |
| A-115 | NH—CH₂CH₂OC(O)H |
| A-116 | N(CH₃)—CH₂CH₂OC(O)H |
| A-117 | N(CH₂CH₃)—CH₂CH₂OC(O)H |
| A-118 | N(n-C₃H₇)—CH₂CH₂OC(O)H |
| A-119 | NH—CH(CH₃)CH₂OC(O)H |
| A-120 | N(CH₃)—CH(CH₃)CH₂OC(O)H |
| A-121 | N(CH₂CH₃)—CH(CH₃)CH₂OC(O)H |
| A-122 | N(n-C₃H₇)—CH(CH₃)CH₂OC(O)H |
| A-123 | NH—CH(C₂H₅)CH₂OC(O)H |
| A-124 | N(CH₃)—CH(C₂H₅)CH₂OC(O)H |
| A-125 | N(C₂H₅)—CH(C₂H₅)CH₂OC(O)H |
| A-126 | N(n-C₃H₇)—CH(C₂H₅)CH₂OC(O)H |
| A-127 | NH—CH(n-C₃H₇)CH₂OC(O)H |
| A-128 | N(CH₃)—CH(n-C₃H₇)CH₂OC(O)H |
| A-129 | N(C₂H₅)—CH(n-C₃H₇)CH₂OC(O)H |
| A-130 | N(n-C₃H₇)CH(n-C₃H₇)CH₂OC(O)H |
| A-131 | NH—CH(i-C₃H₇)CH₂OC(O)H |
| A-132 | N(CH₃)—CH(i-C₃H₇)CH₂OC(O)H |
| A-133 | N(C₂H₅)—CH(i-C₃H₇)CH₂OC(O)H |
| A-134 | N(n-C₃H₇)—CH(i-C₃H₇)CH₂OC(O)H |
| A-135 | NH—CH(n-C₄H₉)CH₂OC(O)H |
| A-136 | N(CH₃)—CH(n-C₄H₉)CH₂OC(O)H |
| A-137 | N(C₂H₅)—CH(n-C₄H₉)CH₂OC(O)H |
| A-138 | N(n-C₃H₇)CH(n-C₄H₉)CH₂OC(O)H |
| A-139 | NH—CH(i-C₄H₉)CH₂OC(O)H |
| A-140 | N(CH₃)—CH(i-C₄H₉)CH₂OC(O)H |
| A-141 | N(C₂H₅)—CH(i-C₄H₉)CH₂OC(O)H |
| A-142 | N(n-C₃H₇)—CH(i-C₄H₉)CH₂OC(O)H |
| A-143 | NH—CH₂CH₂OC(O)CH₃ |
| A-144 | N(CH₃)—CH₂CH₂OC(O)CH₃ |
| A-145 | N(CH₂CH₃)—CH₂CH₂OC(O)CH₃ |
| A-146 | N(n-C₃H₇)—CH₂CH₂OC(O)CH₃ |
| A-147 | NH—CH(CH₃)CH₂OC(O)CH₃ |
| A-148 | N(CH₃)—CH(CH₃)CH₂OC(O)CH₃ |
| A-149 | N(C₂H₅)—CH(CH₃)CH₂OC(O)CH₃ |
| A-150 | N(n-C₃H₇)—CH(CH₃)CH₂OC(O)CH₃ |
| A-151 | NH—CH(C₂H₅)CH₂OC(O)CH₃ |
| A-152 | N(CH₃)—CH(C₂H₅)CH₂OC(O)CH₃ |
| A-153 | N(C₂H₅)—CH(C₂H₅)CH₂OC(O)CH₃ |
| A-154 | N(n-C₃H₇)—CH(C₂H₅)CH₂OC(O)CH₃ |
| A-155 | NH—CH(n-C₃H₇)CH₂OC(O)CH₃ |
| A-156 | N(CH₃)—CH(n-C₃H₇)CH₂OC(O)CH₃ |
| A-157 | N(C₂H₅)—CH(n-C₃H₇)CH₂OC(O)CH₃ |
| A-158 | N(n-C₃H₇)—CH(n-C₃H₇)CH₂OC(O)CH₃ |
| A-159 | NH—CH(i-C₃H₇)CH₂OC(O)CH₃ |
| A-160 | N(CH₃)—CH(i-C₃H₇)CH₂OC(O)CH₃ |
| A-161 | N(C₂H₅)—CH(i-C₃H₇)CH₂OC(O)CH₃ |
| A-162 | N(n-C₃H₇)—CH(i-C₃H₇)CH₂OC(O)CH₃ |
| A-163 | NH—CH(n-C₄H₉)CH₂OC(O)CH₃ |
| A-164 | N(CH₃)—CH(n-C₄H₉)CH₂OC(O)CH₃ |
| A-165 | N(C₂H₅)—CH(n-C₄H₉)CH₂OC(O)CH₃ |
| A-166 | N(n-C₃H₇)—CH(n-C₄H₉)CH₂OC(O)CH₃ |
| A-167 | NH—CH(i-C₄H₉)CH₂OC(O)CH₃ |
| A-168 | N(CH₃)—CH(i-C₄H₉)CH₂OC(O)CH₃ |
| A-169 | N(C₂H₅)CH(i-C₄H₉)CH₂OC(O)CH₃ |
| A-170 | N(n-C₃H₇)—CH(i-C₄H₉)CH₂OC(O)CH₃ |
| A-171 | NH—CH₂CH₂OC(O)C₂H₅₃ |
| A-172 | N(CH₃)—CH₂CH₂OC(O)C₂H₅ |
| A-173 | N(CH₂CH₃)—CH₂CH₂OC(O)C₂H₅ |
| A-174 | N(n-C₃H₇)—CH₂CH₂OC(O)C₂H₅ |
| A-175 | NH—CH(CH₃)CH₂OC(O)C₂H₅ |
| A-176 | N(CH₃)—CH(CH₃)CH₂OC(O)C₂H₅ |
| A-177 | N(C₂H₅)—CH(CH₃)CH₂OC(O)C₂H₅ |
| A-178 | N(n-C₃H₇)—CH(CH₃)CH₂OC(O)C₂H₅ |
| A-179 | NH—CH(C₂H₅)CH₂OC(O)C₂H₅ |
| A-180 | N(CH₃)—CH(C₂H₅)CH₂OC(O)C₂H₅ |
| A-181 | N(C₂H₅)—CH(C₂H₅)CH₂OC(O)C₂H₅ |
| A-182 | N(n-C₃H₇)—CH(C₂H₅)CH₂OC(O)C₂H₅ |
| A-183 | NH—CH(n-C₃H₇)CH₂OC(O)C₂H₅ |
| A-184 | N(CH₃)—CH(n-C₃H₇)CH₂OC(O)C₂H₅ |
| A-185 | N(C₂H₅)—CH(n-C₃H₇)CH₂OC(O)C₂H₅ |
| A-186 | N(n-C₃H₇)—CH(n-C₃H₇)CH₂OC(O)C₂H₅ |
| A-187 | NH—CH(i-C₃H₇)CH₂OC(O)C₂H₅ |
| A-188 | N(CH₃)—CH(i-C₃H₇)CH₂OC(O)C₂H₅ |
| A-189 | N(C₂H₅)—CH(i-C₃H₇)CH₂OC(O)C₂H₅ |
| A-190 | N(n-C₃H₇)—CH(i-C₃H₇)CH₂OC(O)C₂H₅ |
| A-191 | NH—CH(n-C₄H₉)CH₂OC(O)C₂H₅ |
| A-192 | N(CH₃)—CH(n-C₄H₉)CH₂OC(O)C₂H₅ |
| A-193 | N(C₂H₅)—CH(n-C₄H₉)CH₂OC(O)C₂H₅ |
| A-194 | N(n-C₃H₇)—CH(n-C₄H₉)CH₂OC(O)C₂H₅ |
| A-195 | NH—CH(i-C₄H₉)CH₂OC(O)C₂H₅ |
| A-196 | N(CH₃)—CH(i-C₄H₉)CH₂OC(O)C₂H₅ |
| A-197 | N(C₂H₅)CH(i-C₄H₉)CH₂OC(O)C₂H₅ |
| A-198 | N(n-C₃H₇)—CH(i-C₄H₉)CH₂OC(O)C₂H₅ |
| A-199 | NH—CH₂CH₂OC(O)-n-C₃H₇ |
| A-200 | N(CH₃)—CH₂CH₂OC(O)-n-C₃H₇ |
| A-201 | N(CH₂CH₃)—CH₂CH₂OC(O)-n-C₃H₇ |
| A-202 | N(n-C₃H₇)—CH₂CH₂OC(O)-n-C₃H₇ |
| A-203 | NH—CH(CH₃)CH₂OC(O)-n-C₃H₇ |
| A-204 | N(CH₃)—CH(CH₃)CH₂OC(O)-n-C₃H₇ |
| A-205 | N(C₂H₅)—CH(CH₃)CH₂OC(O)-n-C₃H₇ |
| A-206 | N(n-C₃H₇)—CH(CH₃)CH₂OC(O)-n-C₃H₇ |
| A-207 | NH—CH(C₂H₅)CH₂OC(O)-n-C₃H₇ |
| A-208 | N(CH₃)—CH(C₂H₅)CH₂OC(O)-n-C₃H₇ |
| A-209 | N(C₂H₅)—CH(C₂H₅)CH₂OC(O)-n-C₃H₇ |
| A-210 | N(n-C₃H₇)—CH(C₂H₅)CH₂OC(O)-n-C₃H₇ |
| A-211 | NH—CH(n-C₃H₇)CH₂OC(O)-n-C₃H₇ |
| A-212 | N(CH₃)—CH(n-C₃H₇)CH₂OC(O)-n-C₃H₇ |
| A-213 | N(C₂H₅)—CH(n-C₃H₇)CH₂OC(O)-n-C₃H₇ |
| A-214 | N(n-C₃H₇)—CH(n-C₃H₇)CH₂OC(O)-n-C₃H₇ |
| A-215 | NH—CH(i-C₃H₇)CH₂OC(O)-n-C₃H₇ |
| A-216 | N(CH₃)—CH(i-C₃H₇)CH₂OC(O)-n-C₃H₇ |
| A-217 | N(C₂H₅)—CH(i-C₃H₇)CH₂OC(O)-n-C₃H₇ |
| A-218 | N(n-C₃H₇)—CH(i-C₃H₇)CH₂OC(O)-n-C₃H₇ |
| A-219 | NH—CH(n-C₄H₉)CH₂OC(O)-n-C₃H₇ |
| A-220 | N(CH₃)—CH(n-C₄H₉)CH₂OC(O)-n-C₃H₇ |
| A-221 | N(C₂H₅)—CH(n-C₄H₉)CH₂OC(O)-n-C₃H₇ |
| A-222 | N(n-C₃H₇)—CH(n-C₄H₉)CH₂OC(O)-n-C₃H₇ |
| A-223 | NH—CH(i-C₄H₉)CH₂OC(O)-n-C₃H₇ |
| A-224 | N(CH₃)—CH(i-C₄H₉)CH₂OC(O)-n-C₃H₇ |
| A-225 | N(C₂H₅)—CH(i-C₄H₉)CH₂OC(O)-n-C₃H₇ |
| A-226 | N(n-C₃H₇)—CH(i-C₄H₉)CH₂OC(O)-n-C₃H₇ |
| A-227 | NH—CH₂CH₂OC(O)CH(CH₂)₂ |
| A-228 | N(CH₃)—CH₂CH₂OC(O)CH(CH₂)₂ |
| A-229 | N(CH₂CH₃)—CH₂CH₂OC(O)CH(CH₂)₂ |
| A-230 | N(n-C₃H₇)—CH₂CH₂OC(O)CH(CH₂)₂ |
| A-231 | NH—CH(CH₃)CH₂OC(O)CH(CH₂)₂ |
| A-232 | N(CH₃)—CH(CH₃)CH₂OC(O)CH(CH₂)₂ |
| A-233 | N(C₂H₅)—CH(CH₃)CH₂OC(O)CH(CH₂)₂ |
| A-234 | N(n-C₃H₇)—CH(CH₃)CH₂OC(O)CH(CH₂)₂ |
| A-235 | NH—CH(C₂H₅)CH₂OC(O)CH(CH₂)₂ |
| A-236 | N(CH₃)—CH(C₂H₅)CH₂OC(O)CH(CH₂)₂ |
| A-237 | N(C₂H₅)—CH(C₂H₅)CH₂OC(O)CH(CH₂)₂ |
| A-238 | N(n-C₃H₇)—CH(C₂H₅)CH₂OC(O)CH(CH₂)₂ |
| A-239 | NH—CH(n-C₃H₇)CH₂OC(O)CH(CH₂)₂ |
| A-240 | N(CH₃)—CH(n-C₃H₇)CH₂OC(O)CH(CH₂)₂ |
| A-241 | N(C₂H₅)—CH(n-C₃H₇)CH₂OC(O)CH(CH₂)₂ |
| A-242 | N(n-C₃H₇)—CH(n-C₃H₇)CH₂OC(O)CH(CH₂)₂ |
| A-243 | NH—CH(i-C₃H₇)CH₂OC(O)CH(CH₂)₂ |
| A-244 | N(CH₃)—CH(i-C₃H₇)CH₂OC(O)CH(CH₂)₂ |
| A-245 | N(C₂H₅)—CH(i-C₃H₇)CH₂OC(O)CH(CH₂)₂ |
| A-246 | N(n-C₃H₇)—CH(i-C₃H₇)CH₂OC(O)CH(CH₂)₂ |
| A-247 | NH—CH(n-C₄H₉)—CH₂OC(O)CH(CH₂)₂ |
| A-248 | N(CH₃)—CH(n-C₄H₉)CH₂OC(O)CH(CH₂)₂ |
| A-249 | N(C₂H₅)—CH(n-C₄H₉)CH₂OC(O)CH(CH₂)₂ |
| A-250 | N(n-C₃H₇)—CH(n-C₄H₉)CH₂OC(O)CH(CH₂)₂ |
| A-251 | NH—CH(i-C₄H₉)CH₂OC(O)CH(CH₂)₂ |
| A-252 | N(CH₃)—CH(i-C₄H₉)CH₂OC(O)CH(CH₂)₂ |
| A-253 | N(C₂H₅)—CH(i-C₄H₉)CH₂OC(O)CH(CH₂)₂ |
| A-254 | N(n-C₃H₇)—CH(i-C₄H₉)CH₂OC(O)CH(CH₂)₂ |
| A-255 | N[—(CH₂)₂CH═CHCH₂—] |
| A-256 | N[—(CH₂)₂C(CH₃)═CHCH₂—] |
| A-257 | N[—CH(CH₃)CH₂CH═CHCH₂—] |
| A-258 | N[—(CH₂)₂CH(CH₃)(CH₂)₂—] |
| A-259 | N[—(CH₂)₂O(CH₂)₂—] |
| A-260 | N[—(CH₂)₂S(CH₂)₂—] |
| A-261 | N[—(CH₂)₅—] |
| A-262 | N[—(CH₂)₄—] |
| A-263 | N[—CH₂CH═CHCH₂—] |
| A-264 | N[—CH(CH₃)(CH₂)₃—] |
| A-265 | N[—CH₂CH(CH₃)(CH₂)₂—] |
| A-266 | N[—CH(CH₃)(CH₂)₂CH(CH₃)—] |
| A-267 | N[—CH(CH₃)(CH₂)₄—] |
| A-268 | N[—CH₂CH(CH₃)(CH₂)₃—] |
| A-269 | N[—(CH₂)CH(CH₃)CH₂CH(CH₃)CH₂—] |
| A-270 | N[—CH(CH₂CH₃)(CH₂)₄—] |
| A-271 | N[—(CH₂)₂CHOH(CH₂)₂—] |
| A-272 | N[—(CH₂)₆—] |
| A-273 | N[—CH(CH₃)(CH₂)₅—] |
| A-274 | N[—(CH₂)₂N(CH₃)(CH₂)₂—] |
| A-275 | N[—N═CHCH═CH—] |
| A-276 | N[—N═C(CH₃)CH═C(CH₃)—] |
| A-277 | CH₃ |
| A-278 | CH₂CH₃ |
| A-279 | CH₂CH₂CH₃ |
| A-280 | CH(CH₃)₂ |
| A-281 | CH₂CH(CH₃)₂ |
| A-282 | (±)CH(CH₃)CH₂CH₃ |
| A-283 | (R)CH(CH₃)CH₂CH₃ |
| A-284 | (S)CH(CH₃)CH₂CH₃ |
| A-285 | (CH₂)₃CH₃ |
| A-286 | C(CH₃)₃ |
| A-287 | (CH₂)₄CH₃ |
| A-288 | CH(CH₂CH₃)₂ |
| A-289 | CH₂CH₂CH(CH₃)₂ |
| A-290 | (±)CH(CH₃)(CH₂)₂CH₃ |
| A-291 | (R)CH(CH₃)(CH₂)₂CH₃ |
| A-292 | (S)CH(CH₃)(CH₂)₂CH₃ |
| A-293 | (±)CH₂CH(CH₃)CH₂CH₃ |
| A-294 | (R)CH₂CH(CH₃)CH₂CH₃ |
| A-295 | (S)CH₂CH(CH₃)CH₂CH₃ |
| A-296 | (±)CH(CH₃)CH(CH₃)₂ |
| A-297 | (R)CH(CH₃)CH(CH₃)₂ |
| A-298 | (S)CH(CH₃)CH(CH₃)₂ |
| A-299 | (CH₂)₅CH₃ |
| A-300 | (±,±)CH(CH₃)CH(CH₃)CH₂CH₃ |
| A-301 | (±,R)CH(CH₃)CH(CH₃)CH₂CH₃ |
| A-302 | (±,S)CH(CH₃)CH(CH₃)CH₂CH₃ |
| A-303 | (R,±)CH(CH₃)CH(CH₃)CH₂CH₃ |
| A-304 | (S,±)CH(CH₃)CH(CH₃)CH₂CH₃ |
| A-305 | (±)CH₂CH(CH₃)CF₃ |
| A-306 | (R)CH₂CH(CH₃)CF₃ |
| A-307 | (S)CH₂CH(CH₃)CF₃ |
| A-308 | (±)CH₂CH(CF₃)CH₂CH₃ |
| A-309 | (R)CH₂CH(CF₃)CH₂CH₃ |
| A-310 | (S)CH₂CH(CF₃)CH₂CH₃ |
| A-311 | (±,±)CH(CH₃)CH(CH₃)CF₃ |
| A-312 | (±,R)CH(CH₃)CH(CH₃)CF₃ |
| A-313 | (±,S)CH(CH₃)CH(CH₃)CF₃ |
| A-314 | (R,±)CH(CH₃)CH(CH₃)CF₃ |
| A-315 | (S,±)CH(CH₃)CH(CH₃)CF₃ |
| A-316 | (±,±)CH(CH₃)CH(CF₃)CH₂CH₃ |
| A-317 | (±,R)CH(CH₃)CH(CF₃)CH₂CH₃ |
| A-318 | (±,S)CH(CH₃)CH(CF₃)CH₂CH₃ |
| A-319 | (R,±)CH(CH₃)CH(CF₃)CH₂CH₃ |
| A-320 | (S,±)CH(CH₃)CH(CF₃)CH₂CH₃ |
| A-321 | CF₃ |
| A-322 | CF₂CF₃ |
| A-323 | CF₂CF₂CF₃ |
| A-324 | c-C₃H₅ |
| A-325 | (1-CH₃)-c-C₃H₄ |
| A-326 | c-C₅H₉ |
| A-327 | c-C₆H₁₁ |
| A-328 | (4-CH₃)-c-C₆H₁₀ |
| A-329 | CH₂C(CH₃)═CH₂ |
| A-330 | CH₂CH₂C(CH₃)═CH₂ |
| A-331 | CH₂—C(CH₃)₃ |
| A-332 | CH₂—Si(CH₃)₃ |
| A-333 | n-C₆H₁₃ |
| A-334 | (CH₂)₃—CH(CH₃)₂ |
| A-335 | (CH₂)₂—CH(CH₃)—C₂H₅ |
| A-336 | CH₂—CH(CH₃)-n-C₃H₇ |
| A-337 | CH(CH₃)-n-C₄H₉ |
| A-338 | CH₂—CH(C₂H₅)₂ |
| A-339 | CH(C₂H₅)-n-C₃H₇ |
| A-340 | CH₂-c-C₅H₉ |
| A-341 | CH₂CH(CH₃)—CH(CH₃)₂ |
| A-342 | CH(CH₃)—CH₂CH(CH₃)₂ |
| A-343 | CH(CH₃)—CH(CH₃)—C₂H₅ |
| A-344 | CH(CH₃)—C(CH₃)₃ |
| A-345 | (CH₂)₂—C(CH₃)₃ |
| A-346 | CH₂—C(CH₃)₂—C₂H₅ |
| A-347 | 2-CH₃-c-C₅H₈ |
| A-348 | 3-CH₃-c-C₅H₈ |
| A-349 | C(CH₃)₂-n-C₃H₇ |
| A-350 | (CH₂)₆—CH₃ |
| A-351 | (CH₂)₄—CH(CH₃)₂ |
| A-352 | (CH₂)₃—CH(CH₃)—C₂H₅ |
| A-353 | (CH₂)₂—CH(CH₃)-n-C₃H₇ |
| A-354 | CH₂—CH(CH₃)-n-C₄H₉ |
| A-355 | CH(CH₃)-n-C₅H₁₁ |
| A-356 | (CH₂)₃C(CH₃)₃ |
| A-357 | (CH₂)₂CH(CH₃)—CH(CH₃)₂ |
| A-358 | (CH₂)CH(CH₃)—CH₂CH(CH₃)₂ |
| A-359 | CH(CH₃)(CH₂)₂—CH(CH₃)₂ |
| A-360 | (CH₂)₂C(CH₃)₂C₂H₅ |
| A-361 | CH₂CH(CH₃)CH(CH₃)C₂H₅ |
| A-362 | CH(CH₃)CH₂CH(CH₃)C₂H₅ |
| A-363 | CH₂C(CH₃)₂-n-C₃H₇ |
| A-364 | CH(CH₃)CH(CH₃)-n-C₃H₇ |
| A-365 | C(CH₃)₂-n-C₄H₉ |
| A-366 | (CH₂)₂CH(C₂H₅)₂ |
| A-367 | CH₂CH(C₂H₅)-n-C₃H₇ |
| A-368 | CH(C₂H₅)-n-C₄H₉ |
| A-369 | CH₂CH(CH₃)C(CH₃)₃ |
| A-370 | CH(CH₃)CH₂C(CH₃)₃ |
| A-371 | CH₂C(CH₃)₂CH(CH₃)₂ |
| A-372 | CH₂CH(C₂H₅)CH(CH₃)₂ |
| A-373 | CH(CH₃)CH(CH₃)CH(CH₃)₂ |
| A-374 | C(CH₃)₂CH₂CH(CH₃)₂ |
| A-375 | CH(C₂H₅)CH₂CH(CH₃)₂ |
| A-376 | CH(CH₃)C(CH₃)₂C₂H₅ |
| A-377 | CH(CH₃)CH(C₂H₅)₂ |
| A-378 | C(CH₃)₂CH(CH₃)C₂H₅ |
| A-379 | CH(C₂H₅)CH(CH₃)C₂H₅ |
| A-380 | C(CH₃)(C₂H₅)-n-C₃H₇ |
| A-381 | CH(n-C₃H₇)₂ |
| A-382 | CH(n-C₃H₇)CH(CH₃)₂ |
| A-383 | C(CH₃)₂C(CH₃)₃ |
| A-384 | C(CH₃)(C₂H₅)—CH(CH₃)₂ |
| A-385 | C(C₂H₅)₃ |
| A-386 | (3-CH₃)-c-C₆H₁₀ |
| A-387 | (2-CH₃)-c-C₆H₁₀ |
| A-388 | n-C₈H₁₇ |
| A-389 | CH₂C(═NO—CH₃)CH₃ |
| A-390 | CH₂C(═NO—C₂H₅)CH₃ |
| A-391 | CH₂C(═NO-n-C₃H₇)CH₃ |
| A-392 | CH₂C(═NO-i-C₃H₇)CH₃ |
| A-393 | CH(CH₃)C(═NOCH₃)CH₃ |
| A-394 | CH(CH₃)C(═NOC₂H₅)CH₃ |
| A-395 | CH(CH₃)C(═NO-n-C₃H₇)CH₃ |
| A-396 | CH(CH₃)C(═NO-i-C₃H₇)CH₃ |
| A-397 | C(═NOCH₃)C(═NOCH₃)CH₃ |
| A-398 | C(═NOCH₃)C(═NOC₂H₅)CH₃ |
| A-399 | C(═NOCH₃)C(═NO-n-C₃H₇)CH₃ |
| A-400 | C(═NOCH₃)C(═NO-i-C₃H₇)CH₃ |
| A-401 | C(═NOC₂H₅)C(═NOCH₃)CH₃ |
| A-402 | C(═NOC₂H₅)C(═NOC₂H₅)CH₃ |
| A-403 | C(═NOC₂H₅)C(═NO-n-C₃H₇)CH₃ |
| A-404 | C(═NOC₂H₅)C(═NO-i-C₃H₇)CH₃ |
| A-405 | CH₂C(═NO—CH₃)C₂H₅ |
| A-406 | CH₂C(═NO—C₂H₅)C₂H₅ |
| A-407 | CH₂C(═NO-n-C₃H₇)C₂H₅ |
| A-408 | CH₂C(═NO-i-C₃H₇)C₂H₅ |
| A-409 | CH(CH₃)C(═NOCH₃)C₂H₅ |
| A-410 | CH(CH₃)C(═NOC₂H₅)C₂H₅ |
| A-411 | CH(CH₃)C(═NO-n-C₃H₇)C₂H₅ |
| A-412 | CH(CH₃)C(═NO-n-C₃H₇)C₂H₅ |
| A-413 | C(═NOCH₃)C(═NOCH₃)C₂H₅ |
| A-414 | C(═NOCH₃)C(═NOC₂H₅)C₂H₅ |
| A-415 | C(═NOCH₃)C(═NO-n-C₃H₇)C₂H₅ |
| A-416 | C(═NOCH₃)C(═NO-i-C₃H₇)C₂H₅ |
| A-417 | C(═NOC₂H₅)C(═NOCH₃)C₂H₅ |
| A-418 | C(═NOC₂H₅)C(═NOC₂H₅)C₂H₅ |
| A-419 | C(═NOC₂H₅)C(═NO-n-C₃H₇)C₂H₅ |
| A-420 | C(═NOC₂H₅)C(═NO-i-C₃H₇)C₂H₅ |
| A-421 | CH═CHCH₂CH₃ |
| A-422 | CH₂CH═CHCH₃ |
| A-423 | CH₂CH₂CH═CH₂ |
| A-424 | C(CH₃)₂CH₂CH₃ |
| A-425 | CH═C(CH₃)₂ |
| A-426 | C(═CH₂)—CH₂CH₃ |
| A-427 | C(CH₃)═CHCH₃ |
| A-428 | CH(CH₃)CH═CH₂ |
| A-429 | CH═CH-n-C₃H₇ |
| A-430 | CH₂—CH═CH—C₂H₅ |
| A-431 | (CH₂)₂—CH═CHCH₃ |
| A-432 | (CH₂)₃—CH═CH₂ |
| A-433 | CH═CHCH(CH₃)₂ |
| A-434 | CH₂—CH═C(CH₃)₂ |
| A-435 | (CH₂)₂—C(CH₃)═CH₂ |
| A-436 | CH═C(CH₃)—C₂H₅ |
| A-437 | CH₂C(═CH₂)—C₂H₅ |
| A-438 | CH₂C(CH₃)═CHCH₃ |
| A-439 | CH₂CH(CH₃)—CH═CH₂ |
| A-440 | C(═CH₂)—CH₂CH₂CH₃ |
| A-441 | C(CH₃)═CHCH₂CH₃ |
| A-442 | CH(CH₃)—CH═CHCH₃ |
| A-443 | CH(CH₃)—CH₂CH═CH₂ |
| A-444 | C(═CH₂)CH(CH₃)₂ |
| A-445 | C(CH₃)═C(CH₃)₂ |
| A-446 | CH(CH₃)—C(═CH₂)—CH₃ |
| A-447 | C(CH₃)₂—CH═CH₂ |
| A-448 | C(C₂H₅)═CHCH₃ |
| A-449 | CH(C₂H₅)—CH═CH₂ |
| A-450 | CH═CH—CH₂CH₂CH₂CH₃ |
| A-451 | CH₂CH═CHCH₂CH₂CH₃ |
| A-452 | CH₂CH₂CH═CHCH₂CH₃ |
| A-453 | CH₂CH₂CH₂CH═CHCH₃ |
| A-454 | CH₂CH₂CH₂CH₂CH═CH₂ |
| A-455 | CH═CHCH₂CH(CH₃)—CH₃ |
| A-456 | CH₂CH═CH—CH(CH₃)CH₃ |
| A-457 | CH₂CH₂CH═C(CH₃)CH₃ |
| A-458 | CH₂CH₂CH₂C(CH₃)═CH₂ |
| A-459 | CH═CHCH(CH₃)—CH₂—CH₃ |
| A-460 | CH₂—CH═C(CH₃)—CH₂CH₃ |
| A-461 | CH₂CH₂C(═CH₂)—CH₂CH₃ |
| A-462 | CH₂CH₂C(CH₃)═CHCH₃ |
| A-463 | CH₂CH₂CH(CH₃)—CH═CH₂ |
| A-464 | CH═C(CH₃)—CH₂CH₂CH₃ |
| A-465 | CH₂C(═CH₂)—CH₂CH₂CH₃ |
| A-466 | CH₂C(CH₃)═CHCH₂CH₃ |
| A-467 | CH₂CH(CH₃)—CH═CHCH₃ |
| A-468 | CH₂CH(CH₃)—CH₂CH═CH₂ |
| A-469 | C(═CH₂)—CH₂CH₂CH₂CH₃ |
| A-470 | C(CH₃)═CHCH₂CH₂CH₃ |
| A-471 | CH(CH₃)—CH═CH—CH₂CH₃ |
| A-472 | CH(CH₃)—CH₂CH═CHCH₃ |
| A-473 | CH(CH₃)—CH₂CH₂CH═CH₂ |
| A-474 | CH═CHC(CH₃)₃ |
| A-475 | CH═C(CH₃)—CH(CH₃)—CH₃ |
| A-476 | CH₂—C(═CH₂)—CH(CH₃)—CH₃ |
| A-477 | CH₂—C(CH₃)═C(CH₃)—CH₃ |
| A-478 | CH₂—CH(CH₃)—C(═CH₂)—CH₃ |
| A-479 | C(═CH₂)—CH₂—CH(CH₃)—CH₃ |
| A-480 | C(CH₃)═CH—CH(CH₃)—CH₃ |
| A-481 | CH(CH₃)—CH═C(CH₃)—CH₃ |
| A-482 | CH(CH₃)—CH₂—C(═CH₂)—CH₃ |
| A-483 | CH═C(CH₂CH₃)—CH₂CH₃ |
| A-484 | CH₂—C(═CHCH₃)—CH₂CH₃ |
| A-485 | CH₂—CH(CH═CH₂)—CH₂CH₃ |
| A-486 | C(═CHCH₃)—CH₂CH₂CH₃ |
| A-487 | CH(CH═CH₂)—CH₂CH₂CH₃ |
| A-488 | C(CH₂CH₃)═CHCH₂CH₃ |
| A-489 | CH(CH₂CH₃)—CH═CHCH₃ |
| A-490 | CH(CH₂CH₃)—CH₂—CH═CH₂ |
| A-491 | CH₂C(CH₃)₂—CH═CH₂ |
| A-492 | C(═CH₂)—CH(CH₃)—CH₂CH₃ |
| A-493 | C(CH₃)═C(CH₃)—CH₂CH₃ |
| A-494 | CH(CH₃)—C(═CH₂)—CH₂CH₃ |
| A-495 | CH(CH₃)—C(CH₃)═CHCH₃ |
| A-496 | CH(CH₃)—CH(CH₃)—CH═CH₂ |
| A-497 | C(CH₃)₂—CH═CHCH₃ |
| A-498 | C(CH₃)₂—CH₂CH═CH₂ |
| A-499 | C(═CH₂)—C(CH₃)₃ |
| A-500 | C(═CHCH₃)—CH(CH₃)—CH₃ |
| A-501 | CH(CH═CH₂)—CH(CH₃)—CH₃ |
| A-502 | C(CH₂CH₃)═C(CH₃)—CH₃ |
| A-503 | CH(CH₂—CH₃)—C(═CH₂)—CH₃ |
| A-504 | C(CH₃)₂—C(═CH₂)—CH₃ |
| A-505 | C(CH₃)(CH═CH₂)—CH₂CH₃ |
| A-506 | C(CH₃)(CH₂CH₃)—CH₂CH₂CH₃ |
| A-507 | CH(CH₂CH₃)—CH(CH₃)—CH₂CH₃ |
| A-508 | CH(CH₂CH₃)—CH₂CH(CH₃)—CH₃ |
| A-509 | C(CH₃)₂—C(CH₃)₃ |
| A-510 | C(CH₂CH₃)—C(CH₃)₃ |
| A-511 | C(CH₃)(CH₂—CH₃)—CH(CH₃)₂ |
| A-512 | CH(CH(CH₃)₂)—CH(CH₃)₂ |
| A-513 | CH═CHCH₂CH₂CH₂CH₂CH₃ |
| A-514 | CH₂CH═CHCH₂CH₂CH₂CH₃ |
| A-515 | CH₂CH₂CH═CHCH₂CH₂CH₃ |
| A-516 | CH₂CH₂CH₂CH═CHCH₂CH₃ |
| A-517 | CH₂CH₂CH₂CH₂CH═CHCH₃ |
| A-518 | CH₂CH₂CH₂CH₂CH₂CH═CH₂ |
| A-519 | CH═CHCH₂CH₂CH(CH₃)—CH₃ |
| A-520 | CH₂CH═CHCH₂CH(CH₃)—CH₃ |
| A-521 | CH₂CH₂CH═CHCH(CH₃)—CH₃ |
| A-522 | CH₂CH₂CH₂CH═C(CH₃)—CH₃ |
| A-523 | CH₂CH₂CH₂CH₂—C(═CH₂)—CH₃ |
| A-524 | CH═CHCH₂CH(CH₃)—CH₂CH₃ |
| A-525 | CH₂CH═CHCH(CH₃)—CH₂CH₃ |
| A-526 | CH₂CH₂CH═C(CH₃)—CH₂CH₃ |
| A-527 | CH₂CH₂CH₂C(═CH₂)—CH₂CH₃ |
| A-528 | CH₂CH₂CH₂C(CH₃)═CHCH₃ |
| A-529 | CH₂CH₂CH₂CH(CH₃)—CH═CH₂ |
| A-530 | CH═CHCH(CH₃)—CH₂CH₂CH₃ |
| A-531 | CH₂CH═C(CH₃)—CH₂CH₂CH₃ |
| A-532 | CH₂CH₂C(═CH₂)—CH₂CH₂CH₃ |
| A-533 | CH₂CH₂C(CH₃)═CHCH₂CH₃ |
| A-534 | CH₂CH₂CH(CH₃)—CH═CHCH₃ |
| A-535 | CH₂CH₂CH(CH₃)—CH₂CH═CH₂ |
| A-536 | CH═C(CH₃)—CH₂CH₂CH₂CH₃ |
| A-537 | CH₂C(═CH₂)—CH₂CH₂CH₂CH₃ |
| A-538 | CH₂C(CH₃)═CH—CH₂CH₂CH₃ |
| A-539 | CH₂CH(CH₃)—CH═CHCH₂CH₃ |
| A-540 | CH₂CH(CH₃)—CH₂CH═CHCH₃ |
| A-541 | CH₂CH(CH₃)—CH₂CH₂CH═CH₂ |
| A-542 | C(═CH₂)—CH₂CH₂CH₂CH₂CH₃ |
| A-543 | C(CH₃)═CHCH₂CH₂CH₂CH₃ |
| A-544 | CH(CH₃)—CH═CHCH₂CH₂CH₃ |
| A-545 | CH(CH₃)—CH₂CH═CHCH₂CH₃ |
| A-546 | CH(CH₃)—CH₂CH₂CH═CHCH₃ |
| A-547 | CH(CH₃)—CH₂CH₂CH₂CH═CH₂ |
| A-548 | CH═CH—CH₂C(CH₃)₃ |
| A-549 | CH₂CH═CHC(CH₃)₃ |
| A-550 | CH═CH—CH(CH₃)CH(CH₃)₂ |
| A-551 | CH₂CH═C(CH₃)CH(CH₃)₂ |
| A-552 | CH₂CH₂—C(═CH₂)CH(CH₃)₂ |
| A-553 | CH₂CH₂—C(CH₃)═C(CH₃)₂ |
| A-554 | CH₂CH₂CH(CH₃)—C(═CH₂)—CH₃ |
| A-555 | CH═C(CH₃)—CH₂CH(CH₃)₂ |
| A-556 | CH₂C(═CH₂)—CH₂CH(CH₃)₂ |
| A-557 | CH₂C(CH₃)═CHCH(CH₃)₂ |
| A-558 | CH₂CH(CH₃)—CH═C(CH₃)₂ |
| A-559 | CH₂CH(CH₃)—CH₂C(═CH₂)—CH₃ |
| A-560 | C(═CH₂)—CH₂CH₂CH(CH₃)₂ |
| A-561 | C(CH₃)═CHCH₂CH(CH₃)₂ |
| A-562 | CH(CH₃)—CH═CHCH(CH₃)₂ |
| A-563 | CH(CH₃)—CH₂CH═C(CH₃)₂ |
| A-564 | CH(CH₃)—CH₂CH₂C(═CH₂)—CH₃ |
| A-565 | CH═CH—C(CH₃)₂—CH₂CH₃ |
| A-566 | CH₂CH₂C(CH₃)₂—CH═CH₂ |
| A-567 | CH═C(CH₃)—CH(CH₃)—CH₂CH₃ |
| A-568 | CH₂C(═CH₂)—CH(CH₃)—CH₂CH₃ |
| A-569 | CH₂C(CH₃)═C(CH₃)—CH₂CH₃ |
| A-570 | CH₂CH(CH₃)—C(═CH₂)—CH₂CH₃ |
| A-571 | CH₂CH(CH₃)—C(CH₃)═CHCH₃ |
| A-572 | CH₂CH(CH₃)—CH(CH₃)—CH═CH₂ |
| A-573 | C(═CH₂)—CH₂CH(CH₃)—CH₂CH₃ |
| A-574 | C(CH₃)═CHCH(CH₃)—CH₂CH₃ |
| A-575 | CH(CH₃)—CH═C(CH₃)—CH₂CH₃ |
| A-576 | CH(CH₃)—CH₂C(═CH₂)—CH₂CH₃ |
| A-577 | CH(CH₃)—CH₂C(CH₃)═CHCH₃ |
| A-578 | CH(CH₃)—CH₂CH(CH₃)—CH═CH₂ |
| A-579 | CH₂C(CH₃)₂—CH═CHCH₃ |
| A-580 | CH₂C(CH₃)₂—CH₂CH═CH₂ |
| A-581 | C(═CH₂)—CH(CH₃)—CH₂CH₂CH₃ |
| A-582 | C(CH₃)═C(CH₃)—CH₂CH₂CH₃ |
| A-583 | CH(CH₃)—C(═CH₂)—CH₂CH₂CH₃ |
| A-584 | CH(CH₃)—C(CH₃)═CHCH₂CH₃ |
| A-585 | CH(CH₃)—CH(CH₃)—CH═CH—CH₃ |
| A-586 | CH(CH₃)—CH(CH₃)—CH₂CH═CH₂ |
| A-587 | C(CH₃)₂—CH═CH—CH₂CH₃ |
| A-588 | C(CH₃)₂—CH₂CH═CHCH₃ |
| A-589 | C(CH₃)₂—CH₂CH₂CH═CH₂ |
| A-590 | CH═CHCH(CH₂CH₃)—CH₂CH₃ |
| A-591 | CH₂—CH═C(CH₂—CH₃)—CH₂—CH₃ |
| A-592 | CH₂CH₂—C(═CH—CH₃)—CH₂CH₃ |
| A-593 | CH₂CH₂—CH(CH═CH₂)—CH₂CH₃ |
| A-594 | CH═C(CH₂CH₃)—CH₂CH₂CH₃ |
| A-595 | CH₂—C(═CHCH₃)—CH₂CH₂CH₃ |
| A-596 | CH₂—CH(CH═CH₂)—CH₂CH₂CH₃ |
| A-597 | CH₂—C(CH₂CH₃)═CHCH₂CH₃ |
| A-598 | CH₂CH(CH₂CH₃)—CH═CHCH₃ |
| A-599 | CH₂CH(CH₂CH₃)—CHCH═CH₂ |
| A-600 | C(═CHCH₃)—CH₂CH₂CH₂CH₃ |
| A-601 | CH(CH═CH₂)—CH₂CH₂CH₂CH₃ |
| A-602 | C(CH₂CH₃)═CHCH₂CH₂CH₃ |
| A-603 | CH(CH₂CH₃)—CH═CHCH₂CH₃ |
| A-604 | CH(CH₂CH₃)—CH₂CH═CHCH₃ |
| A-605 | CH(CH₂CH₃)—CH₂CH₂CH═CH₂ |
| A-606 | C(═CHCH₂CH₃)—CH₂CH₂CH₃ |
| A-607 | C(CH═CHCH₃)—CH₂CH₂CH₃ |
| A-608 | C(CH₂—CH═CH₂)—CH₂CH₂CH₃ |
| A-609 | CH═C(CH₃)—C(CH₃)₃ |
| A-610 | CH₂C(═CH₂)—C(CH₃)₃ |
| A-611 | CH₂C(CH₃)₂—CH(CH₂)—CH₃ |
| A-612 | C(═CH₂)—CH(CH₃)—CH(CH₃)—CH₃ |
| A-613 | C(CH₃)═C(CH₃)—CH(CH₃)—CH₃ |
| A-614 | CH(CH₃)—C(═CH₂)—CH(CH₃)—CH₃ |
| A-615 | CH(CH₃)—C(CH₃)═C(CH₃)—CH₃ |
| A-616 | CH(CH₃)—CH(CH₃)—C(═CH₂)—CH₃ |
| A-617 | C(CH₃)₂—CH═C(CH₃)—CH₃ |
| A-618 | C(CH₃)₂—CH₂—C(═CH₂)CH₃ |
| A-619 | C(CH₃)₂—C(═CH₂)—CH₂CH₃ |
| A-620 | C(CH₃)₂—C(CH₃)═CHCH₃ |
| A-621 | C(CH₃)₂—CH(CH₃)CH═CH₂ |
| A-622 | CH(CH₂CH₃)—CH₂CH(CH₃)CH₃ |
| A-623 | CH(CH₂CH₃)—CH(CH₃)—CH₂CH₃ |
| A-624 | C(CH₃)(CH₂CH₃)—CH₂CH₂CH₃ |
| A-625 | CH(i-C₃H₇)—CH₂CH₂CH₃ |
| A-626 | CH═C(CH₂CH₃)—CH(CH₃)CH₃ |
| A-627 | CH₂—C(═CHCH₃)—CH(CH₃)CH₃ |
| A-628 | CH₂—CH(CH═CH₂)—CH(CH₃)CH₃ |
| A-629 | CH₂—C(CH₂CH₃)═C(CH₃)CH₃ |
| A-630 | CH₂—CH(CH₂CH₃)—C(═CH₂)CH₃ |
| A-631 | CH₂—C(CH₃)(CH═CH₂)—CH₂CH₃ |
| A-632 | C(═CH₂)—CH(CH₂—CH₃)—CH₂CH₃ |
| A-633 | C(CH₃)═C(CH₂—CH₃)—CH₂CH₃ |
| A-634 | CH(CH₃)—C(═CH—CH₃)—CH₂CH₃ |
| A-635 | CH(CH₃)—CH(CH═CH₂)—CH₂CH₃ |
| A-636 | CH═C(CH₂CH₃)—CH(CH₃)—CH₃ |
| A-637 | CH₂—C(═CHCH₃)—CH(CH₃)—CH₃ |
| A-638 | CH₂—CH(CH═CH₂)—CH(CH₃)—CH₃ |
| A-639 | CH₂—C(CH₂CH₃)═C(CH₃)—CH₃ |
| A-640 | CH₂—CH(CH₂—CH₃)—C(═CH₂)—CH₃ |
| A-641 | C(═CHCH₃)—CH₂—CH(CH₃)—CH₃ |
| A-642 | CH(CH═CH₂)—CH₂—CH(CH₃)—CH₃ |
| A-643 | C(CH₂CH₃)═CH—CH(CH₃)—CH₃ |
| A-644 | CH(CH₂CH₃)CH═C(CH₃)—CH₃ |
| A-645 | CH(CH₂CH₃)CH₂—C(═CH₂)—CH₃ |
| A-646 | C(═CH—CH₃)CH(CH₃)—CH₂CH₃ |
| A-647 | CH(CH═CH₂)CH(CH₃)—CH₂CH₃ |
| A-648 | C(CH₂CH₃)═C(CH₃)—CH₂CH₃ |
| A-649 | CH(CH₂CH₃)—C(═CH₂)—CH₂CH₃ |
| A-650 | CH(CH₂CH₃)—C(CH₃)═CHCH₃ |
| A-651 | CH(CH₂CH₃)—CH(CH₃)—CH═CH₂ |
| A-652 | C(CH₃)—(CH═CH₂)CH₂CH₂CH₃ |
| A-653 | C(CH₃)—(CH₂—CH₃)CH═CHCH₃ |
| A-654 | C(CH₃)—(CH₂—CH₃)CH₂CH═CH₂ |
| A-655 | C[═C(CH₃)—CH₃]CH₂CH₂CH₃ |
| A-656 | CH[C(═CH₂)—CH₃]CH₂CH₂CH₃ |
| A-657 | C(i-C₃H₇)═CHCH₂CH₃ |
| A-658 | CH(i-C₃H₇)—CH═CHCH₃ |
| A-659 | CH(i-C₃H₇)—CH₂CH═CH₂ |
| A-660 | C(═CHCH₃)C(CH₃)₃ |
| A-661 | CH(CH═CH₂)C(CH₃)₃ |
| A-662 | C(CH₃)—(CH═CH₂)—CH(CH₃)CH₃ |
| A-663 | C(CH₃)(CH₂—CH₃)—C(═CH₂)CH₃ |
| A-664 | 2-CH₃-cyclohex-1-enyl |
| A-665 | [2-(═CH₂)]-c-C₆H₉ |
| A-666 | 2-CH₃-cyclohex-2-enyl |
| A-667 | 2-CH₃-cyclohex-3-enyl |
| A-668 | 2-CH₃-cyclohex-4-enyl |
| A-669 | 2-CH₃-cyclohex-5-enyl |
| A-670 | 2-CH₃-cyclohex-6-enyl |
| A-671 | 3-CH₃-cyclohex-1-enyl |
| A-672 | 3-CH₃-cyclohex-2-enyl |
| A-673 | [3-(═CH₂)]-c-C₆H₉ |
| A-674 | 3-CH₃-cyclohex-3-enyl |
| A-675 | 3-CH₃-cyclohex-4-enyl |
| A-676 | 3-CH₃-cyclohex-5-enyl |
| A-677 | 3-CH₃-cyclohex-6-enyl |
| A-678 | 4-CH₃-cyclohex-1-enyl |
| A-679 | 4-CH₃-cyclohex-2-enyl |
| A-680 | 4-CH₃-cyclohex-3-enyl |
| A-681 | [4-(═CH₂)]-c-C₆H₉ |

In addition to the compounds individualized in Tables 1 to 1254, the corresponding derivatives in which X is cyano also form part of the subject matter of the invention.

In addition to the compounds individualized in Tables 1 to 1254, the corresponding derivatives in which X is methyl also form part of the subject matter of the invention.

In addition to the compounds individualized in Tables 1 to 1254, the corresponding derivatives in which X is methoxy also form part of the subject matter of the invention.

The compounds I are suitable for use as fungicides. They have excellent activity against a broad spectrum of phytopathogenic fungi, in particular from the class of the Ascomycetes, Deuteromycetes, Basidiomycetes and Peronosporomycetes (syn. Oomycetes) and Fungi imperfecti. Some of them are systemically active and can be used in crop protection as foliar fungicides, as fungicides for seed dressing and as soil fungicides.

They are particularly important for the control of a large number of fungi on various crop plants, such as wheat, rye, barley, triticale, oats, rice, corn, grass, bananas, cotton, soybeans, coffee, sugarcane, grapevines, fruit and ornamental plants and vegetables, such as cucumbers, beans, tomatoes, potatoes and cucurbits, and also on the seeds of these plants. They can also be used in crops which are tolerant against attack by insects or fungi or herbicide applications due to breeding, including genetic engineering methods. Moreover, they are suitable for controlling Botryosphaeria species, Cylindrocarpon species, Eutypa lata, Neonectria liriodendri and Stereum hirsutum, which attack, inter alia, wood or the roots of grapevines. The compounds I are suitable for controlling Alternaria species on vegetables, rapeseed, sugarbeet, fruit, rice, soybeans and on potatoes (for example, A. solani or A. alternata) and tomatoes (for example, A. solani or A. alternata) and Alternaria ssp. (ear black) on wheat.

The compounds I are suitable for controlling Aphanomyces species on sugarbeet and vegetables.

The compounds I are suitable for controlling Ascochyta species on cereals and vegetables, for example Ascochyta tritici (leaf spot) on wheat.

The compounds I are suitable for controlling Bipolaris and Drechslera species on corn (for example, D. Maydis), cereals, rice and lawns.

The compounds I are suitable for controlling Blumeria graminis (powdery mildew) on cereals (for example, wheat or barley).

The compounds I are suitable for controlling Botrytis cinerea (gray mold) on strawberries, vegetables, flowers, grapevines and wheat (ear mildew).

The compounds I are suitable for controlling Bremia lactucae on lettuce.

The compounds I are suitable for controlling Cercospora species on corn, rice, sugarbeet and, for example, Cercospora sofina (leaf spot) or Cercospora kikuchii (leaf spot) on soybeans.

The compounds I are suitable for controlling Cladosporium herbarum (ear black) in wheat.

The compounds I are suitable for controlling Cochilobolus species on corn, cereals (for example, Cochliobolus sativus) and rice (for example, Cochilobolus miyabeanus).

The compounds I are suitable for controlling Colletotricum species on cotton and, for example, Colletotrichum truncatum (Antracnose) on soybeans.

The compounds I are suitable for controlling corynespora cassiicola (leaf spot) on soybeans.

The compounds I are suitable for controlling Dematophora necatrix (root/stem rot) on soybeans.

The compounds I are suitable for controlling Diaporthe phaseolorum (stem disease) on soybeans.

The compounds I are suitable for controlling Drechslera species, Pyrenophora species on corn, cereals, rice and lawns, on barley (for example, D. teres) and on wheat (for example, D. tritici-repentis).

The compounds I are suitable for controlling Esca on grapevines, caused by Phaeoacremonium chlamydosporium, Ph. Aleophilum, and Formitipora punctata (syn. Phellinus punctatus).

The compounds I are suitable for controlling Elsinoe ampelina on grapevines.

The compounds I are suitable for controlling Epicoccum spp. (ear black) on wheat.

The compounds I are suitable for controlling Exserohilum species on corn.

The compounds I are suitable for controlling Erysiphe cichoracearum and Sphaerotheca fuliginea on cucumbers.

The compounds I are suitable for controlling Fusarium and Verticillium species on various plants: for example, F. graminearum or F. culmorum (root rot) on cereals (for example, wheat or barley) or, for example, F. oxysporum tomatoes and Fusarium solani (stem disease) on soybeans.

The compounds I are suitable for controlling Gaeumanomyces graminis (root black) on cereals (for example, wheat or barley).

The compounds I are suitable for controlling Gibberella species on cereals and rice (for example Gibberella fujikuroi).

The compounds I are suitable for controlling Glomerella cingulata on grapevines and other plants.

The compounds I are suitable for controlling Grainstaining complex on rice.

The compounds I are suitable for controlling Guignardia budwelli on grapevines.

The compounds I are suitable for controlling Helminthosporium species on corn and rice.

The compounds I are suitable for controlling Isariopsis clavispora on grapevines.

The compounds I are suitable for controlling Macrophomina phaseana (root/stem rot) on soybeans.

The compounds I are suitable for controlling Michrodochium nivale (snow mold) on cereals (for example, wheat or barley).

The compounds I are suitable for controlling Microsphaera diffusa (powdery mildew) on soybeans.

The compounds I are suitable for controlling Mycosphaerella species on cereals, bananas and peanuts, such as, for example, M. graminicola on wheat or M. fijiensis on bananas.

The compounds I are suitable for controlling Peronospora species on cabbage (for example, P. brassicae), bulbous plants (for example, P. destructor) and, for example, Peronospora manshurica (downy mildew) on soybeans.

The compounds I are suitable for controlling Phakopsara pachyrhizi (soya rust) and Phakopsara meibomiae (soya rust) on soybeans.

The compounds I are suitable for controlling Phialophora gregata (stem disease) on soybeans.

The compounds I are suitable for controlling Phomopsis species on sunflowers, grapevines (for example, P. viticola) and soybeans (for example, Phomopsis phaseoli).

The compounds I are suitable for controlling Phytophthora species on various plants, for example, P. capsici on bell peppers, Phytophthora megasperma (leaf/stem rot) on soybeans, Phytophthora infestans on potatoes and tomatoes.

The compounds I are suitable for controlling Plasmopara viticola on grapevines.

The compounds I are suitable for controlling Podosphaera leucotricha on apples.

The compounds I are suitable for controlling Pseudocercosporella herpotrichoides (eyespot) on cereals (wheat or barley).

The compounds I are suitable for controlling Pseudoperonospora on various plants, for example, P. cubensis on cucumbers or P. humili on hops.

The compounds I are suitable for controlling Pseudopezicula tracheiphllai on grapevines.

The compounds I are suitable for controlling Puccinia species on various plants, for example, P. triticina, P. striformins, P. hordei or P. graminis on cereals (for example, wheat or barley), or on asparagus (for example, P. asparagi).

The compounds I are suitable for controlling Pyricularia oryzae, Corticium sasakii, Sarocladium oryzae, S. attenuatum, Pyrenophora tritici-repentis (leaf spot) on wheat or Pyrenophora teres (net blotch) on barley.

The compounds I are suitable for controlling Entyloma oryzae on rice.

The compounds I are suitable for controlling Pyricularia grisea on lawns and cereals.

The compounds I are suitable for controlling Pythium spp. on lawns, rice, corn, wheat, cotton, rapeseed, sunflowers, sugarbeet, vegetables and other plants (for example, P. ultiumum or P. aphanidermatum).

The compounds I are suitable for controlling Ramularia colo-cygni (Ramularial sunburn complex/physiological leaf spots) on barley.

The compounds I are suitable for controlling Rhizoctonia species on cotton, rice, potatoes, lawns, corn, rapeseed, potatoes, sugarbeet, vegetables and on various plants for example, Rhizoctonia solani (root/stem rot) on soybeans or Rhizoctonia cerealis (sharp eyspot) on wheat or barley.

The compounds I are suitable for controlling Rhynchosporium secalis on barley (leaf spot), rye and triticale.

The compounds I are suitable for controlling Sclerotinia species on rapeseed and sunflowers, and, for example, Sclerotinia sclerotorum (stem disease) or Sclerotinia rolfsii (stem disease) on soybeans.

The compounds I are suitable for controlling Septoria glycines (leaf spot) on soybeans.

The compounds I are suitable for controlling Septoria tritici (leaf septoria) and Stagonospora nodorum on wheat.

The compounds I are suitable for controlling Erysiphe (syn. Uncinula) necator on grapevines.

The compounds I are suitable for controlling Setospaeria species on corn and lawns.

The compounds I are suitable for controlling Sphacelotheca reilinia on corn.

The compounds I are suitable for controlling Stagonospora nodorum (ear septoria) on wheat.

The compounds I are suitable for controlling Thievallopsis species on soybeans and cotton.

The compounds I are suitable for controlling Tilletia species on cereals.

The compounds I are suitable for controlling Typhula incarnata (snow rot) on wheat or barley.

The compounds I are suitable for controlling Ustilago species on cereals, corn (for example, U. maydis) and sugarcane.

The compounds I are suitable for controlling Venturia species (scab) on apples (for example, V. inaequalis) and pears.

The compounds I are also suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Scierophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds according to the invention and/or their agriculturally acceptable salts are employed by treating the fungi or the plants, seeds or materials to be protected against fungal attack or the soil with a fungicidally effective amount of the active compounds. Application can be both before and after the infection of the materials, plants or seeds by the fungi.

Accordingly, the invention furthermore provides a method for controlling phytopathogenic fungi wherein the fungi or the materials, plants, the soil or seed to be protected against fungal attack are/is treated with an effective amount of at least one compound I according to the invention and/or an agriculturally acceptable salt thereof.

The invention furthermore provides a composition for controlling phytopathogenic fungi, which composition comprises at least one compound according to the invention and/or an agriculturally acceptable salt thereof and at least one solid or liquid carrier.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rates depend on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example finely divided silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable for use as surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctyiphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL, LS)

10 parts by weight of the active compounds are dissolved with 90 parts by weight of water or with a water-soluble solvent. As an alternative, wetters or other auxiliaries are added. The active compound dissolves upon dilution with water. This gives a formulation having an active compound content of 10% by weight.

B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight.

C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is added to 30 parts by weight of water by means of an emulsifying machine (e.g. Ultraturrax) and made into a homogeneous emulsion. Dilution with water gives an emulsion. The formulation has an active compound content of 25% by weight.

E Suspensions (SC, OD, FS)

In an agitated ball mill, 20 parts by weight of the active compounds are comminuted with addition of 10 parts by weight of dispersants and wetters and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of the active compounds are ground finely with addition of 50 parts by weight of dispersants and wetters and made into water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders (WP, SP, SS, WS)

75 parts by weight of the active compounds are ground in a rotor-stator mill with addition of 25 parts by weight of dispersants, wetters and silica gel. Dilution with water gives a stable dispersion or solution of the active compound. The active compound content of the formulation is 75% by weight.

H Gel Formulations (GF)

20 parts by weight of the active compounds, 10 parts by weight of dispersant, 1 part by weight of gelling agent and 70 parts by weight of water or an organic solvent are ground in a ball mill to give a fine suspension. Dilution with water gives a stable suspension with an active compound content of 20% by weight.

2. Products to be applied undiluted

I Dusts (DP, DS)

5 parts by weight of the active compounds are ground finely and mixed intimately with 95 parts by weight of finely divided kaolin. This gives a dustable product with an active compound content of 5% by weight.

J Granules (GR, FG, GG, MG)

0.5 part by weight of the active compounds is ground finely and associated with 99.5 parts by weight of carriers. Current methods are extrusion, spray-drying or the fluidized bed. This gives granules with an active compound content of 0.5% by weight to be applied undiluted.

K ULV Solutions (UL)

10 parts by weight of the active compounds are dissolved in 90 parts by weight of an organic solvent, for example xylene. This gives a product with an active compound content of 10% by weight to be applied undiluted.

Water-soluble concentrates (LS), suspensions (FS), dusts (DS), water-dispersible and water-soluble powders (WS, SS), emulsions (ES), emulsifiable concentrates (EC) and gel formulations (GF) are usually used for the treatment of seed. These formulations can be applied to the seed in undiluted or, preferably, diluted form. The application can be carried out before sowing.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These compositions can be admixed with the compositions according to the invention in a weight ratio of from 1:100 to 100:1, preferably from 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compounds according to the invention in the application form as fungicides can also be present together with other active compounds, for example with herbicides, insecticides, growth regulators, fungicides or else with fertilizers. When mixing the compounds according to the invention or the compositions comprising them with one or more further active compounds, in particular fungicides, it is in many cases possible, for example, to widen the activity spectrum or to prevent the development of resistance. In many cases, synergistic effects are obtained.

The present invention furthermore provides a combination of at least one compound according to the invention and/or an agriculturally acceptable salt thereof and at least one further fungicidal, insecticidal, herbicidal and/or growth-regulating active compound.

The following list of fungicides with which the compounds according to the invention can be applied together is meant to illustrate the possible combinations, but not to limit them:

strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-(2,5-dimethyl-phenyloxymethylene)phenyl)-3-methoxyacrylate; carboxamides
carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoro-methyl-2-methylthiazole-5-carboxamide, N-(3′,4′-dichloro-4-fluorobiphenyl-2-yl)-3-difluoromethyl-1-methylpyrazole-4-carboxamide, N-(3′,4′-dichloro-5-flurobiphenyl-2-yl)-3-difluoromethyl-1-methyl pyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chloro-phenyl)prop-2-ynyloxy]-3-methoxyphenyl)ethyl)-2-methanesulfonylamino-3-methyl-butyramide, N-(2-(4-[3-(4-chlorophenyl)prop-2-ynyloxy]-3-methoxyphenyl)ethyl)-2-ethanesulfonylamino-3-methylbutyramide;

azoles
triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;

nitrogenous heterocyclyl compounds
pyridines: fluazinam, pyrifenox, 3-[5-(4-chlorophenyl)-2,3-dimethylisoxazolidin-3-yl]-pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-S-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methyl-piperidin-1-yl)-6-(2,4,6-trifluorophenyl)-[1,2,4]triazolo[1,5-a]pyrimidine, 6-(3,4-dichlorophenyl)-5-methyl-[1,2,4]triazolo[1,5-a]pyrimidin-7-ylamine, 6-(4-tert-butylphenyl)-5-methyl-[1,2,4]triazolo[1,5-a]pyrimidin-7-ylamine, 5-methyl-6-(3,5,5-trimethylhexyl)-[1,2,4]triazolo[1,5-a]pyrimidin-7-ylamine, 5-methyl-6-octyl-[1,2,4]triazolo[1,5-a]pyrimidin-7-ylamine, 5-ethyl-6-octyl-[1,2,4]triazolo[1,5-a]pyrimidin-2,7-diamine, 6-ethyl-5-octyl-[1,2,4]triazolo[1,5-a]pyrimidin-7-ylamine, 5-ethyl-6-octyl-[1,2,4]triazolo[1,5-a]pyrimidin-7-ylamine, 5-ethyl-6-(3,5,5-trimethylhexyl)-[1,2,4]triazolo[1,5-a]pyrimidin-7-ylamine, 6-octyl-5-propyl-[1,2,4]triazolo[1,5-a]pyrimidin-7-ylamine, 5-methoxy-methyl-6-octyl-[1,2,4]triazolo[1,5-a]pyrimidin-7-ylamine, 6-octyl-5-trifluoro-methyl-[1,2,4]triazolo[1,5-a]pyrimidin-7-ylamine, 5-trifluoromethyl-6-(3,5,5-trimethyl-hexyl)-[1,2,4]triazolo[1,5-a]pyrimidin-7-ylamine, 2-butoxy-6-iodo-3-propylchromen-4-one, N,N-dimethyl-3-(3-bromo-6-fluoro-2-methylindole-1-sulfonyl)-[1,2,4]triazole-1-sulfonamide;

carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino-3-methylbutyrylamino)propionate, 4-fluorophenyl N-(1-(1-(4-cyanophenyl)ethanesulfonyl)but-2-yl)carbamate; other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorus compounds: edifenphos, fosetyl, fosetyl-aluminum, iprobenfos, pyrazophos, tolclofos-methyl, phosphorous acid and its salts;
organochlorine compounds: thiophanate-methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

Accordingly, the present invention furthermore relates to the compositions listed in Table B, where a row of Table B corresponds in each case to a fungicidal composition comprising a compound of the formula I (component 1), which is preferably one of the compounds described herein as being preferred, and the respective further active compound (component 2) stated in the row in question. According to one embodiment of the invention, component 1 in each row of Table B is in each case one of the compounds of the formula I specificially individualized in Tables 1 to 1254.

TABLE B

| Row | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | picoxystrobin |
| B-8 | a compound of the formula I | pyraclostrobin |
| B-9 | a compound of the formula I | trifloxystrobin |
| B-10 | a compound of the formula I | orysastrobin |
| B-11 | a compound of the formula I | methyl (2-chloro-5-[1-(3-methylbenzyloxy- |
| | | imino)ethyl]benzyl)carbamate |
| B-12 | a compound of the formula I | methyl (2-chloro-5-[1-(6-methylpyridin-2-yl- |
| | | methoxyimino)ethyl]benzyl)carbamate |
| B-13 | a compound of the formula I | methyl 2-(ortho-(2,5-dimethylphenyloxy- |
| | | methylene)phenyl)-3-methoxyacrylate |
| B-14 | a compound of the formula I | benalaxyl |
| B-15 | a compound of the formula I | benodanil |
| B-16 | a compound of the formula I | boscalid |
| B-17 | a compound of the formula I | carboxin |
| B-18 | a compound of the formula I | mepronil |
| B-19 | a compound of the formula I | fenfuram |
| B-20 | a compound of the formula I | fenhexamid |
| B-21 | a compound of the formula I | flutolanil |
| B-22 | a compound of the formula I | furametpyr |
| B-23 | a compound of the formula I | metalaxyl |
| B-24 | a compound of the formula I | ofurace |
| B-25 | a compound of the formula I | oxadixyl |
| B-26 | a compound of the formula I | oxycarboxin |
| B-27 | a compound of the formula I | penthiopyrad |
| B-28 | a compound of the formula I | thifluzamide |
| B-29 | a compound of the formula I | tiadinil |
| B-30 | a compound of the formula I | N-(4′-bromobiphenyl-2-yl)-4-difluoromethyl-2- |
| | | methylthiazole-5-carboxamide |
| B-31 | a compound of the formula I | N-(4′-trifluoromethylbiphenyl-2-yl)-4-di- |
| | | fluoromethyl-2-methylthiazole-5-carboxamide |
| B-32 | a compound of the formula I | N-(4′-chloro-3′-fluorobiphenyl-2-yl)-4-di- |
| | | fluoromethyl-2-methylthiazole-5-carboxamide |
| B-33 | a compound of the formula I | N-(3′,4′-dichloro-4-fluorobiphenyl-2-yl)-3-di- |
| | | fluoromethyl-1-methylpyrazole-4-carboxamide |
| B-34 | a compound of the formula I | N-(3′,4′-dichloro-5-fluorobiphenyl-2-yl)-3-di- |
| | | fluoromethyl-1-methylpyrazole-4-carboxamide |
| B-35 | a compound of the formula I | N-(2-cyanophenyl)-3,4-dichloroisothiazole-5- |
| | | carboxamide |
| B-36 | a compound of the formula I | dimethomorph |
| B-37 | a compound of the formula I | flumorph |
| B-38 | a compound of the formula I | flumetover |
| B-39 | a compound of the formula I | fluopicolide (picobenzamid) |
| B-40 | a compound of the formula I | zoxamide |
| B-41 | a compound of the formula I | carpropamid |
| B-42 | a compound of the formula I | diclocymet |
| B-43 | a compound of the formula I | mandipropamid |
| B-44 | a compound of the formula I | N-(2-(4-[3-(4-chlorophenyl)prop-2-ynyloxy]-3- |
| | | methoxyphenyl)ethyl)-2-methanesulfonyl- |
| | | amino-3-methylbutyramide |
| B-45 | a compound of the formula I | N-(2-(4-[3-(4-Chlorophenyl)prop-2-ynyloxy]-3- |
| | | methoxyphenyl)ethyl)-2-ethanesulfonylamino- |
| | | 3-methylbutyramide |
| B-46 | a compound of the formula I | bitertanol |
| B-47 | a compound of the formula I | bromuconazole |
| B-48 | a compound of the formula I | cyproconazole |
| B-49 | a compound of the formula I | difenoconazole |
| B-50 | a compound of the formula I | diniconazole |
| B-51 | a compound of the formula I | enilconazole |
| B-52 | a compound of the formula I | epoxiconazole |
| B-53 | a compound of the formula I | fenbuconazole |
| B-54 | a compound of the formula I | flusilazole |
| B-55 | a compound of the formula I | fluquinconazole |
| B-56 | a compound of the formula I | flutriafol |
| B-57 | a compound of the formula I | hexaconazol |
| B-58 | a compound of the formula I | imibenconazole |
| B-59 | a compound of the formula I | ipconazole |
| B-60 | a compound of the formula I | metconazol |
| B-61 | a compound of the formula I | myclobutanil |
| B-62 | a compound of the formula I | penconazole |
| B-63 | a compound of the formula I | propiconazole |
| B-64 | a compound of the formula I | prothioconazole |
| B-65 | a compound of the formula I | simeconazole |
| B-66 | a compound of the formula I | tebuconazole |
| B-67 | a compound of the formula I | tetraconazole |
| B-68 | a compound of the formula I | triadimenol |
| B-69 | a compound of the formula I | triadimefon |
| B-70 | a compound of the formula I | triticonazole |
| B-71 | a compound of the formula I | cyazofamid |
| B-72 | a compound of the formula I | imazalil |
| B-73 | a compound of the formula I | pefurazoate |
| B-74 | a compound of the formula I | prochloraz |
| B-75 | a compound of the formula I | triflumizole |
| B-76 | a compound of the formula I | benomyl |
| B-77 | a compound of the formula I | carbendazim |
| B-78 | a compound of the formula I | fuberidazole |
| B-79 | a compound of the formula I | thiabendazole |
| B-80 | a compound of the formula I | ethaboxam |
| B-81 | a compound of the formula I | etridiazole |
| B-82 | a compound of the formula I | hymexazole |
| B-83 | a compound of the formula I | fluazinam |
| B-84 | a compound of the formula I | pyrifenox |
| B-85 | a compound of the formula I | 3-[5-(4-chlorophenyl)-2,3-dimethylisoxazo- |
| | | lidin-3-yl]pyridine |
| B-86 | a compound of the formula I | bupirimate |
| B-87 | a compound of the formula I | cyprodinil |
| B-88 | a compound of the formula I | ferimzone |
| B-89 | a compound of the formula I | fenarimol |
| B-90 | a compound of the formula I | mepanipyrim |
| B-91 | a compound of the formula I | nuarimol |
| B-92 | a compound of the formula I | pyrimethanil |
| B-93 | a compound of the formula I | triforine |
| B-94 | a compound of the formula I | fludioxonil |
| B-95 | a compound of the formula I | fenpiclonil |
| B-96 | a compound of the formula I | aldimorph |
| B-97 | a compound of the formula I | dodemorph |
| B-98 | a compound of the formula I | fenpropimorph |
| B-99 | a compound of the formula I | tridemorph |
| B-100 | a compound of the formula I | iprodione |
| B-101 | a compound of the formula I | procymidone |
| B-102 | a compound of the formula I | vinclozolin |
| B-103 | a compound of the formula I | acibenzolar-S-methyl |
| B-104 | a compound of the formula I | anilazin |
| B-105 | a compound of the formula I | captan |
| B-106 | a compound of the formula I | captafol |
| B-107 | a compound of the formula I | dazomet |
| B-108 | a compound of the formula I | diclomezine |
| B-109 | a compound of the formula I | fenoxanil |
| B-110 | a compound of the formula I | folpet |
| B-111 | a compound of the formula I | fenpropidin |
| B-112 | a compound of the formula I | famoxadone |
| B-113 | a compound of the formula I | fenamidone |
| B-114 | a compound of the formula I | octhilinone |
| B-115 | a compound of the formula I | probenazole |
| B-116 | a compound of the formula I | proquinazid |
| B-117 | a compound of the formula I | pyroquilon |
| B-118 | a compound of the formula I | quinoxyfen |
| B-119 | a compound of the formula I | tricyclazole |
| B-120 | a compound of the formula I | 5-chloro-7-(4-methylpiperidin-1-yl)-6-(2,4,6- |
| | | trifluorophenyl)-[1,2,4]triazolo[1,5-a]pyrimidine |
| B-121 | a compound of the formula I | 6-(3,4-dichlorophenyl)-5-methyl- |
| | | [1,2,4]triazolo[1,5-a]pyrimidin-7-ylamine |
| B-122 | a compound of the formula I | 6-(4-tert-butylphenyl)-5-methyl- |
| | | [1,2,4]triazolo[1,5-a]pyrimidin-7-ylamine |
| B-123 | a compound of the formula I | 5-methyl-6-(3,5,5-trimethylhexyl)- |
| | | [1,2,4]triazolo[1,5-a]pyrimidin-7-ylamine |
| B-124 | a compound of the formula I | 5-methyl-6-octyl-[1,2,4]triazolo[1,5-a]- |
| | | pyrimidin-7-ylamine |
| B-125 | a compound of the formula I | 5-ethyl-6-octyl-[1,2,4]triazolo[1,5-a]pyrimidine- |
| | | 2,7-diamine |
| B-126 | a compound of the formula I | 6-ethyl-5-octyl-[1,2,4]triazolo[1,5-a]pyrimidin- |
| | | 7-ylamine |
| B-127 | a compound of the formula I | 5-ethyl-6-octyl-[1,2,4]triazolo[1,5-a]pyrimidin- |
| | | 7-ylamine |
| B-128 | a compound of the formula I | 5-ethyl-6-(3,5,5-trimethylhexyl)-[1,2,4]triazolo- |
| | | [1,5-a]pyrimidin-7-ylamine |
| B-129 | a compound of the formula I | 6-octyl-5-propyl-[1,2,4]triazolo[1,5-a]pyrimidin- |
| | | 7-ylamine |
| B-130 | a compound of the formula I | 5-methoxymethyl-6-octyl-[1,2,4]triazolo[1,5-a]- |
| | | pyrimidin-7-ylamine |
| B-131 | a compound of the formula I | 6-octyl-5-trifluoromethyl-[1,2,4]triazolo[1,5- |
| | | a]pyrimidin-7-ylamine |
| B-132 | a compound of the formula I | 5-trifluoromethyl-6-(3,5,5-trimethylhexyl)- |
| | | [1,2,4]triazolo[1,5-a]pyrimidin-7-ylamine |
| B-133 | a compound of the formula I | 2-butoxy-6-iodo-3-propylchromene-4-one |
| B-134 | a compound of the formula I | N,N-dimethyl-3-(3-bromo-6-fluoro-2-methyl- |
| | | indole-1-sulfonyl)-[1,2,4]triazole-1-sulfon- |
| | | amide |
| B-135 | a compound of the formula I | ferbam |
| B-136 | a compound of the formula I | mancozeb |
| B-137 | a compound of the formula I | maneb |
| B-138 | a compound of the formula I | metiram |
| B-139 | a compound of the formula I | metam |
| B-140 | a compound of the formula I | propineb |
| B-141 | a compound of the formula I | thiram |
| B-142 | a compound of the formula I | zineb |
| B-143 | a compound of the formula I | ziram |
| B-144 | a compound of the formula I | diethofencarb |
| B-145 | a compound of the formula I | flubenthiavalicarb |
| B-146 | a compound of the formula I | iprovalicarb |
| B-147 | a compound of the formula I | propamocarb |
| B-148 | a compound of the formula I | methyl 3-(4-chlorophenyl)-3-(2-isopropoxy- |
| | | carbonylamino-3-methylbutyrylamino)- |
| | | propionate |
| B-149 | a compound of the formula I | 4-fluorophenyl N-(1-(1-(4-cyanophenyl)- |
| | | ethanesulfonyl)but-2-yl)carbamate |
| B-150 | a compound of the formula I | dodine |
| B-151 | a compound of the formula I | iminoctadine |
| B-152 | a compound of the formula I | guazatine |
| B-153 | a compound of the formula I | kasugamycin |
| B-154 | a compound of the formula I | polyoxine |
| B-155 | a compound of the formula I | streptomycin |
| B-156 | a compound of the formula I | validamycin A |
| B-157 | a compound of the formula I | fentin salts |
| B-158 | a compound of the formula I | isoprothiolane |
| B-159 | a compound of the formula I | dithianon |
| B-160 | a compound of the formula I | edifenphos |
| B-161 | a compound of the formula I | fosetyl |
| B-162 | a compound of the formula I | fosetyl-aluminum |
| B-163 | a compound of the formula I | iprobenfos |
| B-164 | a compound of the formula I | pyrazophos |
| B-165 | a compound of the formula I | tolclofos-methyl |
| B-166 | a compound of the formula I | phosphorous acid and its salts |
| B-167 | a compound of the formula I | thiophanate methyl |
| B-168 | a compound of the formula I | chlorothalonil |
| B-169 | a compound of the formula I | dichlofluanid |
| B-170 | a compound of the formula I | tolylfluanid |
| B-171 | a compound of the formula I | flusulfamide |
| B-172 | a compound of the formula I | phthalide |
| B-173 | a compound of the formula I | hexachlorobenzene |
| B-174 | a compound of the formula I | pencycuron |
| B-175 | a compound of the formula I | quintozene |
| B-176 | a compound of the formula I | binapacryl |
| B-177 | a compound of the formula I | dinocap |
| B-178 | a compound of the formula I | dinobuton |
| B-179 | a compound of the formula I | Bordeaux mixture |
| B-180 | a compound of the formula I | copper acetate |
| B-181 | a compound of the formula I | copper hydroxide |
| B-182 | a compound of the formula I | copper oxychloride |
| B-183 | a compound of the formula I | basic copper sulfate |
| B-184 | a compound of the formula I | sulfur |
| B-185 | a compound of the formula I | spiroxamine |
| B-186 | a compound of the formula I | cyflufenamid |
| B-187 | a compound of the formula I | cymoxanil |
| B-188 | a compound of the formula I | metrafenone |

The active compounds II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known [cf. EP-A 226 917; EP-A 10 28 125; EP-A 10 35 122; EP-A 12 01 648; WO 98/46608; WO 99/24413; WO 03/14103; WO 03/053145; WO 03/066609; WO 04/049804 and WO 07/012598].

The present invention furthermore relates to the pharmaceutical use of the azolopyrimidines of the formula I according to the invention, in particular the azolopyrimidines of the formula I described in the above description as being preferred, and/or their pharmaceutically acceptable salts, in particular to their use for treating tumors in mammals such as, for example, man.

### SYNTHESIS EXAMPLES

With appropriate modification of the starting materials, the procedures given in the synthesis examples below were used to obtain further compounds I. The compounds produced in this manner are listed in the Table below, together with physical data.

### Example 1

### Preparation of {5-chloro-6-[2,6-difluoro-4-(3-methoxypropoxy)phenyl]-[1,2,4]triazolo[1,5-a]pyrimidin-7-yl}-(R)-(1,2-dimethylpropyl)amine [I-3]

With stirring at 20 to 25° C., 0.72 g (8 mmol) of 3-methoxypropanol was added to a suspension of 0.16 g (6.7 mmol) of sodium hydride in 6 ml of tetrahydrofuran (THF). After the evolution of gas had ceased, a solution of 0.92 g (2.5 mmol) of [5-chloro-6-(2,4,6-trifluorophenyl)-[1,2,4]triazolo[1,5-a]pyrimidin-7-yl]-(R)-(1,2-dimethylpropyl)amine (prepared analogously to EP-A 550 113) in 3 ml of tetrahydrofuran was added, and the solution was stirred at 60° C. for about 5 hours. The reaction mixture was then diluted with dil. hydrochloric acid and the aqueous phase was extracted with methyl t-butyl ether (MTBE). The combined organic phases were dried and freed from the solvent. The residue gave, after preparative MPLC using acetonitrile/water mixtures on reverse-phase silica gel (RP-18), 0.5 g of the title compound as a light-yellow oil.

¹H-NMR (CDCl₃, δ in ppm): 8.35 (s, 1H); 6.6 (d, 2H); 6.3 (d, broad, 1H); 4.1 (t, 2H); 3.55 (t, 2H); 3.4 (s, 3H); 3.3 (m, 1H); 2.1 (m, 2H); 1.65 (m, 1H); 1.05 (d, 3H); 0.8 (2d, 6H).

### Example 2

### Preparation of 3-{4-[5-chloro-7-(4-methylpiperidin-1-yl)-[1,2,4]triazolo[1,5-a]pyrimidin-6-yl]-3,5-difluorophenoxy}propan-1-ol [I-6]

Step a: 4-[5-Chloro-7-(4-methylpiperidin-1-yl)-[1,2,4]triazolo[1,5-a]pyrimidin-6-yl]-3,5-difluorophenol

A suspension of 22 g (56 mmol) of 5-chloro-6-(2,6-difluoro-4-methoxyphenyl)-7-(4-methylpiperidin-1-yl)-[1,2,4]triazolo[1,5-a]pyrimidine [cf. WO 99/48893] and 11 g (80 mmol) of AlCl₃ in 400 ml of toluene was heated under reflux for 2 hours. A further 11 g of AlCl₃ were then added, and the mixture was heated under reflux for another 2 hours. The reaction mixture was then partitioned between ethyl acetate and water and the organic phase was separated off and extracted with water. The organic phase was then filtered through silica gel, and the solvent was distilled off. The residue was digested with diisopropyl ether. This gave 21.5 g of the title compound as a lightly colored solid which still contained about 50 mol % of toluene.

¹H-NMR (CDCl₃/DMSO-d₆, δ in ppm): 10.2 (s, 1H); 8.4 (s, 1H); 6.6 (d, 2H); 3.75 (m, 2H); 2.85 (t, br, 2H); 1.65 (d, br, 2H); 1.55 (m, 1H); 1.3 (m, 2H).

Step b: 3-{4-[5-Chloro-7-(4-methylpiperidin-1-yl)-[1,2,4]triazolo[1,5-a]pyrimidin-6-yl]-3,5-difluorophenoxy}propan-1-ol [I-6]

A solution of 1.5 g (4 mmol) of the compound prepared in step a, 0.8 g (5.7 mmol) of 3-bromopropanol and 0.8 g (5.7 mmol) of potassium carbonate in 10 ml of N-methyl-pyrrolidone was stirred at 20 to 25° C. for about 15 hours and then at 50° C. for 8 hours. The reaction mixture was then diluted with water and acidified with dil. hydrochloric acid, and the aqueous phase was extracted with MTBE. The combined organic phases were freed from the solvent. The residue gave, after preparative MPLC using acetonitrile/water mixtures on silica gel (RP-18), 0.7 g of the title compound as a light-yellow resin.

¹H-NMR (CDCl₃, δ in ppm): 8.4 (s, 1H); 6.6 (d, 2H); 4.2 (t, 2H); 3.9 (t, 2H); 2.8 (t, br, 2H); 2.2 (s, br, 1H); 2.1 (m, 2H); 1.65 (d, br, 2H); 1.55 (m, 1H); 1.0 (d, 3H).

The HPLC retention times (RT) in the following table were determined using the RP-18 column Chromolith Speed ROD (Merck KgaA, Germany), 50×4.6 mm, with the eluent acetonitrile+0.1% trifluoroacetic acid (TFA)/water+0.1% TFA in a gradient of from 5:95 to 95:5 in 5 minutes at 40° C., flow rate 1.8 ml/min. Mass spectrometry was effected using quadropole electrospray ionization, 80 V (positive mode).

TABLE I
Compounds of the formula I.1 with W = phenyl substituted by Lₘ and P¹; W² = H

| | |
|---|---|
| | I.1a |
| 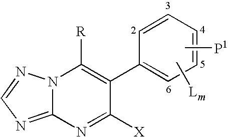 | |

| | | | Posi- | | | Phys. Data (m.p. [° C.]; |
|---|---|---|---|---|---|---|
| | | | tion | | | ¹H-NMR δ [ppm]; HPLC |
| No. | R | Lₘ | P¹ | P¹ | X | (RT [min], MS M + H [m/z]) |
| I-1 | CH₂CH(CH₃)₂ | 2,6-F₂ | 4 | #—OCH(CH₃)C(CH₃)═NOCH₃ | Cl | 113-115 |
| I-2 | (R) NHCH(CH₃)CH(CH₃)₂ | 2,6-F₂ | 4 | #—OCH₂CH₂CH₂N(CH₃)₂ | Cl | 8.35(s); 6.6(d); 6.3(d); 4.1(t); |
| | | | | | | 3.3(s); 2.5(t); 2.3(s); 2.0(m); |
| | | | | | | 1.65(m); 1.05(d); 0.8(2d) |
| I-3 | (R) NHCH(CH₃)CH(CH₃)₂ | 2,6-F₂ | 4 | #—OCH₂CH₂CH₂OCH₃ | Cl | 8.35(s); 6.6(d); 6.3(d); 4.1(t); |
| | | | | | | 3.55(t); 3.4(s); 3.3(m); |
| | | | | | | 2.1(m); 1.65(m); 1.05(d); |
| | | | | | | 0.8(2d) |
| I-4 | (R) NHCH(CH₃)CH(CH₃)₂ | 2,6-F₂ | 4 | 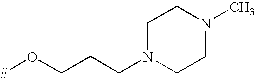 | Cl | 8.35(s); 6.6(d); 6.3(d); 4.1(t); 3.3(s); 2.5(m); 2.3(s); 2.05(m); 1.65(m); 1.1(d); 0.8(2d) |
| I-5 | (R) NHCH(CH₃)CH(CH₃)₂ | 2,6-F₂ | 4 | #—OCH₂CH₂CH₂OH | Cl | 8.35(s); 6.6(d); 6.3(d); 4.2(t); |
| | | | | | | 3.95(t); 3.3(s); 2.4(s); 2.1(m); |
| | | | | | | 1.65(m); 1.1(d); 0.8(2d) |
| I-6 | N[—CH₂CH₂CH(CH₃)CH₂CH₂—] | 2,6-F₂ | 4 | #—OCH₂CH₂CH₂OH | Cl | 8.4(s); 6.6(d); 4.2(t); 3.9(t); |
| | | | | | | 2.8(t); 2.2(s); 2.1(m); 1.65(d); |
| | | | | | | 1.55(m); 1.0(d) |
| I-7 | N[—CH₂CH₂CH(CH₃)CH₂CH₂—] | 2,6-F₂ | 4 | #—OCH₂CH₂CH₂N(CH₃)₂ | OCH₃ | 118-123 |
| I-8 | N[—CH₂CH₂CH(CH₃)CH₂CH₂—] | 2,6-F₂ | 4 | #—OCH₂CH₂CH₂N(CH₃)₂ | CN | 210-220 |
| I-9 | CH₂CH₂CH═CH₂ | 2,6-F₂ | 4 | #—OCH₂CH₂CH₂OCH₃ | CH₃ | CDCl₃: |
| | | | | | | 8.5 (s); 6.7 (d); 5.7 (m) 4.9 |
| | | | | | | (m); 4.2 (t); 3.6 (t); 3.4 (s); |
| | | | | | | 3.1 (t); 2.5 (m); 2.1 (m) |
| I-10 | N[—CH₂CH₂CH(CH₃)CH₂CH₂—] | 2,6-F₂ | 4 | #—OCH₂CH₂CH₂N(CH₃)₂ | CH₃ | CDCl₃: |
| | | | | | | 8.35 (s); 6.6 (d); 4.1 (t); 3.6 |
| | | | | | | (m); 2.75 (m); 2.65 (t); 2.4 |
| | | | | | | (s); 2.3 (s); 2.05 (m); 1.6 (m); |
| | | | | | | 1.5 (m); 1.3 (d); 0.95 (d) |
| I-11 | N[—CH₂CH₂CH(CH₃)CH₂CH₂—] | 2,6-F₂ | 4 | #—OCH₂CH₂OH | Cl | CDCl₃: |
| | | | | | | 8.4 (s); 6.6 (d); 4.15 (d); 4.05 |
| | | | | | | (d); 3.7 (d); 2.8 (t); 1.65 (d); |
| | | | | | | 1.55 (m); 1.3 (m); 0.95 (d) |
| I-12 | N[—CH₂CH₂CH(CH₃)CH₂CH₂—] | 2,6-F₂ | 4 | #—OCH₂CH₂OCH₃ | Cl | CDCl₃: 8.35 (s); 6.6 (d); 4.2 |
| | | | | | | (d); 3.8 (t); 3.7 (d); 3.5 (s); |
| | | | | | | 2.8 (t); 1.65 (d); 1.55 (m); |
| | | | | | | 1.35 (m); 0.95 (d) |
| I-13 | N[—CH₂CH₂CH(CH₃)CH₂CH₂—] | 2,6-F₂ | 4 | #—OCH₂CH₂OCH₂CH₂OCH₃ | Cl | CDCl₃: 8.35 (s); 6.65 (d); 4.2 |
| | | | | | | (t); 3.9 (t); 3.75 (m); 3.6 (t); |
| | | | | | | 3.4 (s); 2.8 (t); 1.65 (d); 1.55 |
| | | | | | | (m); 1.35 (m); 0.95 (d) |
| I-14 | N[—CH₂CH₂CH(CH₃)CH₂CH₂—] | 2,6-F₂ | 4 | #—OCH₂CH₂CH₂OCH₃ | Cl | 91-94 |
| I-15 | N[—CH₂CH₂CH(CH₃)CH₂CH₂—] | 2,6-F₂ | 4 | #—OCH₂CH₂CH₂N(CH₃)₂ | Cl | 117-119 |
| I-16 | (R) NHCH(CH₃)CH(CH₃)₂ | 2,6-F₂ | 4 | #—OCH₂CH₂CH₂NH₂ | Cl | DMSO-d6: |
| | | | | | | 8.6 (s); 8.4 (m); 6.95 (d); 4.15 |
| | | | | | | (t); 3.1 (m); 2.8 (t); 2.6 (t); |
| | | | | | | 2.5 (s); 2.05 (m); 1.6 (m); 1.5 |
| | | | | | | (m); 1.15 (m); 0.85 (d) |
| I-17 | (R) NHCH(CH₃)CH(CH₃)₂ | 2,6-F₂ | 4 | #—OCH₂CH₂CH₂NH₂CH₃^{+ ⊖}OC(O)CH₃ | Cl | 2.431; (439) |
| I-18 | N[—CH₂CH₂CH(CH₃)CH₂CH₂—] | 2,6-F₂ | 4 | #—OCH₂CH₂CH₂NH₂CH₃^{+ ⊖}OC(O)CF₃ | Cl | DMSO-d6: |
| | | | | | | 8.6 (s); 8.4 (m); 6.95 (d); 4.15 |
| | | | | | | (t); 3.1 (m); 2.8 (t); 2.6 (t); |
| | | | | | | 2.5 (s); 2.05 (m); 1.6 (m); |
| | | | | | | 1.5 (m); 1.15 (m); 0.85 (d) |
| I-19 | N[—CH₂CH₂CH(CH₃)CH₂CH₂—] | 2,6-F₂ | 4 | 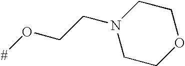 | Cl | CDCl₃: 8.35 (s); 6.6 (d); 4.15 (t); 3.75 (m); 2.85 (m); 2.6 (s); 1.65 (m); 1.55 (m); 1.3 (m); 0.95 (d) |
| I-20 | N[—CH₂CH₂CH(CH₃)CH₂CH₂—] | 2,6-F₂ | 4 | 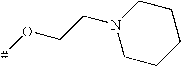 | Cl | CDCl₃: 8.35 (s); 6.6 (d); 4.15 (t); 3.7 (t); 2.8 (m); 2.5 (s); 1.4- 1.8 (m); 1.3 (m); 0.95 (d) |
| I-21 | N(CH₂CH₃)₂ | 2,6-F₂ | 4 | 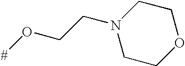 | Cl | 142-144 |
| I-22 | N(CH₂CH₃)₂ | 2,6-F₂ | 4 | 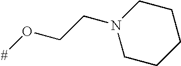 | Cl | 87-90 |
| I-23 | N[—CH₂CH₂CH(CH₃)CH₂CH₂—] | 2,6-F₂ | 4 | 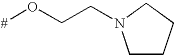 | Cl | CDCl₃: 8.35 (s); 6.6 (d); 4.3 (t); 3.7 (d); 3.25 (s); 3.0 (s); 2.8 (t); 2.0 (s); 1.65 (d); 1.55 (m); 1.35 (m); 0.95 (d) |
| I-24 | N[—CH₂CH₂CH(CH₃)CH₂CH₂—] | 2,6-F₂ | 4 | 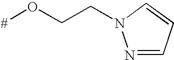 | Cl | CDCl₃: 8.4 (s); 7.6 (s); 7.5 (s); 6.6 (d); 6.3 (s); 4.6 (t); 4.4 (t); 3.7 (d); 2.8 (t); 1.7 (m); 1.6 (m); 1.3 (m); 0.95 (d) |
| I-25 | N[—CH₂CH₂CH(CH₃)CH₂CH₂—] | 2,6-F₂ | 4 | 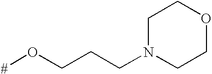 | Cl | 116-119 |
| I-26 | N[—CH₂CH₂CH(CH₃)CH₂CH₂—] | 2,6-F₂ | 4 | 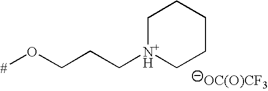 | Cl | CDCl₃: 10.6 (s); 8.5 (s); 6.6 (d); 4.1 (s); 3.7 (m); 3.3 (m); 2.85 (t); 2.75 (m); 2.3 (m); 1.9 (m); 1.3-1.8 (m); 0.95 (d) |
| I-27 | N[—CH₂CH₂CH(CH₃)CH₂CH₂—] | 2,6-F₂ | 4 | 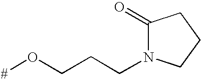 | Cl | 162-165 |
| I-28 | N[—CH₂CH₂CH(CH₃)CH₂CH₂—] | 2,6-F₂ | 4 | 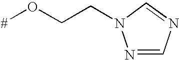 | Cl | 57-63 |
| I-29 | N[—CH₂CH₂CH(CH₃)CH₂CH₂—] | 2,6-F₂ | 4 | 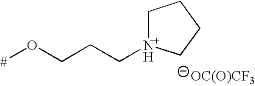 | Cl | 12.0 (s); 8.4 (s); 6.6 (d); 4.1 (t); 3.95 (m); 3.7 (d); 3.4 (m); 2.9 (m); 2.35 (m); 2.15 (m); 1.65 (d); 1.55 (m); 1.35 (m); 0.95 (d) |
| I-30 | N[—CH₂CH₂CH(CH₃)CH₂CH₂—] | 2,6-F₂ | 4 | 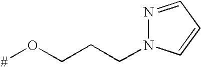 | Cl | 142-144 |
| I-31 | N[—CH₂CH₂CH(CH₃)CH₂CH₂—] | 2,6-F₂ | 4 | 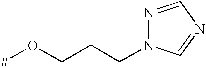 | Cl | 53-56 |
| I-32 | N[—CH₂CH₂CH(CH₃)CH₂CH₂—] | 2,6-F₂ | 4 | 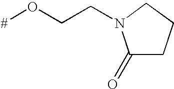 | Cl | CDCl₃: 8.35 (s); 6.6 (d); 4.15 (t); 3.7 (m); 3.6 (t); 3.8 (t); 2.4 (t); 2.1 (m); 1.65 (d); 1.55 (m); 1.35 (m); 0.95 (d) |
| I-33 | N[—CH₂CH₂CH(CH₃)CH₂CH₂—] | 2,6-F₂ | 4 | #—OCH₂CH₂OCH₂CH₂OC(O)CF₃ | Cl | CDCl₃: 8.4 (s); 6.6 (d); 4.55 |
| | | | | | | (t); 4.2 (t); 3.9 (m); 3.7 (d); |
| | | | | | | 2.8 (t); 1.65 (d); 1.55 (m); 1.3 |
| | | | | | | (m); 0.95 (d) |
| I-34 | N[—CH₂CH₂CH(CH₃)CH₂CH₂—] | 2,6-F₂ | 4 | #—OCH₂CH₂OCH₂CH₂OH | Cl | CDCl₃: 8.4 (s); 6.6 (d); 4.2 |
| | | | | | | (t); 3.9 (t); 3.8 (t); 3.7 (m); |
| | | | | | | 2.85 (t); 2.45 (s); 1.65 (d); |
| | | | | | | 1.55 (m); 1.3 (m); 0.95 (d) |
| I-35 | N[—CH₂CH₂CH₂CH₂CH₂—] | 2,6-F₂ | 4 | #—OCH₂CH₂CH₂N(CH₃)₂ | CH₃ | 95-97 |
| I-36 | N[—CH₂CH₂CH₂CH₂CH₂—] | 2,6-F₂ | 4 | #—OCH₂CH₂CH₂NH₂ | CH₃ | CDCl₃: 8.4 (s); 6.6 (d); 4.15 |
| | | | | | | (d); 3.1 (t); 2.9 (s); 2.4 (s); 2.1 |
| | | | | | | (m); 1.8 (m); 1.65 (m); 1.1- |
| | | | | | | 1.4 (m) |
| I-37 | N[—CH₂CH₂CH₂CH₂CH₂—] | 2,6-F₂ | 4 | #—OCH₂CH₂CH₂NHCH₃ | CH₃ | CDCl₃: 8.45 (s); 6.6 (d); 4.1 |
| | | | | | | (t); 3.05 (t); 2.9 (m); 2.65 (s); |
| | | | | | | 2.4 (m); 2.2 (m); 1.8 (m); |
| | | | | | | 1.65 (m); 1.35 (m); 1.2 (m) |
| I-38 | N[—CH₂CH₂CH(CH₃)CH₂CH₂—] | 2-Cl | 4 | #—OCH₂CH₂CH₂OCH₃ | Cl | 117-119 |
| I-39 | N[—CH₂CH₂CH(CH₃)CH₂CH₂—] | 2-F | 3 | #—OCH₂CH₂CH₂OCH₃ | Cl | 120-122 |
| I-40 | N[—CH₂CH₂CH(CH₃)CH₂CH₂—] | 2,6-F₂ | 4 | 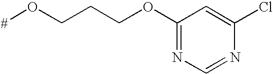 | Cl | CDCl₃: 8.6 (s); 8.4 (s); 6.8 (s); 6.6 (d); 4.6 (t); 4.2 (t); 3.7 (d); 2.8 (t); 2.35 (m); 1.65 (d); 1.55 (m); 1.35 (m); 0.95 (d) |
| I-41 | N[—CH₂CH₂CH(CH₃)CH₂CH₂—] | 2-F | 4 | #—OCH₂CH₂CH₂OCH₃ | Cl | 88-94 |
| I-42 | N[—CH₂CH₂CH(CH₃)CH₂CH₂—] | 2-CH₃ | 4 | #—OCH₂CH₂CH₂OCH₃ | Cl | CDCl₃: 8.4 (s; 7.0 (d); 6.85 |
| | | | | | | (m); 4.1 (t); 3.85 (d); 3.6 (t); |
| | | | | | | 3.5 (d); 3.4 (s); 2.7 (m); 2.2 |
| | | | | | | (s); 2.1 (m); 1.6 (m); 1.5 (m); |
| | | | | | | 1.3 (m); 0.95 (d) |
| I-43 | N[—CH₂CH₂CH(CH₃)CH₂CH₂—] | 2,6-F₂ | 4 | 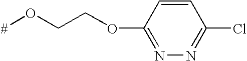 | Cl | CDCl₃: 8.4 (s); 7.2 (d); 6.95 (d); 6.6 (d); 4.55 (t); 4.4 (t); 3.7 (t); 2.8 (d); 1.65 (d); 1.55 (m); 1.3 (m); 0.95 (d) |
| I-44 | N(CH₂CH₃)₂ | 2,6-F₂ | 4 | 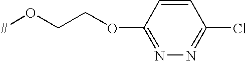 | Cl | 165-168 |
| I-45 | N[—CH₂CH₂CH(CH₃)CH₂CH₂—] | 2-F | 3 | #—OCH₂CH₂CH₂N(CH₃)₂ | Cl | 107-114 |
| I-46 | N(CH₂CH₃)₂ | 2,6-F₂ | 4 | #—OCH₂CH₂CH₂N(CH₃)₂ | Cl | 82-86 |
| I-47 | N[—CH₂CH₂CH(CH₃)CH₂CH₂—] | 2,6-F₂ | 4 | 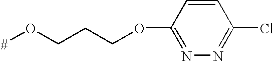 | Cl | 160-161 |
| I-48 | N(CH₂CH₃)₂ | 2,6-F₂ | 4 | 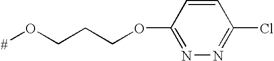 | Cl | CDCl₃: 8.4 (s); 7.4 (d); 7.0 (d); 6.6 (d); 4.7 (t); 4.2 (t); 3.4 (q); 2.4 (m); 1.1 (t) |
| I-49 | N(CH₂CH₃)₂ | 2,6-F₂ | 4 | 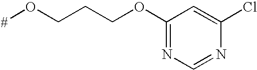 | Cl | 130-133 |
| I-50 | N(CH₂CH₃)₂ | 2,6-F₂ | 4 | #—OCH₂CH₂N(CH₃)₂ | Cl | 79-82 |
| I-51 | N[—CH₂CH₂CH(CH₃)CH₂CH₂—] | 2,6-F₂ | 4 | #—OCH₂CH₂N(CH₃)₂ | Cl | 149-151 |
| I-52 | N[—CH₂CH₂CH(CH₃)CH₂CH₂—] | 2,6-F₂ | 4 | 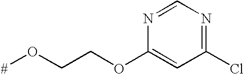 | Cl | 188-190 |
| I-53 | N(CH₂CH₃)₂ | 2,6-F₂ | 4 | 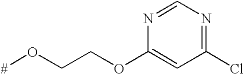 | Cl | 159-161 |
| I-54 | N[—CH₂CH₂CH(CH₃)CH₂CH₂—] | 2-F | 4 | #—OCH₂CH₂CH₂N(CH₃)₂ | Cl | 124-127 |
| I-55 | N[—CH₂CH₂CH(CH₃)CH₂CH₂—] | 2-Cl | 4 | #—OCH₂CH₂CH₂N(CH₃)₂ | Cl | 119-123 |
| I-56 | N[—CH₂CH₂CH₂CH₂CH₂—] | 2,6-F₂ | 4 | #—OCH₂CH₂CH₂N(CH₃)₂ | Cl | 117-119 |
| I-57 | N[—CH₂CH₂CH₂CH₂CH₂—] | 2,6-F₂ | 4 | #—OCH₂CH₂N(CH₃)₂ | Cl | 128-148 |
| I-58 | N[—CH₂CH₂CH(CH₃)CH₂CH₂—] | 2-CH₃ | 4 | #—OCH₂CH₂CH₂N(CH₃)₂ | Cl | CDCl₃: 8.35 (s); 7.0 (d); 6.85 |
| | | | | | | (m); 4.1 (t); 3.85 (d); 3.45 (d); |
| | | | | | | 2.7 (t); 2.6 (t); 2.5 (t); 2.3 (s); |
| | | | | | | 2.15 (s); 1.8 (m); 1.6 (m); 1.5 |
| | | | | | | (m); 1.3 (m); 0.95 (d) |
| I-59 | N[—CH₂CH₂CH(CH₃)CH₂CH₂—] | 2,6-F₂ | 4 | #—OCH₂CH₂CH₂OC(O)CH(CH₂)₂ | Cl | 113-115 |
| I-60 | N[—CH₂CH₂CH(CH₃)CH₂CH₂—] | 2,6-F₂ | 4 | #—OCH₂CH₂OC(O)CH(CH₂)₂ | Cl | CDCl₃: 8.4 (s); 6.6 (d); 4.5 |
| | | | | | | (t); 4.2 (t); 3.7 (d); 2.8 (t); 2.6 |
| | | | | | | (m); 1.65 (d); 1.55 (m); 1.3 |
| | | | | | | (m); 1.2 (d); 0.96 (d) |
| I-61 | N(CH₂CH₃)₂ | 2,6-F₂ | 4 | 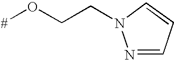 | Cl | 158-160 |
| I-62 | N(CH₂CH₃)₂ | 2,6-F₂ | 4 | 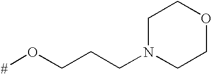 | Cl | 116-119 |
| I-63 | N(CH₂CH₃)₂ | 2,6-F₂ | 4 | 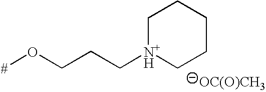 | Cl | CDCl₃: 8.35 (s); 6.6 (d); 4.05 (t); 3.4 (q); 2.55 (m); 2.15 (m); 2.05 (s); 1.75 (m); 1.5 (s); 1.05 (t) |
| I-64 | N(CH₂CH₃)₂ | 2,6-F₂ | 4 | 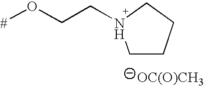 | Cl | CDCl₃: 8.35 (s); 6.6 (d); 4.25 (t); 3.4 (q); 3.2 (t); 2.95 (s); 2.05 (s); 2.95 (s); 1.05 (t) |
| I-65 | N(CH₂CH₃)₂ | 2,6-F₂ | 4 | 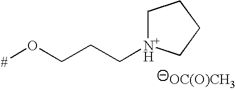 | Cl | CDCl₃: 8.35 (s); 6.6 (d); 4.05 (t); 3.4 (q); 2.9 (m); 2.15 (m); 2.0 (s); 1.9 (s); 1.05 (t) |
| I-66 | N(CH₂CH₃)₂ | 2,6-F₂ | 4 | 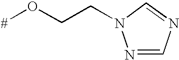 | Cl | 165-168 |
| I-67 | N(CH₂CH₃)₂ | 2,6-F₂ | 4 | 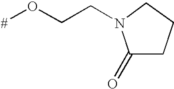 | Cl | 147-150 |
| I-68 | N(CH₂CH₃)₂ | 2,6-F₂ | 4 | 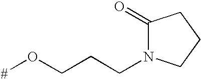 | Cl | 147-149 |
| I-69 | N[—CH₂CH₂OCH₂CH₂—] | 2,6-F₂ | 4 | #—OCH₂CH₂N(CH₃)₂ | Cl | 123-125 |
| I-70 | N[—CH₂CH₂OCH₂CH₂—] | 2,6-F₂ | 4 | #—OCH₂CH₂CH₂N(CH₃)₂ | Cl | 137-139 |
| I-71 | N(CH₂CH₃)₂ | 2,6-F₂ | 4 | 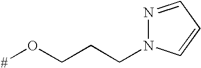 | Cl | 100-103 |
| I-72 | N(CH₂CH₃)₂ | 2,6-F₂ | 4 | 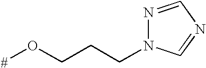 | Cl | 119-121 |
| I-73 | N[—CH₂CH₂CH(CH₃)CH₂CH₂—] | 2,6-F₂ | 4 | #—OCH(CH₃)CH₂OCH₃ | Cl | 119-122 |
| I-74 | N[—CH₂CH₂CH(CH₃)CH₂CH₂—] | 2,6-F₂ | 4 | #—OCH₂CH(CH₃)OCH₃ | Cl | CDCl₃: 8.35 (s); 6.6 (d); 4.0 |
| | | | | | | (m); 3.75 (m); 3.45 (s); 3.75 |
| | | | | | | (m); 3.5 (s); 2.8 (t); 1.55 (m); |
| | | | | | | 1.55 (m); 1.35 (m); 1.3 (d); |
| | | | | | | 0.95 (d) |
| I-75 | N(CH₂CH₃)₂ | 2,6-F₂ | 4 | #—OCH₂CH₂OCH₂CH₂OCH₃ | Cl | CDCl₃: 8.35 (s); 6.6 (d); 4.2 |
| | | | | | | (t); 3.9 (t); 3.75 (t); 3.6 (t); |
| | | | | | | 3.4 (m); 1.05 (t) |
| I-76 | CH₂CH(CH₃)CH₂CH₃ | 2,6-F₂ | 4 | #—OCH₂CH₂CH₂N(CH₃)₂ | CH₃ | CDCl₃: 8.45 (s); 6.65 (d); 4.1 |
| | | | | | | (t); 3.05 (dd); 3.8 (dd); 2.5 |
| | | | | | | (m); 2.3 (s); 2.05 (m); 1.3 |
| | | | | | | (m); 1.1 (m); 0.8 (t); 0.75 (d) |
| I-77 | N(CH₂CH₃)₂ | 2,6-F₂ | 4 | #—OCH₂CH₂OH | Cl | CDCl₃: 8.4 (s); 6.65 (d); 4.15 |
| | | | | | | (t); 4.05 (t); 3.4 (q); 1.1 (t) |
| I-78 | N(CH₂CH₃)₂ | 2,6-F₂ | 4 | #—OCH₂CH₂OCH₃ | Cl | CDCl₃: 8.35 (s); 6.65 (d); |
| | | | | | | 4.15 (t); 3.8 (t); 3.5 (s); |
| | | | | | | 3.4 (q); 1.05 (t) |
| I-79 | N(CH₂CH₃)₂ | 2,6-F₂ | 4 | #—OCH₂CH₂CH₂OH | Cl | CDCl₃: 8.4 (s); 6.6 (d); 4.2 |
| | | | | | | (t); 3.9 (t); 3.4 (q); 2.1 (m); |
| | | | | | | 1.1 (t) |
| I-80 | N(CH₂CH₃)₂ | 2,6-F₂ | 4 | #—OCH₂CH₂CH₂OCH₃ | Cl | CDCl₃: 8.4 (s); 6.6 (d); 4.1 |
| | | | | | | (t); 3.6 (t); 3.45 (q); 3.35 (s); |
| | | | | | | 2.1 (m); 1.1 (t) |
| I-81 | N(CH₂CH₃)₂ | 2,6-F₂ | 4 | #—OCH₂CH(CH₃)OCH₃ | Cl | CDCl₃: 8.4 (s); 6.65 (d); 4.0 |
| | | | | | | (m); 3.75 (m); 3.5 (s); 3.4 (q); |
| | | | | | | 1.3 (d); 1.1 (t) |
| I-82 | N(CH₂CH₃)₂ | 2,6-F₂ | 4 | #—OCH(CH₃)CH₂OCH₃ | Cl | 119-122 |
| I-83 | N[—CH₂CH₂CH(CH₃)CH₂CH₂—] | 2,6-F₂ | 4 | #—OCH₂CH₂CH₂CH₂OH | Cl | CDCl₃: 8.35 (s); 6.6 (d); 4.05 |
| | | | | | | (t); 3.75 (m); 2.8 (t); 2.0 (m); |
| | | | | | | 1.8 (t); 1.65 (d); 1.55 (m); 1.3 |
| | | | | | | (m); 0.95 (d) |
| I-84 | N[—CH₂CH₂CH(CH₃)CH₂CH₂—] | 2,6-F₂ | 4 | #—OCH₂CH₂CH₂NHC(O)CH₂Cl | Cl | 187-189 |
| I-85 | N[—CH₂CH₂CH(CH₃)CH₂CH₂—] | 2,6-F₂ | 4 | #—OCH₂CH₂CH₂NHC(O)CH(CH₃)₂ | Cl | 149-152 |
| I-86 | N[—CH₂CH₂CH(CH₃)CH₂CH₂—] | 2,6-F₂ | 4 | #—OCH₂CH₂CH₂OC(O)CH₂Cl | Cl | 134-136 |
| I-87 | N[—CH₂CH₂CH(CH₃)CH₂CH₂—] | 2,6-F₂ | 4 | #—OCH₂CH₂OC(O)CH₂Cl | Cl | 139-142 |
| I-88 | N[—CH₂CH₂CH(CH₃)CH₂CH₂—] | 2,6-F₂ | 4 | #—OCH₂CH₂CH₂N(CH₃)C(O)CH₂Cl | Cl | CDCl₃: 8.35 (s); 6.6 (m); 4.1 |
| | | | | | | (m); 3.7 (d); 3.6 (m); 3.15 (s, |
| | | | | | | rotamer A); 3.05 (s, rotamer |
| | | | | | | B); 3.8 (t); 2.1 (m); 1.65 (d); |
| | | | | | | 1.55 (m); 1.35 (m); 0.95 (d) |
| I-89 | N[—CH₂CH₂CH(CH₃)CH₂CH₂—] | 2,6-F₂ | 4 | #—O(CH₂)₃N(CH₃)C(O)CH(CH₃)₂ | Cl | 135-137 |
| I-90 | N[—CH₂CH₂CH(CH₃)CH₂CH₂—] | 2,6-F₂ | 4 | #—OCH₂CH₂NHC(O)CH₂Cl | Cl | 138-140 |
| I-91 | N[—CH₂CH₂CH(CH₃)CH₂CH₂—] | 2,6-F₂ | 4 | #—OCH₂CH₂NHC(O)CH(CH₃)₂ | Cl | CDCl₃: 8.4 (s); 6.6 (d); 6.05 |
| | | | | | | (s); 4.2 (t); 3.7 (m); 2.8 (t); |
| | | | | | | 2.4 (m); 1.65 (d); 1.55 (m); |
| | | | | | | 1.35 (m); 1.2 (d); 0.95 (d) |
| I-92 | N[—CH₂CH₂CH(CH₃)CH₂CH₂—] | 2,6-F₂ | 4 | #—OCH₂CH₂N(CH₃)C(O)CH₂Cl | Cl | CDCl₃: 8.35 (s); 6.6 (d); 4.3 |
| | | | | | | (s, rotamer A); 4.2 (m); 4.15 |
| | | | | | | (s, rotamer B); 3.9 (trotamer |
| | | | | | | A); 3.8 (t, rotamer B); 3.7 (d); |
| | | | | | | 3.3 (s, rotamers B); 3.1 (s, |
| | | | | | | rotamer A); 1.65 (m); 1.55 |
| | | | | | | (m); 1.35 (m); 0.95 (d) |
| I-93 | N[—CH₂CH₂CH(CH₃)CH₂CH₂—] | 2,6-F₂ | 4 | #—OCH₂CH₂N(CH₃)C(O)CH(CH₃)₂ | Cl | CDCl₃: 8.35 (s); 6.6 (m); 4.4 |
| | | | | | | (t, rotamer A); 4.1 (t, rotamer |
| | | | | | | B); 3.8 (m); 3.2 (s, rotamer |
| | | | | | | A); 3.05 (s, rotamer B); 3.0 |
| | | | | | | (m, rotamer B); 2.8 (m, |
| | | | | | | rotamer A); 1.65 (d); 1.55 |
| | | | | | | (m); 1.35 (m); 1.2 (d, rotamer |
| | | | | | | B); 1.1 (d, rotamer A); 0.95 |
| | | | | | | (d) |
| I-94 | N(CH₂CH₃)₂ | 2,6-F₂ | 4 | #—OCH₂CH₂CH₂CH₂OH | Cl | CDCl₃: 8.35 (s); 6.6 (d); 4.1 |
| | | | | | | (t); 3.8 (t); 3.4 (q); 1.95 (m); |
| | | | | | | 1.8 (m); 1.05 (t) |
| I-95 | CH₂CH₂CH₂CH₃ | 2-CH₃ | 4 | #—OCH₂CH₂CH₂N(CH₃)₂ | Cl | CDCl₃: |
| | | | | | | 8.5 (s); 7.05 (d); 6.85 (m); |
| | | | | | | 4.15 (t); 3.3 (m); 3.05 (m); |
| | | | | | | 2.9 (s); 2.8 (m); 2.35 (m); 2.1 |
| | | | | | | (s); 1.6 (m); 1.3 (m); 0.8 (t) |
| I-96 | N[—CH₂CH₂CH(CH₃)CH₂CH₂—] | 2,6-F₂ | 4 | #—O(CH₂)₃N(CH₃)C(O)OCH₂CH₃ | Cl | 118-119 |
| I-97 | N[—CH₂CH₂CH(CH₃)CH₂CH₂—] | 2,6-F₂ | 4 | #—OCH₂CH₂CH₂CH₂OH | Cl | 127-129 |
| I-98 | N[—CH₂CH₂CH(CH₃)CH₂CH₂—] | 2,6-F₂ | 4 | 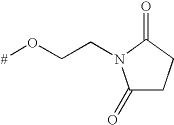 | Cl | 217-221 |
| I-99 | N[—CH₂CH₂CH₂CH₂CH₂—] | 2,6-F₂ | 4 | 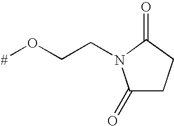 | Cl | 154-156 |
| I-100 | N[—CH₂CH₂CH(CH₃)CH₂CH₂—] | 2,6-F₂ | 4 | 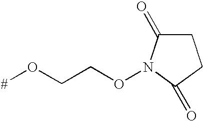 | Cl | 196-198 |
| I-101 | N[—CH₂CH₂CH(CH₃)CH₂CH₂—] | 2,6-F₂ | 4 | 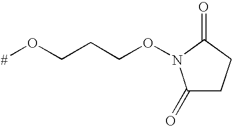 | Cl | 160-162 |
| I-102 | N[—CH₂CH₂CH₂CH₂CH₂—] | 2,6-F₂ | 4 | 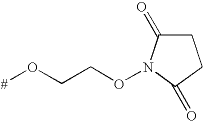 | Cl | 195-197 |
| I-103 | N[—CH₂CH₂CH₂CH₂CH₂—] | 2,6-F₂ | 4 | 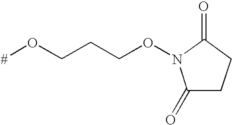 | Cl | 175-178 |
| I-104 | N[—CH₂CH₂CH₂CH₂CH₂—] | 2,6-F₂ | 4 | 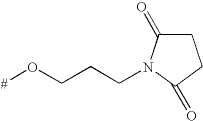 | Cl | 180-183 |
| I-105 | N[—CH₂CH₂CH(CH₃)CH₂CH₂—] | 2,6-F₂ | 4 | 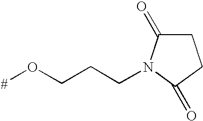 | Cl | 141-143 |
| I-106 | N[—CH₂CH₂CH(CH₃)CH₂CH₂—] | 2,6-F₂ | 4 | #—OCH₂CH₂CH₂NH₃^{+ ⊖}OC(O)CF₃ | Cl | 98-110 |
| I-107 | N[—CH₂CH₂CH(CH₃)CH₂CH₂—] | 2,6-F₂ | 4 | #—OCH₂CH₂N(CH₃)C(O)CH₃ | Cl | 3.098; (479) |
| I-108 | N[—CH₂CH₂CH(CH₃)CH₂CH₂—] | 2,6-F₂ | 4 | #—OCH₂CH₂N(CH₃)C(O)CH₂CH₃ | Cl | 3.290; (493) |
| I-109 | N[—CH₂CH₂CH(CH₃)CH₂CH₂—] | 2,6-F₂ | 4 | #—OCH₂CH₂N(CH₃)C(O)C(O)OCH₃ | Cl | 3.261; (523) |
| I-110 | N[—CH₂CH₂CH(CH₃)CH₂CH₂—] | 2,6-F₂ | 4 | #—OCH₂CH₂N(CH₃)C(O)OCH₂CH₃ | Cl | 3.619; (509) |
| I-111 | N[—CH₂CH₂CH(CH₃)CH₂CH₂—] | 2,6-F₂ | 4 | #—OCH₂CH₂OC(O)CH₂CH₃ | Cl | 3.640; (480) |
| I-112 | N[—CH₂CH₂CH(CH₃)CH₂CH₂—] | 2,6-F₂ | 4 | #—OCH₂CH₂OC(O)C(O)OCH₃ | Cl | 3.374; (510) |
| I-113 | N[—CH₂CH₂CH(CH₃)CH₂CH₂—] | 2,6-F₂ | 4 | #—O(CH₂)₂OC(O)(CH₂)₂C(O)OCH₃ | Cl | 3.468; (538) |
| I-114 | N[—CH₂CH₂CH(CH₃)CH₂CH₂—] | 2,6-F₂ | 4 | #—OCH₂CH₂CH₂OC(O)C(O)OCH₃ | Cl | 3.647; (524) |
| I-115 | N[—CH₂CH₂CH(CH₃)CH₂CH₂—] | 2,6-F₂ | 4 | #—O(CH₂)₃OC(O)(CH₂)₂C(O)OCH₃ | Cl | 3.362; (538) |
| I-116 | N[—CH₂CH₂CH(CH₃)CH₂CH₂—] | 2,6-F₂ | 4 | #—O(CH₂)₂NHC(O)(CH₂)₂C(O)OCH₃ | Cl | 2.857; (523) |
| I-117 | N[—CH₂CH₂CH(CH₃)CH₂CH₂—] | 2,6-F₂ | 4 | #—O(CH₂)₃NHC(O)(CH₂)₂C(O)OH | Cl | 2.962; (537) |
| I-118 | N[—CH₂CH₂CH(CH₃)CH₂CH₂—] | 2,6-F₂ | 4 | #—O(CH₂)₂OC(O)CH₂C(O)OH | Cl | 3.402; (524) |
| I-119 | N[—CH₂CH₂CH(CH₃)CH₂CH₂—] | 2,6-F₂ | 4 | #—O(CH₂)₂OC(O)(CH₂)₂C(O)OH | Cl | 3.124; (524) |
| I-120 | N[—CH₂CH₂CH(CH₃)CH₂CH₂—] | 2,6-F₂ | 4 | #—O(CH₂)₂N(CH₃)C(O)CH₂C(O)OH | Cl | 3.146; (537) |
| I-121 | N[—CH₂CH₂CH(CH₃)CH₂CH₂—] | 2,6-F₂ | 4 | #—O(CH₂)₂N(CH₃)C(O)(CH₂)₂C(O)OH | Cl | 2.962; (537) |
| I-122 | N[—CH₂CH₂CH(CH₃)CH₂CH₂—] | 2,6-F₂ | 4 | #—OCH₂CH₂N(CH₃)CHO | Cl | 3.043; (465) |
| I-123 | N[—CH₂CH₂CH(CH₃)CH₂CH₂—] | 2,6-F₂ | 4 | #—OCH₂CH₂CH₂N(CH₃)CHO | Cl | 3.289; (479) |
| I-124 | N[—CH₂CH₂CH(CH₃)CH₂CH₂—] | 2,6-F₂ | 4 | #—O(CH₂)₃N(CH₃)C(O)(CH₂)₂C(O)OH | Cl | 3.097; (551) |
| I-125 | N[—CH₂CH₂CH(CH₃)CH₂CH₂—] | 2,6-F₂ | 4 | #—OCH₂CH₂CH₂OCHO | Cl | 3.735; (466) |
| I-126 | N[—CH₂CH₂OCH₂CH₂—] | 2,6-F₂ | 4 | 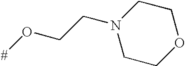 | Cl | 196-198 |
| I-127 | N[—CH₂CH₂OCH₂CH₂—] | 2,6-F₂ | 4 | 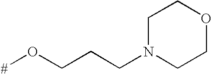 | Cl | 140-143 |
| I-128 | N[—CH₂CH₂CH(CH₃)CH₂CH₂—] | 2,6-F₂ | 4 | #—O(CH₂)₂N(CH₃)C(O)(CH₂)₂C(O)OCH₃ | Cl | 3.239; (551) |
| I-129 | N[—CH₂CH₂CH(CH₃)CH₂CH₂—] | 2,6-F₂ | 4 | #—O(CH₂)₂N(CH₃)C(O)OCH₃ | Cl | 3.442; (495) |
| I-130 | N[—CH₂CH₂CH(CH₃)CH₂CH₂—] | 2,6-F₂ | 4 | #—OCH₂CH₂OCHO | Cl | 3.540; (452) |
| I-131 | N[—CH₂CH₂CH(CH₃)CH₂CH₂—] | 2,6-F₂ | 4 | #—OCH₂CH₂NHCHO | Cl | 2.922; (451) |
| I-132 | N[—CH₂CH₂CH(CH₃)CH₂CH₂—] | 2,6-F₂ | 4 | #—OCH₂CH₂CH₂NHCHO | Cl | 3.046; (465) |
| I-133 | N[—CH₂CH₂CH(CH₃)CH₂CH₂—] | 2,6-F₂ | 4 | #—OCH₂CH₂OC(O)CH₃ | Cl | 3.423; (466) |
| I-134 | N[—CH₂CH₂CH(CH₃)CH₂CH₂—] | 2,6-F₂ | 4 | #—OCH₂CH₂OC(O)OCH₃ | Cl | 2.934; (482) |
| I-135 | N[—CH₂CH₂CH(CH₃)CH₂CH₂—] | 2,6-F₂ | 4 | #—OCH₂CH₂OC(O)OCH₂CH₃ | Cl | 3.630; (496) |
| I-136 | N[—CH₂CH₂CH(CH₃)CH₂CH₂—] | 2,6-F₂ | 4 | #—O(CH₂)₃OC(O)CH₃ | Cl | 3.738; (480) |
| I-137 | N[—CH₂CH₂CH(CH₃)CH₂CH₂—] | 2,6-F₂ | 4 | #—O(CH₂)₃OC(O)CH₂CH₃ | Cl | 3.954; (494) |
| I-138 | N[—CH₂CH₂CH(CH₃)CH₂CH₂—] | 2,6-F₂ | 4 | #—O(CH₂)₃OC(O)CH₂C(O)OCH₃ | Cl | 3.687; (538) |
| I-139 | N[—CH₂CH₂CH(CH₃)CH₂CH₂—] | 2,6-F₂ | 4 | #—O(CH₂)₃OC(O)CH₂CH₂C(O)OCH₃ | Cl | 3.763; (553) |
| I-140 | N[—CH₂CH₂CH(CH₃)CH₂CH₂—] | 2,6-F₂ | 4 | #—O(CH₂)₃OC(O)OCH₃ | Cl | 3.929; (510) |
| I-141 | N[—CH₂CH₂CH(CH₃)CH₂CH₂—] | 2,6-F₂ | 4 | #—OCH₂CH₂NHC(O)CH₃ | Cl | 2.970; (465) |
| I-142 | N[—CH₂CH₂CH(CH₃)CH₂CH₂—] | 2,6-F₂ | 4 | #—OCH₂CH₂NHC(O)CH₂CH₃ | Cl | 3.113; (479) |
| I-143 | N[—CH₂CH₂CH(CH₃)CH₂CH₂—] | 2,6-F₂ | 4 | #—OCH₂CH₂NHC(O)C(O)OCH₃ | Cl | 3.108; (509) |
| I-144 | N[—CH₂CH₂CH(CH₃)CH₂CH₂—] | 2,6-F₂ | 4 | #—OCH₂CH₂NHC(O)CH₂C(O)OCH₃ | Cl | 3.078; (523) |
| I-145 | N[—CH₂CH₂CH(CH₃)CH₂CH₂—] | 2,6-F₂ | 4 | #—OCH₂CH₂NHC(O)CH₂CH₂C(O)OCH₃ | Cl | 3.099; (537) |
| I-146 | N[—CH₂CH₂CH(CH₃)CH₂CH₂—] | 2,6-F₂ | 4 | #—OCH₂CH₂NHC(O)OCH₃ | Cl | 3.249; (481) |
| I-147 | N[—CH₂CH₂CH(CH₃)CH₂CH₂—] | 2,6-F₂ | 4 | #—OCH₂CH₂NHC(O)OCH₂CH₃ | Cl | 3.414; (495) |
| I-148 | N[—CH₂CH₂CH(CH₃)CH₂CH₂—] | 2,6-F₂ | 4 | #—OCH₂CH₂CH₂N(CH₃)C(O)CH₂CH₃ | Cl | 3.427; (507) |
| I-149 | N[—CH₂CH₂CH(CH₃)CH₂CH₂—] | 2,6-F₂ | 4 | #—OCH₂CH₂CH₂N(CH₃)C(O)C(O)OCH₃ | Cl | 3.415; (537) |
| I-150 | N[—CH₂CH₂CH(CH₃)CH₂CH₂—] | 2,6-F₂ | 4 | #—O(CH₂)₃N(CH₃)C(O)CH₂C(O)OCH₃ | Cl | 3.286; (551) |
| I-151 | N[—CH₂CH₂CH(CH₃)CH₂CH₂—] | 2,6-F₂ | 4 | #—O(CH₂)₃N(CH₃)C(O)(CH₂)₂C(O)OCH₃ | Cl | 3.379; (565) |
| I-152 | N[—CH₂CH₂CH(CH₃)CH₂CH₂—] | 2,6-F₂ | 4 | #—OCH₂CH₂CH₂N(CH₃)C(O)OCH₃ | Cl | 3.616; (509) |
| I-153 | N[—CH₂CH₂CH(CH₃)CH₂CH₂—] | 2,6-F₂ | 4 | #—OCH₂CH₂CH₂N(CH₃)C(O)OCH₂CH₃ | Cl | 3.773; (523) |
| I-154 | N[—CH₂CH₂CH(CH₃)CH₂CH₂—] | 2,6-F₂ | 4 | #—OCH₂CH₂CH₂N(CH₃)C(O)CH₃ | Cl | 3.258; (493) |
| I-155 | N[—CH₂CH₂CH(CH₃)CH₂CH₂—] | 2,6-F₂ | 4 | #—OCH₂CH₂CH₂OC(O)OCH₃ | Cl | 3.761; (496) |
| I-156 | N[—CH₂CH₂CH(CH₃)CH₂CH₂—] | 2,6-F₂ | 4 | #—OCH₂CH₂CH₂NHC(O)CH₃ | Cl | 3.081; (479) |
| I-157 | N[—CH₂CH₂CH(CH₃)CH₂CH₂—] | 2,6-F₂ | 4 | #—OCH₂CH₂CH₂NHC(O)CH₂CH₃ | Cl | 3.211; (493) |
| I-158 | N[—CH₂CH₂CH(CH₃)CH₂CH₂—] | 2,6-F₂ | 4 | #—OCH₂CH₂CH₂NHC(O)C(O)OCH₃ | Cl | 3.225; (523) |
| I-159 | N[—CH₂CH₂CH(CH₃)CH₂CH₂—] | 2,6-F₂ | 4 | #—O(CH₂)₃NHC(O)CH₂C(O)OCH₃ | Cl | 3.164; (537) |
| I-160 | N[—CH₂CH₂CH(CH₃)CH₂CH₂—] | 2,6-F₂ | 4 | #—O(CH₂)₃NHC(O)CH₂CH₂C(O)OCH₃ | Cl | 3.204; (551) |
| I-161 | N[—CH₂CH₂CH(CH₃)CH₂CH₂—] | 2,6-F₂ | 4 | #—O(CH₂)₃NHC(O)OCH₃ | Cl | 3.376; (495) |
| I-162 | N[—CH₂CH₂CH(CH₃)CH₂CH₂—] | 2,6-F₂ | 4 | #—O(CH₂)₃NHC(O)OCH₂CH₃ | Cl | 3.530; (509) |
| I-163 | N[—CH₂CH₂CH(CH₃)CH₂CH₂—] | 2,6-F₂ | 4 | #—O(CH₂)₃NHC(O)OC₆H₅ | Cl | 158-160 |
| I-164 | N[—CH₂CH₂CH(CH₃)CH₂CH₂—] | 2,6-F₂ | 4 | #—O(CH₂)₃NHC(O)OCF₃ | N(CH₃)₂ | 3.074; (542) |
| I-165 | N[—CH₂CH₂CH(CH₃)CH₂CH₂—] | 2,6-F₂ | 4 | #—OCH₂CH₂N(CH₃)C(O)C(O)CH₂Cl | Cl | CDCl₃: 8.35 (s); 6.6 (d); 4.7 |
| | | | | | | (s, rotamer A); 4.5 (s, rotamer |
| | | | | | | B); 4.25 (m); 3.9 (m); 3.75 |
| | | | | | | (d); 3.3 (s, rotamer A); 3.2 (s, |
| | | | | | | rotamer B); 2.8 (t); 1.65 (d); |
| | | | | | | 1.55 (m); 1.35 (m); 0.95 (d) |
| I-166 | N[—CH₂CH₂CH(CH₃)CH₂CH₂—] | 2,6-F₂ | 4 | #—O(CH₂)₃NHC(O)CF₃ | Cl | 177-180 |
| I-167 | N[—CH₂CH₂CH(CH₃)CH₂CH₂—] | 2,6-F₂ | 4 | #—O(CH₂)₃N(CH₃)C(O)C(O)CH₂Cl | Cl | 3.782; (555) |
| I-168 | N[—CH₂CH₂CH(CH₃)CH₂CH₂—] | 2,6-F₂ | 4 | 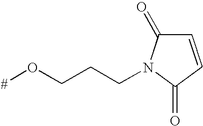 | Cl | 3.593; (517) |
| I-169 | N[—CH₂CH₂CH(CH₃)CH₂CH₂—] | 2,6-F₂ | 4 | #—OCH₂CH₂CH₂NH₄⁺ Cl^{⊖} | Cl | DMSO-d6: 8.65 (s); 8.25 (s); |
| | | | | | | 7.0 (d); 4.2 (t); 3.7 (d); 2.95 |
| | | | | | | (m); 2.85 (t);2.1 (m); 1.6 (m); |
| | | | | | | 1.2 (m); 0.9 (d) |
| I-170 | N[—CH₂CH₂CH(CH₃)CH₂CH₂—] | 2,6-F₂ | 4 | #—OCH₂CH₂CH₂OC(O)N(CH₃)₂ | Cl | 108-111 |
| I-171 | N[—CH₂CH₂CH(CH₃)CH₂CH₂—] | 2,6-F₂ | 4 | 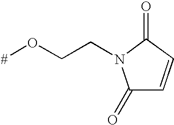 | Cl | 164-166 |
| I-172 | N[—CH₂CH₂CH(CH₃)CH₂CH₂—] | 2,6-F₂ | 4 | #—OCH₂CH₂CH₂N(CH₃)C(O)N(CH₃)₂ | Cl | CDCl₃: 8.35 (s); 6.6 (d); 4.05 |
| | | | | | | (t); 3.75 (t); 3.4 (t); 2.85 (s); |
| | | | | | | 3.8 (s); 2.1 (m); 1.65 (d); 1.55 |
| | | | | | | (m); 1.35 (m); 0.95 (d) |
| I-173 | N[—CH₂CH₂CH(CH₃)CH₂CH₂—] | 2,6-F₂ | 4 | #—OCH₂C(O)OCH₃ | Cl | 3.301, (451) |
| I-174 | N(CH₂CH₃)₂ | 2,6-F₂ | 4 | #—OCH₂C(O)OCH₃ | Cl | 3.110, (425) |
| I-175 | N(CH₂CH₃)₂ | 2,6-F₂ | 4 | #—OCH₂C(O)OH | Cl | 2.787, (411) |
| I-176 | N(CH₂CH₃)₂ | 2,6-F₂ | 4 | #—OCH₂CH₂C(O)OH | Cl | 2.838, (425) |
| I-177 | N[—CH₂CH₂CH(CH₃)CH₂CH₂—] | 2,6-F₂ | 4 | #—OCH₂C(O)OH | Cl | 3.018, (437) |
| I-178 | N[—CH₂CH₂CH(CH₃)CH₂CH₂—] | 2,6-F₂ | 4 | #—OCH₂CH₂C(O)OH | Cl | 3.038, (451) |
| I-179 | N(CH₂CH₃)₂ | 2,6-F₂ | 4 | #—OCH₂CH₂CH₂C(O)OH | Cl | 2.965, (439) |
| I-180 | N[—CH₂CH₂CH(CH₃)CH₂CH₂—] | 2,6-F₂ | 4 | #—OCH₂CH₂CH₂C(O)OH | Cl | 3.292, (465) |
| I-181 | N(CH₂CH₃)₂ | 2,6-F₂ | 4 | #—OCH₂CH₂C(O)OCH₃ | Cl | 3.327, (439) |
| I-182 | N(CH₂CH₃)₂ | 2,6-F₂ | 4 | #—OCH₂CH₂C(O)OCH₂CH₂CH₂OH | Cl | 2.972, (483) |
| I-183 | N(CH₂CH₃)₂ | 2,6-F₂ | 4 | #—OCH₂C(O)NH₂ | Cl | 2.505, (410) |
| I-184 | N[—CH₂CH₂CH(CH₃)CH₂CH₂—] | 2,6-F₂ | 4 | #—OCH₂C(O)NH₂ | Cl | 2.754, (436) |
| I-185 | N(CH₂CH₃)₂ | 2,6-F₂ | 4 | #—OCH₂C(O)NHC(O)CH₃ | Cl | 2.888, (452) |
| I-186 | N(CH₂CH₃)₂ | 2,6-F₂ | 4 | #—OCH₂C(O)NHC(O)CH₂CH₃ | Cl | 3.106, (466) |
| I-187 | N(CH₂CH₃)₂ | 2,6-F₂ | 4 | #—OCH₂C(O)NHC(O)N(CH₃)₂ | Cl | 2.767, (481) |
| I-188 | N[—CH₂CH₂CH(CH₃)CH₂CH₂—] | 2,6-F₂ | 4 | #—OCH₂C(O)NHC(O)CH₃ | Cl | 3.127, (478) |
| I-189 | N[—CH₂CH₂CH(CH₃)CH₂CH₂—] | 2,6-F₂ | 4 | #—OCH₂C(O)NHC(O)CH₂CH₃ | Cl | 3.330, (492) |
| I-190 | N[—CH₂CH₂CH(CH₃)CH₂CH₂—] | 2,6-F₂ | 4 | #—OCH₂C(O)NHOH | Cl | 2.662, (452) |
| I-191 | N(CH₂CH₃)₂ | 2,6-F₂ | 4 | #—OCH₂C(O)NHOH | Cl | 2.385, (426) |
| I-192 | N[—CH₂CH₂CH(CH₃)CH₂CH₂—] | 2,6-F₂ | 4 | #—OCH₂C(O)NHC(O)N(CH₃)₂ | Cl | 2.999, (507) |
| I-193 | N(CH₂CH₃)₂ | 2,6-F₂ | 4 | #—OCH₂C(O)NHC(O)CH₂Cl | Cl | 3.037, (486) |
| I-194 | N(CH₂CH₃)₂ | 2,6-F₂ | 4 | #—OCH₂C(O)N(CH₃)CH₂CH₂OH | Cl | 2.656, (482) |
| I-195 | N[—CH₂CH₂CH(CH₃)CH₂CH₂—] | 2,6-F₂ | 4 | #—OCH₂CH₂CH═NOH | Cl | 3.124, (450) |
| I-196 | N[—CH₂CH₂CH(CH₃)CH₂CH₂—] | 2,6-F₂ | 4 | #—OCH₂CH₂CH═NOCH₃ | Cl | 3.876, (450) |
| I-197 | (S) N[—C*H(CH₂OH)(CH₂)₃—] | 2,6-F₂ | 4 | #—OCH₂CH₂CH₂N(CH₃)₂ | Cl | 8.30(1H); 6.60(1H); 5.45(1H); |
| | | | | | | 4.08(2H); 3.60(1H); 3.50(1H); |
| | | | | | | 3.21(1H); 3.02(1H); 2.55- |
| | | | | | | 1.72(16H) |
| I-198 | N[—CH₂CH₂CH(CH₃)CH₂CH₂—] | 2-F | 4 | #—C(O)N(CH₃)CH₂CH₂OH | Cl | 2.604; (447) |
| I-199 | N[—CH₂CH₂CH₂CH₂CH₂—] | 2,6-F₂ | 4 | #—OCH₂CH₂CH₂NH₃⁺ CF₃COO^{⊖} | Cl | CF₃COOD: 8.95 (s); 7.0 (s); |
| | | | | | | 6.8 (d); 4.35 (t); 3.65 (m); |
| | | | | | | 2.45 (m); 1.85 (m) |
| I-200 | N(CH₂CH₃)₂ | 2,6-F₂ | 4 | #—OCH₂CH₂CH₂NH₃⁺ CF₃COO^{⊖} | Cl | 2.306; |
| | | | | | | (411 = protonated amine) |
| I-201 | N[—CH₂CH₂CH₂CH₂CH₂—] | 2,6-F₂ | 4 | #—OCH₂CH₂CH₂NH₂CH₃⁺ CF₃COO^{⊖} | Cl | 2.421; |
| | | | | | | (437 = protonated amine) |
| I-202 | N(CH₂CH₃)₂ | 2,6-F₂ | 4 | #—OCH₂CH₂CH₂NH₂CH₃⁺ CF₃COO^{⊖} | Cl | 2.353; |
| | | | | | | (425 = protonated amine) |
| I-203 | N[—CH₂CH₂CH₂CH₂CH₂—] | 2,6-F₂ | 4 | #—OCH₂CH₂CH₂OH | Cl | 166-168 |
| I-204 | CH₂CH₂CH₂CH₃ | 2-CH₃ | 4 | #—OCH₂CH₂N(CH₃)₂ | Cl | 2.354; (388) |
| I-205 | CH₂CH₂CH₂CH₃ | 2-CH₃ | 4 | #—OCH₂CH₂CH₂OH | Cl | 3.048; (375) |
| I-206 | CH₂CH₂CH₂CH₃ | 2-CH₃ | 4 | #—OCH₂CH₂CH₂NH₃⁺ CF₃COO^{⊖} | Cl | 2.383; |
| | | | | | | (374 = protonated amine) |
| I-207 | CH₂CH(CH₃)₂ | 2-CH₃ | 4 | #—OCH₂CH₂CH₂NH₃⁺ CF₃COO^{⊖} | Cl | 2.366; |
| | | | | | | (374 = protonated amine) |
| I-208 | N[—CH₂CH₂CH(CH₃)CH₂CH₂—] | 2-Cl | 5 | #—OCH₂CH₂CH₂N(CH₃)₂ | Cl | 116-118 |
| I-209 | CH₂CH₂CH₃ | 2-CH₃ | 4 | #—OCH₂CH₂CH₂NH₃⁺ CF₃COO^{⊖} | Cl | 2.195; |
| | | | | | | (360 = protonated amine) |
| I-210 | CH₂CH₃ | 2-CH₃ | 4 | #—OCH₂CH₂CH₂NH₃⁺ CF₃COO^{⊖} | Cl | 2.048; |
| | | | | | | (346 = protonated amine) |
| I-211 | CH₂CH(CH₃)₂ | 2-CH₃ | 4 | #—OCH₂CH₂CH₂NH₂CH₃⁺ CF₃COO^{⊖} | Cl | 2.400; |
| | | | | | | (388 = protonated amine) |
| I-212 | CH₂CH₂CH₃ | 2-CH₃ | 4 | #—OCH₂CH₂CH₂NH₂CH₃⁺ CF₃COO^{⊖} | Cl | 2.231; |
| | | | | | | (374 = protonated amine) |
| I-213 | N[—CH₂CH₂CH₂CH₂CH₂—] | 2,6-F₂ | 4 | #—OCH₂CH₂CH₂N(CH₃)₂ | Cl | 2.772; (452) |
| I-214 | CH₂CH₂CH₃ | 2-CH₃ | 4 | #—OCH₂CH₂CH₂N(CH₃)₂ | Cl | 2.312; (388) |
| I-215 | CH₂CH(CH₃)₂ | 2-CH₃ | 4 | #—OCH₂CH₂CH₂N(CH₃)₂ | Cl | 2.444; (402) |
| I-216 | CH₂CH₃ | 2-CH₃ | 4 | #—OCH₂CH₂CH₂N(CH₃)₂ | Cl | 2.108; (374) |
| I-217 | CH₂CH₃ | 2-CH₃ | 4 | #—OCH₂CH₂CH₂OH | Cl | 131-133 |
| I-218 | CH₂CH(CH₃)₂ | 2-CH₃ | 4 | #—OCH₂CH₂CH₂OH | Cl | 3.015; (375) |
| I-219 | CH₂CH₂CH₃ | 2-CH₃ | 4 | #—OCH₂CH₂CH₂OH | Cl | 2.841; (361) |
| I-220 | CH₂CH₃ | 2-CH₃ | 4 | #—OCH₂CH₂CH₂NH₂CH₃⁺ CF₃COO^{⊖} | Cl | 2.057; |
| | | | | | | (360 = protonated amine) |
| I-221 | N[—CH₂CH₂CH(CH₃)CH₂CH₂—] | 2,6-F₂ | 4 | 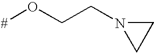 | Cl | 2.501; (449) |
| I-222 | N[—CH₂CH₂CH(CH₃)CH₂CH₂—] | 2,6-F₂ | 4 | #—OCH₂CH₂CH₂NHC(O)O(4-NO₂—C₆H₄) | Cl | 136-138 |
| I-223 | N[—CH₂CH₂CH(CH₃)CH₂CH₂—] | 2,6-F₂ | 4 | #—OCH₂CH₂CH₂NHC(O)N(CH₃)₂ | Cl | 3.256; (508) |
| I-224 | N[—CH₂CH₂CH(CH₃)CH₂CH₂—] | 2,6-F₂ | 4 | #—O(CH₂)₃N(CH₃)C(O)O(4-NO₂—C₆H₄) | Cl | CDCl₃: |
| | | | | | | 8.4 (s); 8.3 (d); 7.35 (m); 6.6 |
| | | | | | | (m); 4.25 (t); 4.9 (t; isomer |
| | | | | | | A); 4.8 (t, isomer B); 3.75 (d); |
| | | | | | | 3.3 (s, isomer B); 3.2 (s, |
| | | | | | | isomer A); 2.8 (t); 1.7 (m); |
| | | | | | | 1.6 (m); 1.35 (m); 0.95 (d) |
| I-225 | N[—CH₂CH₂CH(CH₃)CH₂CH₂—] | 2,6-F₂ | 4 | #—OCH₂CH₂CH₂OC(O)O(4-NO₂—C₆H₄) | Cl | CDCl₃: |
| | | | | | | 8.4 (s); 8.3 (d); 7.4 (d); 6.65 |
| | | | | | | (d); 4.6 (t); 4.2 (t); 3.75 (d); |
| | | | | | | 2.8 (t); 2.3 (m); 1.7 (d); 1.6 |
| | | | | | | (m); 1.35 (m); 0.95 (d) |
| I-226 | N[—CH₂CH₂CH(CH₃)CH₂CH₂—] | 2,6-F₂ | 4 | #—OCH₂CH₂CH₂NHC(O)NHNHC(O)CH₃ | Cl | 2.853; (537) |
| I-227 | N[—CH₂CH₂CH(CH₃)CH₂CH₂—] | 2,6-F₂ | 4 | #—O(CH₂)₃N(CH₃)C(O)O(4-NO₂—C₆H₄) | Cl | CDCl₃: 8.4 (s); 8.25 (m); 7.3 |
| | | | | | | (m); 6.6 (d); 4.15 (m 2H); |
| | | | | | | 3.6-3.7 (m, isomer A); 3.6 (t, |
| | | | | | | isomer B); 3.2 (s, isomer B); |
| | | | | | | 3.1 (s, isomer A); 2.8 (t); 2.2 |
| | | | | | | (m); 1.65 (d); 1.55 (m); 1.35 |
| | | | | | | (m); 0.95 (d) |
| I-228 | N[—CH₂CH₂CH(CH₃)CH₂CH₂—] | 2,6-F₂ | 4 | #—O(CH₂)₃N(CH₃)C(O)O(4-NO₂—C₆H₄) | N(CH₃)₂ | CDCl₃: 8.3 (m); 7.3 (m); 6.6 |
| | | | | | | (d); 4.1 (m); 3.7 (t, isomer A); |
| | | | | | | 3.6 (t, isomer B); 3.3 (d); 3.2 |
| | | | | | | (s, isomer B); 3.2 (s, isomer |
| | | | | | | A); 2.8-3.0 (m); 2.2 (m); 1.55 |
| | | | | | | (d); 1.45 (m); 1.1(m); 0.9 (d) |
| I-229 | N[—CH₂CH₂CH(CH₃)CH₂CH₂—] | 2,6-F₂ | 4 | #—OCH₂CH₂CH₂NHC(O)NH₂ | Cl | CDCl₃: |
| | | | | | | 8.35 (s); 6.6 (d); 5.7 (s); 5.0 |
| | | | | | | (s); 4.1 (t); 3.75 (d); 3.45 (t); |
| | | | | | | 2.8 (t); 2.1 (m); 1.65 (d); 1.55 |
| | | | | | | (m); 1.35 (m); 0.95 (d) |
| I-230 | N[—CH₂CH₂CH(CH₃)CH₂CH₂—] | 2,6-F₂ | 4 | #—OCH₂CH₂CH₂OC(O)NH₂ | Cl | 186-189 |
| I-231 | N[—CH₂CH₂CH(CH₃)CH₂CH₂—] | 2,6-F₂ | 4 | #—OCH₂CH₂CH₂OC(O)NHNHC(O)CH₃ | Cl | CDCl₃: 8.35 (s); 7.8 (s); 6.9 |
| | | | | | | (s); 6.6 (d); 4.4 (t); 4.1(t); |
| | | | | | | 3.75 (d); 2.8 (t); 2.2 (m); 2.1 |
| | | | | | | (s); 1.65 (d); 1.55 (m); 1.35 |
| | | | | | | (m); 0.95 (d) |
| I-232 | N[—CH₂CH₂CH(CH₃)CH₂CH₂—] | 2,6-F₂ | 4 | #—O(CH₂)₃NHC(O)NHCH₂C(O)OCH₃ | Cl | CDCl₃: 8.4 (s); 6.6 (d); 6.0 |
| | | | | | | (s); 6.8 (s); 4.1 (t); 4.05 (s); |
| | | | | | | 3.75 (m); 3.5 (t); 2.8 (t); 2.1 |
| | | | | | | (m); 1.65 (d); 1.55 (m); 1.35 |
| | | | | | | (m); 0.95 (d) |
| I-233 | N[—CH₂CH₂CH(CH₃)CH₂CH₂—] | 2,6-F₂ | 4 | #—OCH₂CH₂CH₂NHC(O)NHCH₃ | Cl | CDCl₃: 8.35 (s); 6.6 (d); 5.2 |
| | | | | | | (s); 4.1 (t); 3.75 (d); 3.4 (t); |
| | | | | | | 2.8 (m); 2.1 (m); 1.7 (d); 1.6 |
| | | | | | | (m); 1.35 (m); 0.95 (d) |
| I-234 | N[—CH₂CH₂CH(CH₃)CH₂CH₂—] | 2,6-F₂ | 4 | #—OCH₂CH₂CH₂N(CH₃)C(O)CF₃ | Cl | 102-113 |
| I-235 | N[—CH₂CH₂CH(CH₃)CH₂CH₂—] | 2,6-F₂ | 4 | #—OCH₂CH₂CH₂OC(O)NHCH₃ | Cl | 163-165 |
| I-236 | N[—CH₂CH₂CH(CH₃)CH₂CH₂—] | 2,6-F₂ | 4 | #—O(CH₂)₃NHC(O)NHNHC(O)C(O)NH₂ | Cl | 2,809; (566) |
| I-237 | N[—CH₂CH₂CH(CH₃)CH₂CH₂—] | 2,6-F₂ | 4 | #—O(CH₂)₃NHC(O)OCH₂C(O)OCH₃ | Cl | CDCl₃: 8.4 (s); 6.6 (d); 5.3 |
| | | | | | | (s); 4.35 (t); 4.1 (t); 4.0 (d); |
| | | | | | | 3.7-3.8 (m); 2.8 (t); 2.2 (m); |
| | | | | | | 1.65 (d); 1.55 (m); 1.35 (m); |
| | | | | | | 0.95 (d) |
| I-238 | N[—CH₂CH₂CH(CH₃)CH₂CH₂—] | 2,6-F₂ | 4 | #—O(CH₂)₃NHC(O)NHCH₂CH₂OH | Cl | 2.891; (524) |
| I-239 | N[—CH₂CH₂CH(CH₃)CH₂CH₂—] | 2,6-F₂ | 4 | #—O(CH₂)₃NHC(O)OCH₂CH₂OH | Cl | CDCl₃: 8.4 (s); 6.6 (d); 5.1 |
| | | | | | | (s); 4.25 (m); 4.1 (t); 3.75 (d); |
| | | | | | | 3.6 (m); 3.4 (m); 2.85 (t); 2.1 |
| | | | | | | (m); 1.65 (d); 1.6 (m); 1.35 |
| | | | | | | (m); 0.95 (d) |
| I-240 | N[—CH₂CH₂CH(CH₃)CH₂CH₂—] | 2,6-F₂ | 4 | #—OCH₂CH₂CH₂NHC(O)NHNHCHO | Cl | 2.821, (523) |
| I-241 | N[—CH₂CH₂CH(CH₃)CH₂CH₂—] | 2,6-F₂ | 4 | #—O(CH₂)₃NHC(O)NHCH₂CH₂N(CH₃)₂ | Cl | 2.733; (551) |
| I-242 | N[—CH₂CH₂CH(CH₃)CH₂CH₂—] | 2,6-F₂ | 4 | #—OCH₂CH₂OC(O)NHCH₃ | Cl | 131-134 |
| I-243 | N[—CH₂CH₂CH(CH₃)CH₂CH₂—] | 2,6-F₂ | 4 | #—OCH₂CH₂OC(O)NHC(O)NHCH₃ | Cl | 3.492; (538) |
| I-244 | N[—CH₂CH₂CH(CH₃)CH₂CH₂—] | 2,6-F₂ | 4 | #—O(CH₂)₂OC(O)NHC(O)O(4-NO₂—C₆H₄) | Cl | 189-191 |
| I-245 | N[—CH₂CH₂CH(CH₃)CH₂CH₂—] | 2,6-F₂ | 4 | #—OCH₂CH₂OC(O)N(CH₃)₂ | Cl | 164-167 |
| I-246 | N[—CH₂CH₂CH(CH₃)CH₂CH₂—] | 2,6-F₂ | 4 | #—OCH₂CH₂CH₂NHC(O)NHCH₂CH₂NH₃⁺ | Cl | 2.639; |
| | | | | CF₃COO^{⊖} | | (523 = protonated amine) |

# = bond to W

TABLE II
Compounds of the formula I.2 with W = phenyl, substituted by Lₘ and P¹; W² = H

| | |
|---|---|
| | I.2a |
| 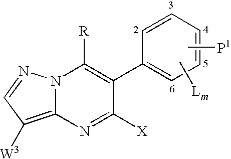 | |

| | | | | | | | Phys. Data (m.p. [° C.]; |
|---|---|---|---|---|---|---|---|
| | | | | | | | ¹H-NMR δ [ppm]; HPLC |
| No. | W³ | R | Lₘ | Position P¹ | P¹ | X | (RT [min], MS M + H [m/z]) |
| II-1 | CN | (R) NHCH(CH₃)CH(CH₃)₂ | 2,6-F₂ | 4 | #-OCH₂CH₂OCH₃ | Cl | 3.879, (450) |

### EXAMPLES OF THE ACTIVITY AGAINST HARMFUL FUNGI

The fungicidal activity of the compounds of the formula I was demonstrated by the following tests:

The active compounds were formulated separately as a stock solution having a concentration of 10 000 ppm in DMSO.

### Use Example 1—Activity against the Septoria Speckled Leaf Blotch Pathogen Septoria tritici in the Microtiter Test

The stock solution is pipetted into a microtiter plate (MTP) and diluted to the stated active compound concentration using a malt-based aqueous nutrient medium for fungi. An aqueous spore suspension of Septoria tritici was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds. In this test, the pathogens which had been treated with 125 ppm of the active compound I-1, I-2, I-3, I-4, I-8, I-26, I-45, I-49, I-61, I-63, I-64, I-65, I-72, I-73, I-75, I-76, I-84, I-90, I-94 to I-98, I-100, I-102, I-103, I-104, I-116, I-117, I-121 or I -143 showed at most 25% growth.

### Use Example 2—Activity against the Gray Mold Pathogen Botrytis cinerea in the Microtiter Test

The stock solution is pipetted into a microtiter plate (MTP) and diluted to the stated active compound concentration using a malt-based aqueous nutrient medium for fungi. An aqueous spore suspension of Botrytis cinerea was then added. The plates were placed in a water vapor-saturated chamber at temperatures of 18° C. Using an absorption photometer, the MTPs were measured at 405 nm on day 7 after the inoculation.

The evaluation was effected analogously to use example 1.

In this test, the pathogens which had been treated with 125 ppm of the active compound I-1, I-2, I-3, I-4, I-5, I-8, I-26, I-39, I-45, I-49, I-52, I-63, I-64, I-65, I-72, I-73, I-75, I-76, I-88, I-90, I-94, I-96, I-97, I-98, I-101, I-102, I-104, I-116, I-117, I-121, I-124, I-143, I-144, I-147, I-151 or I-153 showed at most 9% growth.

### Use Example 3—Activity against the Late Blight Pathogen Phytophthora infestans in the Microtiter Test

The stock solution is pipetted into a microtiter plate (MTP) and diluted to the stated active compound concentration using a pea juice-based aqueous nutrient medium for fungi. An aqueous zoo spore suspension of Phytophthora infestans was then added.

The plates were placed in a water vapor-saturated chamber at temperatures of 18° C. Using an absorption photometer, the MTPs were measured at 405 nm on day 7 after the inoculation.

Evaluation was carried out analogously to use example 1.

In this test, the pathogens which had been treated with 125 ppm of the active compounds I-8, I-26, I-45, I-63, I-64, I-65, I-69, I-76, I-116, I-117, I-121, I-124, I-126, I-143, I-144 or I-151 showed at most 10% growth.

### Use Example 4—Activity against the Rice Blast Pathogen Pyricularia oryzae in the Microtiter Test

The stock solution is pipetted into a microtiter plate (MTP) and diluted to the stated active compound concentration using a malt-based aqueous nutrient medium for fungi. An aqueous spore suspension of Pyricularia oryzae was then added. The plates were placed in a water vapor-saturated chamber at temperatures of 18° C. Using an absorption photometer, the MTPs were measured at 405 nm on day 7 after the inoculation.

Evaluation was carried out analogously to use example 1.

In this test, the pathogens which had been treated with 125 ppm of the active compounds I-26, I-39, I-44, I-45, I-49, I-61, I-63, I-64, I-65, I-69, I-72, I-75, I-76, I-82, I-84, I-88, I-94, I-95, I-97, I-98, I-100, I-101, I-102, I-103, I-104, I-116, I-117, I-121, I-124, I-126, I-143, I-144, I-147, I-151 or I-153 showed at most 10% growth.

Greenhouse Tests

The active compounds were prepared separately as a stock solution comprising 25 mg of active compound which was made up to 10 ml using a mixture of acetone and/or DMSO and the emulsifier Wettol EM 31 (wetting agent having emulsifying and dispersing action based on ethoxylated alkylphenols) in a volume ratio of solvent/emulsifier of 99/1. The mixture was then made up to 100 ml with water. This stock solution was diluted with the solvent/emulsifier/water mixture described to the concentration of active compound stated below.

### Use Example 5—Protective Activity against Puccinia recondite on Wheat (Brown Rust of Wheat)

Leaves of potted wheat seedlings were sprayed to run off point with an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and 65 to 70% relative atmospheric humidity for a further 7 days. The extent of the rust fungus development on the leaves was then determined visually.

In this test, the plants which had been treated with 250 ppm of the compounds I-7, I-9 to I-21, I-23, I-25, I-28 to I-32, I-35 to I-38, I-41, I-42, I-43, I-46, I-48, I-50, I-51, I-53 to I-58, I-74, I-77, I-78, I-79, I-85, I-86, I-89, I-91, I-93, I-107, I-108, I-109, I-113, I-115, I-119, I-120, I-122, I-123, I-125, I-127, I-128, I-129, I-131, I-132, I-139, I-141, I-142, I-145, I-146, I-148, I-149, I-150, I-152, I-154, I-156, I-157, I-159, I-160, I-161 or I-162 showed an infection of at most 20%, whereas the untreated plants were 90% infected.

### Use Example 6—Activity against Net Blotch of Barley Caused by Pyrenophora teres, 1 Day Protective Application

Leaves of potted barley seedlings were sprayed to run off point with an aqueous suspension having the active compound concentration stated below. 24 hours after the spray coating had dried on, the test plants were inoculated with an aqueous spore suspension of Pyrenophora [syn. Drechslera] teres, the net blotch pathogen. The test plants were then placed in a greenhouse at temperatures between 20 and 24° C. and 95 to 100% relative atmospheric humidity. After 6 days, the extent of the development of the disease was determined visually in % of the infected total leaf area.

In this test, the plants which had been treated with 250 ppm of the compounds I-7, I-9 to I-18, I-21, I-24 to I-29, I-31 to I-38, I-40, I-42, I-43, I-51, I-54 to I-57, I-59, I-60, I-62, I-68, I-70, I-71, I-74, I-77, I-78, I-79, I-81, I-83, I-85, I-86, I-87, I-89, I-91, I-93, I-99, I-105, I-107 to I-115, I-118, I-119, I-120, I-122, I-123, I-125, I-129 to I-134, I-138 to I-142, I-146, I-148, I-149, I-152, I-154 to I-157, I-159, I-161 or I-162 showed an infection of at most 20%, whereas the untreated plants were 90% infected.

### Use Example 7—Activity against Gray Mold on Bell Pepper Leaves caused by Botrytis cinerea, 1 Day Protective Application

Bell pepper seedlings were, after 2-3 leaves were well developed, sprayed to run off point with an aqueous suspension having the active compound concentration stated below. The next day, the treated plants were inoculated with a spore suspension of Botrytis cinerea in a 2% strength biomalt solution. The test plants were then placed in a dark climatized chamber at 22 to 24° C. and high atmospheric humidity. After 5 days, the extent of the fungal infection on the leaves could be determined visually in %.

In this test, the plants which had been treated with 250 ppm of the compounds I-9, I-11 to I-18, I-27, I-28, I-31, I-33 to I-38, I-41, I-42, I-43, I-46, I-48, I-50, I-53, I-55 to I-60, I-62, I-66, I-68, I-70, I-71, I-74, I-77 to I-81, I-83, I-86, I-87, I-99, I-105, I-106, I-111 to I-115, I-118, I-123, I-125, I-128, I-130 to I-142, I-146, I-148, I-149, I-150, I-154, I-155, I-159, I-160 or I-172 showed an infection of at most 20%, whereas the untreated plants were 90% infected.

### Use Example 8—Activity against Early Blight of Tomato Caused by Alternaria solani

Leaves of potted tomato plants were sprayed to run off with an aqueous suspension having the active compound concentration stated below. The next day, the leaves were /inoculated with an aqueous spore suspension of Alternaria solani in a 2% strength biomalt solution. The plants were then placed in a water vapor-saturated chamber at temperatures between 20 and 22° C. After 5 days, the disease on the untreated, but infected control plants had developed to such an extent that the infection could be determined visually in %.

In this test, the plants which had been treated with 250 ppm of the compounds I-9, I-11 to I-15, I-17 to I-25, I-27, I-28, I-30 to I-38, I-40, I-41, I-55, I-56, I-59, I-60, I-62, I-68, I-70, I-71, I-74, I-77 to I-81, I-83, I-85, I-86, I-87, I-89, I-93, I-106, I-107, I-108, I-110 to I-115, I-118, I-119, I-122, I-123, I-125, I-127, I-129 to I-142, I-145, I-146, I-148, I-152, I-154 to I-163, I-170 or I-172 showed an infection of at most 20%, whereas the untreated plants were 90% infected.

1-20. (canceled)

21. A compound of the formula I 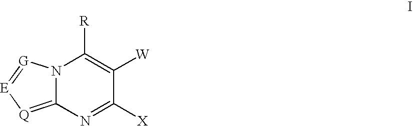
in which the substituents are as defined below:
G, E, Q a) G is N; E is C—W² and Q is N or C—W³;
b) G is C—W¹; E is C—W² and Q is N; or
c) G is C—W¹; E is N and Q is C—W³;
W¹, W², W³ are each independently of one another hydrogen, halogen, cyano, nitro, C₁-C₄-alkyl, C₂-C₆-alkenyl, C₂-C₆-alkynyl, C₁-C₄-haloalkyl, hydroxy-C₁-C₄-alkyl, C₁-C₄-alkoxy-C₁-C₄-alkyl, C₂-C₆-haloalkenyl, C₂-C₆-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-alkylsulfinyl or C₁-C₄-alkylsulfonyl, formyl, thiocarbamoyl, C₁-C₄-alkylcarbonyl, C₁-C₄-alkoxycarbonyl, C₁-C₄-alkylaminocarbonyl, aminocarbonyl, di-(C₁-C₄-alkyl)aminocarbonyl, C₁-C₄-alkoximinocarbonyl, hydroximinoalkyl, or CR¹⁰R¹¹OR¹², C(R¹³)═NR¹⁴;
R¹⁰, R¹¹, R¹² independently of one another are hydrogen, C₁-C₈-alkyl, C₃-C₆-cycloalkyl, C₁-C₈-alkoxy-C₁-C₈-alkyl, C₂-C₈-alkenyl, C₂-C₈-alkynyl, or benzyl;
R¹¹ and R¹² together may be oxy-C₁-C₅-alkylenoxy, where the carbon chain may be substituted by one to three groups selected from the group consisting of methyl, ethyl, hydroxyl, methoxy, ethoxy, hydroxymethyl, methoxymethyl, and ethoxymethyl;
R¹³ is hydrogen or C₁-C₈-alkyl;
R¹⁴ is C₁-C₈-alkyl, C₃-C₆-cycloalkyl, phenyl, or phenylamino, where the phenyl groups may be substituted by one to five groups R^{b};
R is NR¹R² or C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₂-cycloalkenyl, C₃-C₁₂-halocycloalkenyl, phenyl, halophenyl, naphthyl, halonaphthyl or a five-, six-, seven-, eight-, nine- or ten-membered saturated, partially unsaturated or aromatic heterocycle which is attached via carbon, may be partially or fully halogenated and contains one, two, three or four heteroatoms from the group consisting of oxygen, nitrogen and sulfur; where R may contain one, two, three or four identical or different groups R^{a} independently of one another selected from the group consisting of R cyano, nitro, hydroxyl, carboxyl, C₁-C₆-alkyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)O R^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, C₁-C₆-alkylene, oxy-C₁-C₄-alkylene, and oxy-C₁-C₃-alkyleneoxy, where divalent groups may be attached to the same atom or to adjacent atoms, phenyl, naphthyl, a five-, six-, seven-, eight-, nine- or ten-membered saturated, partially unsaturated or aromatic heterocycle which contains one, two, three or four heteroatoms from the group consisting of O, N and S;
R^{Π} is C₁-C₈-alkyl, C₃-C₈-alkenyl, C₃-C₈-alkynyl, phenyl, naphthyl, or a five-, six-, seven-, eight-, nine- or ten-membered saturated, partially unsaturated or aromatic heterocycle which contains one, two, three or four heteroatoms from the group consisting of O, N and S, C₃-C₆-cycloalkyl or C₃-C₆-cycloalkenyl, which groups R^{Π} may be partially or fully halogenated;
where the aliphatic, alicyclic or aromatic groups in the above mentioned groups R^{a} and R^{Π} for their part may carry one, two or three groups R^{b}:
R^{b} is cyano, nitro, hydroxyl, mercapto, amino, carboxyl, alkyl, alkenyl, alkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, where the alkyl groups in these radicals contain 1 to 6 carbon atoms and said alkenyl or alkynyl groups in these radicals contain 2 to 8 carbon atoms; cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, or hetarylthio, where the aryl radicals preferably contain 6 to 10 ring members, and the hetaryl radicals contain 5 or 6 ring members, where the cyclic systems may be partially or fully halogenated and/or may be substituted by alkyl or haloalkyl groups;
R¹, R² independently of one another are hydrogen, C₁-C₁₂-alkyl, C₂-C₁₂-alkenyl, C₂-C₁₂-alkynyl, C₃-C₆-cycloalkyl, C₃-C₆-cycloalkenyl, C₁-C₈-alkoxy, C₂-C₈-alkenyloxy, C₂-C₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle which contains one, two, three or four heteroatoms from the group consisting of O, N, S and Z—Y—(CR⁷R⁸)ₚ—(CR⁵R⁶)_{q}—CR³R⁴-#, in which # is the point of attachment to the nitrogen atom and:
R³, R⁴, R⁵, R⁶, R⁷, R⁸ independently of one another are hydrogen, halogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle which contains one, two, three or four heteroatoms from the group consisting of O, N and S, which cyclic groups may be partially or fully halogenated and/or may be substituted by one or more groups R^{Π},
R⁵ and R³ or R⁷ together with the atoms to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring member and/or may carry one or more substituents R^{a};
R³ may combine with R⁴, R⁵ may combine with R⁶, R⁷ may combine with R⁸, in each case to represent oxygen, thus forming carbonyl groups, and form a C₂-C₅-alkylene or alkenylene or alkynylene chain which may be interrupted by one, two or three heteroatoms from the group consisting of O, N and S, thus forming spiro groups;
R¹ and R³ together with the nitrogen atom to which they are attached may form a five-, six-, seven-, eight-, nine- or ten-membered saturated or partially unsaturated heterocycle which, in addition to carbon atoms, may contain one, two or three further heteroatoms from the group consisting of O, N and S as ring member;
R³, R⁴, R⁵, R⁶, R⁷, R⁸ may independently of one another be partially or fully halogenated;
R¹ to R⁸ may each independently carry one, two, three or four identical or different groups R^{a};
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{Π})NR^{A}R^{B}, C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, or a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may be substituted by one or more groups R^{b};
R^{A}, R^{B} independently of one another are hydrogen, C₂-alkenyl, C₂-alkynyl or one of the groups mentioned under R^{Π}, where R^{A} and R^{B} together with the nitrogen atom to which they are attached or R^{A} and R^{Π} together with the carbon and heteroatoms via which they are attached may also form a three- to ten-membered saturated, partially unsaturated or aromatic mono- or bicyclic ring which, in addition to carbon atoms, may contain one, two or three further heteroatoms from the group consisting of O, N and S as ring member, or may carry one or more oxo groups and/or one or more substituents R^{b}; or
Z and R⁶ or R⁸ may also form a five- or six-membered saturated or partially unsaturated ring which, in addition to carbon atoms and Y, may contain one or two further heteroatoms from the group consisting of N and S as ring member and/or may carry one or more substituents R^{a}, as defined below;
the group Z may be partially or fully halogenated and/or carry one, two or three groups R^{b};
R¹ and R² together with the nitrogen atom to which they are attached may also form a five-, six-, seven-, eight-, nine- or ten-membered saturated, partially unsaturated or aromatic mono- or bicyclic heterocycle which may be partially or fully halogenated and, in addition to carbon atoms, may contain one, two or three further heteroatoms from the group consisting of O, N and S as ring member and which may carry one, two or three substituents selected from the group consisting of R^{a}, Z—Y-# and Z—Y—(CR⁵R⁶)_{q}—CR³R⁴-#, where # is the point of attachment to the heterocycle;
p is zero, 1, 2, 3, 4 or 5;
q is zero or 1;
W is phenyl or five- or six-membered heteroaryl which, in addition to carbon atoms, contains one, two or three further heteroatoms from the group consisting of O, N and S as ring member, where the ring systems, in addition to groups Lₘ, carry at least one substituent P¹,
P1 is Y¹—Y²-T;
Y¹ is CR^{A}R^{B}, C(=T²)O, C(=T²)NR^{A}, O, OC(=T²), NR^{A} or S(O)ᵣ;
Y² is C₁-C₈-alkylene, C₂-C₈-alkenylene, or C₂-C₈-alkynylene, where Y² may be interrupted by one, two or three heteroatoms from the group consisting of NR^{A}, O, and S(O)ᵣ;
r is 0, 1 or 2;
T is YR, YR^{A}, NR^{A}R^{B}, YNR^{A}R^{B}; C(NOR^{A})R^{B}, S(O)ᵣR^{A}, N(NR^{A})-T¹-C(=T²)-T³, T¹-C(=T²)-[(Y²)_{q}—C(=T²)]ₚ-T³, T¹-C(=T²)-[Y²-T¹-C(=T²)]ₚ-T³, T¹-C(=T²)-[T¹-Y²—C(=T²)]ₚ-T3 or
T¹-C(=T²)-[NR^{A}—(NR^{B})_{q}—C(=T²)]ₚ-T³;
T¹ is a direct bond, O, S, or NR^{A};
T² is O, S, or NR^{A};
T³ is R, R^{B}, R^{Π}, YR^{B}, or NR^{A}R^{B};
where the carbon atoms in the group P¹ may be partially or fully halogenated and/or may be substituted by one or more groups R^{b};
L is halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₆-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C₁-C₄-alkylcarbonylamino, C(O)—R^{φ}, C(S)—R^{φ}, S(O)ₙ—R^{φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, or C₃-C₆-cycloalkylcarbonyl, or a five-, six-, seven-, eight-, nine- or ten-membered saturated, partially unsaturated or aromatic heterocycle which contains one, two, three or four heteroatoms from the group consisting of O, N and S;
R^{φ} is hydrogen, C₁-C₄-alkyl, C₁-C₂-haloalkyl, C₁-C₄-alkoxy, C₂-C₄-alkenyloxy, C₂-C₄-alkynyloxy, amino, C₁-C₄-alkylamino, di-C₁-C₄-alkylamino; where the groups R¹⁰⁰ may be substituted by one, two or three identical or different groups R^{b}, as defined above;
n is zero, 1 or 2;
m is zero, 1, 2, 3, 4 or 5;
X is halogen, cyano, C₁-C₄-alkyl, C₁-C₄-haloalkyl, C₁-C₄-alkoxy or C₁-C₄-haloalkoxy;
or an agriculturally acceptable salt thereof.

22. The compound of claim 21, wherein:
R is NR¹R², C₁-C₁₀-alkyl, C₁-C₁₀-haloalkyl, C₂-C₁₀-alkenyl, C₂-C₁₀-ha C₁₀-alkynyl, C₂-C₁₀-haloalkynyl, C₃-C₁₂-cycloalkenyl, C₃-C₁₂-halocycloalkenyl, phenyl, halophenyl, naphthyl, halonaphthyl or a five-, six-, seven-, eight-, nine- or ten-membered saturated, partially unsaturated or aromatic heterocycle which is attached via carbon and contains one, two, three or four heteroatoms from the group consisting of oxygen, nitrogen and sulfur; where R may contain one, two, three or four identical or different groups R^{a} independently of one another selected from the group consisting of:
R^{a} is cyano, nitro, hydroxyl, carboxyl, C₁-C₆-alkyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{Π}, C(O)OR^{Π}, C(S)O R^{Π}, C(O)SR^{Π}, C(S)SR^{Π}, OC(O)OR^{Π}, C₁-C₆-alkylthio, amino, C₁-C₆-alkylamino, di-C₁-C₆-alkylamino, aminocarbonyl, C(O)NHR^{Π}, C(O)NR^{Π}₂, C₁-C₆-alkylene, oxy-C₁-C₄-alkylene, or oxy-C₁-C₃-alkyleneoxy, where divalent groups may be attached to the same atom or to adjacent atoms, phenyl, naphthyl, a five-, six-, seven-, eight-, nine- or ten-membered saturated, partially unsaturated or aromatic heterocycle which contains one, two, three or four heteroatoms from the group consisting of O, N and S;
R^{Π} is C₁-C₈-alkyl, C₃-C₈-alkenyl, C₃-C₈-alkynyl, C₃-C₆-cycloalkyl or C₃-C₆-cycloalkenyl;
where the aliphatic, alicyclic or aromatic groups in the abovementioned groups R^{a} and R^{Π} for their part may be partially or fully halogenated and/or may carry one, two or three groups R^{b}:
R^{b} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, where the alkyl groups in these radicals contain 1 to 6 carbon atoms and said alkenyl or alkynyl groups in these radicals contain 2 to 8 carbon atoms; cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, or hetarylthio, where the aryl radicals preferably contain 6 to 10 ring members, and the hetaryl radicals contain 5 or 6 ring members, where the cyclic systems may be partially or fully halogenated and/or may be substituted by alkyl or haloalkyl groups;
R¹, R² independently of one another are hydrogen, C₁-C₁₂-alkyl, C₂-C₁₂-alkenyl, C₂-C₁₂-alkynyl, C₃-C₈-cycloalkyl, C₃-C₈-halocycloalkyl, C₃-C₆-cycloalkenyl, C₃-C₆-halocycloalkenyl, C₁-C₈alkoxy, C₂-C₈-alkenyloxy, C₂^{-C}₈-alkynyloxy, C₃-C₈-cycloalkoxy, NH₂, C₁-C₈-alkylamino, di-C₁-C₈-alkylamino, phenyl, naphthyl or a five- or six-membered saturated, partially unsaturated or aromatic heterocycle which contains one, two, three or four heteroatoms from the group consisting of O, N and S or Z—Y—(CR⁷R⁸)ₚ—(CR⁵R⁶)_{q}—CR³R⁴-#, in which # is the point of attachment to the nitrogen atom and:
R³, R⁴, R⁵, R⁶, R⁷, R⁸ independently of one another are hydrogen, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₆-halocycloalkyl, C₃-C₆-cycloalkenyl, C₃-C₆-halocycloalkenyl, phenyl, naphthyl or a five- or six-membered saturated, partially unsaturated or aromatic heterocycle which contains one, two, three or four heteroatoms from the group consisting of O, N and S,
R⁵ and R³ or R⁷ together with the atoms to which these radicals are attached may also form a five-, six-, seven-, eight-, nine- or ten-membered saturated or partially unsaturated ring which, in addition to carbon atoms, may contain one, two or three heteroatoms from the group consisting of O, N and S as ring member and/or may carry one or more substituents R^{a};
R³ may combine with R⁴, R⁵ may combine with R⁶, R⁷ may combine with R⁸, in each case to represent oxygen, thus forming carbonyl groups, and form a C₂-C₅-alkylene or alkenylene or alkynylene chain which may be interrupted by one, two or three heteroatoms from the group consisting of O, N and S, thus forming spiro groups;
R¹ and R³ together with the nitrogen atom to which they are attached may form a five-, six-, seven-, eight-, nine- or ten-membered saturated or partially unsaturated heterocycle which, in addition to carbon atoms, may contain one, two or three further heteroatoms from the group consisting of O, N and S as ring member;
R¹ to R⁸ may each independently carry one, two, three or four identical or different groups R^{a};
Y is oxygen or sulfur;
Z is hydrogen, carboxyl, formyl, C₁-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈-alkylthio, C₁-C₈-alkylsulfonyl, C(O)—C₁-C₄-alkylene-NR^{A}C(NR^{Π})NR^{A}R^{B}, C(S)—C₁-C₄-alkylene-NR^{A}C(NR^{Π})NR^{A}R^{B},
C(NR^{Π})—C₁-C₄-alkylene-NR^{A}C(NR^{Π})NR^{A}R^{B}, phenyl, naphthyl, or a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may be substituted by one or more groups R^{b};
R^{A}, R^{B} independently of one another are hydrogen, C₂-alkenyl, C₂-alkynyl or one of the groups mentioned under R^{Π};
R^{A} and R^{B} together with the nitrogen atom to which they are attached or R^{A} and R^{Π} together with the carbon and heteroatoms via which they are attached may also form a five- or six-membered saturated, partially unsaturated or aromatic ring which, in addition to carbon atoms, may contain one, two or three further heteroatoms from the group consisting of O, N and S as ring member and/or may carry one or more substituents R^{a}; or
Z and R⁶ or R⁸ may also form a five- or six-membered saturated or partially unsaturated ring which, in addition to carbon atoms and Y, may contain one or two further heteroatoms from the group consisting of N and S as ring member and/or may carry one or more substituents R^{a}, as defined below; the group Z may be partially or fully halogenated and/or carry one, two or three groups R^{b};
R¹ and R² together with the nitrogen atom to which they are attached may also form a five-, six-, seven-, eight-, nine- or ten-membered saturated, partially unsaturated or heterocycle which, in addition to carbon atoms, may contain one, two or three further heteroatoms from the group consisting of O, N and S as ring member and which contains at least one substituent selected from the group consisting of U—O-#, U—S-# and U—Y—(CR⁵R⁶)_{q}—CR³R⁴-#, where # is the point of attachment to the heterocycle and the heterocycle may furthermore carry one, two or three groups R^{a};
U is hydrogen, carboxyl, formyl, C₅-C₈-alkyl, C₁-C₈-haloalkyl, C₂-C₈-alkenyl, C₂-C₈-haloalkenyl, C₂-C₈-alkynyl, C₂-C₈-haloalkynyl, C₃-C₆-cycloalkyl, C₃-C₈-cycloalkenyl, C(O)R^{Π}, C(O)OR^{Π}, C(S)OR^{Π}, C(O)SR^{Π}, C(S)SR^{Π}, C(NR^{A})SR^{Π}, C(S)R^{Π}, C(NR^{Π})NR^{A}R^{B}, C(NR^{Π})R^{A}, C(NR^{Π})OR^{A}, C(O)NR^{A}R^{B}, C(S)NR^{A}R^{B}, C₁-C₈-alkylsulfinyl, C₁-C₈alkylthio, C₁-C₈-alkylsulfonyl, C(O)—C₁-C₄-alkylene-NR^{A}C(NR^{Π})NR^{A}R^{B}, C(S)—C₁-C₄-alkylene-NR^{A}C(NR^{Π})NR^{A}R^{B}, C(NR^{Π})—C₁-C₄-alkylene-NR^{A}C(NR^{Π})NR^{A}R^{B}, phenyl, naphthyl, or a five-, six-, seven-, eight-, nine- or ten-membered saturated, partially unsaturated or aromatic heterocycle which contains one, two, three or four heteroatoms from the group consisting of O, N and S and which is attached directly or via a carbonyl, thiocarbonyl, C₁-C₄-alkylcarbonyl or C₁-C₄-alkylthiocarbonyl group; where the carbon chains in group Z may carry one, two or three groups R^{b};
p is zero, 1, 2, 3, 4 or 5;
q is zero or 1;
W is phenyl or five- or six-membered heteroaryl which, in addition to carbon atoms, contains one, two or three further heteroatoms from the group consisting of O, N and S as ring member, where the ring systems, in addition to groups Lₘ, carry at least one substituent P¹,
P¹ is Y¹—Y²-T;
Y¹ is CR^{A}R^{B}, C(O)O, C(O)NR^{A}, O, NR^{A} or S(O)ᵣ;
Y² is C₁-C₈-alkylene, C₂-C₈-alkenylene, or C₂-C₈-alkynylene, where Y² may be interrupted by one, two or three heteroatoms from the group consisting of NR^{A}, O, and S(O)ᵣ;
r is 0, 1 or 2;
T is OR^{A}, OC(O)R^{A}, NR^{A}R^{B}, C(O)OR^{A}, C(O)NR^{A}R^{B}, C(NOR^{A})R^{A} or T¹-C(=T²)-T³;
T¹ is O, NR^{A};
T² is O, S, NR^{A};
T³ is R^{A}, OR^{A}, SR^{A}, NR^{A}R^{B};
L is halogen, hydroxyl, cyanato (OCN), cyano, nitro, C₁-C₈-alkyl, C₁-C_{g}-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxyl, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, C₁-C₄-alkylcarbonylamino, C(O)-R^{φ}, C(S)—R^{φ}, S(O)ₙ—R^{φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partially unsaturated or aromatic heterocycle which contains one, two, three or four heteroatoms from the group consisting of O, N and S;
R^{φ} is hydrogen, C₁-C₄-alkyl, C₁-C₄-alkoxy, C₂-C₄-alkenyloxy, C₂-C₄-alkynyloxy, amino, C₁-C₄-alkylamino, di-C₁-C₄-alkylamino; where the groups R^{φ} may be substituted by one, two or three identical or different groups R^{b}, as defined above;
n is zero, 1 or 2;
m is zero, 1, 2, 3, 4 or 5;
X is halogen, cyano, C₁-C₄-haloalkyl, C₁-C₄-alkoxy or C₁-C₄-haloalkoxy;
or an agriculturally acceptable salt thereof.

23. The compound of claim 21, wherein X is halogen.

24. The compound of claim 21, wherein X is methyl.

25. The compound of claim 21, wherein X is methoxy or cyano.

26. The compound of claim 21, which corresponds to the formula I.a.

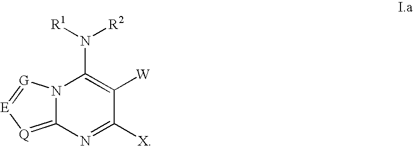

27. The compound of claim 21, which corresponds to the formula I.b 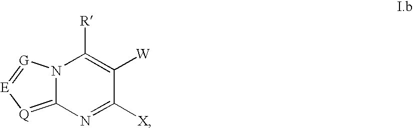
wherein R′ is a group R attached via carbon.

28. The compound of claim 21, wherein W is phenyl substituted by P¹ and Lₘ.

29. The compound of claim 21, wherein P¹ is
Y¹—Y²-T;
Y¹ is CR^{A}R^{B}, C(O)O, C(O)NR^{A}, O, NR^{A} or S(0)ᵣ;
Y² is C₁-C₈-alkylene, C₂-C₈-alkenylene, or C₂-C₈-alkynylene, where Y² may be interrupted by one, two or three heteroatoms from the group consisting of NR^{A}, O, and S(O)ᵣ;
r is 0, 1 or 2;
T is OR^{A}, OC(O)R^{A}, NR^{A}R^{B}, C(O)OR^{A}, C(O)NR^{A}R^{B}, C(NOR^{A})R^{A} or T¹-C(=T²)-T³;
T¹ is O, NR^{A};
T² is O, S, NR^{A};
T³ is R^{A}, OR^{A}, SR^{A}, NR^{A}R^{B}.

30. The compound of claim 21, wherein P¹ is a group attached via oxygen.

31. The compound of claim 21, which corresponds to the formula I.1.

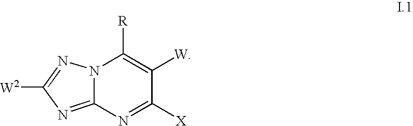

32. A process for preparing a compound claim 26 which corresponds to the formula I.a, wherein an aminoazole of the formula II 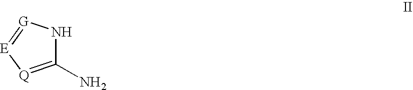
is reacted with a malonate of the formula III 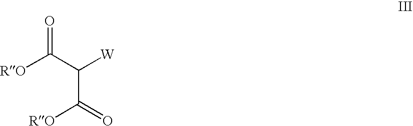
in which R″ is alkyl, to give 7-hydroxyazolopyrimidine of the formula IV 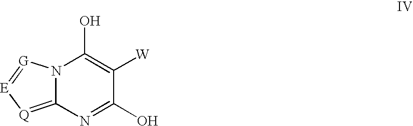
which is halogenated to give a compound of the formula V 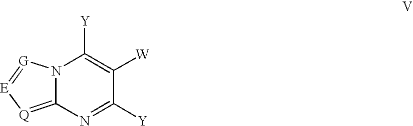
in which Y is chlorine or bromine, and V is reacted with an amine of the formula VI 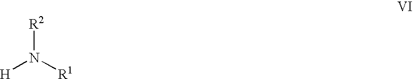
to give a compound of the formula I.a.

33. A process for preparing a compound of claim 26, wherein X is alkyl or haloalkyl, wherein the aminoazole of the formula II is reacted with a ketoester of the formula IIIa 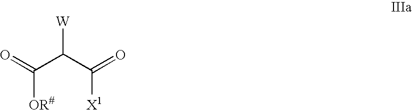
wherein R# is C₁-C₄-alkyl and X¹ is alkyl or haloalkyl, to give a 7-hydroxyazolopyrimidine of the formula IVa 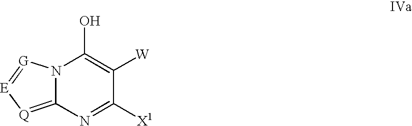
and IVa is converted with a halogenating agent into a 7-haloazolopyrimidine of the formula Va 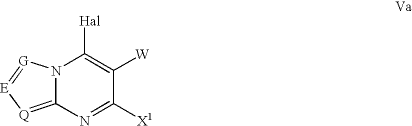
which is reacted with an amine of the formula VI according to claim 7 to give a compound of the formula I.

34. A process for preparing a compound of claim 24 by converting a 5-haloazolopyrimidine of the formula I with a malonate of the formula IIIb 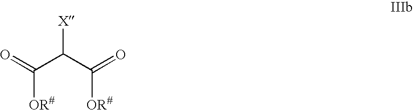
in which X″ is hydrogen or C₁-C₃-alkyl, alkenyl or alkynyl and R^{#} is C₁-C₄-alkyl into an ester of the formula VI 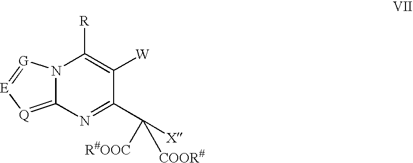
which is decarboxylated to give a compound of the formula I.

35. A process for preparing a compound of claim 31 by substitution of a hydroxyl compound of the formula IX 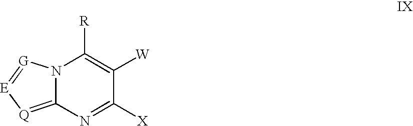
in which the substituents are as defined for formula I and P¹ is a hydroxyl group.

36. A compound of the formula IV, IVa, V, Va, VII or IX.

37. A composition comprising a solid or liquid carrier and a compound of claim 21.

38. The composition of claim 37, comprising a further active compound.

39. Seed, comprising a compound of claim 21 in an amount of from 1 to 1000 g per 100 kg.

40. A method for controlling phytopathogenic harmful fungi wherein the fungi or the materials, plants, soil or seed to be protected against fungal attack are/is treated with an effective amount of a compound of claim 21.
